SEQUENCE LISTING (564 sequences)

SEQ ID NO: 1 - DNA, 4249 bp, Homo sapiens
  misc_feature: SEZ6 isoform 1 mRNA
gatccccggcgccgtcgccaggcgctggccgtggtgctgattctgtcaggcgctggcggcggcagcggcggtgacggctgcggccccgctccctctacccggccggacccggctctgcccccgcgcccaagccccaccaagccccccgccctcccgccgcggtcccagcccagggcgcggccgcaaccagcaccatgcgcccggtagccctgctgctcctgccctcgctgctggcgctcctggctcacggactctctttagaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagctgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcagggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcctgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtggaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaagcagctacacccagcaggggccaccatccacttctcgtgtgcccctggctatgtgctgaagggccaggccagcatcaagtgtgtgcctgggcacccctcgcattggagtgaccccccacccatctgtagggctgcctctctggatgggttctacaacagtcgcagcctggatgttgccaaggcacctgctgcctccagcaccctggatgctgcccacattgcagctgccatcttcttgccactggtggcgatggtgttgttggtaggaggtgtatacttctacttctccaggctccagggaaaaagctccctgcagctgccccgcccccgcccccgcccctacaaccgcattaccatagagtcagcgtttgacaatccaacttacgagactggatctctttcctttgcaggagacgagagaatatgaagtctccatctaggtgggggcagtctagggaagtcaactcagacttgcaccacagtccagcagcaaggctccttgcttcctgctgtccctccacctcctgtatataccacctaggaggagatgccaccaagccctcaagaagttgtgcccttccccgcctgcgatgcccaccatggcctattttcttggtgtcattgcccacttggggcccttcattgggcccatgtcagggggcatctacctgtgggaagaacatagctggagcacaagcatcaacagccagcatcctgagcctcctcatgccctggaccagcctggaacacactagcagagcaggagtacctttctccacatgaccaccatcccgccctggcatggcaacctgcagcaggattaacttgaccatggtgggaactgcaccagggtactcctcacagcgcatcaccaatggccaaaactcctctcaacggtgacctctgggtagtcctggcatgccaacatcagcctcttgggaggtctctagttctctaaagttctggacagttctgcctcctgccctgtcccagtggaggcagtaattctaggagatcctaaggggttcagggggaccctacccccacctcaggttgggcttccctgggcactcatgctccacaccaaagcaggacacgccattttccactgaccaccctataccctgaggaaagggagactttcctccgatgtttatttagctgttgcaaacatcttcaccctaatagtccctcctccaattccagccacttgtcaggctctcctcttgaccactgtgttatgggataaggggagggggtgggcatattctggagaggagcagaggtccaaggacccaggaatttggcatggaacaggtggtaggagagccccagggagacgcccaggagctggctgaaagccactttgtacatgtaatgtattatatggggtctgggctccagccagagaacaatcttttatttctgttgtttccttattaaaatggtgtttttggaaaaaaaaaaaaaaaaaaaaaaaaaaaaaaaaaaaaaaaa

SEQ ID NO: 2 - DNA, 4234 bp, Homo sapiens
  misc_feature: SEZ6 isoform 2 mRNA
gatccccggcgccgtcgccaggcgctggccgtggtgctgattctgtcaggcgctggcggcggcagcggcggtgacggctgcggccccgctccctctacccggccggacccggctctgcccccgcgcccaagccccaccaagccccccgccctcccgccgcggtcccagcccagggcgcggccgcaaccagcaccatgcgcccggtagccctgctgctcctgccctcgctgctggcgctcctggctcacggactctctttagaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagctgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcagggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcctgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtggaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaagcagctacacccagcaggggccaccatccacttctcgtgtgcccctggctatgtgctgaagggccaggccagcatcaagtgtgtgcctgggcacccctcgcattggagtgaccccccacccatctgtagggctgcctctctggatgggttctacaacagtcgcagcctggatgttgccaaggcacctgctgcctccagcaccctggatgctgcccacattgcagctgccatcttcttgccactggtggcgatggtgttgttggtaggaggtgtatacttctacttctccaggctccagggaaaaagctccctgcagctgccccgcccccgcccccgcccctacaaccgcattaccatagagtcagcgtttgacaatccaacttacgagactggagagacgagagaatatgaagtctccatctaggtgggggcagtctagggaagtcaactcagacttgcaccacagtccagcagcaaggctccttgcttcctgctgtccctccacctcctgtatataccacctaggaggagatgccaccaagccctcaagaagttgtgcccttccccgcctgcgatgcccaccatggcctattttcttggtgtcattgcccacttggggcccttcattgggcccatgtcagggggcatctacctgtgggaagaacatagctggagcacaagcatcaacagccagcatcctgagcctcctcatgccctggaccagcctggaacacactagcagagcaggagtacctttctccacatgaccaccatcccgccctggcatggcaacctgcagcaggattaacttgaccatggtgggaactgcaccagggtactcctcacagcgccatcaccaatggccaaaactcctctcaacggtgacctctgggtagtcctggcatgccaacatcagcctcttgggaggtctctagttctctaaagttctggacagttctgcctcctgccctgtcccagtggaggcagtaattctaggagatcctaaggggttcagggggaccctacccccacctcaggttgggcttccctgggcactcatgctccacaccaaagcaggacacgccattttccactgaccaccctataccctgaggaaagggagactttcctccgatgtttatttagctgttgcaaacatcttcaccctaatagtccctcctccaattccagccacttgtcaggctctcctcttgaccactgtgttatgggataaggggagggggtgggcatattctggagaggagcagaggtccaaggacccaggaatttggcatggaacaggtggtaggagagccccagggagacgcccaggagctggctgaaagccactttgtacatgtaatgtattatatggggtctgggctccagccagagaacaatcttttatttctgttgtttccttattaaaatggtgtttttggaaaaaaaaaaaaaaaaaaaaaaaaaaaaaaaaaaaaaaaa

SEQ ID NO: 3 - protein, 994 aa, Homo sapiens
  MISC_FEATURE: SEZ6 isoform 1 precursor protein
  MISC_FEATURE (1)..(19): Leader sequence
  MISC_FEATURE (20)..(994): Mature protein
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 4 - protein, 993 aa, Homo sapiens
  MISC_FEATURE: SEZ6 isoform 2 precursor protein
  MISC_FEATURE (1)..(19): Leader sequence
  MISC_FEATURE (20)..(993): Mature protein
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGETREYEVSI

SEQ ID NO: 5 - DNA, 2925 bp, Homo sapiens
  misc_feature: cDNA sequence of hSCRx17 ORF
ctgagcctggaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagttgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcggggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcccgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtggaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaagcagctacacccagcaggggccaccatccacttctcgtgtgcccctggctatgtgctgaagggccaggccagcatcaagtgtgtgcctgggcacccctcgcattggagtgaccccccacccatctgtagggctgcctctctggatgggttctacaacagtcgcagcctggatgttgccaaggcacctgctgcctccagcaccctggatgctgcccacattgcagctgccatcttcttgccactggtggcgatggtgttgttggtaggaggtgtatacttctacttctccaggctccagggaaaaagctccctgcagctgccccgcccccgcccccgcccctacaaccgcattaccatagagtcagcgtttgacaatccaacttacgagactggatctctttcctttgcaggagacgagagaata

SEQ ID NO: 6 - protein, 975 aa, Homo sapiens
  MISC_FEATURE: hSCRx17 protein
LSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 7 - protein, 994 aa, Homo sapiens
  MISC_FEATURE: hSEZ6 BC146292 protein
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIGECPGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 8 - DNA, 3483 bp, Homo sapiens
  misc_feature: hSCRx17-Fc ORF
atggagacagacacactcctgctatgggtactgctgctctgggttcccgggtccactggtgacggcgcgcctggatccctgagcctggaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagttgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcggggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcccgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtggaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaagcagctacacccagcaggggccaccatccacttctcgtgtgcccctggctatgtgctgaagggccaggccagcatcaagtgtgtgcctgggcacccctcgcattggagtgaccccccacccatctgtagggctgcctctctggatgggttctacaacagtcgcagcctggatgttgccaaggcacctgctgcctccagcaccctggatgctgcccacctggccggccacagatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatccagggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 9 - protein, 1160 aa, Homo sapiens [sequence omitted]

SEQ ID NO: 10 - DNA, 2919 bp, Mus musculus
  misc_feature: cDNA sequence encoding mature murine SEZ6
ctctcctcagaggctccgatcacgggggaaggtcatgccacgggcatcagggagacggatggggagctgaccgcagcccctacacctgagcagtcagaccgaggcgtccacttcgtcaccacagcccctaccctcaagctgctcaaccaccacccacttctggaagaatttcttcaagaggggctagaaagagaggaagcgccgcagcctgcactgcccttccagccggactcacctacacactttactccaagccccctcccccgcctcaccaaccaggacaaccgccccgtctttaccagtccgactccagccgtggctgcagcacccacccagccccactccagggagaaaccttggaacctagaatccaaaccccctgagctttctatcacatcgtcccttcctccagggccgagtatggcagtgcccacactgctcccagaggacagacccagtactacaccccctagccaagcatggactccaactcaggagggtcctggagacatggacagaccttgggttccagaggtcatgtctaagaccacagggcttggtgtcgagggaaccattgccacctccacagcttcaggggatgacgaagagaccactaccaccatcattaccactactgtcaccacagttcagccaccaggcccctgtagctggaatttctcaggcccagagggctctctggattcccccacggcccccagctcaccctctgatgttggcctggactgtttctactatatctctgtctaccctggatatggagtagagatcaaggtggagaacatcagccttcaggaaggggagaccatcaccgtggagggcctggggggccccgatccactgcccttggctaaccagtcgttcctgctgaggggccaggtcatccgcagccccacccaccaagcagccctgaggttccagagcctcccgctacccgctgggcctggcactttccatttccgctaccaagcctatctcctgagctgccactttccccgacgtccagcgtatggagatgtgactgtcaccagtctccacccaggaggcagcgcccacttccattgtgccactggctaccagctcaagggtgccaggttcctcacctgtctcaatgccacccagcccttttgggattcccaagagcctgtttgcattgctgcttgtggtggagtgattcggaatgccaccactggccgcattgtctctcctggcttcccggggaactacagcaacaacctcacctgccactggttgctagaggctccagagagccagcggctgcacctgcactttgaaaaggtctccctggcagaagacgacgacaggctcatcatccgcaatggaaataacgtggaggccccgccggtgtacgactcctatgaggtggaatacctgcccattgagggcctgctcagctctggcagacacttcttcgtggagttcagtactgacagcagtggggcagctgcaggcatggccctgcgctatgaggccttccagcaaggacattgctatgagccctttgtcaaatacggcaacttcagcagcagtgcaccgtcctaccctgtgggtacaactgtggagttcagctgtgaccctggctacaccctggagcagggctccatcatcatcgaatgcgtcgacctccacgacccccagtggaatgagacagagccagcctgccgagccgtgtgcagcggggagatcacagactctgcaggcgtggtgctctctccaaactggccggagccttatggccgagggcaggactgcatctggggtgtgcatgtggaggaggacaagcgcatcatgctggacatccgagtgctgcgcataggctctggggatgtactgaccttctacgatggggatgacctcacagcccgggtcctgggccaatactcagggccccgtggccacttcaagctctttacctccatggccgatgtcaccatccagttccagtcagaccctgggacctcggcgctgggttaccagcaaggatttgtcatccacttctttgaggttccccgcaacgacacatgtccagagctacccgagatccccaacggctggaagaacccatcacagcctgagctggtgcacggcacggtggtcacctatcagtgctaccctggttaccaggtggtgggatccagtattctcatgtgccagtgggacctaagctggagtgaggacctgccttcatgccagagagtgacatcttgccatgacccaggggatgtggagcacagccgacgcctcatatccagccccaagtttcccgtgggagcaactgtgcaatatgtctgtgaccagggttttgtgctgacggggagtgccattctcacctgccatgatcggcaagcaggcagtcccaagtggagtgacagggcccccaagtgtctcttggaacaattcaagccgtgccatggcctcagcgccccggagaatggtgcccgcagccctgagaagcggcttcacccagcaggggccaccatccacttctcctgtgcccctggttatgtgctgaagggccaggccagcatcaaatgcgtgcctggacacccctcgcattggagtgacccaccacccatctgtagggctgcctctctggatgggttctacaacggccgtagcctggatgttgccaaggcacctgccgcctccagtgccctggacgctgctcacctggctgctgccatcttcctaccattggtggccatggtgttgctggtgggaggagtgtacctctatttttccagattccaggggaaaagtcccctgcaacttccccgaactcatcctcgcccctataaccgcatcacggtagagtcagcatttgacaatccaacttatgagactggatctctttcctttgcaggagacgagagaatatga

SEQ ID NO: 11 - protein, 972 aa, Mus musculus
  MISC_FEATURE: mSCRx17 protein
LSSEAPITGEGHATGIRETDGELTAAPTPEQSDRGVHFVTTAPTLKLLNHHPLLEEFLQEGLEREEAPQPALPFQPDSPTHFTPSPLPRLTNQDNRPVFTSPTPAVAAAPTQPHSREKPWNLESKPPELSITSSLPPGPSMAVPTLLPEDRPSTTPPSQAWTPTQEGPGDMDRPWVPEVMSKTTGLGVEGTIATSTASGDDEETTTTIITTTVTTVQPPGPCSWNFSGPEGSLDSPTAPSSPSDVGLDCFYYISVYPGYGVEIKVENISLQEGETITVEGLGGPDPLPLANQSFLLRGQVIRSPTHQAALRFQSLPLPAGPGTFHFRYQAYLLSCHFPRRPAYGDVTVTSLHPGGSAHFHCATGYQLKGARFLTCLNATQPFWDSQEPVCIAACGGVIRNATTGRIVSPGFPGNYSNNLTCHWLLEAPESQRLHLHFEKVSLAEDDDRLIIRNGNNVEAPPVYDSYEVEYLPIEGLLSSGRHFFVEFSTDSSGAAAGMALRYEAFQQGHCYEPFVKYGNFSSSAPSYPVGTTVEFSCDPGYTLEQGSIIIECVDLHDPQWNETEPACRAVCSGEITDSAGVVLSPNWPEPYGRGQDCIWGVHVEEDKRIMLDIRVLRIGSGDVLTFYDGDDLTARVLGQYSGPRGHFKLFTSMADVTIQFQSDPGTSALGYQQGFVIHFFEVPRNDTCPELPEIPNGWKNPSQPELVHGTVVTYQCYPGYQVVGSSILMCQWDLSWSEDLPSCQRVTSCHDPGDVEHSRRLISSPKFPVGATVQYVCDQGFVLTGSAILTCHDRQAGSPKWSDRAPKCLLEQFKPCHGLSAPENGARSPEKRLHPAGATIHFSCAPGYVLKGQASIKCVPGHPSHWSDPPPICRAASLDGFYNGRSLDVAKAPAASSALDAAHLAAAIFLPLVAMVLLVGGVYLYFSRFQGKSPLQLPRTHPRPYNRITVESAFDNPTYETGSLSFAGDERI

SEQ ID NO: 12 - DNA, 2919 bp, Rattus rattus
  misc_feature: cDNA sequence of rSCRx17 ORF
ctctcctcagaggctccaatcacgggggaaggtcaagccacgggcatcagggagatggatggggagctgaccgcagcccctacacctgagcagtcagaccgaggcgtccacttcgtcaccacagcccctaccctcaagctactcaaccaccacccacttctggaggaatttcttcaagaggggctagaagggagagaggaagctccgaggccggcactgcccttccagccagactcacctacaccctttactccaagcccccttccccgcctcaccaaccaggacaaccgccctgtctttaccagtccgacgccagctgtagctgcggcacccacgcagccccactccagaaagaaaccctggaacccagagtcagagcccccggagctttacatcacatctcccctccctccagggccgagtatggcagtgcccacactgcacccagaggacagacccagcactacaccccccagccaagcatggactccaacccaggagggtcctggagacatgggcagaccttgggttccagagatcatgtctaagaccacagggcttggtatcgaggggaccattgccacctccacagcttcaggggatgacgaagagaccaccaccaccaccatcattaccaccgtcaccacaattcagccaccaggcccctgtagctggaatttctcaggcccggagggctctctggattcccctgcggtccccagcgtcccctctgatgttggcctggactgtctctactacatctctgtctaccctggatatggagtcgagatcaaggtgaagaacatcagccttcaggaaggagagaccataaccgtggagggcctgggggggcctgacccactgcccttggctaaccagtctttcctgctgaggggccaggtcatccgcagccccacccaccaggcagccgtgaggttccaaagccttccacttcccgctggacctggtactttccatttccactaccaagcctatctcctgagctgccactttcctcggcgtccagcttatggagatgtgactgtcaccagcctccacccaggaggcagcgcccgcttccactgtgccactggctaccagctaaagggtgccaggttcctcacctgtctcaatgccacccagcccttttgggattcccaagagcctgtctgcattgctgcttgtggaggagtgattcggaatgccaccactggccgcattgtctctcctggctttcccgggaactacagcaacaacctcacctgccactggctgctagaagcccccgagagccagcggctgcacctgcactttgaaaaggtctccctggcagaagatgacgacaggctcatcatccgtaacgggaataacgtggaggccccgccagtgtatgactcctatgaggtggagtacctgcccattgagggcctgctcagttctggcagacacttcttcgtggagttcagtactgacagcagcggggcagccgcaggcatggcactgcgctatgaggccttccagcaaggacattgctatgagccctttgtcaaatacggtaacttcagcagcagcgcaccgtcctaccctgtgggtacgactgtggagttcagctgtgaccctggctacaccctggagcagggttccatcatcatcgaatgcgtcgacctccgtgacccccagtggaatgagacagaaccagcctgccgagccgtgtgcagcggggagatcacagactctgcaggcgtggtgctctctccaaactggccggagccttatggccgagggcaggactgcatctggggtgtgcatgtggaggaggacaagcgcatcatgctggacatccgagtgctgcgcataggctctggggatgtactgaccttctacgatggggatgacctgacagcccgggtcctgggccaatactcagggccccgtggccacttcaagctctttacctccatggctgatgtcaccattcagttccagtcagaccctgggacgtcggcgctgggttaccagcaaggatttgtcatccacttctttgaggtgccccgcaatgacacatgtccagagcttcccgagatccccaacggctggaagaacccatcacagcctgagctggtgcatggcacggtggtcacctatcagtgctaccccggttaccaggtggtgggatccagtattctcatgtgccagtgggacctgagctggagtgaggacctgccctcatgccagagagtgacatcctgccatgacccaggggatgtggagcacagccgacgcctcatatccagcctcaagtttcctgtgggagcaactgtgcagtatatctgtgaccagggttttgtgctcacgggtagcgccatccttacttgccatgatcgtcaagcgggcagtcccaagtggagtgacagggcccccaagtgtctcttggaacagttcaaaccatgtcatggcctcagtgcccctgagaatggtgcccgcagccctgagaagaggctccacccagcaggggccaccattcacttctcctgtgcccctggttatgtgctgaagggccaggccagcatcaaatgcgtgcctggacacccctcacattggagtgatcctccacccatctgtagggctgcttctctggatgggttctacaacggccgtagcctggatgttgccaaggcacctgccacctccagtgccctggatgctgcccacatggcagctgccatctttctaccattggtggccatggtgttgctggtgggaggagtgtacctctatttctccagactccagggaaaaagtcctctgcagcttcccggaactcatcctcgcccctataaccgtatcacggtagagtcagcatttgacaatccaacttatgagaccggatctctttcctttgcaggagacgagagaata

SEQ ID NO: 13 - protein, 973 aa, Rattus rattus
  MISC_FEATURE: rSCRx17 protein
LSSEAPITGEGQATGIREMDGELTAAPTPEQSDRGVHFVTTAPTLKLLNHHPLLEEFLQEGLEGREEAPRPALPFQPDSPTPFTPSPLPRLTNQDNRPVFTSPTPAVAAAPTQPHSRKKPWNPESEPPELYITSPLPPGPSMAVPTLHPEDRPSTTPPSQAWTPTQEGPGDMGRPWVPEIMSKTTGLGIEGTIATSTASGDDEETTTTTIITTVTTIQPPGPCSWNFSGPEGSLDSPAVPSVPSDVGLDCLYYISVYPGYGVEIKVKNISLQEGETITVEGLGGPDPLPLANQSFLLRGQVIRSPTHQAAVRFQSLPLPAGPGTFHFHYQAYLLSCHFPRRPAYGDVTVTSLHPGGSARFHCATGYQLKGARFLTCLNATQPFWDSQEPVCIAACGGVIRNATTGRIVSPGFPGNYSNNLTCHWLLEAPESQRLHLHFEKVSLAEDDDRLIIRNGNNVEAPPVYDSYEVEYLPIEGLLSSGRHFFVEFSTDSSGAAAGMALRYEAFQQGHCYEPFVKYGNFSSSAPSYPVGTTVEFSCDPGYTLEQGSIIIECVDLRDPQWNETEPACRAVCSGEITDSAGVVLSPNWPEPYGRGQDCIWGVHVEEDKRIMLDIRVLRIGSGDVLTFYDGDDLTARVLGQYSGPRGHFKLFTSMADVTIQFQSDPGTSALGYQQGFVIHFFEVPRNDTCPELPEIPNGWKNPSQPELVHGTVVTYQCYPGYQVVGSSILMCQWDLSWSEDLPSCQRVTSCHDPGDVEHSRRLISSLKFPVGATVQYICDQGFVLTGSAILTCHDRQAGSPKWSDRAPKCLLEQFKPCHGLSAPENGARSPEKRLHPAGATIHFSCAPGYVLKGQASIKCVPGHPSHWSDPPPICRAASLDGFYNGRSLDVAKAPATSSALDAAHMAAAIFLPLVAMVLLVGGVYLYFSRLQGKSPLQLPGTHPRPYNRITVESAFDNPTYETGSLSFAGDERI

SEQ ID NO: 14 - DNA, 2997 bp, Macaca fascicularis
  misc_feature: cDNA sequence of cSCRx17 ORF
atggagacagacacactcctgctatgggtactgctgctctgggttccaggttccactggtgacggcgcgccactcagcagcgaagctcccacaatgggcaagggacaggcccccggaattgaagaaaccgatggcgaactcaccgctgcccctacccctgagcaacccgaaaggggagtgcactttgtgaccaccgctcccaccctgaagctgctcaatcaccaccccctcctggaggagtttctgcaggaaggcctggaaaaaggcgacgaggaactcagacctgccctgcccttccaacccgaccctcctaccccctttacacctagccctctccctagactggccaaccaagactccagacctgtgttcaccagccctacacctgctacagctgccgtccctacccaacctcaatccaaggagggaccttggagcctcgagagcgagcctcccgtgctgagaatcacagctcctctccctcctggcccttccatggctgtccccacactcggacctggcgaaaggcccagcacaacacccccctccagagcctggacccctacacaagaaggccctggcgacatgggaaggccttgggtccctgaagtcgtgagccaaggcgccggcatcggaatccagggaaccatcgccagctccacagccagcggagacgatgaggaaacaaccaccacaaccaccatcatcaccaccacaatcacaacagtccagacccccggcccttgcagctggaatttttccggccctgagggatccctggattcccccacagatctgtcctcccctcctgacgtgggcctcgactgtttcttctatatctccgtgtatcctggctacggcgtcgaaatcaaagtccagaacatctccctgagggagggcgaaacagtcaccgtggaaggactgggcggacccgctcctctgcctctcgccaaccaatccttcctcctcaggggccaagtgattagatcccccacacatcaagctgctctcaggttccaaagcctccctccccccgctggacccggaacctttcacttccactaccaagcctatctcctcagctgccatttcccccacaggcccgcttatggagatgtcacagtcacctccctgcatcctggcggctccgctagattccactgcgctaccggataccaactcaagggcgccaggcatctgacatgtctcaatgctacccagcccttctgggacagcaaggagcccgtctgcattgccgcttgcggaggcgtcatcagaaatgccaccaccggcagaatcgtgagccccggcttccctggcaactactccaacaacctgacatgccactggctgctggaagctcctgagggccagagactgcatctgcacttcgagaaggtcagcctggccgaagatgacgacagactcatcatcaggaacggcgacaacgtggaggctccccccgtctatgattcctacgaggtcgagtacctccccatcgagggactgctgtcctccggcaagcattttttcgtggagctgtccacagattccagcggagctgccgccggaatggctctcaggtacgaggctttccaacagggccactgttacgagccctttgtgaagtacggcaacttctccagctccgctcctacctaccccgtcggcacaaccgtcgaatttagctgcgaccctggatacacactcgagcaaggctccatcatcatcgagtgtgtcgacccccacgacccccaatggaacgagacagagcccgcctgtagggccgtgtgtagcggagagattaccgactccgccggagtggtgctctcccctaattggcctgaaccctacggcagaggacaagattgtatttggggcgtccatgtcgaggaggacaagaggattatgctcgacgtgagggtgctgaggattggacctggcgacgtgctcacattctatgacggcgacgatctcaccgccagagtcctgggacaatactccggccctcacagccacttcaagctgttcaccagcatggctgacgtgaccatccagttccagtccgatcctggaacatccgtgctgggataccagcagggcttcgtcatccacttcttcgaggtccccaggaacgacacctgccccgaactgcccgagattcccaacggctggaaatccccctcccaacctgatctcgtgcacggcaccgtcgtcacctaccaatgctaccctggataccaagtcgtcggcagcagcgtgctgatgtgccaatgggacctcacctggagcgaggatctgccctcctgccagagagtcacctcctgccacgatcccggcgatgtggaacactccaggaggctgattagctcccccaagttccctgtcggagccaccgtgcaatacatctgcgaccagggctttgtgctgaccggaaccagcatcctcacatgccacgacaggcaagctggatcccccaagtggtccgatagggcccccaaatgcctcctggaacagctgaagccttgtcatggcctcagcgctcctgaaaacggcgctaggagccccgaaaagaggctccaccctgccggagccaccatccacttttcctgtgcccccggatacgtgctgaagggccaggcctccattaagtgcgtgcccggacatccttcccactggtccgacccccctcccatctgtaaagccgcctccctggacggattctataacagcagaagcctggacgtcgctaaggcccctgctgcttcctccaccctggatgctgctcacatcgctgctgccatctttctgcccctcgtcgccatggtgctgctggtgggaggcgtctacttctacttctccaggctgcagggaaagagctccctgcaactgcctaggacaagacccaggccctacaataggatcacagtcgagagcgccttcgacaaccccacatacgagacaggatccctgagctttgccggagacgagagaatt

SEQ ID NO: 15 - protein, 999 aa, Macaca fascicularis
  MISC_FEATURE: cSCRx17 protein
METDTLLLWVLLLWVPGSTGDGAPLSSEAPTMGKGQAPGIEETDGELTAAPTPEQPERGVHFVTTAPTLKLLNHHPLLEEFLQEGLEKGDEELRPALPFQPDPPTPFTPSPLPRLANQDSRPVFTSPTPATAAVPTQPQSKEGPWSLESEPPVLRITAPLPPGPSMAVPTLGPGERPSTTPPSRAWTPTQEGPGDMGRPWVPEVVSQGAGIGIQGTIASSTASGDDEETTTTTTIITTTITTVQTPGPCSWNFSGPEGSLDSPTDLSSPPDVGLDCFFYISVYPGYGVEIKVQNISLREGETVTVEGLGGPAPLPLANQSFLLRGQVIRSPTHQAALRFQSLPPPAGPGTFHFHYQAYLLSCHFPHRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSAPTYPVGTTVEFSCDPGYTLEQGSIIIECVDPHDPQWNETEPACRAVCSGEITDSAGVVLSPNWPEPYGRGQDCIWGVHVEEDKRIMLDVRVLRIGPGDVLTFYDGDDLTARVLGQYSGPHSHFKLFTSMADVTIQFQSDPGTSVLGYQQGFVIHFFEVPRNDTCPELPEIPNGWKSPSQPDLVHGTVVTYQCYPGYQVVGSSVLMCQWDLTWSEDLPSCQRVTSCHDPGDVEHSRRLISSPKFPVGATVQYICDQGFVLTGTSILTCHDRQAGSPKWSDRAPKCLLEQLKPCHGLSAPENGARSPEKRLHPAGATIHFSCAPGYVLKGQASIKCVPGHPSHWSDPPPICKAASLDGFYNSRSLDVAKAPAASSTLDAAHIAAAIFLPLVAMVLLVGGVYFYFSRLQGKSSLQLPRTRPRPYNRITVESAFDNPTYETGSLSFAGDERI

SEQ ID NO: 16 - DNA, 2781 bp, Homo sapiens
  misc_feature: cDNA sequence of human SEZ6L ECD
ctcgagagggatgctctgcctgagggagatgcttcccctctcggaccttatctgctgcccagcggagctcctgagaggggatcccccggaaaggagcatcccgaagaaagagtggtcacagctccccctagctccagccagagcgctgaggtgctgggagaactggtcctcgacggaacagccccttccgcccatcacgatattcctgccctcagccctctcctccccgaggaagctaggcctaaacacgccctcccccctaaaaagaagctgccttccctcaagcaggtcaattccgccaggaagcagctcagacccaaggccacctccgctgctacagtccaaagagctggatcccagcctgccagccagggactcgatctgctcagcagctccacagaaaaacctggacctcctggcgatcctgaccctattgtggccagcgaagaagctagcgaagtccctctgtggctggacaggaaggagtccgctgtccccaccacacccgctcctctccagatcagccccttcacctcccagccttatgtcgctcatacactgcctcagaggcctgagcctggcgaacctggacctgacatggctcaggaggctcctcaggaggacaccagccctatggccctgatggataagggcgagaatgaactgaccggaagcgccagcgaggaaagccaggagaccaccaccagcacaatcatcaccaccaccgtcatcaccaccgaacaggcccccgctctgtgttccgtgtccttttccaaccccgagggctacattgacagcagcgattaccccctgctccctctcaacaacttcctcgagtgcacctacaatgtgaccgtgtacaccggctacggagtcgaactccaggtgaagtccgtgaacctctccgatggcgaactgctctccattaggggcgtcgatggccctacactcaccgtcctggctaaccaaaccctgctcgtcgaaggccaggtgattaggtcccccaccaacaccatctccgtctacttcaggacctttcaagacgacggactgggaaccttccaactgcattaccaggccttcatgctgtcctgtaatttccccaggagacccgactccggagacgtcaccgtcatggatctgcactccggaggcgtggcccactttcattgtcacctcggctacgagctccagggcgccaagatgctgacatgcatcaacgccagcaaacctcactggtccagccaggagcctatctgtagcgctccttgcggcggagccgtgcacaatgctacaattggcagagtgctcagcccttcctaccctgaaaacaccaacggctcccagttctgcatctggacaatcgaggcccccgaaggccaaaagctgcacctgcactttgagaggctcctgctccacgacaaagacaggatgaccgtgcactccggccagaccaataagtccgccctcctgtatgacagcctgcagacagagtccgtcccttttgaaggcctgctgtccgagggcaataccatcaggattgagttcacatccgaccaagccagggctgctagcaccttcaacattaggtttgaggctttcgaaaagggacactgctacgagccctatattcagaatggcaatttcacaacctccgaccccacctacaatatcggcacaattgtggagtttacctgcgaccctggacacagcctggagcagggacctgccatcatcgaatgcatcaacgtcagggacccctactggaacgacacagaacctctgtgtagggctatgtgcggaggcgaactgagcgctgtggctggagtcgtgctctcccctaactggcccgaaccctatgtggagggcgaagattgcatctggaagatccacgtcggcgaggaaaaaaggatctttctggacatccagttcctgaatctctccaacagcgacatcctgaccatctacgacggagatgaggtcatgccccacattctgggccagtatctcggaaactccggcccccaaaagctctactcctccacccccgacctcacaatccaattccacagcgatcctgctggcctcatctttggaaagggacaaggctttatcatgaattacatcgaggtcagcagaaacgacagctgctccgacctgcctgagatccagaacggatggaagaccacctcccacaccgagctcgtcaggggagctaggatcacataccagtgcgaccccggatacgacatcgtcggctccgataccctgacatgccagtgggatctgagctggagctccgacccccccttttgtgagaagatcatgtactgcaccgaccccggcgaagtcgatcatagcaccaggctcatcagcgatcctgtgctgctcgtcggcacaaccatccaatacacctgtaaccccggattcgtgctcgaaggatcctccctgctcacctgttacagcagggaaaccggcacccccatttggacatccaggctgcctcactgcgtgtccgaagagagcctggcttgcgataatcccggcctgcctgagaacggataccagattctgtacaaaaggctgtacctccccggcgagtccctgaccttcatgtgctacgaaggattcgagctcatgggcgaagtcaccatcaggtgcatcctcggccagccctcccactggaacggacctctccccgtctgtaaggtcaatcaggattccttcgagcacgctctggaagtcgctgaggctgccgccgagacaagcctggaaggcggc

SEQ ID NO: 17 - protein, 964 aa, Homo sapiens
  MISC_FEATURE: Human SEZ6L ECD protein
METDTLLLWVLLLWVPGSTGDHGAPLERDALPEGDASPLGPYLLPSGAPERGSPGKEHPEERVVTAPPSSSQSAEVLGELVLDGTAPSAHHDIPALSPLLPEEARPKHALPPKKKLPSLKQVNSARKQLRPKATSAATVQRAGSQPASQGLDLLSSSTEKPGPPGDPDPIVASEEASEVPLWLDRKESAVPTTPAPLQISPFTSQPYVAHTLPQRPEPGEPGPDMAQEAPQEDTSPMALMDKGENELTGSASEESQETTTSTIITTTVITTEQAPALCSVSFSNPEGYIDSSDYPLLPLNNFLECTYNVTVYTGYGVELQVKSVNLSDGELLSIRGVDGPTLTVLANQTLLVEGQVIRSPTNTISVYFRTFQDDGLGTFQLHYQAFMLSCNFPRRPDSGDVTVMDLHSGGVAHFHCHLGYELQGAKMLTCINASKPHWSSQEPICSAPCGGAVHNATIGRVLSPSYPENTNGSQFCIWTIEAPEGQKLHLHFERLLLHDKDRMTVHSGQTNKSALLYDSLQTESVPFEGLLSEGNTIRIEFTSDQARAASTFNIRFEAFEKGHCYEPYIQNGNFTTSDPTYNIGTIVEFTCDPGHSLEQGPAIIECINVRDPYWNDTEPLCRAMCGGELSAVAGVVLSPNWPEPYVEGEDCIWKIHVGEEKRIFLDIQFLNLSNSDILTIYDGDEVMPHILGQYLGNSGPQKLYSSTPDLTIQFHSDPAGLIFGKGQGFIMNYIEVSRNDSCSDLPEIQNGWKTTSHTELVRGARITYQCDPGYDIVGSDTLTCQWDLSWSSDPPFCEKIMYCTDPGEVDHSTRLISDPVLLVGTTIQYTCNPGFVLEGSSLLTCYSRETGTPIWTSRLPHCVSEESLACDNPGLPENGYQILYKRLYLPGESLTFMCYEGFELMGEVTIRCILGQPSHWNGPLPVCKVNQDSFEHALEVAEAAAETSLEGGLAGHHHHHHHHH

SEQ ID NO: 18 - DNA, 2487 bp, Homo sapiens
  misc_feature: cDNA sequence of human SEZ6L2 ECD
ctgcctctcaaagaggaagagattctccccgagcccggatccgagacacccacagtggcttccgaagccctcgctgaactgctgcacggagccctcctgagaaggggacctgaaatgggctatctccctggctccgacagagatcccacactcgccacacctcctgctggacagaccctcgctgtgccttccctgcccagagccacagaacccggaacaggccctctcacaacagctgtgacccctaacggcgtcagaggagctggacctacagcccctgagctgctgacacctcctcctggcacaaccgctcctcctcctccttcccctgctagccctggaccccctctcggacctgaaggaggcgaggaggagacaaccaccaccattattaccaccaccaccgtgacaaccacagtgaccagccctgtcctgtgcaacaacaacatcagcgaaggcgaaggctatgtggaatcccctgacctgggctcccctgtgtccagaacactcggcctcctggattgcacatactccattcacgtgtaccccggctacggaatcgagattcaggtgcagaccctgaatctgtcccaggaggaggaactgctggtgctggctggcggaggaagccctggcctcgctcctagactcctcgctaactcctccatgctcggcgaaggccaggtcctcagatcccctaccaacaggctgctcctgcacttccagagccccagagtgcctagaggaggcggcttcaggattcactaccaggcctatctcctgagctgtggattccctcccagacccgctcatggcgatgtctccgtcaccgacctccaccccggaggaacagccaccttccactgtgattccggataccagctgcaaggcgaggagaccctgatttgcctcaatggcaccaggcccagctggaacggagagacacctagctgcatggctagctgcggcggaaccatccataatgccaccctcggcaggatcgtcagccctgaacctggcggagctgtgggacctaacctcacatgcagatgggtgatcgaagctgctgaaggcaggagactccacctccacttcgagagggtgtccctggacgaggacaacgacaggctcatggtcagaagcggcggaagccctctcagccctgtgatttacgacagcgacatggacgatgtgcctgagaggggcctcatctccgatgcccaaagcctgtacgtggaactcctctccgagacccccgctaaccccctcctcctgagcctcagattcgaggccttcgaggaggacagatgtttcgctccttttctggcccatggcaacgtgaccacaaccgaccccgagtacagacccggagctctggctaccttcagctgtctgcctggctacgccctcgaacctcccggacctcctaatgccatcgaatgtgtggatcccaccgaaccccattggaacgacaccgagcccgcttgtaaggctatgtgcggcggagaactcagcgaacctgccggagtggtcctctcccctgattggccccagagctattcccccggacaagactgtgtctggggcgtgcacgtccaggaggaaaagaggatcctcctccaggtggagattctgaacgtcagagagggagacatgctgaccctgttcgacggagacggaccttccgccagagtcctcgctcagctgagaggccctcagcccagaaggagactgctcagctccggccccgatctgacactccagtttcaggccccccctggcccccctaatcctggcctgggacagggcttcgtgctccacttcaaggaggtccccaggaatgatacatgccccgaactgcctcctcccgagtggggatggaggacagcttcccatggcgacctgatcaggggaaccgtgctgacatatcagtgtgaacccggctacgagctgctgggaagcgatatcctgacctgtcagtgggatctctcctggagcgctgctccccctgcctgtcagaaaatcatgacctgcgctgaccctggagagatcgctaacggccacaggaccgcttccgacgctggatttcccgtgggctcccacgtgcaatacaggtgcctccccggatactccctcgaaggcgctgccatgctgacatgctacagcagggacaccggcacacccaagtggtccgacagggtgcccaaatgtgctctgaagtacgagccctgtctcaatcccggagtgcccgagaacggataccagaccctgtacaagcaccactatcaggccggcgaatccctgagattcttctgctacgagggcttcgagctcatcggcgaggtgacaattacctgtgtgcccggccatccttcccagtggaccagccagccccctctctgtaaggtcgcctacgaagagctgctcgacaataggaagctggaggtcacccagaccaccgacccttccagacaactggaaggcggc

SEQ ID NO: 19 - protein, 865 aa, Homo sapiens
  MISC_FEATURE: Human SEZ6L2 ECD protein
METDTLLLWVLLLWVPGSTGDGAPLPLKEEEILPEPGSETPTVASEALAELLHGALLRRGPEMGYLPGSDRDPTLATPPAGQTLAVPSLPRATEPGTGPLTTAVTPNGVRGAGPTAPELLTPPPGTTAPPPPSPASPGPPLGPEGGEEETTTTIITTTTVTTTVTSPVLCNNNISEGEGYVESPDLGSPVSRTLGLLDCTYSIHVYPGYGIEIQVQTLNLSQEEELLVLAGGGSPGLAPRLLANSSMLGEGQVLRSPTNRLLLHFQSPRVPRGGGFRIHYQAYLLSCGFPPRPAHGDVSVTDLHPGGTATFHCDSGYQLQGEETLICLNGTRPSWNGETPSCMASCGGTIHNATLGRIVSPEPGGAVGPNLTCRWVIEAAEGRRLHLHFERVSLDEDNDRLMVRSGGSPLSPVIYDSDMDDVPERGLISDAQSLYVELLSETPANPLLLSLRFEAFEEDRCFAPFLAHGNVTTTDPEYRPGALATFSCLPGYALEPPGPPNAIECVDPTEPHWNDTEPACKAMCGGELSEPAGVVLSPDWPQSYSPGQDCVWGVHVQEEKRILLQVEILNVREGDMLTLFDGDGPSARVLAQLRGPQPRRRLLSSGPDLTLQFQAPPGPPNPGLGQGFVLHFKEVPRNDTCPELPPPEWGWRTASHGDLIRGTVLTYQCEPGYELLGSDILTCQWDLSWSAAPPACQKIMTCADPGEIANGHRTASDAGFPVGSHVQYRCLPGYSLEGAAMLTCYSRDTGTPKWSDRVPKCALKYEPCLNPGVPENGYQTLYKHHYQAGESLRFFCYEGFELIGEVTITCVPGHPSQWTSQPPLCKVAYEELLDNRKLEVTQTTDPSRQLEGGLAGHHHHHHHHH

SEQ ID NO: 20 - protein, 107 aa, Mus musculus
  MISC_FEATURE: Murine SC17.1 VL
QIVLTQSPALMSASPGEKVSLTCSANSTVSFMYWYQQKPRSSPTPWIYLTSNLASGVPARFSGSGSGTSYSLTISSMEAEDAATYYCQQWSSNSPITFGAGTKLELK

SEQ ID NO: 21 - protein, 117 aa, Mus musculus
  MISC_FEATURE: Murine SC17.1 VH
DVQLQDSGPGLVKPSQSLSVTCTVTGYSITWGYYWNWIRQFPGNKLEWMGNIHNSGGTNYNPSLKSRISITRDTSKNQFFLQLNSVTTEDTATYYCATTNWDYFDYWGQGTTLTVSS

SEQ ID NO: 22 - protein, 112 aa, Mus musculus
  MISC_FEATURE: Murine SC17.2 VL
DIVMSQSPSSLAVSVGEKVTMSCKSSQSLLYSSNQKSYLAWYQQKPGQSPKLLIYWASTRESGVPDRFTGSGSGTDFTLTISSVQAEDLAVYYCKQSYNLRTFGGGTKLEIK

SEQ ID NO: 23 - protein, 117 aa, Mus musculus
  MISC_FEATURE: Murine SC17.2 VH
QVQLQQSDAELVKPGASVKISCKVSGYTFTDHTIHWMKQRPEQGLEWIGYIYPRDGSTKYNEEFKGKATLTADKSSSTAYMQLNSLTSEDSAVYFCARSYSNYFDYWGQGTTLTVSS

SEQ ID NO: 24 - protein, 111 aa, Mus musculus
  MISC_FEATURE: Murine SC17.9 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYFCQQYYNYPYTFGGGTKLK

SEQ ID NO: 25 - protein, 119 aa, Mus musculus
  MISC_FEATURE: Murine SC17.9 VH
QVQLQQPGAEIVRPGASVKLSCKASGYTFTDYWMNWVKQRPGQGLEWIGAIDPSDSYTSYNPKFKGKATLTVDTSSSSAYMQLSSLTSEDSAVYFCARRGTPGKPLVYWGQGTLVTVSA

SEQ ID NO: 26 - protein, 107 aa, Mus musculus
  MISC_FEATURE: Murine SC17.16 VL
DIQMTQSPASLSVSVGETVTITCRASANINSNLVWYQQKQGKSPQLLVYAATNLADGVPSRFSGSGSGTQYSLKINSLQSEDFGNYYCQHFWGTPRTFGGGTKLEIK

SEQ ID NO: 27 - protein, 116 aa, Mus musculus
  MISC_FEATURE: Murine SC17.16 VH
EVQLQQSGPELMKPGASVKMSCKASGYTFTDYNMYWVKQNQGKSLEWIGEINPNNGGTAYNQKFRGKATLTVDKSSSTAYMELRSLTSEDSAVYYCARYDKGFDYWGQGTTLTVSS

SEQ ID NO: 28 - protein, 111 aa, Mus musculus
  MISC_FEATURE: Murine SC17.38 VL
DIVVTQSPASLAVSLGQRATISCRASESVEYYGTSLMQWFQQKPGQPPKLLIYAASNVESGVPARFSGSGSGTDFSLNIHPVEEDDIAMYFCQQDRKVPWTFGGGTKLEIK

SEQ ID NO: 29 - protein, 120 aa, Mus musculus
  MISC_FEATURE: Murine SC17.38 VH
QVTLKESGPGILQPSQTLSLTCSFSGFSLNTSGMSVGWVRQPSGRGLEWLAPIWWNGDKYYNPALKSRLTISKDTSNNQVFLKIASVVTADTATYFCARIRQYYYAMDYWGQGTSVTVSS

SEQ ID NO: 30 - protein, 112 aa, Mus musculus
  MISC_FEATURE: Murine SC17.3 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPTFGGGTKLEIK

SEQ ID NO: 31 - protein, 118 aa, Mus musculus
  MISC_FEATURE: Murine SC17.3 VH
QVQLQQPGAELVKPGASVKLSCKASGYTFPSYWIHCVKQRPGQGLEWIGVINPSNGRTNYNEKFKNKATLTVDKSSSTAYMQLSSLTSEDSAVYYCVRGGTGYTMDYWGQGTSVTVSS

SEQ ID NO: 32 - protein, 107 aa, Mus musculus
  MISC_FEATURE: Murine SC17.4 VL
DIKMTQSPSSMYASLGERVTITCKASQDINSYLTWFQQKPGKSPKTLIYRANRLIDGVPSRFSGSGSGQDYSLTISSLDYEDMGIYYCLQYDDFPWTFGGGTKLEIK

SEQ ID NO: 33 - protein, 116 aa, Mus musculus
  MISC_FEATURE: Murine SC17.4 VH
QIQLVQSGPELKKPGETVKISCKASGYTFTDYSMHWVKQAPGKGLKWLGWINTETGEPTYSEDFKGRFAFSLETSASTAYLQINNLKNEDTATYFCVKNKGWFAYWGQGTLVTVSA

SEQ ID NO: 34 - protein, 112 aa, Mus musculus
  MISC_FEATURE: Murine SC17.8 VL
DVVMTQTPLSLPVSLGDQASISCRSSQSLVHSNGDTYLHWYLQKPGQSPKLLIYKVSNRFSGVPDRFSGSGSGTDFTLKISRVEAEDLGLYFCSQSTLIPYTFGGGTKLDIK

SEQ ID NO: 35 - protein, 114 aa, Mus musculus
  MISC_FEATURE: Murine SC17.8 VH
QVHLQQSGTEVMKPGASVKISCKATGYTFSSYWIEWIKQRPGHGLEWIGEILPGSGNTNNNEKFKGKATITADTSSNIAYIQLSSLTSEDSAVYYCAGGPAAYWGQGTLVTVSA

SEQ ID NO: 36 - protein, 113 aa, Mus musculus
  MISC_FEATURE: Murine SC17.10 VL
DIVMSQSPSSLAVSVGEKVTMSCKSSQSLLYSSNQKNYLAWYQQKPGQSPKLLIYWASTRESGVPDRFTGSGSGTDFTLTISSVKTEDLALYYCQQYYWFPYTFGGGTKLEIK

SEQ ID NO: 37 - protein, 113 aa, Mus musculus
  MISC_FEATURE: Murine SC17.10 VH
EVQLQQSGAELVKPGASVKLSCTASGFNIKDTYMHWVKQRPEQGLEWIGRIDPANVNTKYDPKFQGKATITADTSSNTAYLQLSSLTSEDTAVYYCVRGNVYWGQGTLVTVSA

SEQ ID NO: 38 - protein, 108 aa, Mus musculus
  MISC_FEATURE: Murine SC17.11 VL
ENVLTQSPAIMSASPGEKVTMTCRASSSVSSSYLHWYQQKSGASPKLWIYSTSNLASGVPARFSGSGSGTSYSLTISSVEAEDAATYYCQQYSDYPFTFGSGTKLVIK

SEQ ID NO: 39 - protein, 122 aa, Mus musculus
  MISC_FEATURE: Murine SC17.11 VH
EVQLQQSGPELVKPGALVMMSCKASGYTFTDYYMHWVKQSHGQSLEWIGEVIPYNDETFYNRKFKDKATLTVDKSSSTAYMELRSLTSEDSAIYYCARRHRYDGFRYAIDYWGQGTSVTVSS

SEQ ID NO: 40 - protein, 112 aa, Mus musculus
  MISC_FEATURE: Murine SC17.14 VL
DVLMTQTPLSLPVSLGDQASISCRSSQSIVHSNGNTYLEWFLQKPGQSPKLLIYKVSNRFSGVPDRFSGSGSGTDFTLKISRVEAEDLGVYYCFQGSHVPYTFGGGTKLEIK

SEQ ID NO: 41 - protein, 118 aa, Mus musculus
  MISC_FEATURE: Murine SC17.14 VH
EVQLQQSGPVLVKPGASVKMSCKASGYTITDYNMNWVKQSHGKSLEWIGVINPYNGNTRYNQMFKGKATLTVDKSSSTAYMELNSLTSEDSAVYYCTRWGTTVVGANWGQGTTLTVSS

SEQ ID NO: 42 - protein, 106 aa, Mus musculus
  MISC_FEATURE: Murine SC17.15 VL
QIVLTQSPALMSASPGEKVTMTCSASSSVNYMYWYQQKPRSSPKPWIYLTSNLASGVPVRFSGSGSGTSYSLTISSMEAEDAATYYCQQWSNNPPTFGSGTKLELK

SEQ ID NO: 43 - protein, 122 aa, Mus musculus
  MISC_FEATURE: Murine SC17.15 VH
DVKLVESGGGLVKLGGSLKLSCAASGFTFSSYAMSWVRQTPEKRLEWVATITSGGGNTYYPDSVKGRFTISRDNAKNTLYLQMSSLKSEDTAMYYCARRDYYGSSYVMFAYWGQGTLVTVSA

SEQ ID NO: 44 - protein, 106 aa, Mus musculus
  MISC_FEATURE: Murine SC17.17 VL
QIVLTQSPAIMSASPGEKVTMTCSASSSVSYMHWYQQKSGTSPKRWIYDTSKLPSGVPARFSGSGSGTSYSLTISSMEAEDAATYYCQQWSSTPPTFGAGTKLELK

SEQ ID NO: 45 - protein, 120 aa, Mus musculus
  MISC_FEATURE: Murine SC17.17 VH
EVQLQQSGPEVMKPGASVKMSCKASGYTFTDYNMHWVKQNQGKSLEWIGEINPNIGGTGYNQKFKGKATLTVHKSSSTAYMELRSLTSEDSAVYYCARTYSYYSYEFAYWGQGTLVTVSA

SEQ ID NO: 46 - protein, 107 aa, Mus musculus
  MISC_FEATURE: Murine SC17.18 VL
DIQMTQSSSYLSVSLGGRVTITCKASDHINNWLAWYQQKPGNAPRLLISGATSLETGVPSRFSGSGSGKDYTLSITSLQTEDVATYYCQQYWSIPLTFGAGTKLELK

SEQ ID NO: 47 - protein, 123 aa, Mus musculus
  MISC_FEATURE: Murine SC17.18 VH
QVTLKESGPGILQPSQTLSLTCSFSGFSLSTSTMGVGWIRQPSGKGLEWLADIWWDDSKYYNPSLKSRLTISKDTSSNQVFLKITSVDTADTATYYCARKGRTARATRGFAYWGHGTLVTVSA

SEQ ID NO: 48 - protein, 109 aa, Mus musculus
  MISC_FEATURE: Murine SC17.19 VL
DIVLTQSPASLAVSLGQRAAISCKPSQSVDYDGDSYMNWYQQKPGQPPKLLIYAASNLESGIPARFSGSGSGTDFTLNIHPVEEEDAATYYCHQINDDPWTFGGGTKLK

SEQ ID NO: 49 - protein, 121 aa, Mus musculus
  MISC_FEATURE: Murine SC17.19 VH
DVQLQESGPGLVKPSQSLSVTCTVTGYSITSSYTWNWIRQFPGNKLEWMGYIHYSGSTNYNPSLRSRISITRDTSKNQFFLQLNSVTTEDTATYYCARSRYYYDAYGFAYWGQGTLVTVSA

SEQ ID NO: 50 - protein, 112 aa, Mus musculus
  MISC_FEATURE: Murine SC17.22 VL
DVVLTQTPLSLPVSLGDQASISCRSSQSIVHINRHTYLGWYLQKPGQSLKLLIYGVSNRFSGVPDRFSGSGSGTDFTLKISRVEAEDMGVYYCFQGTHVPFTFGSGTKLEIK

SEQ ID NO: 51 - protein, 121 aa, Mus musculus
  MISC_FEATURE: Murine SC17.22 VH
QIQMMQSGPELKKPGETVKISCKASGYSFTNYGMNWVKQAPGKGLKWMGWINTYTGEPTYADDFKGRFAFSLETSASTAYLQINNLKNEDMATYFCTRGYYGSSYDALDYWGQGTSVTVSS

SEQ ID NO: 52 - protein, 112 aa, Mus musculus; identical to SEQ ID NO: 22
  MISC_FEATURE: Murine SC17.24 VL

SEQ ID NO: 53 - protein, 117 aa, Mus musculus; identical to SEQ ID NO: 23
  MISC_FEATURE: Murine SC17.24 VH

SEQ ID NO: 54 - protein, 112 aa, Mus musculus
  MISC_FEATURE: Murine SC17.27 VL
DVVMTQTPLTLSVTIGQPASISCKSSQSLLESDGKTYLNWLLQRPGQSPKRLIYLVSKLDSGVPDRFTGSGSGTDFTLKISRVEAEDLGVYYCWQGIQHPRTFGGGTKLEIK

SEQ ID NO: 55 - protein, 113 aa, Mus musculus
  MISC_FEATURE: Murine SC17.27 VH
QVQLQQSGAELVRPGASVTLSCKASGYTFTDYEMHWVKQTPVHGLEWIGGIDPETGGTAYNQKFKGKATLTADKSSSTAYMELRSLTSEDSAVYFCTRWFSYWGPGTLVTVSA

SEQ ID NO: 56 - protein, 107 aa, Mus musculus
  MISC_FEATURE: Murine SC17.28 VL
DILLTQSPAILSVSPGEGVSFSCRASQSIGTSIHWYQQRTNGSPRLLIKYASESISGIPSRFSGSGSGTDFTLRINSLESEDIADYYCQQSNSWPLTFGAGTKLELK

SEQ ID NO: 57 - protein, 117 aa, Mus musculus
  MISC_FEATURE: Murine SC17.28 VH
QVHLPQSRPELVKPGASVKISCKASGYGFTRSYIHWVKQRPGQGLEWIGYISSGSGGTTYNQKFKGKASLTADNPSSTAYMHLSSLTSEDSAIYFCARGGVRYFDVWGAGTTVTVSS

SEQ ID NO: 58 - protein, 107 aa, Mus musculus
  MISC_FEATURE: Murine SC17.29 VL
DIVMTQSHKFMSTSVGDRVSITCKASQDVGTDVAWYQQKPGQSPKLLIYWASTRHTGVPDRFTGSGSGTDFTLTISNVQSEDLADYFCQQYSSYPYTFGGGTKLEIK

SEQ ID NO: 59 - protein, 116 aa, Mus musculus
  MISC_FEATURE: Murine SC17.29 VH
EVQLQQSGPELMKPGASVKMSCKASGYTFTDYNMHWVKQNQGKSLEWIGEINPHNGGTGYNQKFKGKATLTVDKSSSTSYMELRSLTSEDSAVYYCAGGYPAFDYWGQGTTLTVSS

SEQ ID NO: 60 - protein, 108 aa, Mus musculus
  MISC_FEATURE: Murine SC17.30 VL
ENVLTQSPAIVSASPGEKVTMTCRASSSVISSYLHWYQQKSGASPKLWIYSTSNLASGVPARFSGSASGTSYSLTISSVEAEDAATYYCQQYSGYPLTFGAGTKLELK

SEQ ID NO: 61 - protein, 124 aa, Mus musculus
  MISC_FEATURE: Murine SC17.30 VH
EVKLVESEGGLVQPGSSMKLSCTASGFTFSDYYMAWVRQVPEKGLEWVANINYDGSSTYYLDSLKSRFIISRDNAKNILYLQMSSLKSEDTATYYCARDDYYGSSPSYWYFDVWGAGTTVTVSS

SEQ ID NO: 62 - protein, 107 aa, Mus musculus
  MISC_FEATURE: Murine SC17.32 VL
DIQMTQSPASLSASVGETVTMTCRASGNIHNYLVWYQQKQGKSPQLLVYNAKTLADGVPSRFSGSGSGTQYSLKINSLQPEDFGSYYCQHFWSTPPTFGGGTKLEIK

SEQ ID NO: 63 - protein, 119 aa, Mus musculus
  MISC_FEATURE: Murine SC17.32 VH
EVKLEESGGGLVQPGGSMKLSCVASGFTFSNYWMSWVRQSPEKGLEWVAEIRLKSNNYATHYAESVKGRFTISRDDSKSSVFLQMNNLRTEDTGIYYCTRHYYYAMDYWGQGTSVTVSS

SEQ ID NO: 64 - protein, 107 aa, Mus musculus
  MISC_FEATURE: Murine SC17.34 VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGGGTKLEIK

SEQ ID NO: 65 - protein, 118 aa, Mus musculus
  MISC_FEATURE: Murine SC17.34 VH
EVQLQQSGPELVKPGSSVKISCKASGYTFTDYNMDWVKQSHGKRLEWIGYIYPDNGGAGYNQKFKGKATLTVDKSSSTAYMELRSLTSEDSAVYYCSRSITTAWFAYWGQGTLVTVSA

SEQ ID NO: 66 - protein, 108 aa, Mus musculus
  MISC_FEATURE: Murine SC17.35 VL
ENVLTQSPAIMSASPGEKVTLTCRASSSMSSSYLHWYQQKSGASPKLWIYSTSNLASGVPARFSGSGSGTSYSLTISSVEAEDAATYYCQQYSAYPFTFGSGTKLEIK

SEQ ID NO: 67 - protein, 121 aa, Mus musculus
  MISC_FEATURE: Murine SC17.35 VH
EVQLQQSGPELVKPGALVKMSCKASGYTFTDYYIHWVKQSHGKSLEWIGEINPYNGETFYNQKFKGKATLTVDKSSSTAYMELRSLTSEDSAVYYCARRGWYLTGYAMDYWGQGTSVTVSS

SEQ ID NO: 68 - protein, 106 aa, Mus musculus
  MISC_FEATURE: Murine SC17.36 VL
QIVLTQSPALMSASPGEKVTMTCSASSSVSYMYWYQQKPRSSPKPWIYLTSNLASGVPARFSGSGSGTSYSLTISSMEAEDAATYYCQQWSSNPPTFGGGTKLEIK

SEQ ID NO: 69 - protein, 120 aa, Mus musculus
  MISC_FEATURE: Murine SC17.36 VH
EVQLQESGPSLVKPSQSQSLTCSVTGDSITSDYWNWIRKFPGKKVEYMGYINYSGSTYYNPSLKSRISITRDTSKNQYYLQLNSVTSEDTATYYCARTSYYNKFLPFAYWGQGTLVTVSA

SEQ ID NO: 70 - protein, 112 aa, Mus musculus
  MISC_FEATURE: Murine SC17.39 VL
DVLMTQTPLSLPVSLGDQASISCRSSQSLVHRNGNTYFHWYLQKPGQSPKLLIYKVSNRFSGVPDRFSGSGSGTDFTLKISRVEAEDLGVYFCSQSTYVPWTFGGGTKLEIK

SEQ ID NO: 71 - protein, 117 aa, Mus musculus
  MISC_FEATURE: Murine SC17.39 VH
EVQLVESGGGLVQPGGSRKLSCAASGFTFSSYGMHWVRQAPEKGLEWVAYISSNDGTIYYADTVRGRFTISRDNAKNTLFLQMTSLRSEDTAMYYCARPSNWVFDYWGQGTTLTVSS

SEQ ID NO: 72 - protein, 112 aa, Mus musculus
  MISC_FEATURE: Murine SC17.40 VL
DVVMTQTPLSRPVTLGDQASISCRSSQSLVHSNGNTYLHWYLQKPGQSPKLLIYKVSNRFSGVPDRFSGSGSGTDFTLKISRVEAEDLGVYFCSQNTHVPFTFGSGTKLEIK

SEQ ID NO: 73 - protein, 116 aa, Mus musculus
  MISC_FEATURE: Murine SC17.40 VH
QVQLQQPGAEIVRPGASVKLSCKASGYTFTDYWMNWVKQRPGQGLEWIGTIDPSDSYTRYNQKFKGKATLTVDTSFSSAYMQLSSLTSEDSAVYFCASGGRGFGYWGQGTPVTVSV

SEQ ID NO: 74 - protein, 106 aa, Mus musculus
  MISC_FEATURE: Murine SC17.41 VL
QIVLTQSPALMSASPGEKVTMTCSASSSVSYMYWYQQKPRSSPKPWIYLTSNLASGVPTRFSGSGSGTSYSLTISSMGAEDAATYYCQQWNTNPPTFGAGTKLELK

SEQ ID NO: 75 - protein, 121 aa, Mus musculus
  MISC_FEATURE: Murine SC17.41 VH
DVKLVESGGGLVKLGGSLKLSCAASGFTFSSYAMSWVRQTPEKRLEWVATISSGGGNTYYPDSVKGRFTISRDNAKNTLYLQMSSLKSEDTAMYYCARRDYYGTSYVMFAYWGQGTLVTVS

SEQ ID NO: 76 - protein, 106 aa, Mus musculus
  MISC_FEATURE: Murine SC17.42 VL
ENVLTQSPAIMSASPGEKVTMTCSASSSVNYMYWYQQKSSTSPKLWIYDTSKLTSGVPGRFSGSGSGNSYSLTISNMEAEDVATYYCFQGSGYPLTFGSGTKLEIK

SEQ ID NO: 77 - protein, 112 aa, Mus musculus
  MISC_FEATURE: Murine SC17.42 VH
DVKLVESGGGLVRPGGSLKLSCAASGFTFSRYTMSWVRQTPEKRLEWAATINSGGSNTYYPDSVKGRFTISRDNAKNTLFLQMSSLKSEDTAMYYCTNGNHWGQGTTLTVSS

SEQ ID NO: 78 - protein, 106 aa, Mus musculus; identical to SEQ ID NO: 76
  MISC_FEATURE: Murine SC17.45 VL

SEQ ID NO: 79 - protein, 114 aa, Mus musculus
  MISC_FEATURE: Murine SC17.45 VH
QVQLQQPGSVLVRPGDSVKLSCKASGYTFTSYWMHWVKQSPGQGLEWIGEIHPHSGSTNYNEKFKGKATLTVDTSSSTAYVDLSSLTSEDSAVYYCVGGHYDYWGQGTTLTVSS

SEQ ID NO: 80 - protein, 107 aa, Mus musculus
  MISC_FEATURE: Murine SC17.46 VL
SFVMTQTPKFLLVSAGDRVTITCKASQSVNNDVAWYQQKPGQSPKLLIYYASNRYTGVPDRFTGSGYGTDFTFTISTVQAEDLAVYFCQQDYSSPRTFGGGTKLEIK

SEQ ID NO: 81 - protein, 119 aa, Mus musculus
  MISC_FEATURE: Murine SC17.46 VH
QVQLQQPGAELVKPGASMKLSCKASGYTFTSYWINWVKQRPGQGLEWIGNIFPDTTTTNYNEKFKSKATLTVDTSSSTAYMQLSSLTSDDSAVYYCAREYYDGTYDAMDYWGQGTSVTV

SEQ ID NO: 82 - protein, 106 aa, Mus musculus
  MISC_FEATURE: Murine SC17.47 VL
QIVLTQSPAIMSASPGEKVSMTCSASSSVSYMHWYQQKSGTSPKRWIYDTSKLASGVPARFSGSGSGSSYSLTISSMEAEDAATYYCQQWSSTPPTFGGGTKLEIK

SEQ ID NO: 83 - protein, 119 aa, Mus musculus
  MISC_FEATURE: Murine SC17.47 VH
EVQLQQSGPELVKPGASVKISCKASGYSFTDYYMRWVKQSPEKSLEWIGEINPSTGGTTYNQNFKAKATLTVDKSSSTAYMQLKSLTSEDSAVYYCARGGYFLYYFDYWGQGTTLTVSS

SEQ ID NO: 84 - protein, 112 aa, Mus musculus; identical to SEQ ID NO: 54
  MISC_FEATURE: Murine SC17.49 VL

SEQ ID NO: 85 - protein, 113 aa, Mus musculus; identical to SEQ ID NO: 55
  MISC_FEATURE: Murine SC17.49 VH

SEQ ID NO: 86 - protein, 111 aa, Mus musculus
  MISC_FEATURE: Murine SC17.50 VL
DIVLTQSPASLAASLGQRATISCRASQSVSTSSYSYMHWYQQKPGQPPKLLIKYASNLESGVPARFSGSGSGTDFTLNIHPVEEEDTATYYCQHSWEIPWTFGGGTKLEIK

SEQ ID NO: 87 - protein, 121 aa, Mus musculus
  MISC_FEATURE: Murine SC17.50 VH
EVQLVESGGGLVKPGGSLKLSCAASGFTFSDYGMHWVRQAPEKGLEWVAYISSGSRTIYYADTVKGRFTISRDNAKNTLFLQMTSLRSEDTAMYYCARVYYGSTYGYFDVWGTGTTVTVSS

SEQ ID NO: 88 - protein, 111 aa, Mus musculus
  MISC_FEATURE: Murine SC17.53 VL
DIVLTQSPASLAASLGQRATISCRASQSVSTSSYSYMHWYQQKPGHPPKLLIRYASNLESGVPARFSGSGSGTDFTLNIHPVEEEDTATYYCQHSWEIPYTFGGGTKLEIK

SEQ ID NO: 89 - protein, 119 aa, Mus musculus
  MISC_FEATURE: Murine SC17.53 VH
EVQLQQSGPELVKPGASVKISCKASGYTFTDYNMHWVKQSHGKRLEWIGYIHPYNGGSGYNQKFKRKATLTVDNSSNTTYMELRSLTSEDSAVYYCARSYDYDTWFGYWGQGTLVTVRA

SEQ ID NO: 90 - protein, 112 aa, Mus musculus
  MISC_FEATURE: Murine SC17.54 VL
DVVLTQTPLTLSVTIGQPASISCKSSQSLLYSDGKTYLNWLLQRPGQSPKRLIYLVSKLDSGVPDRFTGSGSGTDFTLKISRVEAEDLGLYYCWQGTHFPWTFGGGTKLEIK

SEQ ID NO: 91 - protein, 113 aa, Mus musculus
  MISC_FEATURE: Murine SC17.54 VH
EVKLEESGGGLVQPGGSMKLSCVASGFTFSNYWINWVRQSPEKGLEWVAEIRMKSNNYATHYAESVKGRFTISRDDSKSCVYLQMNNLRPEDTGIYYCTRGGYWGQGTTLTVS

SEQ ID NO: 92 - protein, 113 aa, Mus musculus
  MISC_FEATURE: Murine SC17.56 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYFCQQYYNYPYTFGGGTKLEIK

SEQ ID NO: 93 - protein, 116 aa, Mus musculus
  MISC_FEATURE: Murine SC17.56 VH
QIQLVQSGPELKKPGETVKISCKASGYTFTNYGMNWVKQAPGKGLKWMAWINTYTGEPTYADDFKGRFAFSLETSASTASLQIINLKNEDTATYFCARIGDSSPSDYWGQGTTLTV

SEQ ID NO: 94 - protein, 108 aa, Mus musculus
  MISC_FEATURE: Murine SC17.57 VL
QIVLTQSPAIMSASLGERVTMTCTASSSVSSSYLHWYQQKPGSSPKLWIYSTSNLASGVPPRFSGSGSGTSYSLTISSMEAEDAATYYCHQYHRSPPTFGGGTKLEIK

SEQ ID NO: 95 - protein, 121 aa, Mus musculus
  MISC_FEATURE: Murine SC17.57 VH
QIQLVQSGPELKKPGETVKISCKASDYTFTDFSIHWVRQSPGKGLKWMGWINTETGEPTVAEDFKGRFAFSLETSASTAFLQIYNLKNEDSATYFCARGRYYGHDYAMDYWGQGTSVTVSS

SEQ ID NO: 96 - protein, 107 aa, Mus musculus
  MISC_FEATURE: Murine SC17.59 VL
DIQMTQSPASLSASVGETVTITCRASGNLHNYLAWYQQKQGKSPQLLVYNAKTLADGVPSRFSGSGSGTQYSLKINSLQPEDFGTYFCQHFWSIPPTFGGGTKLEIK

SEQ ID NO: 97 - protein, 119 aa, Mus musculus
  MISC_FEATURE: Murine SC17.59 VH
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRLWDFAMDYWGQGTSVTVSS

SEQ ID NO: 98 - protein, 106 aa, Mus musculus
  MISC_FEATURE: Murine SC17.61 VL
QIVLTQSPAIMSASPGEKVTISCSASSSVSYIYWYQQKPGSSPKPWIYRTSNLASGVPARFSGSGSGTSYSLTISSMEAEDAATYYCQQYHSYPWTFGGGTKLEIK

SEQ ID NO: 99 - protein, 125 aa, Mus musculus
  MISC_FEATURE: Murine SC17.61 VH
QVTLKESGPGILQPSQTLSLTCSFSGFSLSTFGMGVGWIRQPSGKGLEWLAQIWWDDYKYYNPALKSRLTISKDTSKNQVFLKIANVDTADTATYYCARIGYYSGSSRCWYFDVWGTGSTVTVSS

SEQ ID NO: 100 - protein, 107 aa, Mus musculus
  MISC_FEATURE: Murine SC17.63 VL
SIVMTQTPKFLLVSAGDRVAITCKASQSVSNDVAWYQQKPGQSPTLLISYASNRYTGVPDRFTGSGYGTDFTFTISTVQAEDLAVYFCQQGYSSPFTFGGGTKLEIK

SEQ ID NO: 101 - protein, 116 aa, Mus musculus
  MISC_FEATURE: Murine SC17.63 VH
QVQLQQSDAELVKPGASVKISCKAAGYTFTDLTIHWVKQRPEQGLEWIGYIYPGDSNTKYNEKFKGKATLTADKSSSTAYMQLNSLTSEDSVVYFCARMITPYYFDYWGQGTTLTV

SEQ ID NO: 102 - protein, 107 aa, Mus musculus
  MISC_FEATURE: Murine SC17.71 VL
DIQMTQSPASLSASVGETVTIACRASGNIHNYLTWYQQRQGKSPQLLVYNAKTLAVGVPSRFSGSGSGTQYSLKINSLQPEDFGSYYCQHFWNTPPTFGGGTKLEIK

SEQ ID NO: 103 - protein, 119 aa, Mus musculus
  MISC_FEATURE: Murine SC17.71 VH
EVKLEESGGGLVQPGGSMKLSCVASGIIFSNYWMNWVRQSPEKGLEWVAEIRLKSNNYSTHYAESVKGRFTISRDDSKSSVYLQMNNLRAEDTGIYYCTRHYYYAMDYWGQGTSVTVSS

SEQ ID NO: 104 - protein, 107 aa, Mus musculus
  MISC_FEATURE: Murine SC17.72 VL
DIQMTQTTSSLSASLGDRVTISCSASQGISNYLNWYQQKPDGTVKLLIYYTSSLHSGVPSKFSGSGSGTDYSLTISNLEPEDIATYYCQQYSKLPYTFGGGTKLEIK

SEQ ID NO: 105 - protein, 119 aa, Mus musculus
  MISC_FEATURE: Murine SC17.72 VH
EVQLVESGGGLVKPGGSLKLSCAASGFTFSSYGMSWVRQTPEKRLEWVAAINSNGGSTYYPDTVKGRLTISRDNGKNTLYLQMSSLRSEDTALYYCVRDDGYYVFFAYWGQGTLVTVSA

SEQ ID NO: 106 - protein, 107 aa, Mus musculus
  MISC_FEATURE: Murine SC17.74 VL
DIQMTQSSSYLSVSLGGRVTITCKASDHINNWLAWYQQKPGNAPRLLISGATSLETGVPSRFSGSGSGKDYTLSITSLQTEDVATYYCQQYWSTPPTFGAGTKLELK

SEQ ID NO: 107 - protein, 119 aa, Mus musculus
  MISC_FEATURE: Murine SC17.74 VH
QVQLKQSGPGLVAPSQSLSITCTVSGFSLTSYGVDWVRQSPGKGLEWLGVIWGGGSTNYNSALKSRLSITKDNSKSQVFLKMNSLQTDDTAMYYCASGDYDGSLWFAYWGQGTLVTVSA

SEQ ID NO: 108 - protein, 112 aa, Mus musculus
  MISC_FEATURE: Murine SC17.76 VL
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPRTFGGGTKLEIK

SEQ ID NO: 109 - protein, 113 aa, Mus musculus
  MISC_FEATURE: Murine SC17.76 VH
EVQLVESGGDLVKPGGSLKLSCVASGFTFSSYGMSWVRQTPDKRLEWVATISSGGTFTYYPDSVKGRFTVSRDNAKNTLYLQMSSLKSEDTAMYYCSRHGWGWGQGTLVTVSA

SEQ ID NO: 110 - protein, 107 aa, Mus musculus
  MISC_FEATURE: Murine SC17.77 VL
DIQMTQSPASLSASVGETVTITCRASGNIHNYLAWYQQKQGKSPQLLVYNAKALADGVPSRFSGSGSGTQYSLKINSLQPEDFGSYYCQHFWSIPPTFGGGTKLEIK

SEQ ID NO: 111 - protein, 119 aa, Mus musculus
  MISC_FEATURE: Murine SC17.77 VH
EVKLEESGGGLVQPGGSMKLSCVASGFTFSNYWMNWVRQSPEKGLEWVAEIRLKSNNYATHYAESVKGRFTISRDDSKSSVYLQMNNLRVEDTAIYYCTRHYDYAMDYWGQGTSVTVSS

SEQ ID NO: 112 - protein, 107 aa, Mus musculus
  MISC_FEATURE: Murine SC17.79 VL
DIQMTQSPASLSASVGETVTITCRASGNIHNYLAWYQQKQGKSPQLLVYNAKTLADGVPSRFSGSGSGTQYSLRINSLQPEDFGSYYCQHFWSTPPTFGGGTKVEIK

SEQ ID NO: 113 - protein, 119 aa, Mus musculus
  MISC_FEATURE: Murine SC17.79 VH
EVKLEESGGGLVQPGGSMKLSCVASGFTFSDYWMNWVRQSPEKGLELVAEIRLISNNYATHYAESVKGRFTISRDDSKSSVYLQMNNLRAEDTGIYYCTRHYYYALDYWGQGTSVTVSS

SEQ ID NO: 114 - protein, 113 aa, Mus musculus
  MISC_FEATURE: Murine SC17.81 VL
DIVMSQSPSSLTVSVGEKVTLSCKSSQSLLYSTNQKIYLAWYQQKPGQSPKLLIYWASTRESGVPDRFTGSGSGTDFTLAISNVKAEDLAVYYCQQYYSYPYTFGGGTKLEIK

SEQ ID NO: 115 - protein, 113 aa, Mus musculus
  MISC_FEATURE: Murine SC17.81 VH
EVQLQQSGAELVKPGASVKLSCTASGFNINDTYYHWLKQRPEQGLEWIGRIDPANVNTKYDPKFQGKATLTADTSSNTAYLQLSSLTSEDTAVYYCGRGNAYWGQGTLVTVSA

SEQ ID NO: 116 - protein, 104 aa, Mus musculus
  MISC_FEATURE: Murine SC17.82 VL
QIVLTQSPAIMSASLGEEITLTCSASSSVSYMHWYQQKSGTSPKLLIYSTSNLASGVPSRFSGSGSGTFYSLTISSVEAEDAADYYCHQWSSFTFGSGTKLEIK

SEQ ID NO: 117 - protein, 120 aa, Mus musculus
  MISC_FEATURE: Murine SC17.82 VH
EVQLQQSGPELVKPGASVKMSCKASGYTFTDSYMNWVKQSHGKSLEWIGRVNPNNGGASYNHKFKGKATLTVDKSLSTAYMRLNSLTSEDSAVYYCSRSGDLYYYAMDYWGQGTSVTVSS

SEQ ID NO: 118 - protein, 106 aa, Mus musculus
  MISC_FEATURE: Murine SC17.84 VL
QIVLTQSPAIMSASPGEKVTMTCSASSSISYMHWYQQKSGTSPKRWIYDTSKLASGVPARFSGSGSGTSYSLTISNMEAEDAATYYCQQWSSTPPTFGGGTKLEIK

SEQ ID NO: 119 - protein, 117 aa, Mus musculus
  MISC_FEATURE: Murine SC17.84 VH
EVQLQQSGPELMKPGASVKMSCKASGYIFTDYNMHWVKQNQGKSLEWIGEVNPNTGGIGYNQKFKGKATLTVDKSSSTAYMDLRSLTSEDSAVYYCARDGNYCFDYWGQGTTLTVSS

SEQ ID NO: 120 - protein, 111 aa, Mus musculus
  MISC_FEATURE: Murine SC17.85 VL
DIVMTQAAFSNPVTLGTSASISCRSSKSLLHSNGITYLYWYLQKPGQSPQLLIYQMSNLASGVPERFSSSGSGSDFTLRISRVEAEDVGVYYCAQNLEHPTFGGGTKLEIK

SEQ ID NO: 121 - protein, 120 aa, Mus musculus
  MISC_FEATURE: Murine SC17.85 VH
EVQLVESGGDLVKPGGSLKLSCAASGFTFSNYGMSWVRQTPDKRLEWVATISTGGTYTYYPDSVKGRFTISRDNAKNTLYLQMSSLKSEDTAMYYCVGQSYSDYVSFAYWGQGTQVTVSA

SEQ ID NO: 122 - protein, 112 aa, Mus musculus
  MISC_FEATURE: Murine SC17.87 VL
DVVMTQTPLSLPVSLGDQASISCRSSQSLVHSNGNTYLHWYLQKPGQSPKLLISKVSNRFSGVPDRFSGSGSGTDFTLKISRVEAEDLGVYFCSQSTHVPPMFGGGTRLEIK

SEQ ID NO: 123 - protein, 115 aa, Mus musculus
  MISC_FEATURE: Murine SC17.87 VH
EVQLQQSGAELLKPGASVKLSCTASGLNIKDYYIHWVYQRPEQGLEWIGRIDPESDNTLYDPKFQGKASITADTSSNTAYLQLSSLTSEDTAVYYCTTNTPFAYWGQGTLVTVST

SEQ ID NO: 124 - protein, 112 aa, Mus musculus
  MISC_FEATURE: Murine SC17.89 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 125 - protein, 120 aa, Mus musculus
  MISC_FEATURE: Murine SC17.89 VH
QVQLQQSGAELVRPGTSVKVSCKTSGYAFTNYLIEWVKQRPGQGLEWIGVINPGSGGTNYNEKFKVKATLTADKSSSTAYMQLTSLTSDDSAVYFCTRRDGYFFPWFAYWGQGTLVTVSA

SEQ ID NO: 126 - protein, 113 aa, Mus musculus
  MISC_FEATURE: Murine SC17.90 VL
DIVMSQSPSSLAVSVGEKVTMSCKSSQSLLYSSNQKNYLAWYQQKPGQSPKLLIYWASTRKSGVPDRFTGSGSGTDFTLTISSVKAEDLAVYYCHQYYSYPLTFAAGTKLELK

SEQ ID NO: 127 - protein, 115 aa, Mus musculus
  MISC_FEATURE: Murine SC17.90 VH
QVQLQQPGSVLVRPGASVKLSCKASGYTFTSYWMHWVKQRPGQGLEWIGEIHPNNGSTNYNEKFKGKATLTVDTSSSTAYVDLSSLTSEDSAVYYCARWTLFTYWGQGTLVTVSA

SEQ ID NO: 128 - protein, 112 aa, Mus musculus
  MISC_FEATURE: Murine SC17.91 VL
DVVMTQTPLSLPVSLGDQASISCRSSQSLVHSNGNTYLLWYLQKPGQSPKLLIYKVSNRFSGVPDRFSGSGSGTDFALKISRVEAEDLGVYFCSQSTHVPWTFGGGTKLEIK

SEQ ID NO: 129 - protein, 117 aa, Mus musculus
  MISC_FEATURE: Murine SC17.91 VH
EVQLVESGGGLVKPGGSRKLSCAASGFTFSDYGMHWVRQAPEKGLEWVAYISRGSSTIHYADTVKGRFTISRDNAKNTLFLQMTSLRSEDTAMYYCARPFNWYFDVWGAGTTVTVSS

SEQ ID NO: 130 - protein, 113 aa, Mus musculus
  MISC_FEATURE: Murine SC17.93 VL
DIVMSQSPSSLAVSVGEKVTMTCKSSQSLLYSSNQKNYLAWYQQKPGQSPKLLIYWASTRESGVPDRFIGSGSGTDFTLTISSVKAEDLAIYYCQQYYRYPLTFGAGTKLELK

SEQ ID NO: 131 - protein, 121 aa, Mus musculus
  MISC_FEATURE: Murine SC17.93 VH
QVQLQQPGAELVKPGASVMLSCKASGYTFTSYWVHWVKQRPGQGLEWIGVINPRNGRNNYNEKFKTKATLTVDKSSSTAYMQLSSPTSEDSAVYYCAREDYDGGDYAMDYWGQGTSVTVSS

SEQ ID NO: 132 - protein, 107 aa, Mus musculus
  MISC_FEATURE: Murine SC17.95 VL
DIQMTQTTSSLSASLGDRVTISCSASQGINNYLNWYQQKPDGTVTLLIYYTSSLHSGVPSRFSGSGSGTDYSLTISNLEPEDIATYYCQQYSKLPWTFGGGTKLEIK

SEQ ID NO: 133 - protein, 121 aa, Mus musculus
  MISC_FEATURE: Murine SC17.95 VH
EVELQQSGPELVKPGASVKISCKTSGNTYTEYTMQWVKLSHGKSLEWIGGINPNNGITSYNQKFKGKATLTVDKSSSTAYMELRSLKSEDSAVYYCARAGLGNYVWAMDYWGQGASVTVSS

SEQ ID NO: 134 - protein, 112 aa, Mus musculus
  MISC_FEATURE: Murine SC17.97 VL
DVVMTQTPLSLPVSLGDQASISCRSSQSLVHNNGNTYLHWYLQKPGQSPNLLIYKVSNRFSGVPDRFSGSGSGTDFTLKISIVEAEDLGLYFCSQSTHVPRTFGGGTKLEIK

SEQ ID NO: 135 - protein, 119 aa, Mus musculus
  MISC_FEATURE: Murine SC17.97 VH
QVQLPQSGAELAKPGASVKISCKASGFTFTSYWMHWVKQRPGQGLEWIGYINPSTDYTEYNQKFKDKATLTADKSSSTAYMQLGSLTSEDSAVYYCARSSYGSSPFDYWGQGSTLTVSS

SEQ ID NO: 136 - protein, 113 aa, Mus musculus
  MISC_FEATURE: Murine SC17.99 VL
DIVMSQSPSSLAVSVGEKVTMNCESSQSLLYSSNQKNYLAWYQQKPGQSPKLLIYWASTRDSGVPDRFTGSGSGTDFTLTISSVRAEDPAVYYCQQYYSYPLTFGAGTKLELR

SEQ ID NO: 137 - protein, 116 aa, Mus musculus
  MISC_FEATURE: Murine SC17.99 VH
EVKLEESGGGLVQPGGSMKLSCAASGFTFSDAWMDWVRQSPEKGLEWVAEIRSKANNHATYYAESVKGRFTISRDDSKSSAYLQMNSLRAEDTGIYYCVSTGTSYWGQGTLVTVSA

SEQ ID NO: 138 - protein, 106 aa, Mus musculus
  MISC_FEATURE: Murine SC17.102 VL
QIVLTQSPAIMSASPGEKVTMTCSASSSVSYMHWYQQKSGTSPKRWIYDTSKLASGVPPRFSGRGSGTSYSLTISSMEAEDAATYYCQHWSSNPPTFGAGTKLEMK

SEQ ID NO: 139 - protein, 116 aa, Mus musculus
  MISC_FEATURE: Murine SC17.102 VH
EVQLQQSGPELMKPGASVKMSCKASGDTFTDYNIHWVKQNQGKSLEWIGEVNPNIGGIGYNQKFKGKATLTVDKSSSTAYMELRSLTSEDSAVYYCAMGRWYFDVWGAGTTVTVSS

SEQ ID NO: 140 - protein, 112 aa, Mus musculus
  MISC_FEATURE: Murine SC17.114 VL
DVVMTQSPLSLPVSLGDQASISCRSSQSLVHSNGNTYLHWYLQKPGQSPKLLIYKVSSRFSGVPDRFSGSGSGTDFTLKISRVEAEDLGVYFCSQSTHVPFTFGSGTKLEIK

SEQ ID NO: 141 - protein, 115 aa, Mus musculus
  MISC_FEATURE: Murine SC17.114 VH
EVQLQQSGPEMVKPGASVKISCKASGYTFTDYYMHWVKQSHGKSLEWIGRVNTNNGGTSYDQKFEGKATLTVDKSSSTAYMELNSLTSEDSAVYYCVIPAWFAYWGQGTLVTVSA

SEQ ID NO: 142 - protein, 112 aa, Mus musculus
  MISC_FEATURE: Murine SC17.115 VL
DIVMTQSPLSLPVSLGDQASISCRSSQSLVHSNGNTYLHWYLQKPGQSPKLLIYRVSNRFSGVPDRFSGSGSGTDFTLTISRVEAEDLGVYFCSQSTHLPRTFGGGTKLEIK

SEQ ID NO: 143 - protein, 114 aa, Mus musculus
  MISC_FEATURE: Murine SC17.115 VH
QVQLQQSGSVLVRPGASVKLSCKASGYTFTSYWMHWVKQRPGQGLEWIGEIHPNSGNTNYNEKFKGKATLTVDTSSSTAYVDLSSLTSEDSAVYYCAGGNYDYWGQGTTLTVSS

SEQ ID NO: 144 - protein, 111 aa, Mus musculus
  MISC_FEATURE: Murine SC17.120 VL
DIVLTQSPASLAVSLGQRATISCRASESVDSYGNSFMHWYQQKPGQPPKVLIYRASNLESGIPARFSGSGSRTDFTLTINPVEDEDVATYYCQQSNEDPYTFGGGTKLEIK

SEQ ID NO: 145 - protein, 117 aa, Mus musculus
  MISC_FEATURE: Murine SC17.120 VH
EVQLEQSGTVLARPGASVKMSCKASGYTFTSYWMHWVKQRPGQGLEWIGAFYPGNSGTYYNQKFKDKAKLTAVTSASTAYMELSSLTNEDSAVYYCSRSGSGRFAYWGQGTLVTVSA

SEQ ID NO: 146 - protein, 106 aa, Mus musculus
  MISC_FEATURE: Murine SC17.121 VL
QIVLTQSPAIMSASPGEKVTMTCSASSSVSYMHWYQQKSGTSPKRWIYDTSKLASGVPARFSGSGSGTSYSLTISSMETEDAATYYCQQWSNTPPTFGSVTKLEIK

SEQ ID NO: 147 - protein, 116 aa, Mus musculus
  MISC_FEATURE: Murine SC17.121 VH
EVQLQQSGPELMKPGASVKMSCKASGYTFTDHNIHWVKQHQGKSLEWIGEINPNTGGTGYNQKFQGKATMTVDKSSSTAYMELRSLTSEDSAVYYCVRGLYFFDYWGQGTTLTVSS

SEQ ID NO: 148 - protein, 112 aa, Mus musculus
  MISC_FEATURE: Murine SC17.122 VL
DIVITQDDLSNPVTSGESVSISCRSSKSLLYKDGKTYLNWFLQRPGQSPQLLIYLMSTRASGVSDRFSGSGSGTDFTLEISRVKAEDVGVYYCQQLVEYPRTFGGGTKLEIK

SEQ ID NO: 149 - protein, 113 aa, Mus musculus
  MISC_FEATURE: Murine SC17.122 VH
EVHLVESGGDLVKPGGSLKLSCAASGFTFSSYGMSWVRQTPDKRLEWVATISSGGTYTYYPDSVKGRFTISRDNAKNTLYLQMSSLKSEDTAMYYCSRHGWGWGQGTLVTVSA

SEQ ID NO: 150 - protein, 106 aa, Mus musculus
  MISC_FEATURE: Murine SC17.140 VL
QIVLTQSPAIMSASPGEKVTMTCSASSSVSYMHWYQQKSGTSPKRWIYDTSKLASGVPARFSGSGSGTSYSLTISSMEAEDAATYYCQQWSSTPPTFGSGTKLEIK

SEQ ID NO: 151 - protein, 120 aa, Mus musculus
  MISC_FEATURE: Murine SC17.140 VH
EVQLQQSGPELMKPGASVKMSCKASGYTFTDYNMHWVKQNQGKSLEWIGEINPNTGGTGYNQKFKGKATLTVDKFSSTAFIELRSLTSEDSAIYYCTRGGYDHYWYFDVWGAGTTVTVSS

SEQ ID NO: 152 - protein, 111 aa, Mus musculus
  MISC_FEATURE: Murine SC17.151 VL
DIVLTQFPASLAVSLGQRATIPCRASESVDSYGNSFMHWFQQKPGQPPKLLIYRASNLESEIPARFSGSGSGTDFTLTINPVEADDVATYYCQQSHEDPYTFGGGTKMEIK

SEQ ID NO: 153 - protein, 117 aa, Mus musculus
  MISC_FEATURE: Murine SC17.151 VH
EVQLQQSGTVLARPGASVKMSCKASGYTFTSYWMHWVKQRPGQGLEWIGAIYPGKNDTTYNQKFKGKAKLTAVTSASTLYMELSSLTNEDSAVYYCTRSGKGYFAYWGQGTLVTVSA

SEQ ID NO: 154 - protein, 112 aa, Mus musculus
  MISC_FEATURE: Murine SC17.156 VL
DVVMTQSPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 155 - protein, 118 aa, Mus musculus
  MISC_FEATURE: Murine SC17.156 VH
QVTLKESGPGILQPSQTLSLTCSFSGFSLSTSGMGVSWIRKTSGKGLEWLAHIFWDDDKWYNPSLKSRLTISKATSSNQVFLILTSVDTADTATYYCATFYGLYFAYWGQGTTLTVSS

SEQ ID NO: 156 - protein, 113 aa, Mus musculus
  MISC_FEATURE: Murine SC17.161 VL
DIVMSQSPSSLAVSVGEKVTMNCESSQSLLYNSNQKNYLAWYQQKPGQSPKLLIYWASTRDSGVPDRFTGSGSGTDFTLTISSVRADDPAVYYCQQYFNYPLTFGAGTKLELK

SEQ ID NO: 157 - protein, 116 aa, Mus musculus
  MISC_FEATURE: Murine SC17.161 VH
EVKLEESGGGLVQPGGSMKLSCAASGFTFSDAWMDWVRQSPEKGLEWVAEIRSKPNNHATYYAESVKGRFTISRDDSKSSAYLQMNSLRAEDTGIYYCVSTGTSYWGQGTLVTVSA

SEQ ID NO: 158 - protein, 106 aa, Mus musculus; identical to SEQ ID NO: 118
  MISC_FEATURE: Murine SC17.166 VL

SEQ ID NO: 159 - protein, 117 aa, Mus musculus; identical to SEQ ID NO: 119
  MISC_FEATURE: Murine SC17.166 VH

SEQ ID NO: 160 - protein, 107 aa, Mus musculus
  MISC_FEATURE: Murine SC17.187 VL
DIKMTQSPSSMYASLGERVTLTCKASQDINSYLSWFQQKPGKSPETLIYRANRLIDGVPSRFSGSGSGQDYSLTISSLEYEDMGIYYCLQYDEFPPTFGGGTKLEIK

SEQ ID NO: 161 - protein, 118 aa, Mus musculus
  MISC_FEATURE: Murine SC17.187 VH
EVHLQQSGPELVNPGSSVKISCKAAGYTFTDYNMDWVKQSHGKRLEWIGNIYPNNGGAGYNQNFKDKATLTVDKSSSTAYMELRSLTSEDSAVYYCARSITAAWFAYWGQGTLVTVSA

SEQ ID NO: 162 - protein, 106 aa, Mus musculus
  MISC_FEATURE: Murine SC17.191 VL
QIVLTQSPAIMSASPGEKVTMTCSASSSVSYMHWYQQKSGTSPKRWIYDTSKLASGVPARFTGSGSGTSYSLTISSMEAEDAATYYCQQWSSSPPTFGAGTKLELK

SEQ ID NO: 163 - protein, 120 aa, Mus musculus
  MISC_FEATURE: Murine SC17.191 VH
EVQLQQSGPELMKPGASVKMSCKASGYTFTDYNMHWVKQNQGKSLEWIGEINPNTGGTGYNQKFKDKATLTVDKSSSTAYMELRSLTSEDSAVYYCARIPSLRRYYFDYWGQGTTLTVSS

SEQ ID NO: 164 - protein, 111 aa, Mus musculus
  MISC_FEATURE: Murine SC17.193 VL
DLVLTQSPASLAVSLGQRATISCRASESVSTSGYSYMHWYQQKPGQPPKLLIYLASNLESGVPARFSGSGSGTDFTLNIHPVEEEDATTYYCQHSRELPYTFGGGTKLEIK

SEQ ID NO: 165 - protein, 122 aa, Mus musculus
  MISC_FEATURE: Murine SC17.193 VH
QVTLKESGPGILQPSQTLSLTCSFSGFSLITYGIGVGWIRQPSGKGLEWLAHIWWNDNKYYNTALKSRLTISKDTSNNQVFLKIANVDTADTATYYCARMVYYDYDGGFAYWGQGTLVTVSA

SEQ ID NO: 166 - protein, 111 aa, Mus musculus
  MISC_FEATURE: Murine SC17.199 VL
DIVLTQSPASLAVSLGQRATISCRASESVDSYGNSFMHWYQQKPGQPPKPLIYRASNLESGIPARFSGSGSRTDFTLTINPVEADDVATYYCQQSNEDPYTFGGGTKLEIK

SEQ ID NO: 167 - protein, 116 aa, Mus musculus
  MISC_FEATURE: Murine SC17.199 VH
EVQLQQSGTVLARPGASVRMSCKASGYTFTSYWMHWVKQRPGQGLEWIGAIYPGNSDTSYNHKFKGKAKLTAVTSASTAYMELSSLTNEDSAVYYCTRSGTGWFAYWGQGTLVTVS

SEQ ID NO: 168 - protein, 111 aa, Mus musculus
  MISC_FEATURE: Murine SC17.200 VL
DIVLTQSPASLAVSLGQRATIFCRASQSVDYNGISYMHWFQQKPGQPPKLLIYAASNVQSGIPARFSGSGSGTDFTLNIHPVEEEDAATFYCQQSIEDPPTFGGGTKLEIK

SEQ ID NO: 169 - protein, 115 aa, Mus musculus
  MISC_FEATURE: Murine SC17. VH
  MISC_FEATURE: Murine SC17.200 VH
QVQLQQSGPELVKPGASVKISCKASGYAFSSSWINWVKQRPGQGLEWIGRIYPGEGDTNYSGNFEGKATLTADKSSTTAYMQLSSLTSVDSAVYFCTRGLVMDYWGQGTALTVSS

SEQ ID NO: 170 - protein, 107 aa, Artificial Sequence
  Humanized hSC17.16 VL
DIQMTQSPSSLSASVGDRVTITCRASANINSNLVWYQQKPGKAPKLLIYAATNLADGVPSRFSGSGSGTDFTLTISSLQPEDFATYYCQHFWGTPRTFGGGTKLEIK

SEQ ID NO: 171 - protein, 116 aa, Artificial Sequence
  Humanized hSC17.16 VH
QVQLVQSGAEVKKPGASVKVSCKASGYTFTDYNMYWVRQAPGQGLEWMGEINPNNGGTAYNQKFRGKVTMTRDTSISTAYMELSRLRSDDTAVYYCARYDKGFDYWGQGTTVTVSS

SEQ ID NO: 172 - protein, 106 aa, Artificial Sequence
  Humanized hSC17.17 VL
EIVLTQSPATLSLSPGERATLSCSASSSVSYMHWYQQKPGQAPRLLIYDTSKLPSGIPARFSGSGSGTDFTLTISSLEPEDFAVYYCQQWSSTPPTFGQGTKLEIK

SEQ ID NO: 173 - protein, 120 aa, Artificial Sequence
  Humanized hSC17.17 VH
QVQLVQSGAEVKKPGASVKVSCKASGYTFTDYNMHWVRQAPGQGLEWMGEINPNIGGTGYNQKFKGRVTMTRDTSISTAYMELSRLRSDDTAVYYCARTYSYYSYEFAYWGQGTLVTVSS

SEQ ID NO: 174 - protein, 112 aa, Artificial Sequence
  Humanized hSC17.24 VL
DIVMTQSPDSLAVSLGERATINCKSSQSLLYSSNQKSYLAWYQQKPGQPPKLLIYWASTRESGVPDRFSGSGSGTDFTLTISSLQAEDVAVYYCKQSYNLRTFGGGTKVEIK

SEQ ID NO: 175 - protein, 117 aa, Artificial Sequence
  Humanized hSC17.24 VH
EVQLVQSGAEVKKPGATVKISCKVSGYTFTDHTIHWVRQAPGKGLEWIGYIYPRDGSTKYNEEFKGRVTITADTSTDTAYMELSSLRSEDTAVYYCARSYSNYFDYWGQGTTVTVSS

SEQ ID NO: 176 - protein, 107 aa, Artificial Sequence
  Humanized hSC17.28 VL
EIVLTQSPDFQSVTPKEKVTITCRASQSIGTSIHWYQQKPDQSPKLLIKYASESISGVPSRFSGSGSGTDFTLTINSLEAEDAATYYCQQSNSWPLTFGQGTKLEIK

SEQ ID NO: 177 - protein, 117 aa, Artificial Sequence
  Humanized hSC17.28 VH
QVQLVQSGAEVKKPGASVKVSCKASGYTFTRSYIHWVRQAPGQGLEWMGYISSGSGGTTYNQKFKGRVTSTRDTSISTAYMELSRLRSDDTAVYYCARGGVRYFDVWGQGTTVTVSS

SEQ ID NO: 178 - protein, 107 aa, Artificial Sequence
  Humanized hSC17.34 VL
DIQMTQSPSSLSASVGDRVTITCKASQDINSYLSWFQQKPGKAPKSLIYRANRLVDGVPSRFSGSGSGTDYTLTISSLQPEDFATYYCLQYDEFPPTFGQGTKLEIK

SEQ ID NO: 179 - protein, 118 aa, Artificial Sequence
  Humanized hSC17.34 VH
QVQLVQSGAEVKKPGASVKVSCKASGYTFTDYNMDWVRQAPGQRLEWIGYIYPDNGGAGYNQKFKGRVTITVDTSASTAYMELSSLRSEDTAVYYCSRSITTAWFAYWGQGTLVTVSS

SEQ ID NO: 180 - protein, 107 aa, Artificial Sequence
  Humanized hSC17.46 VL
AIQMTQSPSSLSASVGDRVTITCKASQSVNNDVAWYQQKPGKAPKLLIYYASNRYTGVPSRFSGSGSGTDFTLTISSLQPEDFATYFCQQDYSSPRTFGQGTKLEIK

SEQ ID NO: 181 - protein, 121 aa, Artificial Sequence
  Humanized hSC17.46 VH
QVQLVQSGAEVKKPGASVKVSCKASGYTFTSYWINWVRQAPGQGLEWIGNIFPDTTTTNYNEKFKGRVTLTRDTSISTAYMELSRLRSDDTAVYYCAREYYDGTYDAMDYWGQGTLVTVSS

SEQ ID NO: 182 - protein, 111 aa, Artificial Sequence
  Humanized hSC17.151 VL
EIVLTQSPATLSLSPGERATLSCRASESVDSYGNSFMHWYQQKPGQAPRLLIYRASNLESGIPARFSGSGSGTDFTLTISSLEPEDFAVYYCQQSHEDPYTFGQGTKLEIK

SEQ ID NO: 183 - protein, 117 aa, Artificial Sequence
  Humanized hSC17.151 VH
QVQLVQSGAEVKKPGASVKVSCKASGYTFTSYWMHWVRQAPGQGLEWMGAIYPGKSDTTYNQKFKGRVTMTRDTSTSTVYMELSSLRSEDTAVYYCARSGKGYFAYWGQGTLVTVSS

SEQ ID NO: 184 - protein, 113 aa, Artificial Sequence
  Humanized hSC17.155 VL
DIVMTQSPDSLAVSLGERATINCKSSQSLLYSSNQKNYLAWYQQKPGQPPKLLIYWASTRKSGVPDRFSGSGSGTDFTLTISSLQAEDVAVYYCHQYYSYPLTFGQGTKLEIK

SEQ ID NO: 185 - protein, 113 aa, Artificial Sequence
  Humanized hSC17.155 VH
QVQLVQSGAEVKKPGASVKVSCKASGYTFNSYWMHWVRQAPGQGLEWMGEIHPNNGSTNYNEKFKGRVTMTRDTSTSTVYMELSSLRSEDTAVYYCARWTLFTYWGQGTLVTV

SEQ ID NO: 186 - protein, 112 aa, Artificial Sequence
  Humanized hSC17.156 VL
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPPTFGGGTKLEIK

SEQ ID NO: 187 - protein, 118 aa, Artificial Sequence
  Humanized hSC17.156 VH
QVTLKESGPVLVKPTETLTLTCTVSGFSLSTSGMGVSWIRQPPGKALEWLAHIFWDDDKWYNPSLKSRLTISKDTSKSQVVLTMTNMDPVDTATYYCATFYGLYFAYWGQGTLVTVSS

SEQ ID NO: 188 - protein, 113 aa, Artificial Sequence
  Humanized hSC17.161 VL
DIVMTQSPDSLAVSLGERATINCESSQSLLYNSNQKNYLAWYQQKPGQPPKLLIYWASTRESGVPDRFSGSGSGTDFTLTISSLQAEDVAVYYCQQYFNYPLTFGQGTKLEIK

SEQ ID NO: 189 - protein, 116 aa, Artificial Sequence
  Humanized hSC17.161 VH
QVQLVQSGAEVKKPGASVKVSCKASGFTFSDAWMDWVRQAPGQRLEWMGEIRSKPNNHATYYAESVKGRVTITRDTSASTAYMELSSLRSEDTAVYYCARTGTSYWGQGTLVTVSS

SEQ ID NO: 190 - protein, 111 aa, Artificial Sequence
  Humanized hSC17.200 VL
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIK

SEQ ID NO: 191 - protein, 115 aa, Artificial Sequence
  Humanized hSC17.200 VH
EVQLVQSGAEVKKPGESLKISCKGSGYSFTSSWINWVRQMPGKGLEWMGRIYPGEGDTNYSGNFEGQVTISADKSISTAYLQWSSLKASDTAMYYCTRGLVMDYWGQGTLVTVSS

SEQ ID NO: 192 - protein, 111 aa, Artificial Sequence
  Humanized hSC17.200 VL
EIVLTQSPATLSLSPGERATLSCRASQSVDYDGISYMHWYQQKPGQAPRLLIYAASNVQSGIPARFSGSGSGTDFTLTISSLEPEDFAVYYCQQSIEDPPTFGGGTKVEIK

SEQ ID NO: 193 - protein, 113 aa, Artificial Sequence
  Humanized hSC17.155 VH1
QVQLVQSGAEVKKPGASVKVSCKASGYTFDSYWMHWVRQAPGQGLEWMGEIHPNNGSTNYNEKFKGRVTMTRDTSTSTVYMELSSLRSEDTAVYYCARWTLFTYWGQGTLVTV

SEQ ID NO: 194 - protein, 113 aa, Artificial Sequence
  Humanized hSC17.155 VH2
QVQLVQSGAEVKKPGASVKVSCKASGYTFTSYWMHWVRQAPGQGLEWMGEIHPNNGSTNYNEKFKGRVTMTRDTSTSTVYMELSSLRSEDTAVYYCARWTLFTYWGQGTLVTV

SEQ ID NO: 195 - protein, 113 aa, Artificial Sequence
  Humanized hSC17.155 VH3
QVQLVQSGAEVKKPGASVKVSCKASGYTFNYYWMHWVRQAPGQGLEWMGEIHPNNGSTNYNEKFKGRVTMTRDTSTSTVYMELSSLRSEDTAVYYCARWTLFTYWGQGTLVTV

SEQ ID NO: 196 - protein, 113 aa, Artificial Sequence
  Humanized hSC17.155 VH4
QVQLVQSGAEVKKPGASVKVSCKASGYTFNSYWMHWVRQAPGQGLEWMGEIHPNDGSTNYNEKFKGRVTMTRDTSTSTVYMELSSLRSEDTAVYYCARWTLFTYWGQGTLVTV

SEQ ID NO: 197 - protein, 113 aa, Artificial Sequence
  Humanized hSC17.155 VH5
QVQLVQSGAEVKKPGASVKVSCKASGYTFNSYWMHWVRQAPGQGLEWMGEIHPNGGSTNYNEKFKGRVTMTRDTSTSTVYMELSSLRSEDTAVYYCARWTLFTYWGQGTLVTV

SEQ ID NO: 198 - protein, 113 aa, Artificial Sequence
  Humanized hSC17.155 VH6
QVQLVQSGAEVKKPGASVKVSCKASGYTFNSYWMHWVRQAPGQGLEWMGEIHPNSGSTNYNEKFKGRVTMTRDTSTSTVYMELSSLRSEDTAVYYCARWTLFTYWGQGTLVTV

SEQ ID NO: 199 - protein, 116 aa, Artificial Sequence
  Humanized hSC17.161 VH1
EVQLVESGGGLVQPGGSLRLSCAASGFTFSDAWMDWVRQAPGKGLEWVGEIRSKPNNHATYYAESVKGRFTISRDDSKNSLYLQMNSLKTEDTAVYYCARTGTSYWGQGTLVTVSS

SEQ ID NO: 200 - protein, 4 aa, Homo sapiens
  MISC_FEATURE: C-terminal cytoplasmic domain motif
NPTY

SEQ ID NO: 201 - protein, 9 aa, Artificial Sequence
  His tag
HHHHHHHHH

SEQ ID NO: 202-219 - skipped (18 entries)

SEQ ID NO: 220 - DNA, 321 bp, Mus musculus
  misc_feature: Murine SC17.1 VL
caaattgttctcacccagtctccagcactcatgtctgcatctccaggggaaaaggtctccctgacctgcagtgccaactcaactgtaagtttcatgtactggtaccagcagaagccaagatcctcccccacaccctggatttatctcacatccaacctggcttctggagtccctgctcgcttcagtggcagtgggtctgggacctcttactctcttacaatcagcagcatggaggctgaagatgctgccacttattactgccagcagtggagtagtaactcacccatcacgttcggtgctgggaccaagctggagctgaaa

SEQ ID NO: 221 - DNA, 351 bp, Mus musculus
  misc_feature: Murine SC17.1 VH
gatgtgcagcttcaggactcaggacctggcctggtgaaaccttctcagtctctgtccgtcacctgcactgtcactggctactccatcacctggggttattactggaactggatccggcagtttccaggaaacaaactggagtggatgggtaacatacacaacagtggtggcactaactacaacccatctctcaagagtcgaatctctatcactcgagacacatccaagaaccagttcttcctgcagttgaattctgtgactactgaggacacagccacatattactgtgcaaccacaaactgggactactttgactactggggccaaggcaccactctcacagtctcctca

SEQ ID NO: 222 - DNA, 336 bp, Mus musculus
  misc_feature: Murine SC17.2 VL
gacattgtgatgtcacagtctccatcctccctggctgtgtcagttggagagaaggtcactatgagctgcaagtccagtcagagccttttatatagtagcaatcaaaagagctacttggcctggtaccagcagaaaccagggcagtctcctaaactgttaatctactgggcatccactagggaatctggggtccctgaccgcttcacaggcagtggatcagggacagatttcactctcaccatcagcagtgtgcaggctgaagacctggccgtttattactgcaagcaatcttataatcttcggacgttcggtggaggcaccaagctggaaatcaaa

SEQ ID NO: 223 - DNA, 351 bp, Mus musculus
  misc_feature: Murine SC17.2 VH
caggttcagctgcagcagtctgacgctgagttggtgaaacctggagcttcagtgaagatatcctgcaaggtttctggctacaccttcactgaccatactattcactggatgaagcagaggcctgaacagggcctggaatggattggatatatttatcctagagatggtagtactaagtacaatgaggagttcaagggcaaggccacattgactgcagacaaatcctccagcacagcctacatgcagctcaacagcctgacatctgaggactctgcagtctatttctgtgcaagatcatatagtaactactttgactactggggccaaggcaccactctcacagtctcctca

SEQ ID NO: 224 - DNA, 333 bp, Mus musculus
  misc_feature: Murine SC17.9 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttatttctgtcagcaatattataactatccgtacacgttcggaggggggaccaagctgaaa

SEQ ID NO: 225 - DNA, 357 bp, Mus musculus
  misc_feature: Murine SC17.9 VH
caggtccaactgcagcaacctggggctgaaattgtgaggcctggggcttcagtgaagctgtcctgcaaggcttctggctacacctttaccgactattggatgaactgggtaaaacagaggcctggacaaggccttgagtggatcggagcaattgatccttctgatagttatactagctacaatccaaaattcaagggcaaggccacattgactgtagacacctcctccagctcagcctacatgcagctcagcagcctgacatctgaggactctgcggtctatttctgtgcaagaagaggaacccctggtaaaccccttgtttactggggccaagggactctggtcactgtctctgca

SEQ ID NO: 226 - DNA, 321 bp, Mus musculus
  misc_feature: Murine SC17.16 VL
gacatccagatgactcagtctccagcctccctatctgtatctgtgggagaaactgtcaccatcacatgtcgagcaagtgcgaatattaacagtaatttagtatggtatcagcagaaacagggaaaatctcctcagctcctggtctatgctgcaacaaacttagcggatggtgtgccatcacggttcagtggcagtggatcaggcacacagtattccctcaagatcaacagcctgcagtctgaagattttgggaattactactgtcaacatttttggggtactcctcggacgttcggtggaggcaccaagctggaaatcaaa

SEQ ID NO: 227 - DNA, 348 bp, Mus musculus
  misc_feature: Murine SC17.16 VH
gaggtccagctgcaacagtctggacctgagctaatgaagcctggggcttcagtgaagatgtcctgcaaggcttctggatacacattcactgactacaacatgtactgggtgaagcagaaccaaggaaagagcctagagtggataggagaaattaatcctaacaatggtggtactgcctacaaccagaagttcagaggcaaggccacgttgactgtagacaagtcctccagcacagcctacatggagctccgcagcctgacatctgaggactctgcagtctattactgtgcaagatatgataaggggtttgactactggggccaaggcaccactctcacagtctcctca

SEQ ID NO: 228 - DNA, 333 bp, Mus musculus
  misc_feature: Murine SC17.38 VL
gacattgtggtcacccaatctccagcttctttggctgtgtctctggggcagagagccaccatctcctgcagagccagtgaaagtgttgaatattatggcacaagtttaatgcagtggttccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaagataggaaggttccttggacgttcggtggaggcaccaagctggaaatcaaa

SEQ ID NO: 229 - DNA, 360 bp, Mus musculus
  misc_feature: Murine SC17.38 VH
caggttactctgaaagagtctggccctgggatattgcagccctcccagaccctcagtctaacttgttctttctctgggttttcactgaacacatctggtatgagtgtaggctgggttcgtcagccttcagggaggggtctggaatggctggcccccatttggtggaatggtgataagtactataacccagccctgaaaagccggctcacaatctccaaggatacctccaacaaccaggttttcctcaagatcgccagtgtggtcactgcagatactgccacatacttctgtgctcgaatacggcaatattactatgctatggactactggggtcaaggaacctcagtcaccgtctcctca

SEQ ID NO: 230 - DNA, 336 bp, Mus musculus
  misc_feature: Murine SC17.3 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgacgttcggtggaggcaccaagctggaaatcaaa

SEQ ID NO: 231 - DNA, 354 bp, Mus musculus
  misc_feature: Murine SC17.3 VH
caggtccaactgcagcagcctggggctgagcttgtgaagcctggggcttcagtgaagctgtcctgcaaggcttctggctacaccttccccagctactggatacactgtgtgaagcagaggcctggacaaggccttgagtggattggagtgattaatcctagcaacggtcgtactaactacaatgagaagttcaagaacaaggccacactgactgtagacaaatcctccagcacagcctacatgcaactcagcagcctgacatctgaggactctgcggtctattactgtgtcaggggggggacgggctatactatggactactggggtcaaggaacctcagtcaccgtctcctca

SEQ ID NO: 232 - DNA, 321 bp, Mus musculus
  misc_feature: Murine SC17.4 VL
gacatcaagatgacccagtctccatcttccatgtatgcctctctaggagagagagtcactatcacttgcaaggcgagtcaggacattaatagctatttaacctggttccagcagaaaccagggaaatctcctaagaccctgatctatcgtgcaaacagattgatagatggggtcccatcaaggttcagtggcagtggatctgggcaagattattctctcaccatcagcagcctggattatgaagatatgggaatttattattgtctacagtatgatgactttccgtggacgttcggtggaggcaccaagctggaaatcaaa

SEQ ID NO: 233 - DNA, 348 bp, Mus musculus
  misc_feature: Murine SC17.4 VH
cagatccagttggtgcagtctggacctgagctgaagaagcctggagagacagtcaagatctcctgcaaggcttctggttataccttcacagactattcaatgcactgggtgaagcaggctccaggaaagggtttaaagtggctgggctggataaacactgagactggcgagccaacatattcagaagacttcaagggacggtttgccttctctttggaaacctctgccagcactgcctatttgcagatcaacaacctcaaaaatgaagacacggctacttatttctgtgttaaaaataagggctggtttgcttattggggccaagggactctggtcactgtctctgca

SEQ ID NO: 234 - DNA, 336 bp, Mus musculus
  misc_feature: Murine SC17.8 VL
gatgttgtgatgacccaaactccactctccctgcctgtcagtcttggagatcaagcctccatctcttgcagatctagtcagagccttgtacacagtaatggagacacctatttacattggtacctgcagaagccaggccagtctccaaaactcctgatctacaaagtttccaaccgattttctggggtcccagacaggttcagtggcagtggatcagggacagatttcacactcaagatcagcagagtggaggctgaggatctgggactttatttctgctctcaaagtacacttattccgtacacgttcggaggggggaccaagctggacataaaa

SEQ ID NO: 235 - DNA, 342 bp, Mus musculus
  misc_feature: Murine SC17.8 VH
caggttcacctgcagcagtctggaactgaagtgatgaagcctggggcctcagtgaagatatcctgcaaggctactggctacacattcagtagctactggatagagtggataaagcagaggcctggacatggccttgagtggattggagagattttgcctggaagtggtaatactaacaacaatgagaagttcaagggcaaggccacaatcactgcagatacatcctccaatatagcctacatacaattaagcagcctgacatctgaggactctgccgtctattactgtgcgggaggcccggcggcttactggggccaagggactctggtcactgtctctgca

SEQ ID NO: 236 - DNA, 339 bp, Mus musculus
  misc_feature: Murine SC17.10 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaagactgaagacctggcactttattactgtcagcaatattattggtttccgtacacgttcggaggggggaccaagctggaaataaaa

SEQ ID NO: 237 - DNA, 339 bp, Mus musculus
  misc_feature: Murine SC17.10 VH
gaggttcagctgcagcagtctggggcagaacttgtgaagccaggggcctcagtcaagttgtcctgcacagcttctggcttcaacattaaagacacctatatgcactgggtgaagcagaggcctgaacagggcctggagtggattggaaggattgatcctgcgaatgttaatactaaatatgacccgaagttccagggcaaggccactataacagcagacacatcctccaacacagcctacctgcagctcagcagcctgacatctgaggacactgccgtctattactgtgttagggggaatgtttactggggccaagggactctggtcactgtctctgca

SEQ ID NO: 238 - DNA, 324 bp, Mus musculus
  misc_feature: Murine SC17.11 VL
gaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtgattacccattcacgttcggctcggggacaaagttggtaataaaa

SEQ ID NO: 239 - DNA, 366 bp, Mus musculus
  misc_feature: Murine SC17.11 VH
gaggtccagctgcagcagtctggacctgagctggtgaaacctggggctttagtgatgatgtcctgcaaggcttctggatacacattcactgactactacatgcactgggtgaagcagagccatggacagagccttgagtggattggagaggttattccttacaatgatgaaactttctacaaccggaagttcaaggacaaggccacattgactgtagacaaatcctctagtacagcctacatggagctccggagcctgacatctgaggactctgcaatctattattgtgcaagaagacataggtacgacgggtttcgttatgctatagactactggggtcaaggaacctcagtcaccgtctcctca

SEQ ID NO: 240 - DNA, 336 bp, Mus musculus
  misc_feature: Murine SC17.14 VL
gatgttttgatgacccaaactccactctccctgcctgtcagtcttggagatcaagcctccatctcttgcagatctagtcagagcattgtccatagtaatggaaacacctatttagagtggttcctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 241 - DNA, 354 bp, Mus musculus
  misc_feature: Murine SC17.14 VH
gaggtccagctgcaacagtctggacctgtgctggtgaagcctggggcttcagtgaagatgtcctgtaaggcttctggatacacaatcactgactacaatatgaactgggtgaagcagagccatggaaagagccttgagtggattggagttattaatccttacaacggtaatactagatataaccagatgttcaagggcaaggccacattgactgttgacaagtcctccagcacagcctacatggagctcaacagcctgacatctgaggactctgcagtctattactgtacaagatggggtactacggtggtaggtgcgaactggggccaaggcaccactctcacagtctcctca

SEQ ID NO: 242 - DNA, 318 bp, Mus musculus
  misc_feature: Murine SC17.15 VL
caaattgttctcacccagtctccagcactcatgtctgcatctccaggggagaaggtcaccatgacctgcagtgccagctcaagtgtaaattacatgtactggtaccagcagaagccaagatcctcccccaaaccctggatttatctcacatccaacctggcttctggagtccctgttcgcttcagtggcagtgggtctgggacctcttactctctcacaatcagcagcatggaggctgaagatgctgccacttactactgccagcagtggagtaataacccacccacgttcggttctgggaccaagctggagctgaaa

SEQ ID NO: 243 - DNA, 366 bp, Mus musculus
  misc_feature: Murine SC17.15 VH
gacgtgaagctcgtggagtctgggggaggcttagtgaagcttggagggtccctgaaactctcctgtgcagcctctggattcactttcagtagctatgccatgtcttgggttcgccagactccggagaagaggctggagtgggtcgcaaccattactagtggtggtggtaacacctactatccagacagtgtgaagggtcgattcaccatctccagagacaatgccaagaacaccctgtacctgcaaatgagcagtttgaagtctgaggacacggccatgtattactgtgcaagaagggattactacggtagtagttacgttatgtttgcttattggggccaagggactctggtcactgtctctgca

SEQ ID NO: 244 - DNA, 318 bp, Mus musculus
  misc_feature: Murine SC17.17 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactgccttctggagtccctgctcgcttcagtggcagtgggtctgggacctcttactctctcacaatcagcagcatggaggctgaagatgctgccacttattactgccagcagtggagtagtaccccacccacgttcggtgctgggaccaagctggagctgaaa

SEQ ID NO: 245 - DNA, 360 bp, Mus musculus
  misc_feature: Murine SC17.17 VH
gaggtccagctgcaacagtctggacctgaggtaatgaagcctggggcttcagtgaagatgtcctgcaaggcttctggatacacattcactgactacaacatgcactgggtgaagcagaaccaaggaaagagcctagagtggataggagaaattaatcctaacattggtggtactggctacaaccagaagttcaaaggcaaggccacattgactgtacacaagtcctccagcacagcctacatggagctccgcagcctgacatctgaggactctgcagtctattactgtgcaagaacctatagttactatagttacgagtttgcttactggggccaagggactctggtcactgtctctgca

SEQ ID NO: 246 - DNA, 321 bp, Mus musculus
  misc_feature: Murine SC17.18 VL
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtattccgctcacgttcggtgcggggaccaagctggagctgaaa

SEQ ID NO: 247 - DNA, 369 bp, Mus musculus
  misc_feature: Murine SC17.18 VH
caggttactctgaaagagtctggccctgggatattgcagccctcccagaccctcagtctgacttgttctttctctgggttttcactgagcacttctactatgggtgtaggctggattcgtcagccttcaggaaagggtctagagtggctggcagacatttggtgggatgacagtaagtactataatccatccctgaagagccggctcacaatctccaaggatacctccagcaaccaggtattcctcaagatcaccagtgtggacactgcagatactgccacttactactgtgcgcgaaagggaaggacagctcgggctacgagagggtttgcttactggggccacgggactctggtcactgtctctgca

SEQ ID NO: 248 - DNA, 327 bp, Mus musculus
  misc_feature: Murine SC17.19 VL
gacattgtgctgacccaatctccagcttctttggctgtgtctctagggcagagggccgccatctcttgcaagcccagccaaagtgttgattatgatggtgatagttatatgaactggtaccaacagaaaccaggccagccacccaaactcctcatttatgctgcatccaatctagaatctgggatcccagccaggtttagtggcagtgggtctgggacagacttcaccctcaacatccatcctgtggaggaggaggatgctgcaacctattactgtcaccaaattaatgacgatccgtggacgttcggtggaggcaccaagctgaaa

SEQ ID NO: 249 - DNA, 363 bp, Mus musculus
  misc_feature: Murine SC17.19 VH
gatgtgcagcttcaggagtcaggacctggcctggtgaaaccttctcagtctctgtctgtcacctgcactgtcactggctactccatcaccagtagttatacctggaactggatccggcagtttccaggaaacaaactggagtggatgggctacatacattacagtggtagcactaactacaacccatctctcagaagtcgaatctctattactcgagacacgtccaagaaccagttcttcctgcagttgaattctgtgactactgaggacacagccacatattactgtgcaagatcccgttattactacgatgcttacgggtttgcttactggggccaagggactctggtcactgtctctgca

SEQ ID NO: 250 - DNA, 336 bp, Mus musculus
  misc_feature: Murine SC17.22 VL
gatgttgtgttgacccaaactccactctccctgcctgtcagtcttggagatcaagcctccatctcttgcagatctagtcagagcattgtacacattaatagacacacctacttaggatggtacctgcagaaaccaggccagtcgctaaagctcctgatatatggggtttccaaccgattttctggggtcccagacaggttcagtggcagtggatcagggacagatttcacactcaagatcagcagagtggaggctgaggatatgggagtttattactgctttcaaggtacacatgttccattcacgttcggctcggggacaaagttggaaataaaa

SEQ ID NO: 251 - DNA, 363 bp, Mus musculus
  misc_feature: Murine SC17.22 VH
cagatccagatgatgcagtctggacctgagctgaagaagcctggagagacagtcaagatctcctgcaaggcttctgggtattccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacatggctacatatttctgtacaagaggttactacggtagtagctacgatgctttggactactggggtcaaggaacctcagtcaccgtctcctca

SEQ ID NO: 252 - DNA, 336 bp, Mus musculus; identical to SEQ ID NO: 222
  misc_feature: Murine SC17.24 VL

SEQ ID NO: 253 - DNA, 351 bp, Mus musculus; identical to SEQ ID NO: 223
  misc_feature: Murine SC17.24 VH

SEQ ID NO: 254 - DNA, 336 bp, Mus musculus
  misc_feature: Murine SC17.27 VL
gatgttgtgatgacccagactccactcactttgtcggttaccattggacaaccagcctccatctcttgcaagtcaagtcagagcctcttagaaagtgatggaaagacatatttgaattggttgttacagaggccaggccagtctccaaagcgcctaatctatctggtgtctaaactggactctggagtccctgacaggttcacgggcagtggatcagggacagatttcacactgaaaatcagcagagtggaggctgaggatttgggagtttattattgctggcaaggtatacaacatcctcggacgttcggtggaggcaccaagctggaaatcaaa

SEQ ID NO: 255 - DNA, 339 bp, Mus musculus
  misc_feature: Murine SC17.27 VH
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggaggtattgatcctgaaactggtggtactgcctacaatcagaagttcaagggcaaggccacactgactgcagacaaatcctccagcacagcctacatggagctccgcagcctgacatctgaggactctgccgtctacttctgtacaagatggttttcttactggggcccagggactctggtcactgtctctgca

SEQ ID NO: 256 - DNA, 321 bp, Mus musculus
  misc_feature: Murine SC17.28 VL
gacatcttgctgactcagtctccagccatcctgtctgtgagtccaggagaaggagtcagtttctcctgcagggccagtcagagcattggcacaagcatacactggtatcagcaaagaacaaatggttctccaagacttctcataaagtatgcttctgagtctatctctgggatcccttctaggtttagtggcagtgggtcagggacagattttactcttcgcatcaacagtctggagtctgaagatattgcagattattactgtcaacaaagtaatagctggccactcacgttcggtgctgggaccaagctggagctgaaa

SEQ ID NO: 257 - DNA, 351 bp, Mus musculus
  misc_feature: Murine SC17.28 VH
caggtccacctgccgcagtctagacctgaactggtgaagcctggagcttcagtgaagatatcctgcaaggcttctggctacggcttcacacgcagctatatacactgggtgaagcagaggcctggacagggcctagagtggattggatatatttcttctggaagtggtggtactacctacaatcagaagtttaagggcaaggcctcactgactgcagacaatccctccagcactgcctacatgcatctcagtagcctgacatctgaggactctgcgatctatttctgtgcaagagggggggtacggtacttcgatgtctggggcgcagggaccacggtcaccgtctcctca

SEQ ID NO: 258 - DNA, 321 bp, Mus musculus
  misc_feature: Murine SC17.29 VL
gacattgtgatgacccagtctcacaaattcatgtccacatcagtaggagacagggtcagcatcacctgcaaggccagtcaggatgtgggtactgatgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccgtacacgttcggaggggggacaaagctggaaataaaa

SEQ ID NO: 259 - DNA, 348 bp, Mus musculus
  misc_feature: Murine SC17.29 VH
gaggtccagctgcaacagtctggacctgagctaatgaagcctggggcttcagtgaagatgtcctgcaaggcttctggatacacattcactgactacaacatgcactgggtgaagcagaaccaaggaaagagcctagagtggattggagaaattaatcctcacaatggtggtactggctacaaccagaagttcaaaggcaaggccacattgactgtagacaagtcctccagcacatcctacatggagctccgcagcctgacatctgaggactctgcagtctattactgtgcaggcggttacccggcctttgactactggggccaaggcaccactctcacagtctcctca

SEQ ID NO: 260 - DNA, 324 bp, Mus musculus
  misc_feature: Murine SC17.30 VL
gaaaatgtgctcacccagtctccagcaatcgtgtctgcatctccaggggaaaaggtcaccatgacctgcagggccagctcaagtgtaatttccagttacttgcactggtaccagcagaagtcaggtgcctcccccaaactctggatttatagcacatccaacttggcttctggagtccctgctcgcttcagtggcagtgcgtctgggacctcttactctctcacaatcagcagtgtggaggctgaagatgctgccacttattactgccagcagtacagtggttacccgctcacgttcggtgctgggaccaagctggagctgaaa

SEQ ID NO: 261 - DNA, 372 bp, Mus musculus
  misc_feature: Murine SC17.30 VH
gaagtgaagctggtggagtctgagggaggcttagtgcagcctggaagttccatgaaactctcctgcacagcctctggattcactttcagtgactattacatggcttgggtccgccaggttccagaaaagggtctagaatgggttgcaaacattaattatgatggtagtagcacttactatctggactccttgaagagccgtttcatcatctcgagagacaatgcaaagaacattctatacctgcaaatgagcagtctgaagtctgaggacacagccacgtattactgtgcaagagatgattattacggtagtagcccaagctactggtacttcgatgtctggggcgcagggaccacggtcaccgtctcctca

SEQ ID NO: 262 - DNA, 321 bp, Mus musculus
  misc_feature: Murine SC17.32 VL
gacatccagatgactcagtctccagcctccctatctgcatctgtgggagaaactgtcaccatgacatgtcgagcaagtgggaatattcacaattatttagtatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactcctccgacgttcggtggaggcaccaagctggaaatcaaa

SEQ ID NO: 263 - DNA, 357 bp, Mus musculus
  misc_feature: Murine SC17.32 VH
gaagtgaaacttgaggagtctggaggaggcttggtgcaacctggaggatccatgaaactctcctgtgttgcctctggattcactttcagtaactactggatgagctgggtccgccagtctccagagaaggggcttgagtgggttgctgaaattagattgaaatctaataattatgcaacacattatgcggagtctgtgaaagggaggttcaccatctcaagagacgattccaaaagtagtgtcttcctgcaaatgaacaacttaagaactgaagacactggcatttattactgtaccaggcactattactatgctatggactactggggtcaaggaacctcagtcaccgtctcctca

SEQ ID NO: 264 - DNA, 321 bp, Mus musculus
  misc_feature: Murine SC17.34 VL
gacatcaagatgacccagtctccatcttccatgtatgcatctctaggagagagagtcactatcacctgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagtagcctggagtatgaagatatgggaatttattattgtctacagtatgatgagtttcctccgacgttcggtggaggcaccaagctggaaatcaaa

SEQ ID NO: 265 - DNA, 354 bp, Mus musculus
  misc_feature: Murine SC17.34 VH
gaggtccagctacaacagtctggacctgagctggtgaagcctgggtcttcagtgaagatatcctgcaaggcttctggatacacattcactgactacaacatggactgggtgaagcagagccatggaaagagacttgagtggattggatatatttatcctgacaatggtggtgctggctacaaccagaagttcaagggcaaggccacattgactgtagacaagtcctccagcacagcctacatggagctccgcagcctgacatctgaggactctgcagtctattactgttcaagatccattactacggcttggtttgcttactggggccaagggactctggtcactgtctctgca

SEQ ID NO: 266 - DNA, 324 bp, Mus musculus
  misc_feature: Murine SC17.35 VL
gaaaatgtgctcacccagtctccagcaatcatgtctgcatctccaggggaaaaggtcaccctgacctgcagggccagctcaagtatgagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtgcttacccattcacgttcggctcggggacaaagttggaaataaaa

SEQ ID NO: 267 - DNA, 363 bp, Mus musculus
  misc_feature: Murine SC17.35 VH
gaggtccagctgcagcagtctggacctgagctagtgaaacctggggctttagtgaagatgtcctgcaaggcttctggatacacattcactgactactacatacactgggtgaagcagagccatggaaagagccttgagtggattggagaaattaatccttacaatggtgagactttctacaaccagaagttcaagggcaaggccacattgactgtagacaaatcctctagtacagcctacatggaactccggagcctgacatctgaggactctgcagtctattattgtgcaagaaggggatggtatctaacaggctatgctatggactactggggtcaaggaacctcagtcaccgtctcctca

SEQ ID NO: 268 - DNA, 318 bp, Mus musculus
  misc_feature: Murine SC17.36 VL
caaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccacccacgttcggaggggggaccaagctggaaataaaa

SEQ ID NO: 269 - DNA, 360 bp, Mus musculus
  misc_feature: Murine SC17.36 VH
gaggtgcagcttcaggagtcaggacctagcctcgtgaaaccttctcagtctcagtccctcacctgttctgtcactggcgactccatcaccagtgattactggaactggatccggaaattcccagggaagaaagttgagtacatggggtacataaactacagtggtagcacttactacaatccatctctcaaaagtcgaatctccatcactcgagacacatccaagaaccagtactacctgcagttgaactctgtgacttctgaggacacagccacatattactgtgcacgtacctcgtactataataagtttctaccatttgcttactggggccaagggactctggtcactgtctctgca

SEQ ID NO: 270 - DNA, 336 bp, Mus musculus
  misc_feature: Murine SC17.39 VL
gatgttttaatgacccaaactccactctccctgcctgtcagtcttggagatcaagcctccatctcttgcagatctagtcagagtcttgtacacagaaatggaaacacctattttcattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacatatgttccgtggacgttcggtggaggcaccaagctggaaatcaaa

SEQ ID NO: 271 - DNA, 351 bp, Mus musculus
  misc_feature: Murine SC17.39 VH
gaggtgcagctggtggagtctgggggaggcttagtgcagcctggagggtcccggaaactctcctgtgcagcctctggattcactttcagtagctatggaatgcactgggtccgtcaggctccagagaaggggctggagtgggtcgcatatattagtagtaacgatggtaccatctactatgcagacacagtgaggggccgattcaccatctccagagacaatgccaagaacaccctgttcttgcaaatgaccagtctaaggtctgaggacacggccatgtattactgtgcaagaccttctaactgggtctttgactactggggccaaggcaccactctcacagtctcctca

SEQ ID NO: 272 - DNA, 336 bp, Mus musculus
  misc_feature: Murine SC17.40 VL
gatgttgtgatgacccaaactccactctcccggcctgtcactcttggagatcaagcctccatctcttgcagatctagtcagagccttgtacacagtaatggaaacacctatttacattggtacctgcagaagccaggccagtctccaaagctcctgatctacaaagtttccaaccgattttctggggtccctgacaggttcagtggcagtggatcagggacagatttcacactcaagatcagcagagtggaggctgaggatctgggagtttatttctgctctcaaaatacacatgttccattcacgttcggctcggggacaaagttggaaataaaa

SEQ ID NO: 273 - DNA, 348 bp, Mus musculus
  misc_feature: Murine SC17.40 VH
caggtccaactgcagcagcctggggctgaaattgtgaggcctggggcttcagtgaagctgtcctgcaaggcttctggctacacctttaccgactattggatgaactgggtgaagcagaggcctggacaaggccttgagtggatcggaacaattgatccttctgatagttatactcgttacaatcaaaagttcaagggcaaggccacattgactgtagacacatccttcagctcagcctacatgcagctcagcagcctgacatctgaggactctgcggtctatttctgtgcaagtgggggacgggggtttggttactggggccaagggactccggtcactgtctctgta

SEQ ID NO: 274 - DNA, 318 bp, Mus musculus
  misc_feature: Murine SC17.41 VL
caaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacatccaacctggcttctggagtccctactcgcttcagtggcagtgggtctgggacctcttactctctcacaatcagcagcatgggggctgaagatgctgccacttattactgccagcagtggaatactaacccacccacgttcggtgctgggaccaagctggagctgaaa

SEQ ID NO: 275 - DNA, 363 bp, Mus musculus
  misc_feature: Murine SC17.41 VH
gacgtgaagctcgtggagtctgggggaggcttagtgaagcttggagggtccctgaaactctcctgtgcagcctctggattcactttcagtagctatgccatgtcttgggttcgccagactccggagaagaggctggagtgggtcgcaaccattagtagtggtggtggtaacacctactatccagacagtgtgaagggtcgattcaccatctccagagacaatgccaagaacaccctgtacctgcaaatgagcagtttgaagtctgaggacacggccatgtattactgtgcaagaagggattactacggtactagctacgttatgtttgcttactggggccaagggactctggtcactgtctct

SEQ ID NO: 276 - DNA, 318 bp, Mus musculus
  misc_feature: Murine SC17.42 VL
gaaaatgttctcacccagtctccagcaatcatgtctgcatctccaggggaaaaggtcaccatgacctgtagtgccagctcaagtgtaaattacatgtactggtaccagcagaagtcaagcacctcccccaaactctggatttatgacacatccaaactgacttctggagtcccaggtcgcttcagtggcagtgggtctggaaactcttactctctcacgatcagcaacatggaggctgaagatgttgccacttattactgttttcaggggagtgggtacccactcacgttcggctcggggacaaaattggaaataaaa

SEQ ID NO: 277 - DNA, 336 bp, Mus musculus
  misc_feature: Murine SC17.42 VH
gacgtgaagctggtggagtcggggggaggcttagtgaggcctggagggtccctgaaactctcctgtgcagcctctggattcactttcagtagatataccatgtcttgggttcgccagacaccggagaagaggctggagtgggccgcaaccattaatagtggtggtagtaacacctactatccagacagtgtgaagggccgattcaccatctccagagacaatgccaagaacaccctgttcctgcaaatgagcagtctgaagtctgaggacacagccatgtattactgtacaaatggtaaccactggggccaaggcaccactctcacagtctcctca

SEQ ID NO: 278 - DNA, 318 bp, Mus musculus; identical to SEQ ID NO: 276
  misc_feature: Murine SC17.45 VL

SEQ ID NO: 279 - DNA, 342 bp, Mus musculus
  misc_feature: Murine SC17.45 VH
caggtgcaactgcagcagcctgggtctgtgctggtgaggcctggagattcagtgaagctgtcgtgcaaggcttctggctacacattcaccagctactggatgcactgggtgaagcagagccctggacaaggccttgagtggattggagagattcatcctcatagtggtagtactaactacaatgagaagttcaagggcaaggccacactgactgtagacacatcctccagcacagcctacgtggatctcagcagcctgacatctgaggactctgcggtctattactgtgtaggtggtcactacgactactggggccaaggcaccactctcacagtctcctca

SEQ ID NO: 280 - DNA, 321 bp, Mus musculus
  misc_feature: Murine SC17.46 VL
agttttgtgatgacccaaactcccaaattcctgcttgtatcagcaggagacagggttaccataacctgcaaggccagtcagagtgtgaataatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctcctcggacgttcggtggaggcaccaagctggaaatcaaa

SEQ ID NO: 281 - DNA, 357 bp, Mus musculus
  misc_feature: Murine SC17.46 VH
caggtccaactgcagcagcctggtgctgagcttgtgaagcctggggcctcaatgaagctgtcctgcaaggcttctggctacactttcaccagctactggataaactgggtgaagcagaggcctggacaaggccttgagtggattggaaatatttttcctgatactactactactaactacaatgagaagttcaagagcaaggccacactgactgtagacacatcctccagcacagcctatatgcagctcagcagcctgacatctgacgactctgcggtctattattgtgcaagggagtactacgatggtacctacgatgctatggattactggggtcaaggaacctcagtcaccgtc

SEQ ID NO: 282 - DNA, 318 bp, Mus musculus
  misc_feature: Murine SC17.47 VL
caaattgttctcacccagtctccagcaatcatgtctgcatctccaggggagaaggtctccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggtcctcttactctctcacaatcagcagcatggaggctgaagatgctgccacttattactgccagcagtggagtagcaccccccccacgttcggaggggggaccaagctggaaataaaa

SEQ ID NO: 283 - DNA, 357 bp, Mus musculus
  misc_feature: Murine SC17.47 VH
gaggtccagctgcagcagtctggacctgagctggtgaagcctggggcttcagtgaagatatcctgcaaggcttctggttactcattcactgactactacatgcgctgggtgaagcaaagtcctgaaaagagccttgagtggattggagagattaatcctagcactggtggtactacctacaaccagaacttcaaggccaaggccacattgactgtagacaaatcctccagcacagcctacatgcagctcaagagcctgacatctgaggactctgcagtctattactgtgcaagagggggttacttcttgtactactttgactactggggccaaggcaccactctcacagtctcctca

SEQ ID NO: 284 - DNA, 336 bp, Mus musculus; identical to SEQ ID NO: 254
  misc_feature: Murine SC17.49 VL

SEQ ID NO: 285 - DNA, 339 bp, Mus musculus; identical to SEQ ID NO: 255
  misc_feature: Murine SC17.49 VH

SEQ ID NO: 286 - DNA, 333 bp, Mus musculus
  misc_feature: Murine SC17.50 VL
gacattgtgctgacacagtctcctgcttccttagctgcatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtggacgttcggtggaggcaccaagctggaaatcaaa

SEQ ID NO: 287 - DNA, 363 bp, Mus musculus
  misc_feature: Murine SC17.50 VH
gaggtgcagctggtggagtctgggggaggcttagtgaagcctggagggtccctgaaactctcctgtgcagcctctggattcactttcagtgactatggaatgcactgggttcgtcaggctccagagaaggggctggagtgggttgcatacattagtagtggcagtagaaccatctactatgcagacacagtgaagggccgattcaccatctccagagacaatgccaagaacaccctgttcctgcaaatgaccagtctgaggtctgaggacacggccatgtattactgtgcaagggtttactacggaagtacctacgggtatttcgatgtctggggcacagggaccacggtcaccgtctcctca

SEQ ID NO: 288 - DNA, 333 bp, Mus musculus
  misc_feature: Murine SC17.53 VL
gacattgtgctgacacagtctcctgcttccttagctgcatctctggggcagagggccaccatctcatgcagggccagtcaaagtgtcagtacatctagctatagttatatgcactggtaccaacagaagccaggacatccacccaaactcctcatcaggtatgcatccaacctagagtctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaa

SEQ ID NO: 289 - DNA, 357 bp, Mus musculus
  misc_feature: Murine SC17.53 VH
gaggtccagcttcagcagtcaggacctgagctggtgaaacctggggcctcagtgaagatatcctgcaaggcttctggatacacattcactgactacaacatgcactgggtgaagcagagccatggaaagcgccttgagtggattggatatattcatccttacaatggtggtagtggctacaaccagaagttcaagaggaaggccacattgactgtagacaattcctccaacacaacctacatggagctccgcagcctgacatctgaggactctgcagtctattactgtgcaagatcttatgattacgacacctggtttggttactggggccaagggactctggtcactgtccgtgca

SEQ ID NO: 290 - DNA, 336 bp, Mus musculus
  misc_feature: Murine SC17.54 VL
gatgttgtgctgacccagactccactcactttgtcggttaccattggacaaccagcctccatctcttgcaagtcaagtcagagcctcttatatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggactttattattgctggcaaggtacacattttccgtggacgttcggtggaggcaccaagctggaaatcaaa

SEQ ID NO: 291 - DNA, 339 bp, Mus musculus
  misc_feature: Murine SC17.54 VH
gaagtgaaacttgaggagtctggaggaggcttggtgcaacctggaggatccatgaaactctcctgtgttgcctctggattcactttcagtaactactggataaactgggtccgccagtctccagagaaggggcttgagtgggttgctgaaatcagaatgaaatctaataattatgcaacacattatgcggagtctgtgaaagggaggttcaccatctcaagagatgattccaaaagttgtgtctacctgcaaatgaacaacttaagacctgaagacactggcatttattactgtaccagggggggctactggggccaaggcaccactctcaccgtctcc

SEQ ID NO: 292 - DNA, 339 bp, Mus musculus
  misc_feature: Murine SC17.56 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttatttctgtcagcaatattataactatccgtacacgttcggaggggggaccaagctggaaataaaa

SEQ ID NO: 293 - DNA, 348 bp, Mus musculus
  misc_feature: Murine SC17.56 VH
cagatccagttggtgcagtctggacctgaactgaagaagcctggagagacagtcaagatctcctgcaaggcttctgggtataccttcacaaactatggaatgaactgggtgaagcaggctccaggaaagggtttaaagtggatggcctggataaacacctacactggagagccaacatatgctgatgacttcaagggacggtttgccttctctttggaaacctctgccagcactgcctctttgcagatcatcaacctcaaaaatgaggacacggctacatatttctgtgcaaggatcggcgatagtagtccctctgactactggggccagggcaccactctcacagtc

SEQ ID NO: 294 - DNA, 324 bp, Mus musculus
  misc_feature: Murine SC17.57 VL
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccacctcgcttcagtggcagtgggtctgggacctcttactctctcacaatcagcagcatggaggctgaagatgctgccacttattactgccaccagtatcatcgttccccaccgacgttcggtggaggcaccaagctggaaatcaaa

SEQ ID NO: 295 - DNA, 363 bp, Mus musculus
  misc_feature: Murine SC17.57 VH
cagatccagttggtgcagtctggacctgaactgaagaagcctggagagacagtcaagatctcctgcaaggcttctgattataccttcacagacttttcaatacactgggtgaggcagtctccaggaaagggtttaaagtggatgggctggataaacactgagactggtgagccaacagttgcagaagacttcaagggacggtttgccttctctttggagacctctgccagcactgcctttttgcagatctacaacctcaaaaatgaggactcggcaacatatttctgtgctagggggcgttactacggccatgactatgctatggactactggggtcaaggaacctcagtcaccgtctcctca

SEQ ID NO: 296 - DNA, 321 bp, Mus musculus
  misc_feature: Murine SC17.59 VL
gacatccagatgactcagtctccagcctccctatctgcatctgtgggagaaactgtcaccatcacatgtcgagcaagtgggaatcttcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggacttatttctgtcaacatttttggagtattcctcccacgttcgggggggggaccaagctggaaataaaa

SEQ ID NO: 297 - DNA, 357 bp, Mus musculus
  misc_feature: Murine SC17.59 VH
gaagtgaagcttgaggagtctggaggaggcttggtgcaacctgggggatccatgaaactctcctgtgttgcctctggattcactttcagtaactattggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccagactctgggactttgctatggactactggggtcaaggaacctcagtcaccgtctcctca

SEQ ID NO: 298 - DNA, 318 bp, Mus musculus
  misc_feature: Murine SC17.61 VL
caaattgttctcacccagtctccagcaatcatgtctgcatctccaggggagaaggtcaccatatcctgcagtgccagctcaagtgtaagttacatatactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccgtggacgttcggtggaggcaccaagctggaaatcaaa

SEQ ID NO: 299 - DNA, 375 bp, Mus musculus
  misc_feature: Murine SC17.61 VH
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacagatttggtgggatgattataagtactataacccagccctgaagagtcggctcacaatctccaaggatacctccaaaaaccaggtattcctcaagatcgccaatgtggacactgcagatactgccacatactactgtgctcgaatcggatattactccggtagtagccgttgctggtacttcgatgtctggggcacagggagcacggtcaccgtctcctca

SEQ ID NO: 300 - DNA, 321 bp, Mus musculus
  misc_feature: Murine SC17.63 VL
agtattgtgatgacccagactcccaaattcctgcttgtatcagcaggagacagggttgccataacctgcaaggccagtcagagtgtgagtaatgatgtagcttggtaccaacagaagccagggcagtctcctacactgctgatatcctatgcatccaatcgctacactggagtccctgatcgcttcactggcagtggatatgggacggatttcactttcaccatcagcactgtgcaggctgaagacctggcagtttatttctgtcagcagggttatagctctccgttcacgttcggaggggggaccaagctggaaataaaa

SEQ ID NO: 301 - DNA, 348 bp, Mus musculus
  misc_feature: Murine SC17.63 VH
caggttcagctgcaacagtctgacgctgagttggtgaaacctggggcttcagtgaagatatcctgcaaggctgctggctacaccttcactgaccttactattcactgggtgaaacagaggcctgaacagggcctggagtggattggatatatttatcctggagatagtaatactaagtacaatgagaagttcaagggcaaggccacattgactgcagataaatcctccagcactgcctatatgcagctcaacagcctgacatctgaggattctgtagtgtatttctgtgcaagaatgattactccttactactttgactactggggccaaggcaccactctcacagtc

SEQ ID NO: 302 - DNA, 321 bp, Mus musculus
  misc_feature: Murine SC17.71 VL
gacatccagatgactcagtctccagcctccctatctgcctctgtgggagaaactgtcaccatcgcatgtcgagcaagtgggaatattcacaattatttaacatggtatcagcagagacagggaaaatctcctcagctcctggtctataatgcaaaaaccttagcagttggtgtgccatcaaggttcagtggcagtggctcaggaacacaatattctctcaagatcaacagcctgcagcctgaagattttgggagttattactgtcaacatttttggaatactcctccgacgttcggtggaggcaccaagctggaaatcaaa

SEQ ID NO: 303 - DNA, 357 bp, Mus musculus
  misc_feature: Murine SC17.71 VH
gaagtgaagcttgaggagtctggaggaggcttggtgcaacctggaggatccatgaaactctcctgtgttgcctctggaatcattttcagtaactactggatgaattgggtccgccagtctccagagaaggggcttgagtgggttgctgaaattagattgaaatctaataattattcaacacattatgcggagtctgtgaaagggaggttcaccatctcaagagatgattccaaaagtagtgtctacctgcaaatgaacaacttaagagctgaagacactggcatttattactgtaccaggcactattactatgctatggactactggggtcaaggaacctcagtcaccgtctcctca

SEQ ID NO: 304 - DNA, 321 bp, Mus musculus
  misc_feature: Murine SC17.72 VL
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaagttcagtggcagtgggtctgggacagattattctctcaccatcagcaacctggaacctgaagatatcgccacttactattgtcagcagtatagtaagcttccgtacacgttcggaggggggaccaagctggaaataaaa

SEQ ID NO: 305 - DNA, 357 bp, Mus musculus
  misc_feature: Murine SC17.72 VH
gaggtgcagctggtggagtctgggggaggcttagtgaagcctggagggtccctgaaactctcctgtgcagcctctggattcactttcagtagctatggcatgtcttgggttcgccagactccggagaagaggctggagtgggtcgcagccattaatagtaatggtggtagcacctactatccagacactgtgaagggccgactcaccatctccagagacaatggcaagaacaccctgtacctgcaaatgagcagtctgaggtctgaggacacagccttgtattactgtgtaagggatgatggttactacgttttctttgcttactggggccaagggactctggtcactgtctctgca

SEQ ID NO: 306 - DNA, 321 bp, Mus musculus
  misc_feature: Murine SC17.74 VL
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactcctcccacgttcggtgctgggaccaagctggagctgaaa

SEQ ID NO: 307 - DNA, 357 bp, Mus musculus
  misc_feature: Murine SC17.74 VH
caggtgcagctgaagcagtcaggacctggcctagtggcgccctcacagagcctgtccatcacatgcactgtctctgggttctcattaaccagctatggtgtagactgggttcgccagtctccaggaaagggtctggagtggctgggagtgatatggggtggtggaagcacaaattataattcagctctcaaatccagactgagcatcaccaaggacaactccaagagccaagttttcttaaaaatgaacagtctgcaaactgatgacacagccatgtactactgtgccagtggagactacgatggtagcctctggtttgcttactggggccaagggactctggtcactgtctctgca

SEQ ID NO: 308 - DNA, 336 bp, Mus musculus
  misc_feature: Murine SC17.76 VL
gatattgtgataacccaggatgaactctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttgtagagtatcctcggacgttcggtggaggcaccaagctggaaatcaaa

SEQ ID NO: 309 - DNA, 339 bp, Mus musculus
  misc_feature: Murine SC17.76 VH
gaggtgcagctggtggagtctgggggagacttagtgaagcctggagggtccctgaaactctcctgtgtagcctctggattcactttcagtagctatggcatgtcttgggttcgccagactccagacaagaggctggagtgggtcgcaaccattagtagtggtggtactttcacctactatccagacagtgtgaaggggcgattcaccgtctccagagacaatgccaagaacaccctgtacctgcaaatgagcagtctgaagtctgaggacacagccatgtattactgttcaagacatgggtggggctggggccaagggactctggtcactgtctctgca

SEQ ID NO: 310 - DNA, 321 bp, Mus musculus
  misc_feature: Murine SC17.77 VL
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaagccttagcagatggtgtgccatcaaggttcagtggcagtggatcaggaacacaatattctctcaagatcaacagcctgcagcctgaagattttgggagttattactgtcaacatttttggagtattcctccgacgttcggtggaggcaccaagctggaaatcaaa

SEQ ID NO: 311 - DNA, 357 bp, Mus musculus
  misc_feature: Murine SC17.77 VH
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagttgaagacactgccatttattactgtaccaggcactatgactatgctatggactactggggtcaaggaacctcagtcaccgtctcctca

SEQ ID NO: 312 - DNA, 321 bp, Mus musculus
  misc_feature: Murine SC17.79 VL
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaggatcaacagcctgcagcctgaagattttgggagttattactgtcaacatttttggagtactcctccgacgttcggtggaggcaccaaggtggaaatcaaa

SEQ ID NO: 313 - DNA, 357 bp, Mus musculus
  misc_feature: Murine SC17.79 VH
gaagtgaagcttgaggagtctggaggaggcttggtacaacctggaggatccatgaaactctcctgtgttgcctctggattcactttcagtgactactggatgaactgggtccgccagtctccagagaaggggcttgagttggttgctgaaattagattgatatctaataattatgcaacacattatgcggagtctgtgaaagggaggttcaccatctcaagagatgattccaaaagtagtgtctacctgcaaatgaacaacttaagagctgaagacactggcatttattactgtaccaggcactattactatgctttggactactggggtcaaggaacctcagtcaccgtctcctca

SEQ ID NO: 314 - DNA, 339 bp, Mus musculus
  misc_feature: Murine SC17.81 VL
gacattgtgatgtcacagtctccatcctccctaactgtgtcagttggagagaaggttactttgagctgcaagtccagtcagagccttttatatagtaccaatcaaaagatctacttggcctggtaccagcagaaaccagggcagtctcctaaactgctgatttactgggcatccactagggaatctggggtccctgatcgcttcacaggcagtggatctgggacagatttcactctcgccatcagcaatgtgaaggctgaagacctggcagtttattactgtcagcaatattatagctatccgtacacgttcggaggggggaccaagctggaaataaaa

SEQ ID NO: 315 - DNA, 339 bp, Mus musculus
  misc_feature: Murine SC17.81 VH
gaggttcagctgcagcagtctggggcagagcttgtgaagccaggggcctcagtcaagttgtcctgcacagcttctggcttcaacattaatgacacctattaccattggttgaagcagaggcctgaacagggcctggagtggattggaaggattgatcctgcgaatgttaatactaaatatgacccgaagttccagggcaaggccactttaacagcagacacatcctccaacacagcctacctgcagctcagcagcctgacatctgaggacactgccgtctattactgtggtagggggaatgcttactggggccaagggactctggtcactgtctctgca

SEQ ID NO: 316 - DNA, 312 bp, Mus musculus
  misc_feature: Murine SC17.82 VL
caaattgttctcacccagtctccagcaatcatgtctgcatctctaggggaggagatcaccctaacctgcagtgccagttcgagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtagtttcacgttcggctcggggacaaagttggaaataaaa

SEQ ID NO: 317 - DNA, 360 bp, Mus musculus
  misc_feature: Murine SC17.82 VH
gaggtccagctgcaacagtctggacctgagctggtgaagcctggggcttcagtgaagatgtcctgtaaggcttctggatacacattcactgactcctacatgaactgggtgaagcagagtcatggaaagagccttgagtggattggacgtgttaatcctaacaatggtggtgctagctacaaccacaagttcaagggcaaggccacattgacagtagacaaatccctcagcacagcctacatgcgcctcaacagcctgacatctgaggactctgcggtctattactgttcaagatctggagacctttattactatgctatggactactggggtcaaggaacctcagtcaccgtctcctca

SEQ ID NO: 318 - DNA, 318 bp, Mus musculus
  misc_feature: Murine SC17.84 VL
caaattgttctcacccagtctccagcaatcatgtctgcatctccaggggagaaggtcaccatgacctgcagtgccagctcaagtataagttacatgcactggtaccagcagaagtcaggcacctcccccaaaagatggatttatgacacatccaaactggcttctggagtccctgctcgcttcagtggcagtgggtctgggacctcttactctctcacaatcagcaacatggaggctgaagatgctgccacttattactgccagcagtggagtagtaccccacccacgttcggaggggggaccaagctggaaataaaa

SEQ ID NO: 319 - DNA, 351 bp, Mus musculus
  misc_feature: Murine SC17.84 VH
gaggtccagttgcaacagtctggacctgagctaatgaagcctggggcttcagtgaagatgtcctgcaaggcttctggatatatatttactgactacaacatgcactgggtgaagcagaaccaaggaaagagcctagagtggataggagaagttaatcctaacactggtggtattggctacaatcagaaattcaaaggcaaggccacattgactgtagacaagtcctccagcacagcctacatggacctccgcagcctgacatctgaggactctgcagtctattactgtgcaagagatggcaattattgctttgactactggggccaaggcaccactctcacagtctcctca

SEQ ID NO: 320 - DNA, 333 bp, Mus musculus
  misc_feature: Murine SC17.85 VL
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagagaggttcagtagcagtgggtcaggatctgatttcacactgagaatcagcagagtggaggctgaggatgtgggtgtttattactgtgctcaaaatctagaacatccgacgttcggtggaggcaccaagctggaaatcaaa

SEQ ID NO: 321 - DNA, 360 bp, Mus musculus
  misc_feature: Murine SC17.85 VH
gaggtgcagctggtggagtctgggggagacttagtgaagcctggagggtccctgaaactctcctgtgcagcctctggattcactttcagtaactatggcatgtcttgggttcgccagactccagacaagaggctggagtgggtcgcaaccattagtactggtggtacttacacctactatccagacagtgtgaaggggcgattcaccatctccagagacaatgccaagaacaccctgtacctgcaaatgagcagtctgaagtctgaggacacagccatgtattactgtgtaggacagtcctatagtgactacgtctcgtttgcttattggggccaagggactcaggtcactgtctctgca

SEQ ID NO: 322 - DNA, 336 bp, Mus musculus
  misc_feature: Murine SC17.87 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctccaaagtttccaaccgattttctggggtcccagacaggttcagtggcagtggatcagggacagatttcactctcaagatcagcagagtggaggctgaagatctgggagtttatttctgctctcaaagtacacatgttcctcccatgttcggaggggggaccaggctggaaataaaa

SEQ ID NO: 323 - DNA, 345 bp, Mus musculus
  misc_feature: Murine SC17.87 VH
gaggttcagctgcagcagtctggggctgagcttctgaagccaggggcctcagtcaagttgtcctgcacagcttctggcctcaacattaaagactactatatacactgggtgtaccagaggcctgaacagggcctggagtggattggaaggattgatcctgagagtgataatactttatatgacccgaagttccagggcaaggccagtataacagcagacacatcctccaacacagcctacctgcagctcagcagcctgacatctgaggacactgccgtctattactgtactactaataccccttttgcttactggggccaagggactctggtcactgtctctaca

SEQ ID NO: 324 - DNA, 336 bp, Mus musculus
  misc_feature: Murine SC17.89 VL
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattattgctttcaaggttcacatgttccattcacgttcggctcggggacaaagttggaaataaaa

SEQ ID NO: 325 - DNA, 360 bp, Mus musculus
  misc_feature: Murine SC17.89 VH
caggtccagttgcaacagtctggagctgaactggtaaggcctgggacttcagtgaaggtgtcctgcaagacttctggatacgccttcactaattacttgatagagtgggtaaagcagaggcctggacagggccttgagtggattggggtgattaatcctggaagtggtggtactaactacaatgagaagttcaaggtcaaggcaacactgactgcagacaaatcctccagcactgcctacatgcagctcaccagcctgacatctgatgactctgcggtctatttctgtacaagaagggatggttacttctttccctggtttgcttactggggccaagggactctggtcactgtctctgca

SEQ ID NO: 326 - DNA, 339 bp, Mus musculus
  misc_feature: Murine SC17.90 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactaggaaatctggggtccctgatcgcttcacaggcagtggatctgggacagatttcactctcaccatcagcagtgtgaaggctgaagacctggcagtttattactgtcatcaatattatagctatccgctcacgttcgctgctgggaccaagctggagctgaaa

SEQ ID NO: 327 - DNA, 345 bp, Mus musculus
  misc_feature: Murine SC17.90 VH
caggtgcaactgcagcagcctgggtctgtgctggtgaggcctggagcttcagtgaagctgtcctgcaaggcttctggctacacattcaccagctactggatgcactgggtgaagcagaggcctggacaaggccttgagtggattggagagattcatcctaataatggtagtactaactacaatgagaagttcaagggcaaggccacactgactgtagacacatcctccagcacagcctacgtggatctcagcagcctgacatctgaggactctgcggtctattactgtgcaagatggactttgtttacttactggggccaagggactctggtcactgtctctgca

SEQ ID NO: 328 - DNA, 336 bp, Mus musculus
  misc_feature: Murine SC17.91 VL
gatgttgtgatgacccaaactccactctccctgcctgtcagtcttggagatcaagcctccatctcttgcagatctagtcagagccttgtacacagtaatggaaacacctatttactttggtacctgcagaagccaggccagtctccaaagctcctgatctacaaagtttccaaccgattttctggggtcccagacaggttcagtggcagtggatcagggacagatttcgcactcaagatcagcagagtggaggctgaggatctgggagtttatttctgctctcaaagtacacatgttccgtggacgttcggtggaggcaccaagctggaaatcaaa

SEQ ID NO: 329 - DNA, 351 bp, Mus musculus
  misc_feature: Murine SC17.91 VH
gaggtgcagctggtggagtctgggggaggcttagtgaagcctggagggtcccggaaactctcctgtgcagcctctggattcactttcagtgactatggaatgcactgggtccgtcaggctccagagaaggggctggagtgggttgcatacattagtcgtggcagtagtaccatccactatgcagacacagtgaagggccgattcaccatctccagagacaatgccaagaacaccctgttcctgcaaatgaccagtctaaggtctgaggacacagccatgtattactgtgcaaggcctttcaactggtacttcgatgtctggggcgcagggacaacggtcaccgtctcctca

SEQ ID NO: 330 - DNA, 339 bp, Mus musculus
  misc_feature: Murine SC17.93 VL
gacattgtgatgtcacagtctccatcctccctagctgtgtcagttggagagaaggttactatgacctgcaagtccagtcagagccttttatatagtagcaatcaaaagaactacttggcctggtaccagcagaaaccagggcagtctcctaaactactaatttactgggcatccactagggaatctggggtccctgatcgcttcataggcagtggctctgggacagatttcactctcaccatcagcagtgtgaaggctgaagacctggcaatttattactgtcagcaatattatcgctatccgctcacgttcggtgctgggaccaaactggagctgaaa

SEQ ID NO: 331 - DNA, 363 bp, Mus musculus
  misc_feature: Murine SC17.93 VH
caggtccaactgcagcagcctggggctgagcttgtgaagcctggggcttcagtgatgctgtcctgcaaggcttctggctacaccttcaccagctactgggtacactgggtgaagcagaggcctggacaaggccttgagtggattggagtgattaatcctagaaacggtcgtaacaattacaatgagaagttcaagaccaaggccacactgactgtagacaaatcatccagcacagcctacatgcaactcagcagcccgacatctgaggactctgcggtctattactgtgcacgagaggattacgacgggggggactatgctatggactactggggtcaaggaacctcagtcaccgtctcctca

SEQ ID NO: 332 - DNA, 321 bp, Mus musculus
  misc_feature: Murine SC17.95 VL
gatatccagatgacacagactacatcctccctgtcggcctctctgggagacagggtcaccatcagttgcagtgcaagtcagggcattaacaattatttaaactggtatcagcagaaaccagatggaactgttacactcctgatctattacacatcaagtttacactcaggagtcccatccaggttcagtggcagtgggtctgggacagattattctctcaccatcagcaacctggaacctgaagatattgccacttactattgtcagcagtatagtaagcttccgtggacgttcggtggaggcaccaagctggaaatcaaa

SEQ ID NO: 333 - DNA, 363 bp, Mus musculus
  misc_feature: Murine SC17.95 VH
gaggtcgagctgcaacagtctggacctgagctggtgaagccgggggcttcagtgaagatatcctgcaagacttccggaaacacatacactgaatacaccatgcagtgggtgaagctgagccatggaaagagccttgagtggattggaggtattaatcctaacaatggtattactagttacaaccagaagttcaagggcaaggccacattgactgtagacaagtcctccagcacagcctacatggagctccgcagcctgaaatctgaggattctgcagtctattactgtgcaagagcgggacttggtaactacgtttgggctatggactactggggtcaaggagcctcagtcaccgtctcctca

SEQ ID NO: 334 - DNA, 336 bp, Mus musculus
  misc_feature: Murine SC17.97 VL
gatgttgtgatgacccaaactccactctccctgcctgtcagtcttggagatcaagcctccatctcttgcagatctagtcagagccttgtacacaataatggaaacacctatttacattggtacctgcagaagccaggccagtctccaaacctcctgatctacaaagtttccaaccgattttctggggtcccagacaggttcagtggcagtggatcagggacagatttcacactcaagatcagcatagtggaggctgaggatctgggactttatttctgctctcaaagtacacatgttcctcggacgttcggtggaggcaccaagctggaaatcaaa

SEQ ID NO: 335 - DNA, 357 bp, Mus musculus
  misc_feature: Murine SC17.97 VH
caggtccagcttccgcagtctggggctgaactggcaaaacctggggcctcagtgaaaatctcctgcaaggcttctggcttcacctttacttcctactggatgcactgggtaaaacagaggcctggacagggtctggaatggattggatacattaatcctagcactgattatactgagtacaatcagaagttcaaggacaaggccacattgactgcagacaaatcctccagcacagcctacatgcaactgggcagcctgacatctgaggactctgcagtctattactgtgcaagatcttcctacggtagtagcccctttgattattggggccaaggctccactctcacagtctcctca

SEQ ID NO: 336 - DNA, 339 bp, Mus musculus
  misc_feature: Murine SC17.99 VL
gacattgtgatgtcacagtctccatcctccctagctgtgtctgttggagagaaggttactatgaactgcgagtccagtcagagccttttatatagtagcaatcaaaagaactacttggcctggtaccagcagaaaccagggcagtctcctaaactgctgatttactgggcatccactagggattctggggtccctgatcgcttcacaggcagtggatctgggacagatttcactctcaccatcagcagtgtgagggctgaagacccggcagtttattactgtcagcaatattatagctatccgctcacgttcggtgctgggaccaagctggagctgaga

SEQ ID NO: 337 - DNA, 348 bp, Mus musculus
  misc_feature: Murine SC17.99 VH
gaagtgaagcttgaggagtctggaggaggcttggtgcaacctggaggatccatgaaactctcttgcgctgcctctggattcacttttagtgacgcctggatggactgggtccgccagtctccagagaaggggcttgagtgggttgctgaaataagaagcaaagctaataatcatgcaacatactatgctgagtctgtgaaagggaggttcaccatctcaagagatgattccaaaagtagtgcctacctgcaaatgaacagcttaagagctgaagacactggcatttattattgtgtttcaacagggacttcttactggggccaagggactctggtcactgtctctgca

SEQ ID NO: 338 - DNA, 318 bp, Mus musculus
  misc_feature: Murine SC17.102 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctcctcgcttcagtggccgtgggtctgggacctcttactctctcacaatcagcagcatggaggctgaagatgctgccacttattactgccagcattggagtagtaacccacccacgttcggtgctgggaccaagctggagatgaaa

SEQ ID NO: 339 - DNA, 348 bp, Mus musculus
  misc_feature: Murine SC17.102 VH
gaggtccagctgcaacagtctggacctgagctaatgaagcctggggcttcagtgaagatgtcctgcaaggcttctggagacacattcactgactacaacatacactgggtgaagcagaaccaaggaaagagcctagagtggataggagaagttaatcctaacattggtggtattggctataaccagaagttcaaaggcaaggccacattgactgtagacaagtcctccagcacagcctacatggagctccgcagcctgacatctgaggactctgcagtctattactgtgcaatggggaggtggtacttcgatgtctggggcgcagggaccacggtcaccgtctcctca

SEQ ID NO: 340 - DNA, 336 bp, Mus musculus
  misc_feature: Murine SC17.114 VL
gatgttgtgatgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccagccgattttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaa

SEQ ID NO: 341 - DNA, 345 bp, Mus musculus
  misc_feature: Murine SC17.114 VH
gaggtccagctgcagcagtctggacctgagatggtgaagcctggggcttcagtgaagatatcctgcaaggcttctggatacacattcactgactactacatgcactgggtgaaacagagccatggaaagagccttgagtggattggacgtgttaatactaacaatggtggaactagctacgaccagaagttcgagggcaaggccacattgactgttgacaaatcttccagcacagcctacatggagctcaacagcctgacatctgaggactctgcggtctattactgtgtaatccctgcctggtttgcttactggggccaagggactctggtcactgtctctgca

SEQ ID NO: 342 - DNA, 336 bp, Mus musculus
  misc_feature: Murine SC17.115 VL
gatattgtgatgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacagagtttccaaccgattttctggggtcccagacaggttcagtggcagtggatcagggacagatttcacactcacgatcagcagagtggaggctgaggatctgggagtttatttctgctctcaaagtacacatcttcctcggacgttcggtggaggcaccaagctggagatcaaa

SEQ ID NO: 343 - DNA, 342 bp, Mus musculus
  misc_feature: Murine SC17.115 VH
caggtgcaactgcagcagtctgggtctgtgctggtgaggcctggagcttcagtgaagctgtcctgcaaggcttctggctacacattcaccagctactggatgcactgggtgaagcagaggcctggacaaggccttgagtggattggagagattcatcctaatagtgggaatactaattacaatgagaagttcaagggcaaggccacactgactgtagacacatcctccagcacagcctacgtggatctcagcagcctgacatctgaggactctgcggtctattattgtgcaggtggtaactacgactactggggccaaggcaccactctcacagtctcctca

SEQ ID NO: 344 - DNA, 333 bp, Mus musculus
  misc_feature: Murine SC17.120 VL
gacattgtgctgacccaatctccagcttctttggctgtatctctagggcagagggccaccatatcctgcagagccagtgaaagtgttgatagttatggcaatagttttatgcactggtaccagcagaaaccaggacagccacccaaagtcctcatctatcgtgcatccaacctagaatctgggatccctgccaggttcagtggcagtgggtctaggacagacttcaccctcaccattaatcctgtggaggatgaagatgttgcaacctattactgtcagcaaagtaatgaggatccgtacacgttcgggggggggaccaagctggaaataaaa

SEQ ID NO: 345 - DNA, 351 bp, Mus musculus
  misc_feature: Murine SC17.120 VH
gaggttcagctcgagcagtctgggactgtgctggcaaggcctggggcttcagtgaagatgtcctgcaaggcttctggctacacctttaccagctactggatgcactgggtgaaacagaggcctggacagggtctggaatggattggcgctttttatcctggaaacagtggtacttattacaaccaaaaattcaaggacaaggccaaactgactgcagtcacatctgccagcactgcctacatggagctcagcagcctgacaaatgaggactctgcggtctattactgttcaagatcagggtcaggaaggtttgcttactggggccaagggactctggtcactgtctctgca

SEQ ID NO: 346 - DNA, 318 bp, Mus musculus
  misc_feature: Murine SC17.121 VL
caaattgttctcacccagtctccagcaatcatgtctgcatctccaggggagaaggtcaccatgacctgcagtgccagctcaagtgtgagttacatgcactggtaccagcagaagtcaggcacctcccccaaaagatggatttatgacacatccaaactggcttctggagtccctgctcgcttcagtggcagtgggtctgggacctcttactctctcacaatcagcagcatggagactgaagatgctgccacttattactgccagcagtggagtaataccccacccacgttcggctcggtgacaaagttggaaataaaa

SEQ ID NO: 347 - DNA, 348 bp, Mus musculus
  misc_feature: Murine SC17.121 VH
gaggtccagctgcaacagtctggacctgagctaatgaagcctggggcttcagtgaagatgtcctgcaaggcttctggatacacattcactgaccacaacatacactgggtgaaacagcaccaaggaaagagcctagagtggataggagaaattaatcctaacactggtggtactggctacaaccagaagttccaaggcaaggccacaatgactgtagacaagtcctccagcacagcctacatggaactccgcagcctgacatctgaggactctgcagtctattactgtgttagaggactgtacttctttgactactggggccaaggcaccactctcacagtctcctca

SEQ ID NO: 348 - DNA, 336 bp, Mus musculus
  misc_feature: Murine SC17.122 VL
gatattgtgataacccaggatgatctctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttgtagagtatcctcggacgttcggtggaggcaccaagctggaaatcaaa

SEQ ID NO: 349 - DNA, 339 bp, Mus musculus
  misc_feature: Murine SC17.122 VH
gaggtgcacctggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtacttacacctactatccagacagtgtgaaggggcgattcaccatctccagagacaatgccaagaacaccctgtatctgcaaatgagcagtctgaagtctgaggacacagccatgtattactgttcaagacatgggtggggctggggccaagggactctggtcactgtctctgca

SEQ ID NO: 350 - DNA, 318 bp, Mus musculus
  misc_feature: Murine SC17.140 VL
caaattgttctcacccagtctccagcaatcatgtctgcatctccaggggagaaggtcaccatgacctgcagtgccagctcaagtgttagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaccccacccacgttcggctcggggacaaagttggaaataaaa

SEQ ID NO: 351 - DNA, 360 bp, Mus musculus
  misc_feature: Murine SC17.140 VH
gaggtccagctgcaacagtctggacctgagctaatgaagcctggggcttcagtgaagatgtcctgcaaggcttctggatacacattcactgactacaacatgcactgggtgaagcagaaccaaggaaagagcctagagtggataggagaaattaatcccaacactggtggtactggctacaaccagaagttcaaaggcaaggccacattgactgtagacaagttttccagcacagccttcattgagctccgcagcctgacatctgaggactctgcaatctattactgtacaagagggggttacgaccactattggtacttcgatgtctggggcgcagggaccacggtcaccgtctcctca

SEQ ID NO: 352 - DNA, 333 bp, Mus musculus
  misc_feature: Murine SC17.151 VL
gacattgtgctgacccaatttccagcttctttggctgtgtctctagggcagagggccaccataccctgcagagccagtgaaagtgttgatagttatggcaatagttttatgcactggttccagcagaaaccaggacagccacccaaactcctcatctatcgtgcatccaacctagaatctgagatccctgccaggttcagtggcagtgggtctgggacagacttcaccctcaccattaatcctgtggaggctgatgatgttgcaacctattactgtcagcaaagtcatgaggatccgtacacgttcggaggggggaccaagatggaaataaaa

SEQ ID NO: 353 - DNA, 351 bp, Mus musculus
  misc_feature: Murine SC17.151 VH
gaggttcagctgcagcagtctgggactgtgctggcaaggcctggggcttcagtgaagatgtcctgcaaggcttctggctatacctttaccagctactggatgcactgggtaaaacagaggcctggacagggtctggaatggattggcgctatttatcctggaaagaatgatactacctacaaccagaagttcaagggcaaggccaaactgactgcagtcacatctgccagcactttatacatggagctcagcagcctgacaaatgaggactctgcggtctattactgtacaagatctggaaagggttactttgcttactggggccaagggactctggtcactgtctctgca

SEQ ID NO: 354 - DNA, 336 bp, Mus musculus
  misc_feature: Murine SC17.156 VL
gatgttgtgatgacccagtctccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaaactggaaatcaaa

SEQ ID NO: 355 - DNA, 354 bp, Mus musculus
  misc_feature: Murine SC17.156 VH
caggttactctgaaagagtctggccctgggatattgcagccctcccagaccctcagtctgacttgttctttctctgggttttcactgagcacttctggtatgggtgtgagctggattcgtaagacttcaggaaagggtctggaatggctggcacacattttctgggatgatgacaagtggtataatccatccctgaagagccggctcacaatctccaaggctacctccagcaaccaggtattcctcatactcaccagtgtggatactgccgatactgccacatactactgtgctaccttctatggtctctactttgcctactggggccaaggcaccactctcacagtctcctca

SEQ ID NO: 356 - DNA, 339 bp, Mus musculus
  misc_feature: Murine SC17.161 VL
gacattgtgatgtcacagtctccatcctccctagctgtgtctgttggagagaaggttactatgaactgcgagtccagtcagagccttttatataatagcaatcaaaagaactacttggcctggtaccagcagaaaccagggcagtctcctaaactgctgatttactgggcatccactagggattctggggtccctgatcgcttcacaggcagtggatctgggacagatttcactctcaccatcagcagtgtgagggctgatgacccggcagtttattactgtcagcaatattttaactatccgctcacgttcggtgctgggaccaagctggagctgaaa

SEQ ID NO: 357 - DNA, 348 bp, Mus musculus
  misc_feature: Murine SC17.161 VH
gaagtgaagcttgaggagtctggaggaggcttggtgcaacctggaggatccatgaaactctcttgcgctgcctctggattcacttttagtgacgcctggatggactgggtccgccagtctccagagaaggggcttgagtgggttgctgaaataagaagcaaacctaataatcatgcaacatactatgctgagtctgtgaaagggaggttcaccatctcaagagatgattccaaaagtagtgcctacctgcaaatgaacagcttaagagctgaagacactggcatttattactgtgtttcaacagggacttcttactggggccaagggactctggtcactgtctctgca

SEQ ID NO: 358 - DNA, 318 bp, Mus musculus; identical to SEQ ID NO: 318
  misc_feature: Murine SC17.166 VL

SEQ ID NO: 359 - DNA, 351 bp, Mus musculus; identical to SEQ ID NO: 319
  misc_feature: Murine SC17.166 VH

SEQ ID NO: 360 - DNA, 321 bp, Mus musculus
  misc_feature: Murine SC17.187 VL
gacatcaagatgacccagtctccatcttccatgtatgcatctctaggagagagagtcactctcacttgcaaggcgagtcaggacattaatagctatttaagctggttccagcagaaaccagggaaatctcctgagaccctgatctatcgtgcaaacagattgatagatggggtcccatcaaggttcagtggcagtggatctgggcaagattattctctcaccatcagcagcctggagtatgaagatatggggatttattattgtctacagtatgatgagtttcctccgacgttcggtggaggcaccaagctggaaatcaaa

SEQ ID NO: 361 - DNA, 354 bp, Mus musculus
  misc_feature: Murine SC17.187 VH
gaggtccacctacaacagtctggacctgaactggtgaaccctgggtcttcagtgaagatatcctgcaaggctgctggatacacattcactgactacaacatggactgggtgaagcagagccatggaaagagacttgagtggattggaaatatttatcctaacaatggtggtgctggatacaaccagaacttcaaggacaaggccacattgactgtagacaagtcctccagcacagcctacatggagctccgcagcctgacatctgaggactctgcagtctattactgtgcaagatccattactgcggcttggtttgcttactggggccaagggactctggtcactgtctctgca

SEQ ID NO: 362 - DNA, 318 bp, Mus musculus
  misc_feature: Murine SC17.191 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcactggcagtgggtctgggacctcttactctctcacaatcagcagcatggaggctgaagatgctgccacttattactgccagcagtggagtagtagcccacccacgttcggtgctgggaccaagctggaactgaaa

SEQ ID NO: 363 - DNA, 360 bp, Mus musculus
  misc_feature: Murine SC17.191 VH
gaggtccagctgcaacagtctggacctgagctaatgaagcctggggcttcagtgaagatgtcctgcaaggcttctggatacacattcactgactacaacatgcactgggtgaagcagaaccaaggaaagagcctagagtggataggagaaattaatcctaacactggtggtactggctacaaccagaagttcaaagacaaggccacattgactgtagacaagtcctccagcacagcctacatggagctccgcagcctgacatctgaggactctgcagtctattactgtgcaagaattccctccctgagacgatactactttgactactggggccaaggcaccactctcacagtctcctca

SEQ ID NO: 364 - DNA, 333 bp, Mus musculus
  misc_feature: Murine SC17.193 VL
gaccttgtgctgacacagtctcctgcttccttagctgtatctctggggcagagggccaccatctcatgcagggccagcgaaagtgtcagtacatctggctatagttatatgcactggtaccaacagaaaccaggacagccacccaaactcctcatctatcttgcatccaacctcgaatctggggtccctgccaggttcagtggcagtgggtctgggacagacttcaccctcaacatccatcctgtggaggaggaggatgctacaacctattactgtcagcacagtagggagcttccgtacacgttcggaggggggaccaagctggaaataaaa

SEQ ID NO: 365 - DNA, 366 bp, Mus musculus
  misc_feature: Murine SC17.193 VH
caggttactctgaaagagtctggccctgggatattgcagccctcccagaccctcagtctgacttgttctttctctgggttttcactgatcacttatggtataggagtaggctggattcgtcagccttcagggaagggtctggagtggctggcacacatttggtggaatgataataagtactataacacagccctgaagagccggctcacaatctccaaggatacctccaacaaccaggtattcctcaagatcgccaatgtggacactgcagatactgccacatactactgtgctcgaatggtctactatgattacgacggggggtttgcttactggggccaagggactctggtcactgtctctgca

SEQ ID NO: 366 - DNA, 333 bp, Mus musculus
  misc_feature: Murine SC17.199 VL
gacattgtgctgacccaatctccagcttctttggctgtgtctctagggcagagggccaccatatcctgcagagccagtgaaagtgttgatagttatggcaatagttttatgcactggtaccagcagaaaccaggacagccacccaaacccctcatttatcgtgcatccaacctagaatctgggatccctgccagattcagtggcagtgggtctaggacagacttcaccctcaccattaatcctgtggaggctgatgatgttgcaacctattactgtcagcaaagtaatgaggatccgtacacgttcggaggggggaccaagctggaaataaaa

SEQ ID NO: 367 - DNA, 348 bp, Mus musculus
  misc_feature: Murine SC17.199 VH
gaggtgcagctgcagcagtctgggactgtgctggcaaggcctggggcttcagtaaggatgtcctgcaaggcttctggctacacctttaccagctactggatgcactgggtaaaacaaaggcctggacagggtctggaatggattggcgctatttatcctggaaatagtgatactagctacaaccataagttcaagggcaaggccaaactgactgcagtcacatctgccagcactgcctacatggagctcagcagcctgacaaatgaggactctgcggtctattactgtacaagatctgggacgggctggtttgcttactggggccaagggactctggtcactgtctct

SEQ ID NO: 368 - DNA, 333 bp, Mus musculus
  misc_feature: Murine SC17.200 VL
gacattgtgctgacccaatctccagcttctttggctgtgtctctaggacagagagccactatcttctgcagagccagccagagtgtcgattataatggaattagttatatgcactggttccaacaaaaaccaggacagccacccaaactcctcatctatgctgcatccaacgttcaatctgggatccctgccaggttcagtggcagtgggtctgggacagacttcaccctcaacatccatcctgtggaggaggaagatgctgcaaccttttactgtcagcaaagtattgaggatcctccgacgttcggtggaggcaccaagctggaaatcaaa

SEQ ID NO: 369 - DNA, 345 bp, Mus musculus
  misc_feature: Murine SC17.200 VH
caggtccagctgcagcagtctggacctgagctggtgaaacctggggcctcagtgaagatttcctgcaaagcttctggctacgcattcagtagttcttggattaactgggtgaagcagaggcctggacagggtcttgagtggattggacggatttatcctggagaaggtgatactaactacagtgggaatttcgagggcaaggccacactgactgcagacaaatcctccaccacagcctacatgcagctcagcagtctgacctctgtggactctgcggtctatttctgtacaagaggactagtcatggactactggggccaaggcaccgctctcacagtctcctca

SEQ ID NO: 370 - DNA, 321 bp, Artificial Sequence
  Humanized hSC17.16 VL
gacatccagatgacccagtctccatcctccctgtctgcatctgtaggagacagagtcaccatcacttgccgggcaagtgcgaacattaacagcaatttagtttggtatcagcagaaaccagggaaagcccctaagctcctgatctatgctgcaaccaatttggcagatggggtcccatcaaggttcagtggcagtggatctgggacagatttcactctcaccatcagcagtctgcaacctgaagattttgcaacttactactgtcaacatttttggggtactcctcggacgttcggtggaggcaccaagctggaaatcaaa

SEQ ID NO: 371 - DNA, 348 bp, Artificial Sequence
  Humanized hSC17.16 VH
caggtgcagctggtgcagtctggggctgaggtgaagaagcctggggcctcagtgaaggtctcctgcaaggcttctggatacaccttcaccgactacaatatgtactgggtgcgacaggcccctggacaagggcttgagtggatgggagagatcaaccctaacaatggtggcacagcctataatcagaagtttaggggcaaggtcaccatgaccagggacacgtccatcagcacagcctacatggagctgagcaggctgagatctgacgacacggccgtgtattactgtgcgagatatgataaggggtttgactactggggccaaggcaccactgtcacagtctcctca

SEQ ID NO: 372 - DNA, 318 bp, Artificial Sequence
  Humanized hSC17.17 VL
gaaattgtgttgacacagtctccagccaccctgtctttgtctccaggggaaagagccaccctctcctgcagtgccagtagcagtgttagctacatgcattggtaccaacagaaacctggccaggctcccaggctcctcatctatgatacatccaaattgcccagtggcatcccagccaggttcagtggcagtgggtctgggacagacttcactctcaccatcagcagcctagagcctgaagattttgcagtttattactgtcagcagtggagtagtaccccacccacgttcggtcaggggaccaagctggagattaaa

SEQ ID NO: 373 - DNA, 360 bp, Artificial Sequence
  Humanized hSC17.17 VH
caggtgcagctggtgcagtctggggctgaggtgaagaagcctggggcctcagtgaaggtctcctgcaaggcttctggatacaccttcaccgactacaatatgcactgggtgcgacaggcccctggacaagggcttgagtggatgggagagatcaaccctaacattggtggcacaggctataaccagaagtttaagggcagggtcaccatgaccagggacacgtccatcagcacagcctacatggagctgagcaggctgagatctgacgacacggccgtgtattactgtgcgagaacctatagttactatagttacgagtttgcttactggggccaagggactctggtcactgtctcttca

SEQ ID NO: 374 - DNA, 336 bp, Artificial Sequence
  Humanized hSC17.24 VL
gacatcgtgatgacccagtctccagactccctggctgtgtctctgggcgagagggccaccatcaactgcaagtccagccagagtcttctctacagctccaaccagaagagctacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtaagcaatcttataatcttcggacgttcggtggaggcaccaaggtggaaatcaaa

SEQ ID NO: 375 - DNA, 351 bp, Artificial Sequence
  Humanized hSC17.24 VH
gaagtgcagctggtgcagtctggggctgaggtgaagaagcctggggccaccgtgaagatatcctgcaaggtgtctggatacaccttcacagaccacactatacactgggtgcgacaggcccctggaaaggggcttgagtggattggatacatctaccctcgtgatggtagcacaaaatacaacgaggagttcaaaggcagagtcaccatcaccgccgacacgtccacggacacagcctacatggagctgagcagcctgagatctgaggacacggccgtgtattactgtgcgagatcatatagtaactactttgactactggggccaaggcaccactgtcacagtctcctca

SEQ ID NO: 376 - DNA, 321 bp, Artificial Sequence
  Humanized hSC17.28 VL
gaaattgtgctgactcagtctccagactttcagtctgtgactccaaaggagaaagtcaccatcacctgccgggccagtcagagcattggtactagcatacactggtaccagcagaaaccagatcagtctccaaagctcctcatcaagtatgcttccgagtccatctcaggggtcccctcgaggttcagtggcagtggatctgggacagatttcaccctcaccatcaatagcctggaagctgaagatgctgcaacgtattactgtcagcaaagtaatagctggccactcacgttcggtcaagggaccaagctggagataaaa

SEQ ID NO: 377 - DNA, 351 bp, Artificial Sequence
  Humanized hSC17.28 VH
caggtgcagctggtgcagtctggggctgaggtgaagaagcctggggcctcagtgaaggtctcctgcaaggcttctggatacaccttcaccagaagctatatccactgggtgcgacaggcccctggacaagggcttgagtggatgggatacatcagcagtggcagtggtggcacaacctataaccagaagtttaagggcagggtcaccagtaccagggacacgtccatcagcacagcctacatggagctgagcaggctgagatctgacgacacggccgtgtattactgtgcgagagggggggtacggtacttcgatgtctggggccaagggaccacggtcaccgtctcctca

SEQ ID NO: 378 - DNA, 321 bp, Artificial Sequence
  Humanized hSC17.34 VL
gacatccagatgacccagtctccatcctcactgtctgcatctgtaggagacagagtcaccatcacttgtaaggcgagtcaggacattaatagttatttatcctggtttcagcagaaaccagggaaagcccctaagtccctgatctatagagcaaacagattggtagatggggtcccatcaaggttcagcggcagtggatctgggacagattacactctcaccatcagcagcctgcagcctgaagattttgcaacttattactgcctacagtatgatgagtttcctccgacgttcggtcagggcaccaagctggaaatcaaa

SEQ ID NO: 379 - DNA, 354 bp, Artificial Sequence
  Humanized hSC17.34 VL
caggtccagcttgtgcagtctggggctgaggtgaagaagcctggggcctcagtgaaggtttcctgcaaggcttctggatacaccttcactgactataatatggattgggtgcgccaggcccccggacaaaggcttgagtggattggatacatctaccctgacaatggtggcgcaggatataatcagaagttcaagggcagagtcaccattaccgtggacacatccgcgagcacagcctacatggagctgagcagcctgagatctgaagacacggctgtgtattactgttcaagatccattactacggcttggtttgcttactggggccaagggactctggtcactgtctcttca

SEQ ID NO: 380 - DNA, 321 bp, Artificial Sequence
  Humanized hSC17.46 VL
gccatccagatgacccagtctccatcctccctgtctgcatctgtaggagacagagtcaccatcacttgcaaggcaagtcagagcgttaataatgatgtagcctggtatcagcagaaaccagggaaagcccctaagctcctgatctattatgcatccaatcgatatactggggtcccatcaaggttcagcggcagtggatctggcacagatttcactctcaccatcagcagcctgcagcctgaagattttgcaacttatttctgtcagcaggattatagctctcctcggacgttcggtcaggggaccaagctggaaataaag

SEQ ID NO: 381 - DNA, 363 bp, Artificial Sequence
  Humanized hSC17.46 VH
caggtgcagctggtgcagtctggggctgaggtgaagaagcctggggcctcagtgaaggtctcctgcaaggcttctggatacaccttcaccagctactggatcaactgggtgcgacaggcccctggacaagggcttgagtggattggaaacatcttccctgacactactaccacaaactataacgagaagtttaagggcagggtcaccctgaccagggacacgtccatcagcacagcctacatggagctgagcaggctgagatctgacgacacggccgtgtattactgtgcgagagagtactacgatggtacctacgatgctatggattactggggtcaaggaaccctagtcaccgtctcctca

SEQ ID NO: 382 - DNA, 333 bp, Artificial Sequence
  Humanized hSC17.151 VL
gagatcgtgctgacccagagccctgctacactgtccctgtcccctggagagagggccacactctcctgcagggcttccgagtccgtggattcctacggcaactccttcatgcactggtaccagcagaaacccggccaggcccctaggctgctgatctacagggcctccaacctggagtccggcatccctgctaggttctccggatccggctccggcaccgactttaccctgaccatctcctccctggagcccgaggacttcgccgtgtactactgccagcagtcccacgaggacccctacaccttcggccagggcaccaagctggagatcaag

SEQ ID NO: 383 - DNA, 351 bp, Artificial Sequence
  Humanized hSC17.151 VH
caggtccagctggtgcagagcggcgctgaggtgaagaagcctggcgccagcgtgaaggtgtcctgcaaagccagcggctacaccttcacctcctactggatgcattgggtgaggcaggctcctggccaaggactggagtggatgggcgccatctaccccggcaagtccgacaccacctacaaccagaagttcaagggcagggtgaccatgacacgggacacctccacctccaccgtgtacatggagctgtcctccctgaggtccgaggacaccgccgtgtactactgcgccaggtccggcaagggctatttcgcctactggggccagggcacactggtgaccgtgtcctcc

SEQ ID NO: 384 - DNA, 339 bp, Artificial Sequence
  Humanized hSC17.155 VL
gacatcgtgatgacccagtctccagactccctggctgtgtctctgggcgagagggccaccatcaactgcaagtccagccagagtttattatacagctccaaccaaaagaactacttagcttggtaccagcagaaaccaggacagcctcctaagctgctcatttactgggcatctacccggaaatccggggtccctgaccgattcagtggcagcgggtctgggacagatttcactctcaccatcagcagcctgcaggctgaagatgtggcagtttattactgtcatcaatattatagctatccgctcacgttcggtcaaggcaccaagctggaaatcaaa

SEQ ID NO: 385 - DNA, 339 bp, Artificial Sequence
  Humanized hSC17.155 VH
caggtgcagctggtgcagtctggggctgaggtgaagaagcctggggcctcagtgaaggtttcctgcaaggcatctggatacaccttcaacagctactggatgcactgggtgcgacaggcccctggacaagggcttgagtggatgggagaaatccaccctaataatggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 386 - DNA, 336 bp, Artificial Sequence
  Humanized hSC17.156 VL
gacatcgtgatgacccagacccctctgtccctgcctgtgacccctggagaacccgccagcatctcctgcaggtcctcccagtccatcgtgcactccaacggcaacacctacctggagtggtacctgcagaagcccggacagtccccccagctgctgatctacaaggtgtccaataggttttccggagtgcccgacaggttctccggatccggatccggcaccgacttcaccctgaagatctccagggtggaggccgaggacgtgggagtgtactactgcttccagggcagccacgtgccccctacattcggaggcggcaccaagctggagatcaag

SEQ ID NO: 387 - DNA, 354 bp, Artificial Sequence
  Humanized hSC17.156 VH
caggtcaccctgaaggagtccggccccgtgctggtgaaacccaccgagaccctcaccctgacctgcaccgtctccggcttctccctgtccacctccggcatgggagtgtcctggatcaggcagccccctggaaaggctctggagtggctggcccacatcttctgggacgacgacaagtggtacaacccctccctgaagtccaggctgaccatctccaaggacacctccaagtcccaggtggtgctgaccatgaccaacatggaccccgtggacaccgccacctactactgcgctaccttctacggcctgtacttcgcctactggggccagggaaccctggtgaccgtgtcctcc

SEQ ID NO: 388 - DNA, 339 bp, Artificial Sequence
  Humanized hSC17.161 VL
gacatcgtgatgacccagtcccccgattccctggctgtgagcctgggagagagggccaccatcaactgcgagtcctcccagtccctgctgtacaactccaaccagaagaactacctggcctggtaccagcagaagcccggacagccccccaagctgctgatctactgggcttccacaagggagtccggagtgcccgatcggttcagcggatccggatccggcaccgacttcaccctcaccatcagctccctgcaagccgaggacgtggccgtgtactactgccagcagtacttcaactaccctctgaccttcggccagggcaccaagctggagatcaag

SEQ ID NO: 389 - DNA, 348 bp, Artificial Sequence
  Humanized hSC17.161 VH
caggtgcagctggtccagtccggagctgaggtgaagaagcccggcgcctccgtgaaggtgtcctgcaaggccagcggcttcaccttctccgatgcctggatggactgggtgaggcaggctcctggccaaaggctggagtggatgggcgagatcaggtccaagcccaacaaccacgccacctactacgccgagagcgtgaagggcagggtgaccatcacaagggatacatccgcctccaccgcctacatggagctgtcctccctgaggtccgaggacaccgccgtgtactactgtgccaggaccggaacctcctactggggccagggcacactggtgaccgtgtcctcc

SEQ ID NO: 390 - DNA, 333 bp, Artificial Sequence
  Humanized hSC17.200 VL
gaaattgtgttgacacagtctccagccaccctgtctttgtctccaggggaaagagccaccctctcctgcagggccagtcagagtgttgactataatggaattagctacatgcactggtaccaacagaaacctggccaggctcccaggctcctcatctatgctgcatccaacgtgcagagtggcatcccagccaggttcagtggcagtgggtctgggacagacttcactctcaccatcagcagcctagagcctgaagattttgcagtttattactgtcagcagagtattgaggatcctccgacgttcggtggaggcaccaaggtggaaatcaaa

SEQ ID NO: 391 - DNA, 345 bp, Artificial Sequence
  Humanized hSC17.200 VH
gaggtgcagctggtgcagtctggagcagaggtgaaaaagcccggggagtctctgaagatctcctgtaagggttctggatacagctttaccagctcctggatcaactgggtgcgccagatgcccgggaaaggcctggagtggatggggagaatctatcctggtgagggtgataccaactacagcgggaacttcgaaggccaggtcaccatctcagccgacaagtccatcagcaccgcctacctgcagtggagcagcctgaaggcctcggacaccgccatgtattactgtacaagaggactagtcatggactactggggccaaggcacccttgtcacagtctcgagc

SEQ ID NO: 392 - DNA, 333 bp, Artificial Sequence
  Humanized hSC17.200 VL1
gaaattgtgttgacacagtctccagccaccctgtctttgtctccaggggaaagagccaccctctcctgcagggccagtcagagtgttgactatgatggaattagctacatgcactggtaccaacagaaacctggccaggctcccaggctcctcatctatgctgcatccaacgtgcagagtggcatcccagccaggttcagtggcagtgggtctgggacagacttcactctcaccatcagcagcctagagcctgaagattttgcagtttattactgtcagcagagtattgaggatcctccgacgttcggtggaggcaccaaggtggaaatcaaa

SEQ ID NO: 393 - DNA, 339 bp, Artificial Sequence
  Humanized hSC17.155 VH1
caggtgcagctggtgcagtctggggctgaggtgaagaagcctggggcctcagtgaaggtttcctgcaaggcatctggatacaccttcgacagctactggatgcactgggtgcgacaggcccctggacaagggcttgagtggatgggagaaatccaccctaataatggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 394 - DNA, 339 bp, Artificial Sequence
  Humanized hSC17.155 VH2
caggtgcagctggtgcagtctggggctgaggtgaagaagcctggggcctcagtgaaggtttcctgcaaggcatctggatacaccttcaccagctactggatgcactgggtgcgacaggcccctggacaagggcttgagtggatgggagaaatccaccctaataatggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 395 - DNA, 339 bp, Artificial Sequence
  Humanized hSC17.155 VH3
caggtgcagctggtgcagtctggggctgaggtgaagaagcctggggcctcagtgaaggtttcctgcaaggcatctggatacaccttcaactactactggatgcactgggtgcgacaggcccctggacaagggcttgagtggatgggagaaatccaccctaataatggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 396 - DNA, 339 bp, Artificial Sequence
  Humanized hSC17.155 VH4
caggtgcagctggtgcagtctggggctgaggtgaagaagcctggggcctcagtgaaggtttcctgcaaggcatctggatacaccttcaacagctactggatgcactgggtgcgacaggcccctggacaagggcttgagtggatgggagaaatccaccctaatgatggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 397 - DNA, 339 bp, Artificial Sequence
  Humanized hSC17.155 VH5
caggtgcagctggtgcagtctggggctgaggtgaagaagcctggggcctcagtgaaggtttcctgcaaggcatctggatacaccttcaacagctactggatgcactgggtgcgacaggcccctggacaagggcttgagtggatgggagaaatccaccctaatggtggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 398 - DNA, 339 bp, Artificial Sequence
  Humanized hSC17.155 VH6
caggtgcagctggtgcagtctggggctgaggtgaagaagcctggggcctcagtgaaggtttcctgcaaggcatctggatacaccttcaacagctactggatgcactgggtgcgacaggcccctggacaagggcttgagtggatgggagaaatccaccctaatagtggtagcacaaactacaacgagaagttcaagggcagagtcaccatgaccagggacacgtccacgagcacagtctacatggagctgagcagcctgagatctgaggacacggccgtgtattactgtgcgagatggactttgtttacttactggggccaagggactctggtcactgtc

SEQ ID NO: 399 - DNA, 348 bp, Artificial Sequence
  Humanized hSC17.161 VH1
gaggtgcagctggtggaatccggaggcggcctggtgcaacctggaggatccctcaggctgtcctgtgccgcttccggattcaccttctccgatgcctggatggactgggtgaggcaggcccctggcaaaggactggaatgggtgggcgagatcaggtccaaacccaacaaccacgccacctactacgccgagtccgtgaagggcaggttcaccatctccagggacgactccaagaactccctgtacctgcagatgaactccctgaagaccgaggacaccgccgtgtactactgcgctaggaccggcacctcctattggggacagggcaccctggtgaccgtgtcctcc

SEQ ID NO: 400 - protein, 218 aa, Artificial Sequence
  Humanized hSC17.200 light chain
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - protein, 444 aa, Artificial Sequence
  Humanized hSC17.200 heavy chain
EVQLVQSGAEVKKPGESLKISCKGSGYSFTSSWINWVRQMPGKGLEWMGRIYPGEGDTNYSGNFEGQVTISADKSISTAYLQWSSLKASDTAMYYCTRGLVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 402 - protein, 218 aa, Artificial Sequence
  Humanized hSC17.200vL1 light chain
EIVLTQSPATLSLSPGERATLSCRASQSVDYDGISYMHWYQQKPGQAPRLLIYAASNVQSGIPARFSGSGSGTDFTLTISSLEPEDFAVYYCQQSIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 403 - protein, 107 aa, Homo sapiens [sequence omitted]

SEQ ID NO: 404 - protein, 329 aa, Homo sapiens
  MISC_FEATURE: IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 405 - protein, 11 aa, Mus musculus
  MISC_FEATURE: hSC17.16 CDRL1
RASANINSNLV

SEQ ID NO: 406 - protein, 7 aa, Mus musculus
  MISC_FEATURE: hSC17.16 CDRL2
AATNLAD

SEQ ID NO: 407 - protein, 9 aa, Mus musculus
  MISC_FEATURE: hSC17.16 CDRL3
QHFWGTPRT

SEQ ID NO: 408 - protein, 5 aa, Mus musculus
  MISC_FEATURE: hSC17.16 CDRH1
DYNMY

SEQ ID NO: 409 - protein, 17 aa, Mus musculus
  MISC_FEATURE: hSC17.16 CDRH2
EINPNNGGTAYNQKFRG

SEQ ID NO: 410 - protein, 7 aa, Mus musculus
  MISC_FEATURE: hSC17.16 CDRH3
YDKGFDY

SEQ ID NO: 411 - protein, 10 aa, Mus musculus
  MISC_FEATURE: hSC17.17 CDRL1
SASSSVSYMH

SEQ ID NO: 412 - protein, 7 aa, Mus musculus
  MISC_FEATURE: hSC17.17 CDRL2
DTSKLPS

SEQ ID NO: 413 - protein, 9 aa, Mus musculus
  MISC_FEATURE: hSC17.17 CDRL3
QQWSSTPPT

SEQ ID NO: 414 - protein, 5 aa, Mus musculus
  MISC_FEATURE: hSC17.17 CDRH1
DYNMH

SEQ ID NO: 415 - protein, 17 aa, Mus musculus
  MISC_FEATURE: hSC17.17 CDRH2
EINPNIGGTGYNQKFKG

SEQ ID NO: 416 - protein, 11 aa, Mus musculus
  MISC_FEATURE: hSC17.17 CDRH3
TYSYYSYEFAY

SEQ ID NO: 417 - protein, 17 aa, Mus musculus
  MISC_FEATURE: hSC17.24 CDRL1
KSSQSLLYSSNQKSYLA

SEQ ID NO: 418 - protein, 7 aa, Mus musculus
  MISC_FEATURE: hSC17.24 CDRL2
WASTRES

SEQ ID NO: 419 - protein, 8 aa, Mus musculus
  MISC_FEATURE: hSC17.24 CDRL3
KQSYNLRT

SEQ ID NO: 420 - protein, 5 aa, Mus musculus
  MISC_FEATURE: hSC17.24 CDRH1
DHTIH

SEQ ID NO: 421 - protein, 17 aa, Mus musculus
  MISC_FEATURE: hSC17.24 CDRH2
YIYPRDGSTKYNEEFKG

SEQ ID NO: 422 - protein, 8 aa, Mus musculus
  MISC_FEATURE: hSC17.24 CDRH3
SYSNYFDY

SEQ ID NO: 423 - protein, 11 aa, Mus musculus
  MISC_FEATURE: hSC17.28 CDRL1
RASQSIGTSIH

SEQ ID NO: 424 - protein, 7 aa, Mus musculus
  MISC_FEATURE: hSC17.28 CDRL2
YASESIS

SEQ ID NO: 425 - protein, 9 aa, Mus musculus
  MISC_FEATURE: hSC17.28 CDRL3
QQSNSWPLT

SEQ ID NO: 426 - protein, 5 aa, Mus musculus
  MISC_FEATURE: hSC17.28 CDRH1
RSYIH

SEQ ID NO: 427 - protein, 17 aa, Mus musculus
  MISC_FEATURE: hSC17.28 CDRH2
YISSGSGGTTYNQKFKG

SEQ ID NO: 428 - protein, 8 aa, Mus musculus
  MISC_FEATURE: hSC17.28 CDRH3
GGVRYFDV

SEQ ID NO: 429 - protein, 11 aa, Mus musculus
  MISC_FEATURE: hSC17.34 CDRL1
KASQDINSYLS

SEQ ID NO: 430 - protein, 7 aa, Mus musculus
  MISC_FEATURE: hSC17.34 CDRL2
RANRLVD

SEQ ID NO: 431 - protein, 9 aa, Mus musculus
  MISC_FEATURE: hSC17.34 CDRL3
LQYDEFPPT

SEQ ID NO: 432 - protein, 5 aa, Mus musculus
  MISC_FEATURE: hSC17.34 CDRH1
DYNMD

SEQ ID NO: 433 - protein, 17 aa, Mus musculus
  MISC_FEATURE: hSC17.34 CDRH2
YIYPDNGGAGYNQKFKG

SEQ ID NO: 434 - protein, 9 aa, Mus musculus
  MISC_FEATURE: hSC17.34 CDRH3
SITTAWFAY

SEQ ID NO: 435 - protein, 11 aa, Mus musculus
  MISC_FEATURE: hSC17.46 CDRL1
KASQSVNNDVA

SEQ ID NO: 436 - protein, 7 aa, Mus musculus
  MISC_FEATURE: hSC17.46 CDRL2
YASNRYT

SEQ ID NO: 437 - protein, 9 aa, Mus musculus
  MISC_FEATURE: hSC17.46 CDRL3
QQDYSSPRT

SEQ ID NO: 438 - protein, 5 aa, Mus musculus
  MISC_FEATURE: hSC17.46 CDRH1
SYWIN

SEQ ID NO: 439 - protein, 17 aa, Mus musculus
  MISC_FEATURE: hSC17.46 CDRH2
NIFPDTTTTNYNEKFKG

SEQ ID NO: 440 - protein, 12 aa, Mus musculus
  MISC_FEATURE: hSC17.46 CDRH3
EYYDGTYDAMDY

SEQ ID NO: 441 - protein, 15 aa, Mus musculus
  MISC_FEATURE: hSC17.151 CDRL1
RASESVDSYGNSFMH

SEQ ID NO: 442 - protein, 7 aa, Mus musculus
  MISC_FEATURE: hSC17.151 CDRL2
RASNLES

SEQ ID NO: 443 - protein, 9 aa, Mus musculus
  MISC_FEATURE: hSC17.151 CDRL3
QQSHEDPYT

SEQ ID NO: 444 - protein, 5 aa, Mus musculus
  MISC_FEATURE: hSC17.151 CDRH1
SYWMH

SEQ ID NO: 445 - protein, 17 aa, Mus musculus
  MISC_FEATURE: hSC17.151 CDRH2
AIYPGKSDTTYNQKFKG

SEQ ID NO: 446 - protein, 8 aa, Mus musculus
  MISC_FEATURE: hSC17.151 CDRH3
SGKGYFAY

SEQ ID NO: 447 - protein, 17 aa, Mus musculus
  MISC_FEATURE: hSC17.155 and hSC17.155vH1-6 CDRL1
KSSQSLLYSSNQKNYLA

SEQ ID NO: 448 - protein, 7 aa, Mus musculus
  MISC_FEATURE: hSC17.155 and hSC17.155vH1-6 CDRL2
WASTRKS

SEQ ID NO: 449 - protein, 9 aa, Mus musculus
  MISC_FEATURE: hSC17.155 and hSC17.155vH1-6 CDRL3
HQYYSYPLT

SEQ ID NO: 450 - protein, 5 aa, Mus musculus
  MISC_FEATURE: hSC17.155 and hSC17.155vH1, vH2 and vH4-6 CDRH1
SYWMH

SEQ ID NO: 451 - protein, 17 aa, Mus musculus
  MISC_FEATURE: hSC17.155 and hSC17.155vH1-3 CDRH2
EIHPNNGSTNYNEKFKG

SEQ ID NO: 452 - protein, 6 aa, Mus musculus
  MISC_FEATURE: hSC17.155 and hSC17.155vH1-6 CDRH3
WTLFTY

SEQ ID NO: 453 - protein, 16 aa, Mus musculus
  MISC_FEATURE: hSC17.156 CDRL1
RSSQSIVHSNGNTYLE

SEQ ID NO: 454 - protein, 7 aa, Mus musculus
  MISC_FEATURE: hSC17.156 CDRL2
KVSNRFS

SEQ ID NO: 455 - protein, 9 aa, Mus musculus
  MISC_FEATURE: hSC17.156 CDRL3
FQGSHVPPT

SEQ ID NO: 456 - protein, 7 aa, Mus musculus
  MISC_FEATURE: hSC17.156 CDRH1
TSGMGVS

SEQ ID NO: 457 - protein, 16 aa, Mus musculus
  MISC_FEATURE: hSC17.156 CDRH2
HIFWDDDKWYNPSLKS

SEQ ID NO: 458 - protein, 8 aa, Mus musculus
  MISC_FEATURE: hSC17.156 CDRH3
FYGLYFAY

SEQ ID NO: 459 - protein, 17 aa, Mus musculus
  MISC_FEATURE: hSC17.161 and hSC17.161vL1 CDRL1
ESSQSLLYNSNQKNYLA

SEQ ID NO: 460 - protein, 7 aa, Mus musculus
  MISC_FEATURE: hSC17.161 and hSC17.161vL1 CDRL2
WASTRES

SEQ ID NO: 461 - protein, 9 aa, Mus musculus
  MISC_FEATURE: hSC17.161 and hSC17.161vL1 CDRL3
QQYFNYPLT

SEQ ID NO: 462 - protein, 5 aa, Mus musculus
  MISC_FEATURE: hSC17.161 and hSC17.161vL1 CDRH1
DAWMD

SEQ ID NO: 463 - protein, 19 aa, Mus musculus
  MISC_FEATURE: hSC17.161 and hSC17.161vL1 CDRH2
EIRSKPNNHATYYAESVKG

SEQ ID NO: 464 - protein, 5 aa, Mus musculus
  MISC_FEATURE: hSC17.161 and hSC17.161vL1 CDRH3
TGTSY

SEQ ID NO: 465 - protein, 15 aa, Mus musculus
  MISC_FEATURE: hSC17.200 CDRL1
RASQSVDYNGISYMH

SEQ ID NO: 466 - protein, 7 aa, Mus musculus
  MISC_FEATURE: hSC17.200 and hSC17.200vL1 CDRL2
AASNVQS

SEQ ID NO: 467 - protein, 9 aa, Mus musculus
  MISC_FEATURE: hSC17.200 and hSC17.200vL1 CDRL3
QQSIEDPPT

SEQ ID NO: 468 - protein, 5 aa, Mus musculus
  MISC_FEATURE: hSC17.200 and hSC17.200vL1 CDRH1
SSWIN

SEQ ID NO: 469 - protein, 17 aa, Mus musculus
  MISC_FEATURE: hSC17.200 and hSC17.200vL1 CDRH2
RIYPGEGDTNYSGNFEG

SEQ ID NO: 470 - protein, 6 aa, Mus musculus
  MISC_FEATURE: hSC17.200 and hSC17.200vL1 CDRH3
GLVMDY

SEQ ID NO: 471 - protein, 30 aa, Homo sapiens
  MISC_FEATURE: hSC17.155vH1 FR1
QVQLVQSGAEVKKPGASVKVSCKASGYTFD

SEQ ID NO: 472 - protein, 30 aa, Homo sapiens
  MISC_FEATURE: hSC17.155vH2 FR1
QVQLVQSGAEVKKPGASVKVSCKASGYTFT

SEQ ID NO: 473 - protein, 5 aa, Mus musculus
  MISC_FEATURE: hSC17.155vH3 CDRH1
YYWMH

SEQ ID NO: 474 - protein, 17 aa, Mus musculus
  MISC_FEATURE: hSC17.155vH4 CDRH2
EIHPNDGSTNYNEKFKG

SEQ ID NO: 475 - protein, 17 aa, Mus musculus
  MISC_FEATURE: hSC17.155vH5 CDRH2
EIHPNGGSTNYNEKFKG

SEQ ID NO: 476 - protein, 17 aa, Mus musculus
  MISC_FEATURE: hSC17.155vH6 CDRH2
EIHPNSGSTNYNEKFKG

SEQ ID NO: 477 - protein, 30 aa, Homo sapiens
  MISC_FEATURE: hSC17.161vH1 FR1
EVQLVESGGGLVQPGGSLRLSCAASGFTFS

SEQ ID NO: 478 - protein, 14 aa, Homo sapiens
  MISC_FEATURE: hSC17.161vH1 FR2
WVRQAPGKGLEWVG

SEQ ID NO: 479 - protein, 32 aa, Homo sapiens
  MISC_FEATURE: hSC17.161vH1 FR3
RFTISRDDSKNSLYLQMNSLKTEDTAVYYCAR

SEQ ID NO: 480 - protein, 15 aa, Mus musculus
  MISC_FEATURE: hSC17.200vL1 CDRL1
RASQSVDYDGISYMH

SEQ ID NO: 481-499 - skipped (19 entries)

SEQ ID NO: 500 - protein, 329 aa, Homo sapiens
  MISC_FEATURE: C220S IgG1 heavy constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 501 - protein, 328 aa, Homo sapiens
  MISC_FEATURE: C220delta IgG1 heavy constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 502 - protein, 106 aa, Homo sapiens
  MISC_FEATURE: C214delta Kappa light chain constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 503 - protein, 107 aa, Homo sapiens
  MISC_FEATURE: C214S Kappa light chain constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 504 - protein, 105 aa, Homo sapiens
  MISC_FEATURE: Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 505 - protein, 104 aa, Homo sapiens
  MISC_FEATURE: C214delta Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTES

SEQ ID NO: 506 - protein, 105 aa, Homo sapiens
  MISC_FEATURE: C214S Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTESS

SEQ ID NO: 507 - protein, 214 aa, Mus musculus
  MISC_FEATURE: SC16.56 ss1 and ss2 full length light chain
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 508 - protein, 447 aa, Mus musculus
  MISC_FEATURE: SC16.56 ss3 and ss4 full length heavy chain
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 509 - protein, 447 aa, Mus musculus
  MISC_FEATURE: SC16.56 ss1 full length heavy chain
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 510 - protein, 446 aa, Mus musculus
  MISC_FEATURE: SC16.56 ss2 full length heavy chain
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 511 - protein, 213 aa, Mus musculus
  MISC_FEATURE: SC16.56 ss3 full length light chain
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 512 - protein, 214 aa, Mus musculus
  MISC_FEATURE: SC16.56 ss4 full length light chain
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 513 - protein, 218 aa, Mus musculus; identical to SEQ ID NO: 400
  MISC_FEATURE: SC17.200 ss1 and ss2 full length light chain

SEQ ID NO: 514 - protein, 444 aa, Mus musculus; identical to SEQ ID NO: 401
  MISC_FEATURE: SC17.200 ss3 and ss4 full length heavy chain

SEQ ID NO: 515 - protein, 444 aa, Mus musculus
  MISC_FEATURE: SC17.200 ss1 full length heavy chain
EVQLVQSGAEVKKPGESLKISCKGSGYSFTSSWINWVRQMPGKGLEWMGRIYPGEGDTNYSGNFEGQVTISADKSISTAYLQWSSLKASDTAMYYCTRGLVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 516 - protein, 443 aa, Mus musculus
  MISC_FEATURE: SC17.200 ss2 full length heavy chain
EVQLVQSGAEVKKPGESLKISCKGSGYSFTSSWINWVRQMPGKGLEWMGRIYPGEGDTNYSGNFEGQVTISADKSISTAYLQWSSLKASDTAMYYCTRGLVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 517 - protein, 217 aa, Mus musculus
  MISC_FEATURE: SC17.200 ss3 full length light chain
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 518 - protein, 218 aa, Mus musculus
  MISC_FEATURE: SC17.200 ss4 full length light chain
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 519 - protein, 106 aa, Artificial Sequence
  hSC16.13 light chain variable region
DIQMTQSPSSLSASVGDRVTITCSASSSVSYMYWYQQKPGKAPKLLIYLTSNLASGVPSRFSGSGSGTDFTLTISSLQPEDFATYYCQQWRSNPFTFGQGTKLEIK

SEQ ID NO: 520 - protein, 107 aa, Artificial Sequence
  hSC16.15 light chain variable region
AIQLTQSPSSLSASVGDRVTITCRASENIYYNLAWYQQKPGKAPKLLIYTANSLEDGVPSRFSGSGSGTDFTLTISSLQPEDFATYFCKQAYDVPPTFGGGTKLEIK

SEQ ID NO: 521 - protein, 106 aa, Artificial Sequence
  hSC16.25 light chain variable region
EIVLTQSPDFQSVTPKEKVTITCSASSSVSYMHWYQQKPDQSPKLLIKDSSKLASGVPSRFSGSGSGTDFTLTINSLEAEDAATYYCQQWSSNPLTFGQGTKLEIK

SEQ ID NO: 522 - protein, 107 aa, Artificial Sequence
  hSC16.34 light chain variable region
DIQMTQSPSSLSASVGDRVTITCKASQSVSNDVAWYQQKPGKVPKLLIYYASNRYSGVPSRFSGSGSGTDFTLTISSLQPEDVATYFCQQDYSSPWTFGGGTKVEIK

SEQ ID NO: 523 - protein, 107 aa, Artificial Sequence
  hSC16.56 light chain variable region
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 524 - protein, 124 aa, Artificial Sequence
  hSC16.13 heavy chain variable region
QITLKESGPTLVKPTQTLTLTCTFSGFSLSTSGMGVGWIRQPPGKALEWLAHIWWDDVKRYSPSLKSRLTITKDTSKNQVVLTMTNMDPVDTATYYCARIVSFDNDVVSAMDYWGQGTLVTVSS

SEQ ID NO: 525 - protein, 117 aa, Artificial Sequence
  hSC16.15 heavy chain variable region
QVQLVQSGAEVKKPGASVKVSCKASGYTFTRYWIHWIRQAPGQGLEWMGYINPTTVYTEFNQNFKDRVTMTRDTSTSTVYMELSSLRSEDTAVYYCARGGSNFFDYWGQGTTVTVSS

SEQ ID NO: 526 - protein, 124 aa, Artificial Sequence
  hSC16.25 heavy chain variable region
QITLKESGPTLVKPTQTLTLTCTFSGFSLSTSGMGVGWIRQPPGKALEWLTDIWWDDNKYYNPSLKSRLTITKDTSKNQVVLTMTNMDPVDTATYYCARRVNYYYDPYYAMDYWGQGTLVTVSS

SEQ ID NO: 527 - protein, 118 aa, Artificial Sequence
  hSC16.34 heavy chain variable region
QVQLVQSGAEVKKPGASVKVSCKASGYTFTNYGMNWVRQAPGQRLEWMGWINTYTGDPTYADDFKGRVTITRDTSASTAYMELSSLRSEDTAVYYCARIGGNSPSDYWGQGTTVTVSS

SEQ ID NO: 528 - protein, 118 aa, Artificial Sequence
  hSC16.56 heavy chain variable region
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 529 - protein, 112 aa, Mus musculus
  MISC_FEATURE: SC10.17 light chain variable region
DVLMTQTPLSLPVSLGDQASISCRSSQSIVHSDGNTYLEWYLRKPGQSPRLLIYKVSNRFSGVPDRFSGSGSGTDFTLKISRVEAEDLGVYYCFQGSHAPWTFGGGTKLEIK

SEQ ID NO: 530 - protein, 120 aa, Mus musculus
  MISC_FEATURE: SC10.17 heavy chain variable region
DVQLVESGGGLVQPGGSRKLSCAASGFTFSSYGMHWVRQAPETGLEWVAYITTRSSTIYYAATVKGRFTISRDNARNTLFLQMTSLRSEDTAMYYCTREPLTGYYAMDYWGQGTSVTVSS

SEQ ID NO: 531 - protein, 112 aa, Artificial Sequence
  hSC10.17 light chain variable region
DVVMTQSPLSLPVTLGQPASISCRSSQSIVHSDGNTYLEWYQQRPGQSPRRLIYKVSNRFSGVPDRFSGSGSGTDFTLKISRVEAEDVGVYYCFQGSHAPWTFGGGTKVEIK

SEQ ID NO: 532 - protein, 120 aa, Artificial Sequence
  hSC10.17 heavy chain variable region
EVQLVESGGGLVQPGGSLRLSCAASGFTFSSYGMHWVRQAPGKGLEWVAYITTRSSTIYYADSVKGRFTISRDNAKNSLYLQMNSLRAEDTAVYYCTREPLTGYYAMDYWGQGTSVTVSS

SEQ ID NO: 533 - DNA, 337 bp, Mus musculus
  misc_feature: SC10.17 light chain variable region
gatgttttgatgacccaaactccactctccctgcctgtcagtcttggagatcaagcctccatctcttgcagatctagtcagagcattgtacatagtgatggaaacacctatttagaatggtacctgcggaaaccaggccagtctccaagactcctgatctacaaagtttccaaccgattttctggggtcccagacaggttcagtggcagtggatcagggacagatttcacactcaagatcagcagagtggaggctgaggatctgggagtttattactgctttcaaggttcacatgctccgtggacgttcggtggaggcaccaagctggaaatcaaac

SEQ ID NO: 534 - DNA, 360 bp, Mus musculus
  misc_feature: SC10.17 heavy chain variable region
gatgtgcagctggtggagtctgggggaggcttagtgcagcctggagggtcccggaaactctcctgtgcagcctctggattcactttcagtagctacggaatgcactgggttcgtcaggctccagagacggggctggagtgggtcgcatacattactactcgcagtagtaccatctactatgcagccacagtgaagggccgattcaccatctccagagacaatgccaggaacaccctgttcctgcaaatgaccagtctaaggtctgaggacacggccatgtattactgtactagagaacccctaactggatactatgctatggactactggggtcaaggaacctcagtcaccgtctcctca

SEQ ID NO: 535 - DNA, 337 bp, Artificial Sequence
  hSC10.17 light chain variable region
gatgttgtgatgactcagtctccactctccctgcccgtcacccttggacagccggcctccatctcctgcaggtctagtcaaagcatcgtacacagtgatggaaacacctacttggaatggtatcagcagaggccaggccaatctccaaggcgcctaatttataaggtttctaaccggttctctggggtcccagacagattcagcggcagtgggtcaggcactgatttcacactgaaaatcagcagggtggaggctgaggatgttggggtttattactgctttcaaggttcacatgctccgtggacgttcggtggaggcaccaaggtggaaatcaaac

SEQ ID NO: 536 - DNA, 361 bp, Artificial Sequence
  hSC10.17 heavy chain variable region
gaggtgcagctggtggagtctgggggaggcttggtacagcctggggggtccctgagactctcctgtgcagcctctggattcaccttcagtagctatggcatgcactgggtccgccaggctccagggaaggggctggagtgggttgcatacattactactagaagtagtaccatatactacgcagactctgtgaagggccgattcaccatctccagagacaatgccaagaactcactgtatctgcaaatgaacagcctgagagccgaggacacggctgtgtattactgtactagagaacccctaactggatactatgctatggactactggggtcaaggaacctcagtcaccgtctcctcag

SEQ ID NO: 537 - protein, 213 aa, Mus musculus
  MISC_FEATURE: SC17.17 ss1 and ss2 full length light chain
EIVLTQSPATLSLSPGERATLSCSASSSVSYMHWYQQKPGQAPRLLIYDTSKLPSGIPARFSGSGSGTDFTLTISSLEPEDFAVYYCQQWSSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 538 - protein, 449 aa, Mus musculus
  MISC_FEATURE: SC17.17 ss3 and ss4 full length heavy chain
QVQLVQSGAEVKKPGASVKVSCKASGYTFTDYNMHWVRQAPGQGLEWMGEINPNIGGTGYNQKFKGRVTMTRDTSISTAYMELSRLRSDDTAVYYCARTYSYYSYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 539 - protein, 449 aa, Mus musculus
  MISC_FEATURE: SC17.17 ss1 full length heavy chain
QVQLVQSGAEVKKPGASVKVSCKASGYTFTDYNMHWVRQAPGQGLEWMGEINPNIGGTGYNQKFKGRVTMTRDTSISTAYMELSRLRSDDTAVYYCARTYSYYSYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 540 - protein, 448 aa, Mus musculus
  MISC_FEATURE: SC17.17 ss2 full length heavy chain
QVQLVQSGAEVKKPGASVKVSCKASGYTFTDYNMHWVRQAPGQGLEWMGEINPNIGGTGYNQKFKGRVTMTRDTSISTAYMELSRLRSDDTAVYYCARTYSYYSYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 541 - protein, 212 aa, Mus musculus
  MISC_FEATURE: SC17.17 ss3 full length light chain
EIVLTQSPATLSLSPGERATLSCSASSSVSYMHWYQQKPGQAPRLLIYDTSKLPSGIPARFSGSGSGTDFTLTISSLEPEDFAVYYCQQWSSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 542 - protein, 213 aa, Mus musculus
  MISC_FEATURE: SC17.17 ss4 full length light chain
EIVLTQSPATLSLSPGERATLSCSASSSVSYMHWYQQKPGQAPRLLIYDTSKLPSGIPARFSGSGSGTDFTLTISSLEPEDFAVYYCQQWSSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 543 - protein, 449 aa, Mus musculus
  MISC_FEATURE: SC10.17ss3 full length heavy chain
EVQLVESGGGLVQPGGSLRLSCAASGFTFSSYGMHWVRQAPGKGLEWVAYITTRSSTIYYADSVKGRFTISRDNAKNSLYLQMNSLRAEDTAVYYCTREPLTGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 544 - protein, 219 aa, Mus musculus
  MISC_FEATURE: SC10.17ss3 full length light chain
DVVMTQSPLSLPVTLGQPASISCRSSQSIVHSDGNTYLEWYQQRPGQSPRRLIYKVSNRFSGVPDRFSGSGSGTDFTLKISRVEAEDVGVYYCFQGSHAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 545-549 - skipped (5 entries)

SEQ ID NO: 550 - protein, 107 aa, Artificial Sequence
  ss5 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGDC

SEQ ID NO: 551 - protein, 108 aa, Artificial Sequence
  ss6 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEEC

SEQ ID NO: 552 - protein, 107 aa, Artificial Sequence
  ss7 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGCE

SEQ ID NO: 553 - protein, 106 aa, Artificial Sequence
  ss8 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRCE

SEQ ID NO: 554 - protein, 107 aa, Artificial Sequence
  ss9 light chain constant region protein
RTVAAPSVFIFPPSCEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 555 - protein, 107 aa, Artificial Sequence
  ss10 light chain constant region protein
RTVAAPSVFIFPPSDEQLKSGTASVVCLLNNFYPREAKVQWKVDNALQSGNSQESVTEQDSKDSTYSLSSTLTLSKADYEKHCVYACEVTHQGLSSPVTKSFNRGES

SEQ ID NO: 556 - protein, 107 aa, Artificial Sequence
  ss11 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVCKSFNRGES

SEQ ID NO: 557 - protein, 107 aa, Artificial Sequence
  ss12 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKCFNRGES

SEQ ID NO: 558 - protein, 107 aa, Artificial Sequence
  ss13 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFCRGES

SEQ ID NO: 559 - protein, 107 aa, Artificial Sequence
  ss14 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNCGES

SEQ ID NO: 560 - protein, 107 aa, Artificial Sequence
  ss15 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRCES

SEQ ID NO: 561 - protein, 107 aa, Artificial Sequence
  ss16 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGCS

SEQ ID NO: 562 - protein, 104 aa, Artificial Sequence
  ss17 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNC

SEQ ID NO: 563 - protein, 105 aa, Artificial Sequence
  ss18 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNCE

SEQ ID NO: 564 - protein, 105 aa, Artificial Sequence
  ss19 light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRC

(19) United States
(12) Patent Application Publication
(10) Pub. No.: US 2018/0112004 A1
(43) Pub. Date: Apr. 26, 2018

(54) ENGINEERED SITE-SPECIFIC ANTIBODIES AND METHODS OF USE

(71) Applicant: ABBVIE STEMCENTRX LLC, NORTH CHICAGO, IL (US)

(72) Inventors: DAVID LIU, SAN FRANCISCO, CA (US); VIKRAM NATWARSINHJI SISODIYA, SAN FRANCISCO, CA (US); JOSEPH LEUNG, ALBANY, CA (US)

(73) Assignee: ABBVIE STEMCENTRX LLC, NORTH CHICAGO, IL (US)

(21) Appl. No.: 15/555,368

(22) PCT Filed: Mar. 4, 2016
(86) PCT No.: PCT/US16/20876

§ 371 Date: Sep. 1, 2017

Related U.S. Application Data
(60) Provisional application No. 62/128,424, filed on Mar. 4, 2015.

(51) Int. Cl. C07K 16/30; C07K 16/28; A61P 35/00; A61K 47/68; A61K 31/40; A61K 31/7034; A61K 31/427

(52) U.S. Cl. CPC C07K 16/30; C07K 16/2896; A61P 35/00; A61K 47/6851; A61K 47/6803; A61K 2039/505; A61K 31/7034; A61K 31/427; C07K 2317/24; C07K 2317/40; A61K 31/40

(57) ABSTRACT

Provided are novel antibody drug conjugates (ADCs), and methods of using such ADCs to treat proliferative disorders.

### CROSS REFERENCED APPLICATION

Priority is claimed to U.S. Provisional Application No. 62/128,424 filed on Mar. 4, 2015, which is incorporated herein by reference in its entirety.

### SEQUENCE LISTING

The instant application contains a sequence listing which has been submitted in ASCII format via EFS-Web and is hereby incorporated by reference in its entirety. Said ASCII copy, created on Mar. 3, 2016, is named “sc0004pct_030316.txt” and is 554 KB (567,402 bytes) in size.

### FIELD OF THE INVENTION

This application generally relates to novel compounds comprising site-specific antibodies or immunoreactive fragments thereof having one or more unpaired cysteine residues conjugated to cytotoxins and use of the same for the treatment or prophylaxis of cancer and any recurrence or metastasis thereof.

### BACKGROUND OF THE INVENTION

Many commonly employed cancer therapeutics tend to induce substantial toxicity due to their inability to selectively target proliferating tumor cells. Rather, these traditional chemotherapeutic agents act non-specifically and often damage or eliminate normally proliferating healthy tissue along with the tumor cells. Quite often this unintended cytotoxicity limits the dosage or regimen that the patient can endure, thereby effectively limiting the therapeutic index of the agent. As a result, numerous attempts have made to target cytotoxic therapeutic agents to the tumor site with varying degrees of success. One promising area of research has involved the use of antibodies to direct cytotoxic agents to the tumor so as to provide therapeutically effective localized drug concentrations.

In this regard it has long been recognized that the use of targeting monoclonal antibodies (“mAbs”) conjugated to selected cytotoxic agents provides for the delivery of relatively high levels of such cytotoxic payloads directly to the tumor site while reducing the exposure of normal tissue to the same. While the use of such antibody drug conjugates (“ADCs”) has been extensively explored in a laboratory or preclinical setting, their practical use in the clinic is much more limited. In certain cases these limitations were the result of combining weak or ineffective toxins with tumor targeting molecules that were not sufficiently selective or failed to effectively associate with the tumor. In other instances the molecular constructs proved to be unstable upon administration or were cleared from the bloodstream too quickly to accumulate at the tumor site in therapeutically significant concentrations. While such instability may be the result of linker selection or conjugation procedures, it may also be the result of overloading the targeting antibody with toxic payloads (i.e., the drug to antibody ratio or “DAR” is too high) thereby creating unstable conjugate species in the drug preparation. In some instances construct instability, whether from design or from unstable DAR species, has resulted in unacceptable non-specific toxicity as the potent cytotoxic payload is prematurely leached from the drug conjugate and accumulates at the site of injection or in critical organs as the body attempts to clear the untargeted payload. As such, relatively few ADCs have been approved by the Federal Drug Administration to date though several such compounds are presently in clinical trials. Accordingly, there remains a need for stable, relatively homogeneous antibody drug conjugate preparations that exhibit a favorable therapeutic index.

### SUMMARY OF THE INVENTION

These and other objectives are provided for by the present invention which, in a broad sense, is directed to novel methods, compounds, compositions and articles of manufacture that provide improved site-specific antibodies and conjugates which exhibit a favorable pharmacokinetic and pharmacodynamic properties. The benefits provided by the present invention are broadly applicable in the field of antibody therapeutics and diagnostics and may be used in conjunction with antibodies that react with a variety of targets. As will be discussed in detail below, the disclosed site-specific conjugates comprise engineered antibody constructs having one or more unpaired cysteines which may be preferentially conjugated to therapeutic or diagnostic payloads using novel selective reduction techniques. Such site-specific conjugate preparations are relatively stable when compared with conventional conjugated preparations and substantially homogenous as to average DAR distribution and payload position. As shown in the appended Examples the stability and homogeneity of disclosed site-specific conjugate preparations (regarding both average DAR distribution and payload positioning) provide for a favorable toxicity profile that contributes to an improved therapeutic index.

In one embodiment the invention is directed to site-specific engineered antibodies comprising one or more unpaired cysteine residues. Those of skill in the art will appreciate that the unpaired cysteine residues provide site(s) for the selective and controlled conjugation of pharmaceutically active moieties to produce engineered conjugates in accordance with the teachings herein.

In another embodiment the invention is directed to a site-specific engineered IgG1 isotype antibody comprising at least one unpaired cysteine residue. In some embodiments the unpaired cysteine residue(s) will comprise heavy/light chain interchain residues as opposed to heavy/heavy chain interchain residues. In other embodiments the unpaired cysteine residue will be generated from an intrachain disulfide bond (or “intrachain disulfide bridge”).

In another embodiment the invention provides an engineered antibody wherein the C214 residue (numbered according to the EU index of Kabat) of the light chain comprising the site-specific engineered antibody is substituted with another residue or deleted. In a further embodiment the invention provides an engineered antibody wherein the C220 residue (numbered according to the EU index of Kabat) of the heavy chain comprising the engineered antibody is substituted with another residue or deleted.

In other selected embodiments the engineered antibodies will comprise one or more unpaired cysteine residue(s) wherein the unpaired cysteine residue(s) are not a native interchain disulfide bond cysteine(s). Yet other aspects of the invention are directed to engineered antibodies comprising one or more unpaired cysteine residue(s) wherein the unpaired cysteine residue(s) are exclusive of cysteines that form native interchain disulfide bonds. In certain other aspects the unpaired cysteine will not comprise a native cysteine.

In selected embodiments the unpaired cysteine will be present in the CL domain of the antibody light chain and in certain aspects in the C-terminal region of the CL domain. In other selected embodiments the free cysteine residue will be incorporated in one of the exposed loop structures of the constant region of an antibody light chain. In such embodiments the free cysteine residue will be positioned (by incorporation or substitution) in residues 121-128, residues 182-191 or residues 201-213 (Kabat numbering). In other preferred embodiments the free cysteine residue will comprise (by incorporation or substitution) an exposed residue on the beta sheet of the CL region.

In certain selected aspects the site-specific antibodies of the instant invention will comprise a light chain constant region comprising at least one non-native cysteine. In such embodiments the light chain constant region may comprise a kappa light chain constant region comprising at least one non-native cysteine or a lambda light chain constant region comprising at least one non-native cysteine. Still other embodiments will comprise a kappa light chain constant region comprising at least one non-native cysteine at residue position 122, 190, 206, 208, 210, 211, 212 or 213. In other preferred embodiments the site-specific engineered antibody will comprise a heavy chain and a light chain wherein the light chain comprises a light chain constant region having an amino acid sequence selected from the group consisting of SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563 and SEQ ID NO: 564.

In other embodiments the engineered antibodies will comprise an unpaired cysteine residue located on the antibody heavy chain. In yet other embodiments the free cysteine residue will be positioned within the CH1 domain. In still other embodiments the free cysteine residue will be positioned in the CH2 domain. In yet other embodiments the free cysteine residue will be positioned in the CH3 domain.

In preferred embodiments the site-specific engineered antibody will immunospecifically react with an antigenic marker present on tumorigenic cells. Accordingly, in particularly preferred embodiments the present invention is directed to an engineered antibody comprising one or more unpaired cysteine residues wherein the engineered antibody immunospecifically reacts with a determinant selected from the group of DLL3, SEZ6 and CD324 which are each known to be tumor markers.

In a related embodiment site-specific antibodies are used to fabricate engineered conjugates wherein the free cysteine(s) are conjugated to a therapeutic or diagnostic agent. In this regard the invention comprises an antibody drug conjugate of the formula:

Ab-[L-D]n or a pharmaceutically acceptable salt thereof wherein
Ab comprises an antibody comprising one or more unpaired cysteine residue(s) wherein the unpaired cysteine residue(s) are exclusive of cysteines that form native interchain disulfide bonds;
L comprises an optional linker;
D comprises a drug; and
n is an integer from about 1 to about 12

In addition to the foregoing antibody drug conjugates the invention further provides pharmaceutical compositions generally comprising the disclosed ADCs and methods of using such ADCs to diagnose or treat disorders, including cancer, in a patient. In particularly preferred embodiments the engineered antibodies or conjugates will associate with a determinant selected from the group consisting of DLL3, SEZ6 and CD324.

In a related embodiment the invention is directed to a method of killing, reducing the frequency or inhibiting the proliferation of tumor cells or tumorigenic cells comprising treating said tumor cells or tumorigenic cells with a site-specific ADC of the instant invention. In a related embodiment the invention provides a method of treating cancer comprising administering to a subject a pharmaceutical composition comprising a site-specific conjugate of the instant invention.

In another embodiment the present invention comprises a method of preparing an antibody drug conjugate of the invention comprising the steps of:
a) providing an engineered antibody comprising one or more unpaired cysteine residue(s) wherein the unpaired cysteine residue(s) are exclusive of cysteines that form native interchain disulfide bonds;
b) selectively reducing the engineered antibody; and
c) conjugating the selectively reduced engineered antibody to a drug.

In a related preferred embodiment the step of selectively reducing the antibody comprises the step of contacting the antibody with a stabilizing agent. In yet another embodiment the process may further comprise the step of contacting the antibody with a mild reducing agent.

As indicated such conjugates may be used for the treatment, management, amelioration or prophylaxis of proliferative disorders or recurrence or progression thereof. Selected embodiments of the present invention provide for the use of such site-specific conjugates for the immunotherapeutic treatment of malignancies preferably comprising a reduction in tumor initiating cell frequency. The disclosed ADCs may be used alone or in conjunction with a wide variety of anti-cancer compounds such as chemotherapeutic or immunotherapeutic agents (e.g., therapeutic antibodies) or biological response modifiers. In other selected embodiments, two or more discrete site-specific antibody drug conjugates may be used in combination to provide enhanced anti-neoplastic effects.

Beyond the therapeutic uses discussed above it will also be appreciated that the engineered conjugates of the instant invention may be used to detect, diagnose or classify disorders and, in particular, proliferative disorders. They may also be used in the prognosis and/or theragnosis of such disorders. In some embodiments the site-specific conjugates may be administered to the subject and detected or monitored in vivo. Those of skill in the art will appreciate that such modulators may be labeled or associated with effectors, markers or reporters as disclosed below and detected using any one of a number of standard techniques (e.g., MRI, CAT scan, PET scan, etc.).

Thus, in some embodiments the invention will comprise a method of diagnosing, detecting or monitoring a proliferative disorder in vivo in a subject in need thereof comprising the step of administering an engineered conjugate.

In other instances the conjugates may be used in an in vitro diagnostic setting using art-recognized procedures (e.g., immunohistochemistry or “IHC”). As such, a preferred embodiment comprises a method of diagnosing a proliferative disorder in a subject in need thereof comprising the steps of:
a. obtaining a tissue sample from said subject;
b. contacting the tissue sample with at least one site-specific conjugate; and
c. detecting or quantifying the site-specific conjugate associated with the sample.

Such methods may be easily discerned in conjunction with the instant application and may be readily performed using generally available commercial technology such as automatic plate readers, dedicated reporter systems, etc. In selected embodiments the engineered conjugate will be associated with tumor perpetuating cells (i.e., cancer stem cells) present in the sample. In other preferred embodiments the detecting or quantifying step will comprise a reduction of cancer stem cell frequency which may be monitored as described herein.

The present invention also provides kits or devices and associated methods that employ the site-specific conjugates disclosed herein, and pharmaceutical compositions of engineered conjugates as disclosed herein, which are useful for the treatment of proliferative disorders such as cancer. To this end the present invention preferably provides an article of manufacture useful for treating such disorders comprising a receptacle containing an site-specific antibody drug conjugate and instructional materials for using the conjugates to treat, ameliorate or prevent a proliferative disorder or progression or recurrence thereof. In selected embodiments the devices and associated methods will comprise the step of contacting at least one circulating tumor cell.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1 is a depiction of the structure of the human IgG1 antibody showing the intrachain and interchain disulfide bonds.

FIGS. 2A and 2B provide, in a tabular form, contiguous amino acid sequences (SEQ ID NOS: 519-528) of light and heavy chain variable regions of a number of humanized exemplary DLL3 antibodies compatible with the disclosed antibody drug conjugates isolated, cloned and engineered as described in the Examples herein.

FIGS. 3A and 3B provide, in a tabular form, contiguous amino acid sequences (SEQ ID NOS: 170-199) of light and heavy chain variable regions of a number of humanized exemplary SEZ6 antibodies compatible with the disclosed antibody drug conjugates isolated, cloned and engineered as described in the Examples herein.

FIG. 4 depicts, in a tabular form, contiguous amino acid sequences (SEQ ID NOS: 529-532) of light and heavy chain variable regions of murine and humanized exemplary CD324 antibodies compatible with the disclosed antibody drug conjugates isolated, cloned and engineered as described in the Examples herein.

FIGS. 5A and 5B provide amino acid sequences of light and heavy chains (SEQ ID NOS: 507-512) of exemplary site-specific anti-DLL3 antibodies produced in accordance with the instant teachings.

FIGS. 6A and 6B provide amino acid sequences of light and heavy chains (SEQ ID NOS: 513-518) of exemplary site-specific anti-SEZ6 antibodies produced in accordance with the instant teachings.

FIG. 7 depicts the amino acid sequences of the light and heavy chains (SEQ ID NOS: 543-544) of an exemplary CD324ss3 site-specific antibody produced in accordance with the instant teachings.

FIG. 8 shows the binding properties of native and site-specific constructs fabricated as set forth herein.

FIG. 9 is a schematic representation depicting the process of conjugating an engineered antibody to a cytotoxin.

FIGS. 10A and 10B are graphical representations showing the conjugation percentages of site-specific antibody light and heavy chains conjugated using reducing agents as determined using RP-HPLC.

FIGS. 11A and 11B are graphical representations showing the DAR distribution of site-specific antibody constructs conjugated using reducing agents as determined using HIC.

FIGS. 12A and 12B show the conjugation percentages of site-specific antibody light and heavy chains conjugated using stabilizing agents or reducing agents as determined using RP-HPLC.

FIGS. 13A and 13B are graphical representations showing the DAR distribution of site-specific antibody constructs conjugated using stabilization or reducing agents as determined using HIC.

FIGS. 14A and 14B show the DAR distribution of site-specific antibody constructs conjugated using stabilization and/or mild reducing agents as determined using HIC.

FIG. 15 depicts DAR distribution of site-specific antibody constructs conjugated using various stabilization agents as determined using HIC.

FIG. 16 depicts amino acid sequences of exemplary light chain constant regions (SEQ ID NOS: 502, 503 and 550-564) that may be used to fabricate site-specific antibodies produced in accordance with the instant teachings.

FIG. 17 provides a graphical representation showing the DAR distribution of various selectively conjugated site-specific antibody constructs as determined using HIC.

FIG. 18 shows the conjugation percentages of antibody light and heavy chains derived from various selectively conjugated site-specific antibody constructs as determined using RP-HPLC.

FIG. 19 illustrates the DAR distribution of various selectively conjugated site-specific antibody constructs as determined using HIC.

FIG. 20 depicts the conjugation percentages of antibody light and heavy chains derived from various selectively conjugated site-specific antibody constructs as determined using RP-HPLC.

FIG. 21 shows the DAR distribution of various selectively conjugated site-specific antibody constructs as determined using HIC.

FIG. 22 depicts the conjugation percentages of antibody light and heavy chains derived from various selectively conjugated site-specific antibody constructs as determined using RP-HPLC.

FIG. 23 shows the DAR distribution of various selectively conjugated site-specific antibody constructs as determined using HIC.

FIG. 24 shows the conjugation percentages of antibody light and heavy chains derived from various selectively conjugated site-specific antibody constructs as determined using RP-HPLC.

FIG. 25 illustrates the DAR distribution of various selectively conjugated site-specific antibody constructs as determined using HIC.

FIG. 26 illustrates the ability of site-specific calicheamicin conjugates to kill DLL3⁺ cells in vitro.

FIG. 27 shows the ability of site-specific dolastatin conjugates to kill DLL3⁺ cells in vitro.

### DETAILED DESCRIPTION OF THE INVENTION

### I. Introduction

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described. Finally, for the purposes of the instant disclosure all identifying sequence Accession numbers may be found in the NCBI Reference Sequence (RefSeq) database and/or the NCBI GenBank archival sequence database unless otherwise noted.

Initially it is important to note that the site-specific antibodies and site-specific conjugates of the instant invention are not limited to any particular target or antigen. Rather, as any existing antibody or any antibody that may be generated as described herein may be converted to a site-specific antibody, the advantages conferred by the present invention are broadly applicable and may be used in conjunction with any target antigen (or determinant). More specifically, the beneficial properties imparted by the use of unpaired cysteine conjugation sites and selective reduction of the same (e.g., enhanced conjugate stability and reduced non-specific toxicity) are broadly applicable to therapeutic and diagnostic antibodies irrespective of the particular target. Accordingly, while certain non-limiting determinants have been used for the purposes of explanation and demonstration of the benefits of the instant invention, they are in no way restrictive as to the scope of the same.

In any event it will be appreciated the site-specific antibody conjugates of the instant invention have been found to exhibit favorable characteristics that make them particularly suitable for use as therapeutic compounds and compositions. In this regard the conjugates described herein immunospecifically react with determinants that have been found to be associated with various proliferative disorders and demonstrated to be effective therapeutic targets. Additionally, in certain preferred embodiments the constructs of the instant invention provide for selective conjugation at specific cysteine positions derived from disrupted native disulfide bond(s) obtained through molecular engineering techniques. In other preferred embodiments, and as will be discussed in more detail below, the unpaired or free cysteines may be engineered into any residue site present in the selected antibody or immunoreactive fragment thereof (i.e., such sites do not require disruption of a naturally occurring native disulfide bond).

Whether introducing free cysteines at preselected sites or disrupting native disulfide bonds, engineering of the antibodies as described herein provides for regulated stoichiometric conjugation that allows the drug to antibody ratio (“DAR”) to largely be fixed with precision resulting in the generation of substantially DAR homogeneous preparations. Moreover the disclosed site-specific constructs further provide preparations that are substantially homogeneous with regard to the position of the payload on the antibody. Selective conjugation of the engineered constructs using stabilization agents as described herein increases the desired DAR species percentage and, along with the fabricated unpaired or free cysteine site, imparts conjugate stability and homogeneity that reduces non-specific toxicity caused by the inadvertent leaching of cytotoxin. This reduction in toxicity provided by selective conjugation of free cysteines and the relative homogeneity (both in conjugation positions and DAR) of the preparations also provides for an enhanced therapeutic index that allows for increased cytotoxin payload levels at the tumor site. Additionally, the resulting site-specific conjugates may optionally be purified using various chromatographic methodology to provide highly homogeneous site-specific conjugate preparations comprising desired DAR species (e.g., DAR=2) of greater than 75%, 80%, 85%, 90% or even 95%. Such conjugate homogeneity may further increase the therapeutic index of the disclosed preparations by limiting unwanted higher DAR conjugate impurities (which may be relatively unstable) that could increase toxicity.

It will be appreciated that the favorable properties exhibited by the disclosed engineered conjugate preparations is predicated, at least in part, on the ability to specifically direct the conjugation and largely limit the fabricated conjugates in terms of conjugation position and absolute DAR. Unlike most conventional ADC preparations the present invention does not rely entirely on partial or total reduction of the antibody to provide random conjugation sites and relatively uncontrolled generation of DAR species. Rather, the present invention provides one or more predetermined unpaired (or free) cysteine sites by engineering the targeting antibody to disrupt one or more of the naturally occurring (i.e., “native”) interchain or intrachain disulfide bonds or to introduce a cysteine residue at any position. To this end it will be appreciated that, in selected embodiments, a cysteine residue may be incorporated anywhere along the antibody (or immunoreactive fragment thereof) heavy or light chain or appended thereto using standard molecular engineering techniques. In other preferred embodiments disruption of native disulfide bonds may be effected in combination with the introduction of a non-native cysteine (which will then comprise a free cysteine) that may then be used as conjugation sites.

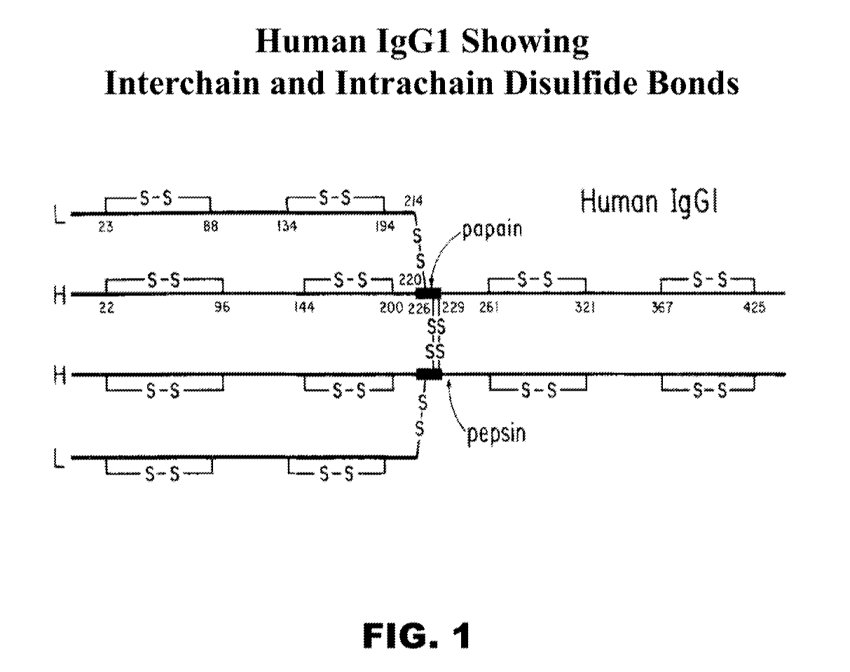

With regard to the introduction or addition of a cysteine residue or residues to provide a free cysteine (as opposed to disrupting a naturally occurring or native disulfide bond or bridge) compatible position(s) on the antibody or antibody fragment may readily be discerned by on skilled in the art. Note that in the instant invention the terms “disulfide bond” and “disulfide bridge” are equivalent and may be used interchangeably unless otherwise dictated by context. Thus in certain embodiments the free or unpaired cysteine will not comprise a native interchain disulfide bond cysteine but rather will be provided through the introduction of a cysteine residue in a non-native position or by the disruption of an intrachain disulfide bond. In selected embodiments the disclosed engineered antibodies will comprise one or more unpaired cysteine residue(s) wherein the unpaired cysteine residue(s) are exclusive of cysteines that form native interchain disulfide bonds. By way of example such embodiments comprising an IgG1 engineered antibody would not comprise a free cysteine residue a position C214 on the light chains or at positions C220, C226 or C229 (all Kabat numbering) of the heavy chains (see generally FIG. 1). Of course the positions of naturally occurring or native disulfide bonds are well known for all classes (i.e. IgA, IgD, IgE, IgG, and IgM) and all subclasses (i.e., IgG1, IgG2, IgG3, IgG4, IgA1, and IgA2) of antibodies and in each case one skilled in the art would readily be able to discern those cysteines that form native interchain disulfide bonds.

In other selected embodiments the unpaired cysteine residue(s) may be introduced in the heavy chain. More particularly one or more unpaired cysteine residues may be placed in the CH1 domain, the CH2 domain or the CH3 domain or any combination thereof depending on the desired DAR, the antibody construct, the selected payload and the antibody target. In other preferred embodiments the cysteine residues may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein.

Thus, as used herein, the terms “free cysteine” or “unpaired cysteine” may be used interchangeably unless otherwise dictated by context and shall mean any cysteine constituent of an antibody, whether naturally present or specifically incorporated in a selected residue position using molecular engineering techniques and not bound to another cysteine in the same antibody under normal physiological conditions. Thus, in certain preferred embodiments the free cysteine may comprise a naturally occurring cysteine whose native interchain or intrachain disulfide bridge partner has been substituted, eliminated or otherwise altered to disrupt the naturally occurring disulfide bond under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as a non-natural intramolecular disulfide bond (oxidized) with another free cysteine on the same antibody depending on the oxidation state of the system. As discussed in more detail below, mild reduction of this antibody construct will provide thiols available for site-specific conjugation. In particularly preferred embodiments the free or unpaired cysteines (whether naturally occurring or incorporated) will be subject to selective reduction and subsequent conjugation to provide the disclosed homogenous DAR compositions.

More specifically, the resulting free cysteines may then be selectively reduced using the novel techniques disclosed herein without substantially disrupting intact native disulfide bridges, to provide reactive thiols predominantly at the selected cysteine sites. These manufactured thiols are then subject to directed conjugation with the disclosed drug-linker compounds without substantial non-specific conjugation. That is, the engineered constructs and, optionally, the selective reduction techniques disclosed herein largely eliminate non-specific, random conjugation of the toxin payloads. Significantly this provides preparations that are substantially homogeneous in both DAR species distribution and conjugate position on the targeting antibody. As discussed below the elimination of relatively high DAR contaminants can, in and of itself, reduce non-specific toxicity and expand the therapeutic index of the preparation. Moreover, such selectivity allows the payloads to largely be placed in particularly advantageous predetermined positions (such as the terminal region of the light chain constant region) where the payload is somewhat protected until it reaches the tumor but is suitably presented and processed once it reaches the target. Thus, design of the engineered antibody to facilitate specific payload positioning may also be used to reduce the non-specific toxicity of the disclosed preparations. Finally, the ability to selectively and reproducibly direct conjugation of the antibody greatly simplifies characterization of the resulting composition thereby facilitating drug development.

As discussed below and shown in the Examples, creation of these predetermined free cysteine sites may be achieved using art-recognized molecular engineering techniques introduce a cysteine at a preselected site on the antibody or to remove, alter or replace one of the constituent cysteine residues of the disulfide bond. Using these techniques one skilled in the art will appreciate that any antibody class or isotype may be engineered to exhibit one or more free cysteine(s) capable of being selectively conjugated in accordance with the instant invention. Moreover, the selected antibody maybe engineered to specifically exhibit 1, 2, 3, 4, 5, 6, 7, 8, 9, 10, 11 or even 12 free cysteines depending on the desired DAR. More preferably the selected antibody will be engineered to contain 2 or 4 free cysteines and even more preferably to contain 2 free cysteines. It will also be appreciated that the free cysteines may be positioned in engineered antibody to facilitate delivery of the selected cytotoxin to the target while reducing non-specific toxicity. In this respect selected embodiments of the invention comprising IgG1 antibodies will position the payload on the CH1 domain and more preferably on the C-terminal end of the domain. In other preferred embodiments the constructs will be engineered to position the payload on the light chain constant region and more preferably at the C-terminal end of the constant region. In yet other selected aspects the constructs may be engineered to selectively position the payload on the CH2 or CH3 domain.

Significantly, limiting payload conjugation to the engineered free cysteines may also be facilitated by selective reduction of the construct using novel stabilization agents a set forth below. “Selective reduction” as used herein will mean exposure of the engineered constructs to reducing conditions that reduce the free cysteines (thereby providing reactive thiols) without substantially disrupting intact native disulfide bonds. In general selective reduction may be effected using any reducing agents, or combinations thereof that provide the desired thiols without disrupting the intact disulfide bonds. In certain preferred embodiments, and as set forth in the Examples below, selective reduction may be effected using a stabilizing agent and mild reducing conditions to prepare the engineered construct for conjugation. As discussed in more detail herein compatible stabilizing agents will generally facilitate reduction of the free cysteines and allow the desired conjugation to proceed under less stringent reducing conditions. This allows a substantial majority of the native disulfide bonds to remain intact and markedly reduces the amount of non-specific conjugation thereby limiting unwanted contaminants and potential toxicity. The relatively mild reducing conditions may be attained through the use of a number of systems but preferably comprises the use of thiol containing compounds. It will be appreciated that one skilled in the art could readily derive compatible reducing systems in view of the instant disclosure.

### II. Determinants

Those skilled in the art will appreciate that the engineered antibodies or conjugates may be generated from any antibody that specifically recognizes or associates with any relevant determinant. As used herein “determinant” means any detectable trait, property, marker or factor that is identifiably associated with, or specifically found in or on a particular cell, cell population or tissue. Determinants may be morphological, functional or biochemical in nature and are generally phenotypic. In certain preferred embodiments the determinant is a protein that is differentially modified with regard to its physical structure and/or chemical composition or a protein that is differentially expressed (up- or down-regulated) by specific cell types or by cells under certain conditions (e.g., during specific points of the cell cycle or cells in a particular niche). For the purposes of the instant invention the determinant preferably comprises a cell surface antigen, or a protein(s) which is differentially expressed by aberrant cells as evidenced by chemical modification, form of presentation (e.g., splice variants), timing or amount. In certain embodiments a determinant may comprise a SEZ6, DLL3 or CD324 protein, or any of their variants, isoforms or family members, and specific domains, regions or epitopes thereof. An “immunogenic determinant” or “antigenic determinant” or “immunogen” or “antigen” means any fragment, region or domain of a polypeptide that can stimulate an immune response when introduced into an immunocompetent animal and is recognized by the antibodies produced from the immune response. Determinants contemplated herein may identify a cell, cell subpopulation or tissue (e.g., tumors) by their presence (positive determinant) or absence (negative determinant).

As discussed herein and set forth in the Examples below, selected embodiments of the invention may comprise complete or partial variable regions from murine antibodies that immunospecifically bind to a selected determinant and which can be considered “source” antibodies. In such embodiments, antibodies contemplated by the invention may be derived from such “source” antibodies through optional modification of the constant region or the epitope-binding amino acid sequences of the source antibody. In one embodiment an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire variable region) are combined with or incorporated into an acceptor antibody sequence to provide the derivative antibody (e.g. chimeric, CDR grafted or humanized antibodies). Significantly, these derivative antibodies may comprise the site-specific antibodies of the instant invention wherein, for example, the antigen binding region of a donor antibody is associated with an constant region comprising one or more unpaired cysteines. These “derived” (e.g. chimeric, humanized or site-specific constructs) antibodies can be generated using standard molecular biology techniques for various reasons such as, for example, to provide a free cysteine; to improve affinity for the determinant; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification. Of course, it will be appreciated that the source antibodies (e.g., murine antibodies) may be engineered to provide the desired conjugation sites without undergoing further modifications to the antibody structure.

Again it must be emphasized that the site-specific conjugation technology set forth herein is generally applicable in the field of antibody therapeutics or diagnostics and may work with any existing antibody or any antibody that may be generated regardless of the antibody target. In this context certain non-limiting determinants used to demonstrate the benefits provided by the instant invention are set forth below.

CD324 (also known as E-cadherin, epithelial cadherin or CDH1) is a member of the classical subfamily of cadherins, and as such is a calcium-dependent cell-cell adhesion glycoprotein that mediates homotypic (i.e., epithelial-epithelial) cell-cell adhesion. The intracellular portions of CD324 interact with various proteins inside the cell, including α-catenin, β-catenin and p120, which themselves interact with the actin filaments of the cytoskeleton (Perez-Moreno et al, 2003). CD324 is thought to act as a bridge between the cell-adhesion machinery and the cytoskeleton, and provide cells with a compass that orients them in tissues such as stratified epithelia. With respect to the development of cancer, disturbance of the expression of CD324 is one of the main events in the early and late steps of tumorigenesis and metastasis. Inactivating germline mutations of CDH1 that result in structurally altered CD324 proteins or complete loss of CD324 expression have been correlated with gastric, breast, colorectal, thyroid, and ovarian cancers. Well-differentiated tumors have long been known to exhibit a strong staining pattern of CD324/catenin compared to poorly differentiated ones. Accordingly CD324 has been used by pathologists as a significant prognostic marker to diagnose different kinds of cancer by immunohistochemistry. Reports about the functional role of CD324 in providing mechanical support for cells, regulating cell localization and motility phenotypes, and its links to differentiation status of the cell make CD324 a very intriguing target for the development of anti-cancer therapeutics. The CD324 gene is transcribed and spliced into a 4815 bp mature mRNA transcript which has an open reading frame encoding a pre-proprotein of 882 amino acids including a signal peptide. CD324 orthologs are well conserved between different species and the sequence homology among the various members of the cadherin family is generally high. The CD324 protein is composed of four extracellular cadherin repeats (EC1-EC4) of approximately 110 amino acids, a membrane-proximal extracellular domain (EC5) that is less closely related to the other cadherin repeats, a transmembrane domain, and a highly conserved intracellular domain that can be further subdivided into the juxtamembrane domain (JMD) and a highly-phosphorylated β-catenin binding domain (CBD). Calcium ions bind at sites between the EC repeats of cadherins, conferring a rigid rod-like structure to the extracellular portion of these proteins.

SEZ6 (also known as seizure related 6 homolog) is a type I transmembrane protein originally cloned from mouse cerebrum cortex-derived cells treated with the convulsant pentylentetrazole (Shimizu-Nishikawa, 1995, PMID: 7723619). SEZ6 has two isoforms, one of approximately 4210 bases (NM 178860) encoding a 994 amino acid protein (NP 849191), and one of approximately 4194 bases (NM 001098635) encoding a 993 amino acid protein (NP 001092105). These differ only in the final ten amino acid residues in their ECDs. SEZ6 has two other family members: SEZ6L and SEZ6L2. The term “SEZ6 family”, refers to SEZ6, SEZ6L, SEZ6L2 and their various isoforms. The mature SEZ6 protein is composed of a series of structural domains: a cytoplasmic domain, a transmembrane domain and an extracellular domain comprising a unique N-terminal domain, followed by two alternating Sushi and CUB-like domains, and three additional tandem Sushi domain repeats. Mutations in the human SEZ6 gene have been linked to febrile seizures, a convulsion associated with a rise in body temperature and the most common type of seizure in childhood (Yu et al., 2007, PMID:17086543). Analysis of the structural modules of the SEZ6 protein identified by homology and sequence analysis suggest a possible role in signaling, cell-cell communication, and neural development. Anti-SEZ6 humanized antibodies were generated, as described below, from antibodies that had been isolated from mice immunized with a SEZ6 antigen.

As set forth in the Examples below particularly preferred determinants for the engineered conjugates of the instant invention comprise SEZ6, CD324 and DLL3. DLL3 (also known as Delta-like Ligand 3 or SCDO1) is a member of the Delta-like family of Notch DSL ligands. Representative DLL3 protein orthologs include, but are not limited to, human (Accession Nos. NP_058637 and NP_982353), chimpanzee (Accession No. XP_003316395), mouse (Accession No. NP_031892), and rat (Accession No. NP_446118). In humans, the DLL3 gene consists of 8 exons spanning 9.5 kBp located on chromosome 19q13. Alternate splicing within the last exon gives rise to two processed transcripts, one of 2389 bases (Accession No. NM_016941) and one of 2052 bases (Accession No. NM_203486). The former transcript encodes a 618 amino acid protein (Accession No. NP_058637), whereas the latter encodes a 587 amino acid protein (Accession No. NP_982353). These two protein isoforms of DLL3 share overall 100% identity across their extracellular domains and their transmembrane domains, differing only in that the longer isoform contains an extended cytoplasmic tail containing 32 additional residues at the carboxy terminus of the protein.

In general, DSL ligands are composed of a series of structural domains: a unique N-terminal domain, followed by a conserved DSL domain, multiple tandem epidermal growth factor (EGF)-like repeats, a transmembrane domain, and a cytoplasmic domain not highly conserved across ligands but one which contains multiple lysine residues that are potential sites for ubiquitination by unique E3 ubiquitin ligases. The DSL domain is a degenerate EGF-domain that is necessary but not sufficient for interactions with Notch receptors. Additionally, the first two EGF-like repeats of most DSL ligands contain a smaller protein sequence motif known as a DOS domain that co-operatively interacts with the DSL domain when activating Notch signaling.

The extracellular region of the DLL3 protein comprises six EGF-like domains, a single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3. The DSL domain and the N-terminal domain comprise part of the DLL3 protein as defined by a distinct amino acid sequence. Note that for the purposes of the instant disclosure the respective EGF-like domains may be termed EGF1 to EGF6 with EGF1 being closest to the N-terminal portion of the protein. In regard to the structural composition of the protein one significant aspect of the instant invention is that the disclosed DLL3 antibodies may be generated, fabricated, engineered or selected so as to react with a selected domain, motif or epitope. In certain cases such site specific antibodies may provide enhanced reactivity and/or efficacy depending on their primary mode of action.

DLL3 antibodies compatible with the instant invention and that may be used as source antibodies are disclosed in PCT Application No. US2013/0027391 which is incorporated herein by reference as to the disclosed antibodies.

More generally engineered antibodies contemplated by the invention can be derived from “source” antibodies through optional modification of the epitope-binding amino acid sequences of the source antibody and the introduction of site-specific free cysteine residues. In one embodiment an engineered antibody is “derived” from a source antibody if selected amino acids in the source antibody are altered through deletion, mutation, substitution, integration or combination to produce the engineered antibody comprising at least one free cysteine residue. In another embodiment, a “derived” antibody is one in which fragments of the source antibody (e.g., one or more CDRs) are combined with or incorporated into an acceptor antibody sequence comprising one or more free cysteine residues to provide the derivative antibody (e.g. chimeric or humanized antibodies). These “derived” antibodies can be generated for various reasons such as, for example, to improve affinity for the target; to improve production and yield in cell culture; to reduce immunogenicity in vivo; to reduce toxicity; to facilitate conjugation of an active moiety; or to create a multispecific antibody. Most importantly they provide for the site-specific conjugation of one or more pharmaceutically active moieties. Besides molecular engineering such antibodies may also be derived from source antibodies through modification of the mature molecule (e.g., glycosylation patterns or pegylation) by chemical means or post-translational modification.

While the invention is directed generally to any engineered antibody capable of specifically binding to a determinant, engineered anti-SEZ6, engineered anti-DLL3 and engineered anti-CD324 antibodies shall be used as illustrative examples of embodiments of the invention.

### III. Cell Binding Agents

1. Antibody Structure

As alluded to above, particularly preferred embodiments of the instant invention comprise the disclosed conjugates with a cell binding agent in the form of a site-specific antibody, or immunoreactive fragment thereof, that preferentially associates with one or more epitopes on a selected determinant. In this regard antibodies, and site-specific variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

Note that, for the purposes of the instant application it will be appreciated that the terms “modulator” and “antibody” may be used interchangeably unless otherwise dictated by context. Similarly, for discussion purposes the embodiments of the invention may be couched in terms of one determinant or the other. However, unless otherwise specified or required by context. such designations are merely for the purpose of explanation and not limiting as to the general concepts being described or the scope of the invention. Accordingly, the terms “anti-DLL3 conjugate” and “DLL3 conjugate”, or simply “conjugate”, all refer to the site-specific conjugates set forth herein and may be used interchangeably unless otherwise dictated by context.

An “antibody” or “intact antibody” typically refers to a Y-shaped tetrameric protein comprising two heavy (H) and two light (L) polypeptide chains held together by covalent disulfide bonds and non-covalent interactions. Human light chains comprise a variable domain (VL) and a constant domain (CL) wherein the constant domain may be readily classified as kappa or lambda based on amino acid sequence and gene loci. Each heavy chain comprises one variable domain (VH) and a constant region, which in the case of IgG, IgA, and IgD, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

There are two types of native disulfide bridges or bonds in immunoglobulin molecules: interchain and intrachain disulfide bonds. The location and number of interchain disulfide bonds vary according to the immunoglobulin class and species. While the invention is not limited to any particular class or subclass of antibody, the IgG1 immunoglobulin shall be used for illustrative purposes only. Interchain disulfide bonds are located on the surface of the immunoglobulin, are accessible to solvent and are usually relatively easily reduced. In the human IgG1 isotype there are four interchain disulfide bonds, one from each heavy chain to the light chain and two between the heavy chains. The interchain disulfide bonds are not required for chain association. As is well known the cysteine rich IgG1 hinge region of the heavy chain has generally been held to consist of three parts: an upper hinge, a core hinge, and a lower hinge. Those skilled in the art will appreciate that that the IgG1 hinge region contain the cysteines in the heavy chain that comprise the interchain disulfide bonds (two heavy/heavy, two heavy/light), which provide structural flexibility that facilitates Fab movements.

The interchain disulfide bond between the light and heavy chain of IgG1 are formed between C214 of the kappa or lambda light chain and C220 in the upper hinge region of the heavy chain (FIG. 1). The interchain disulfide bonds between the heavy chains are at positions C226 and C229. (all numbered per the EU index according to Kabat, et al., infra.)

As used herein the term “antibody” may be construed broadly and includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a DLL3 determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

In selected embodiments and as discussed in more detail below, the CL domain may comprise a kappa CL domain exhibiting a free cysteine. In other embodiments the source antibody may comprise a lambda CL domain exhibiting a free cysteine. As the sequences of all human IgG

CL domains are well known, one skilled in the art may easily analyze both lambda and kappa sequences in accordance with the instant disclosure and employ the same to provide compatible antibody constructs. Similarly, for the purposes of explanation and demonstration the following discussion and appended Examples will primarily feature the IgG1 type antibodies. As with the light chain constant region, heavy chain constant domain sequences from different isotypes (IgM, IgD, IgE, IgA) and subclasses (IgG1, IgG2, IgG3, IgG4, IgA1, IgA2) are well known and characterized. Accordingly, one skilled in the art may readily exploit antibodies comprising any isotype or subclass and conjugate each with the disclosed drugs as taught herein to provide the site-specific antibody drug conjugates of the present invention.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. ScFv fragments (for single chain fragment variable), which can be obtained by genetic engineering, associates in a single polypeptide chain, the VH and the VL region of an antibody, separated by a peptide linker.

As used herein, the assignment of amino acids to each domain, framework region and CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. The amino acid residues which comprise CDRs as defined by Kabat, Chothia, MacCallum (also known as Contact) and AbM as obtained from the Abysis website database (infra.) are set out below.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc, Natl. Acad. Sci. USA 63(1): 78-85 describing the amino acid sequence of myeloma protein Eu, which reportedly was the first human IgG1 sequenced. The Eu index of Edelman is also set forth in Kabat et al., 1991 (supra.). Thus, the terms “EU index as set forth in Kabat” or “EU index of Kabat” or “EU index according to Kabat” or simply “EU index” in the context of the heavy chain refers to the residue numbering system based on the human IgG1 Eu antibody of Edelman et al. as set forth in Kabat et al., 1991 (supra.). The numbering system used for the light chain constant region amino acid sequence is similarly set forth in Kabat et al., 1991.

Exemplary wild-type kappa CL and IgG1 heavy chain constant region amino acid sequences compatible with the instant invention are set forth, respectively, as SEQ ID NOS: 403 and 404 in the appended sequence listing. Similarly, an exemplary wild-type lambda CL light chain constant region is set forth as SEQ ID NO: 504 in the appended sequence listing. Those of skill in the art will appreciate that such light chain constant region sequences, engineered as disclosed herein to provide unpaired cysteines (e.g., see SEQ ID NOS: 502, 503, 505, 506 and 550-564), may be joined with light chain variable regions using standard molecular biology techniques. These engineered light chains are then paired with wild-type or engineered heavy chains to provide full-length antibodies that may be incorporated in the antibody conjugates of the instant invention. In this regard SEQ ID NOS: 502, 503, 505 and 506 will preferably be paired with wild-type IgG1 heavy chains (e.g., those comprising SEQ ID NO: 404) to provide a free cysteine on the heavy chain while light chains comprising SEQ ID NOS: 550-564 are preferably paired with engineered heavy chains (e.g., those comprising SEQ ID NOS: 500 and 501 which have been engineered to delete or replace C220) to provide a free cysteine on the light chain. Of course, where more than two free cysteines per antibody are desired light chains comprising CL domains set forth in SEQ ID NOS: 550-564 may be paired with wild type heavy chains having a cysteine at position 220 (e.g., SEQ ID NO: 404).

In any event the site-specific antibodies or immunoglobulins of the invention may comprise, or be derived from, any antibody that specifically recognizes or immunospecifically associates with any determinant. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In certain preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a particular protein (e.g., CD324, SEZ6 or DLL3) or any of its splice variants, isoforms or family members, or specific domains, regions or epitopes thereof. An “antigen”, “immunogenic determinant”, “antigenic determinant” or “immunogen” means any protein or any fragment, region, domain or epitope thereof that can stimulate an immune response when introduced into an immunocompetent animal and is recognized by antibodies produced from the immune response of the animal. The presence or absence of the determinants contemplated herein may be used to identify a cell, cell subpopulation or tissue (e.g., tumors, tumorigenic cells or CSCs).

As set forth below in the Examples, selected embodiments of the invention comprise murine antibodies that immunospecifically bind to SEZ6, which can be considered “source” antibodies. In other embodiments, antibodies contemplated by the invention may be derived from such “source” antibodies through optional modification of the constant region (i.e., to provide site-specific antibodies) or the epitope-binding amino acid sequences of the source antibody. In one embodiment an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire variable region) are combined with or incorporated into an acceptor antibody sequence to provide the derivative antibody (e.g. chimeric, CDR grafted or humanized antibodies). These “derived” (e.g. humanized or CDR-grafted) antibodies can be generated using standard molecular biology techniques for various reasons such as, for example, to improve affinity for the determinant; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification. Of course, as discussed extensively herein these derived antibodies may be further engineered to provide the desired site-specific antibodies comprising one or more free cysteines.

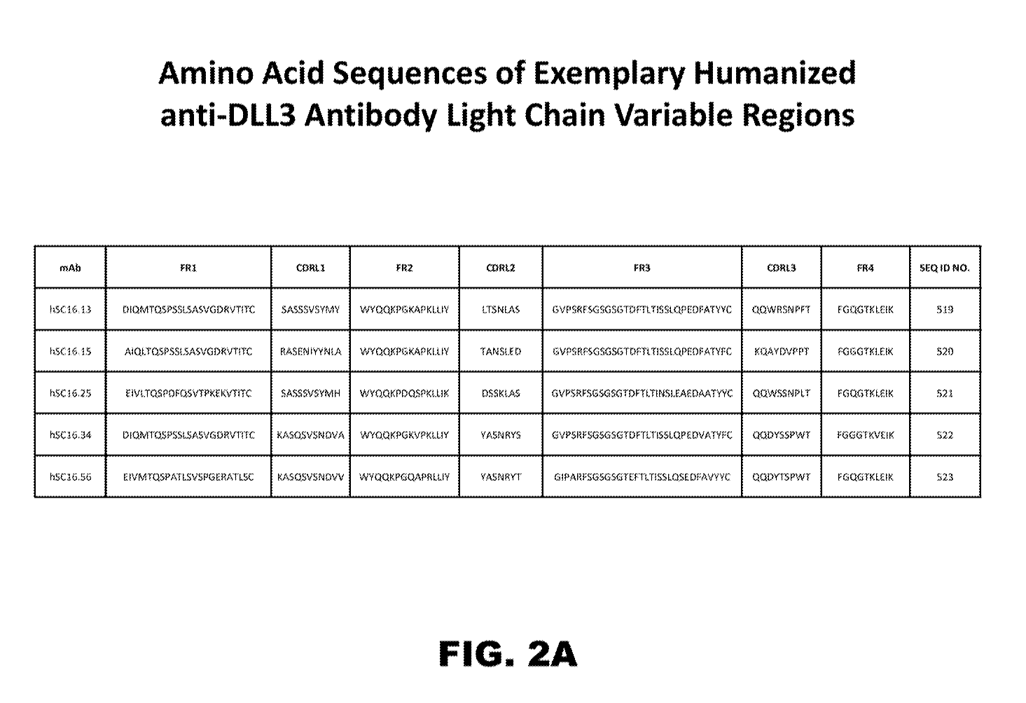

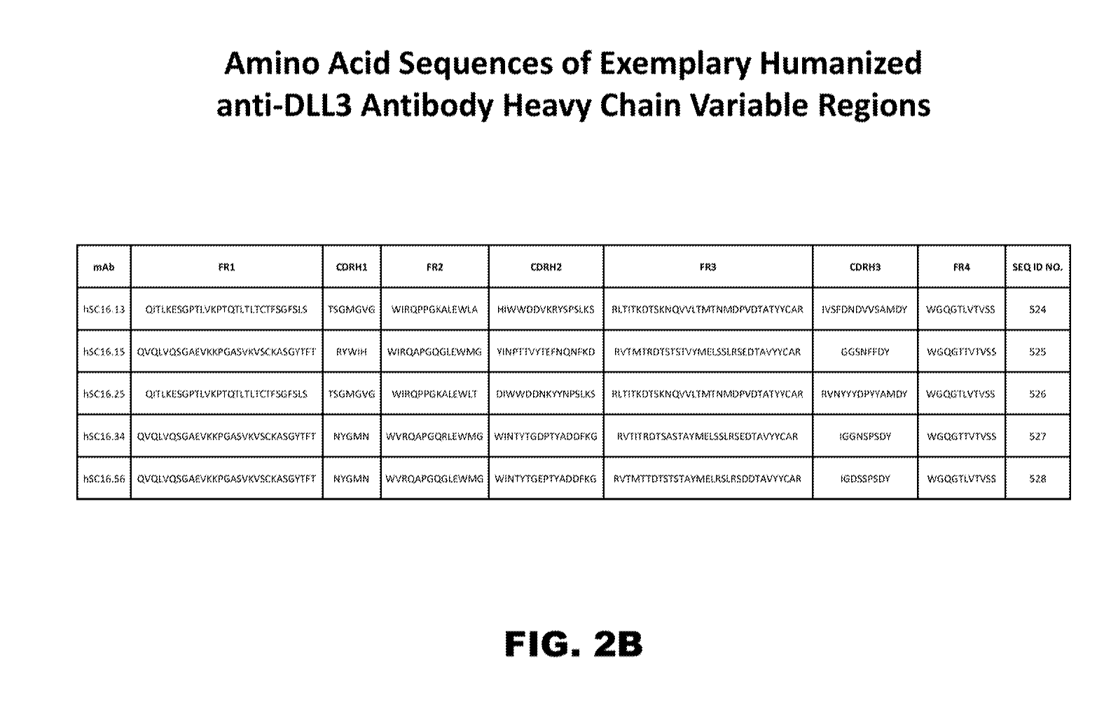

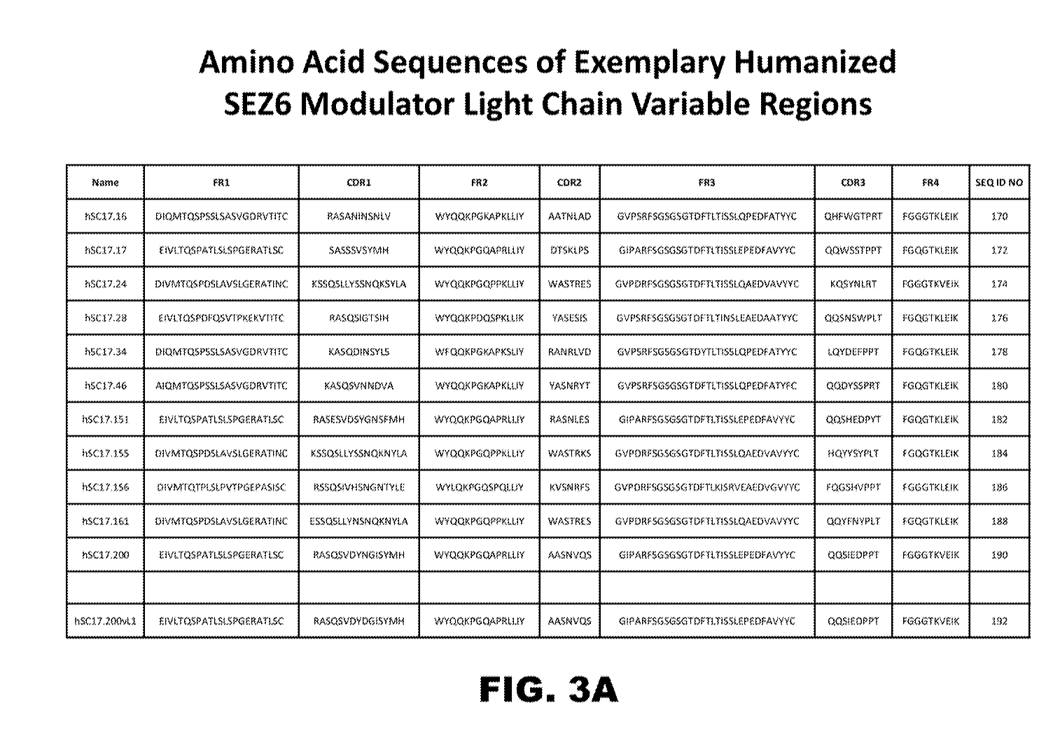

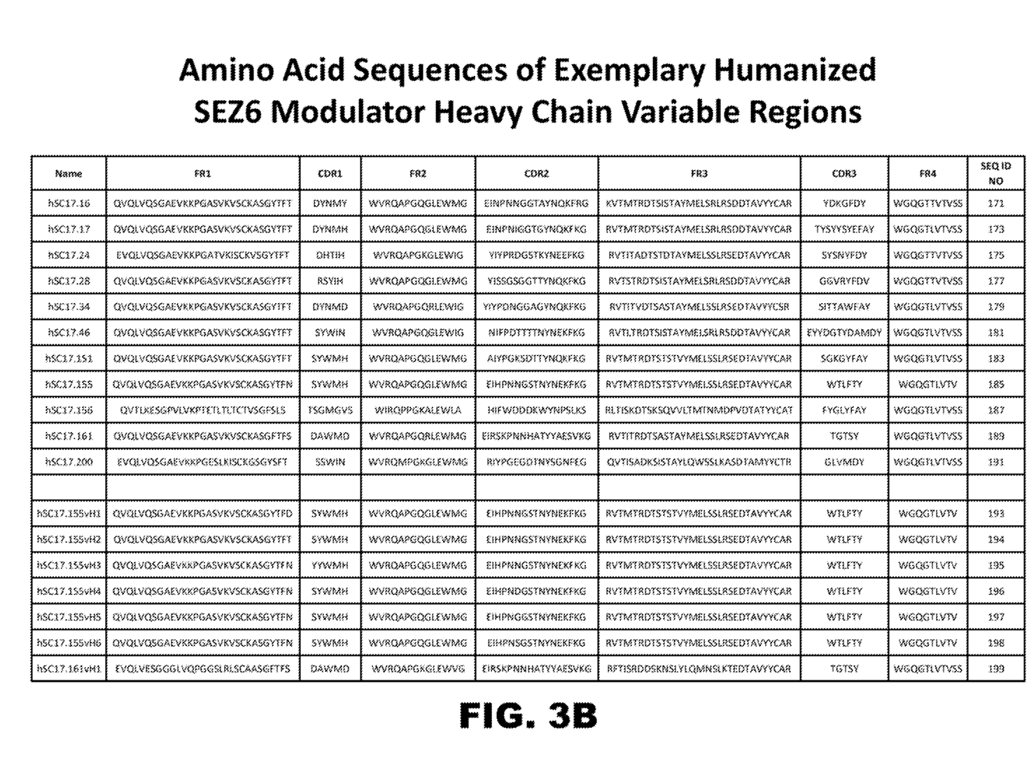

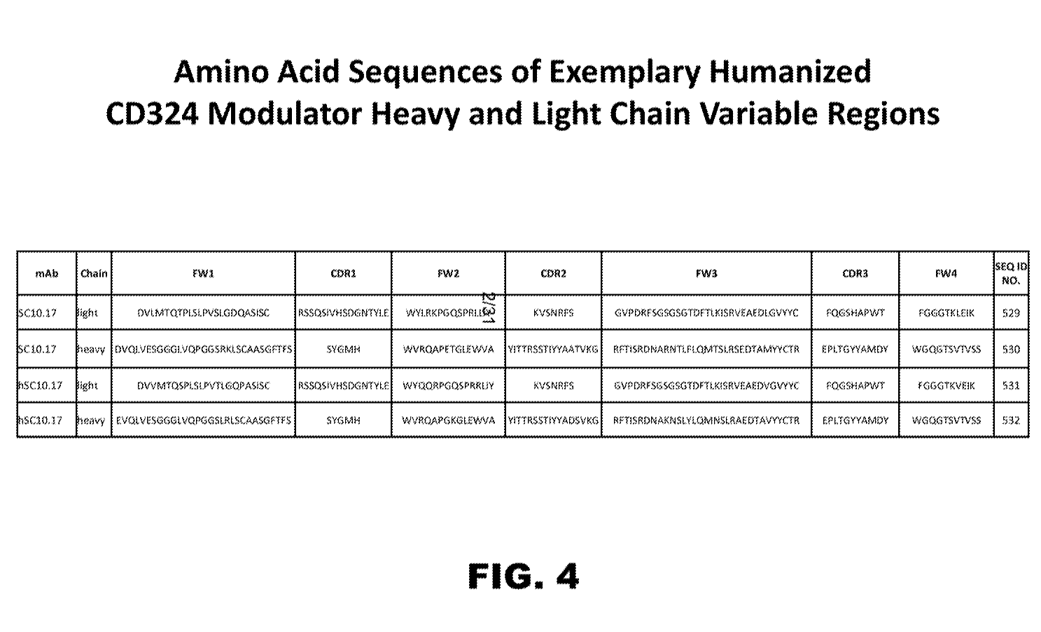

In the context of the instant invention it will be appreciated that any of the disclosed light and heavy chain CDRs derived from the murine variable region amino acid sequences set forth in the appended sequence listing (anti-SEZ6 antibodies) may be combined with acceptor antibodies or rearranged to provide optimized anti-human SEZ6 (e.g. humanized or chimeric anti-hSEZ6) site-specific antibodies in accordance with the instant teachings. That is, one or more of the CDRs derived or obtained from the contiguous light chain variable region amino acid sequences set forth in the appended sequence listing (together SEQ ID NOS: 20-169) may be incorporated in a site-specific construct and, in particularly preferred embodiments, in a CDR grafted or humanized site-specific antibody that immunospecifically associates with one or more SEZ6 isoforms or family members. Examples of “derived” light and heavy chain variable region amino acid sequences of such humanized modulators are also set forth in FIGS. 2A and 2B for anti-DLL3 antibodies (SEQ ID NOS: 519-528), FIGS. 3A and 3B for anti-SEZ6 antibodies (SEQ ID NOS: 170-199) and FIG. 4 for an anti-CD324 antibodies (SEQ ID NOS: 531 and 532).

In FIGS. 2A and 2B, 3A and 3B and 4 the annotated CDRs and framework sequences are defined as per Kabat using a proprietary Abysis database. However, as discussed herein one skilled in the art could readily define, identify, derive and/or enumerate the CDRs as defined by Kabat et al., Chothia et al., ABM or MacCallum et al. for each respective heavy and light chain sequence set forth in the appended sequence listing. Accordingly, each of the subject CDRs and antibodies comprising CDRs defined by all such nomenclature are expressly included within the scope of the instant invention. More broadly, the terms “variable region CDR amino acid residue” or more simply “CDR” includes amino acids in a CDR as identified using any sequence or structure based method as set forth above. Within this context Kabat CDRs for the exemplary humanized antibodies in FIGS. 3A and 3B are provided in the appended sequence listing as SEQ ID NOS: 405-470.

Another aspect of the invention comprises site-specific anti-SEZ6 antibodies obtained or derived from SC17.1, SC17.2, SC17.3, SC17.4, SC17.8, SC17.9, SC17.10, SC17.11, SC17.14, SC17.15, SC17.16, SC17.17, SC17.18, SC17.19, SC17.22, SC17.24, SC17.27, SC17.28, SC17.29, SC17.30, SC17.32, SC17.34, SC17.35, SC17.36, SC17.38, SC17.39, SC17.40, SC17.41, SC17.42, SC17.45, SC17.46, SC17.47, SC17.49, SC17.50, SC17.53, SC17.54, SC17.56, SC17.57, SC17.59, SC17.61, SC17.63, SC17.71, SC17.72, SC17.74, SC17.76, SC17.77, SC17.79, SC17.81, SC17.82, SC17.84, SC17.85, SC17.87, SC17.89, SC17.90, SC17.91, SC17.93, SC17.95, SC17.97, SC17.99, SC17.102, SC17.114, SC17.115, SC17.120, SC17121, SC17.122, SC17.140, SC17.151, SC17.156, SC17.161, SC17.166, SC17.187, SC17.191, SC17.193, SC17.199 and SC17.200; or any of the above-identified antibodies, or chimeric or humanized versions thereof. In other embodiments the ADCs of the invention will comprise a SEZ6 antibody having one or more CDRs, for example, one, two, three, four, five, or six CDRs, from any of the aforementioned modulators. The annotated sequence listing provides the individual SEQ ID NOS for the heavy and light chain variable regions for each of the aforementioned anti-SEZ6 antibodies.

2. Site-Specific Antibodies

Based on the instant disclosure one skilled in the art could readily fabricate engineered constructs as described herein. As used herein, “engineered antibody” “engineered construct” or “site-specific antibody” means an antibody, or immunoreactive fragment thereof, wherein at least one amino acid in either the heavy or light chain is incorporated, deleted, altered or substituted (preferably with another amino acid) to provide at least one free cysteine. Similarly, an “engineered conjugate” or “site-specific conjugate” shall be held to mean an antibody drug conjugate comprising an engineered antibody and at least one cytotoxin conjugated to the unpaired or free cysteine(s). As discussed above, the incorporation, deletion, alteration or substation may generate a free cysteine from disruption of a native disulfide bridge or from the introduction or addition of a cysteine to the amino acid sequence. Thus, in certain embodiments the unpaired cysteine residue will comprise an unpaired intrachain residue. In other preferred embodiments the free cysteine residue will comprise an unpaired interchain cysteine residue. The engineered antibody can be of various isotypes, for example, IgG, IgE, IgA or IgD; and within those classes the antibody can be of various subclasses, for example, IgG1, IgG2, IgG3 or IgG4. With regard to such IgG constructs the light chain of the antibody can comprise either a kappa or lambda isotype each incorporating a C214 or, as set forth in SEQ ID NOS: 550-564 a cysteine another position of the CL domain that, in each case, may be unpaired due to a lack of a C220 residue in the IgG1 heavy chain.

In selected embodiments the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an intrachain cysteine residue is one naturally paired with another cysteine in the same heavy or light chain. In one embodiment the deleted or substituted interchain cysteine residue is in involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In one particularly preferred embodiment an interchain cysteine is replaced with a serine.

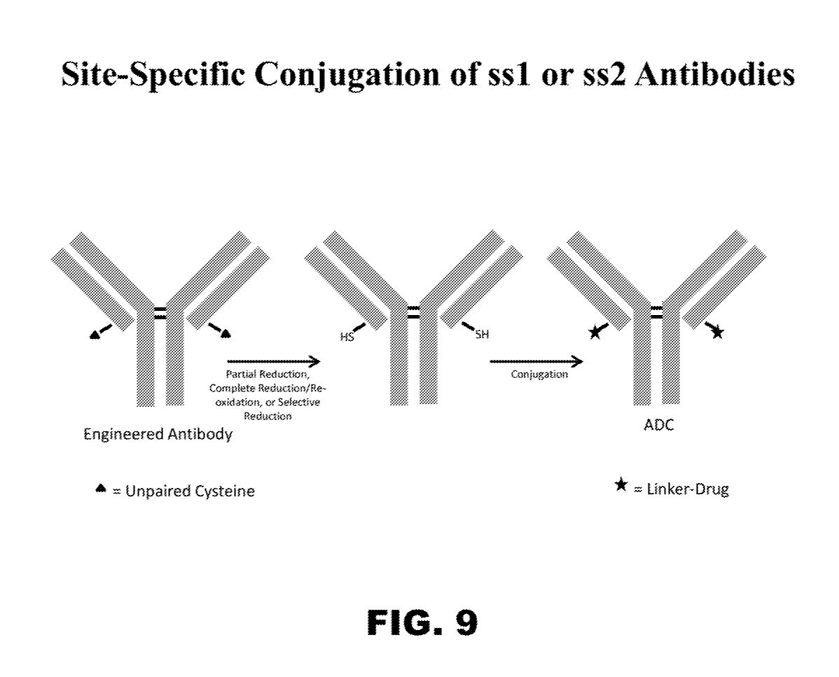

In some embodiments contemplated by the invention the deleted or substituted cysteine residue is on the light chain (either kappa or lambda) thereby leaving a free cysteine on the heavy chain (e.g., C220 in SEQ ID NO: 404). In other embodiments the deleted or substituted cysteine residue is on the heavy chain leaving the free cysteine on the light chain constant region (e.g., see SEQ ID NOS: 403, 504 and 550-564). FIG. 1 depicts the cysteines involved in the native or naturally occurring interchain and intrachain disulfide bonds (or bridges) in an exemplary IgG1/kappa antibody. As previously indicated in each case the amino acid residues of the constant regions are numbered based on the EU index according to Kabat. As shown in an exemplary embodiment in FIG. 9, deletion or substitution of a single cysteine in either the light or heavy chain of an intact antibody results in an engineered antibody having two unpaired cysteine residues.

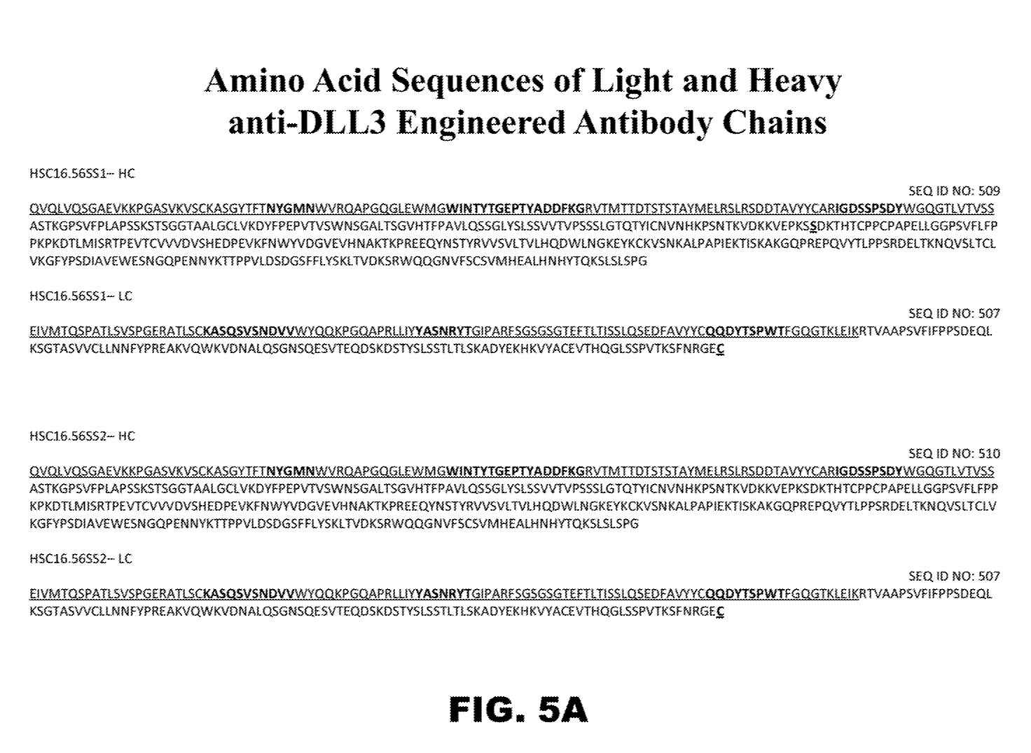

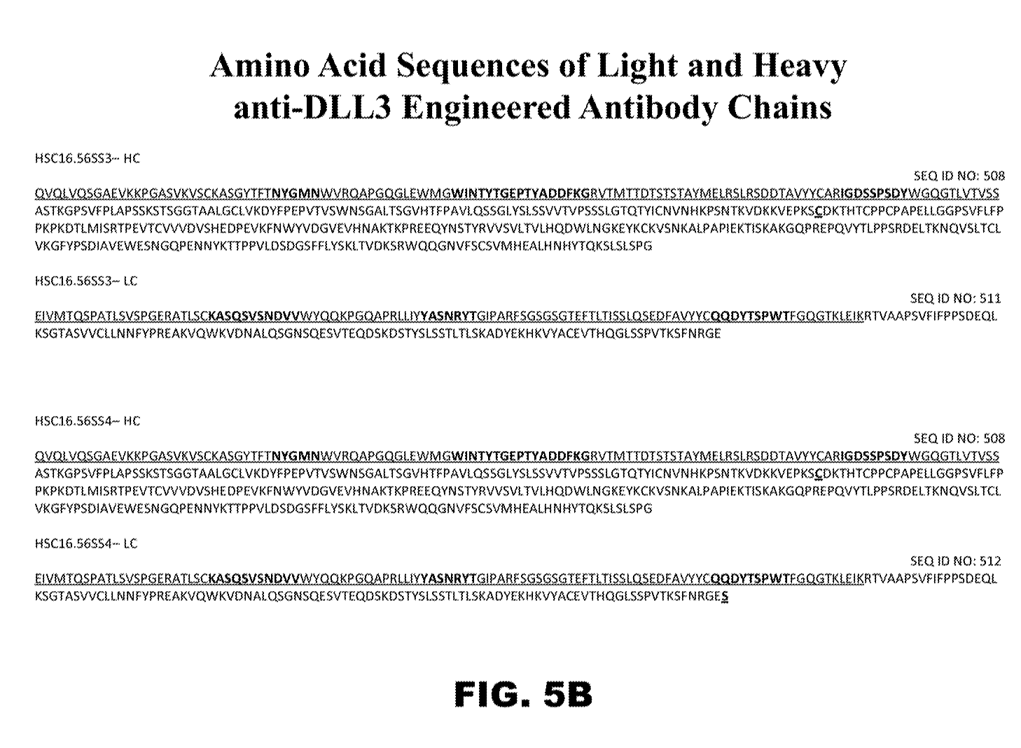

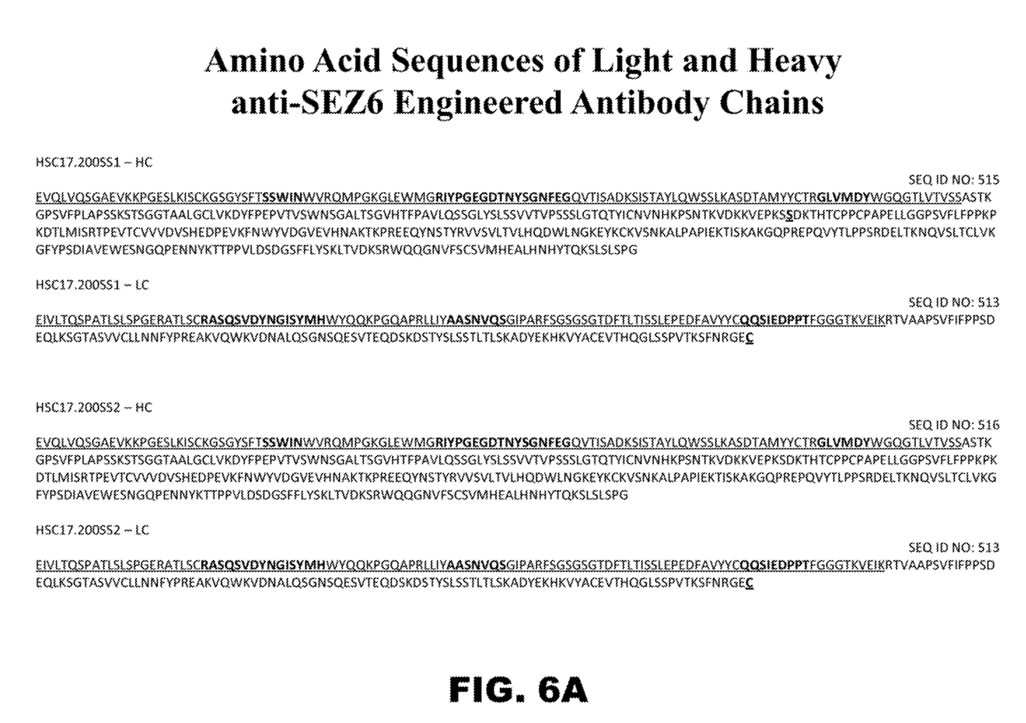

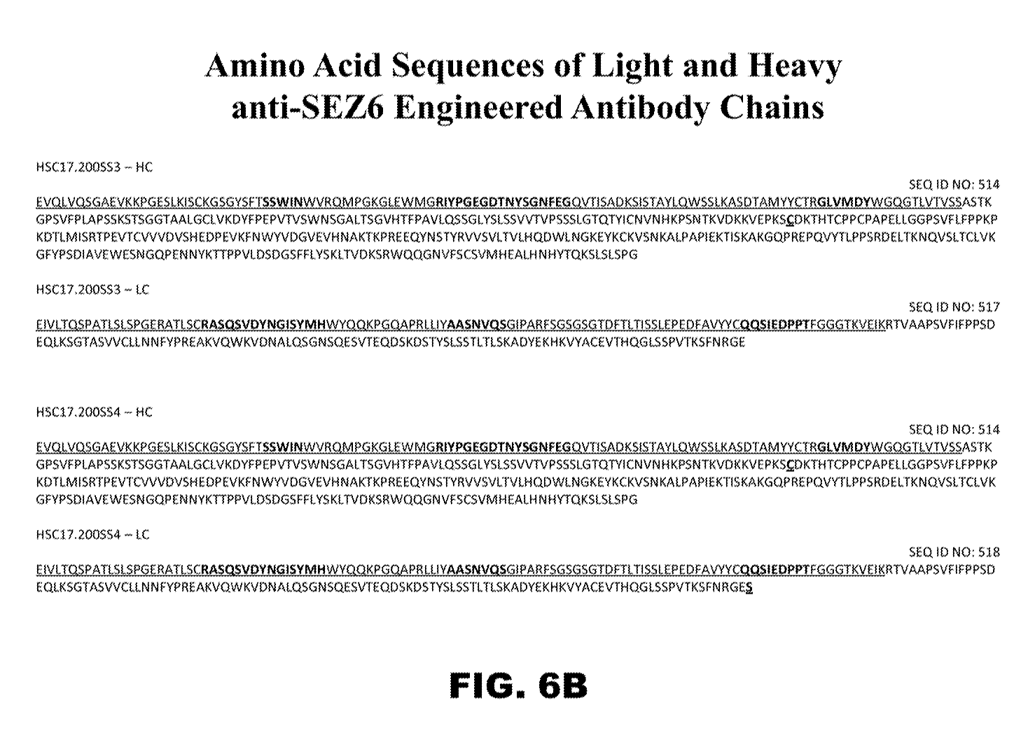

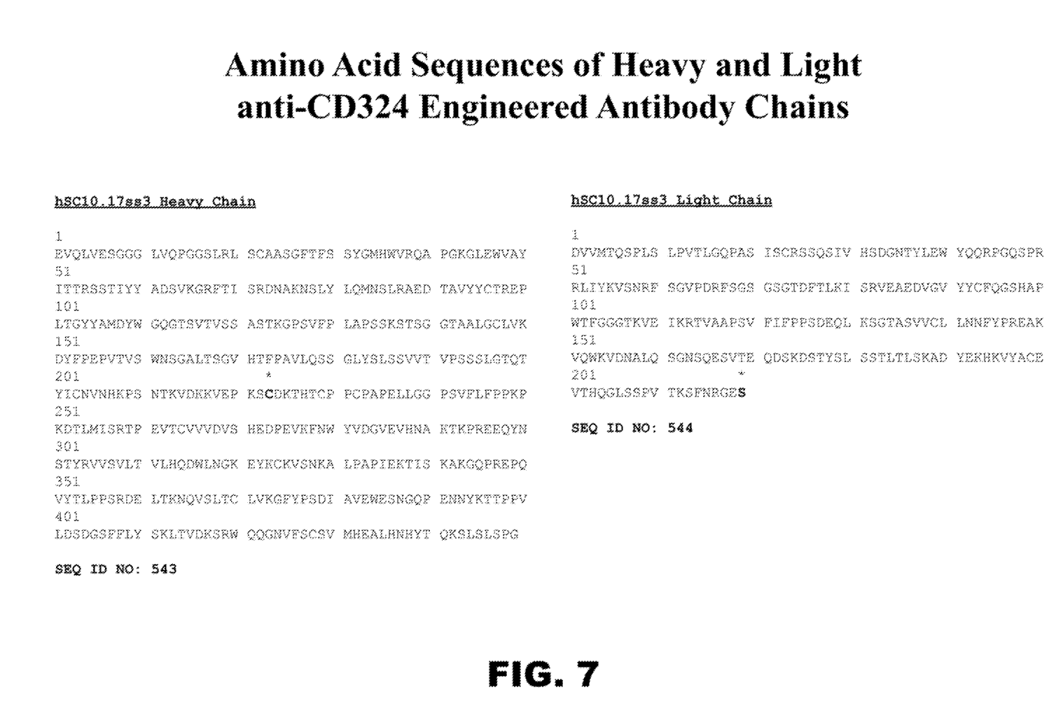

In particularly preferred embodiments the cysteine at position 214 (C214) of the IgG light chain (kappa or lambda) is deleted or substituted. In another preferred embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-engineered constructs provided as per Examples 6-8 respectively are shown in FIGS. 5A and 5B using the exemplary anti-DLL3 antibody SC16.56, FIGS. 6A and 6B using the exemplary anti-SEZ6 antibody SC17.200 and FIG. 7 for the exemplary anti-CD324 antibody SC10.17. Additional examples are also provided for the exemplary anti-SEZ6 antibody SC17.17 in the appended sequence listing (SEQ ID NOS: 537-542).

A summary of exemplary constructs is shown in Table 2 immediately below where all numbering is according to Kabat and WT stands for “wild-type” or native constant region sequences without alterations. It will be appreciated that the particular substitutions shown in Table 2 (e.g., C220S) are illustrative only and that any compatible residue (e.g., a natural or non-natural amino acid) that provides the desired free cysteine may be substituted instead (e.g., C220G or C220A). Note that, while many of the referenced sequences are generally derived from kappa CL, exemplary lambda light chains comprising C214 may also be used as set forth herein to provide the desired free cysteines. Also, as used herein delta (4) shall designate the deletion of an amino acid residue (e.g., C2144 indicates that the cysteine at position 214 has been deleted).

TABLE 2

| | Antibody | | Const. Reg. |
|---|---|---|---|
| Designation | Component | Alteration | SEQ ID NO: |
| ss1 | Heavy Chain | C220S | 500 |
| | Light Chain | WT | 403 or 504 |
| ss2 | Heavy Chain | C220Δ | 501 |
| | Light Chain | WT | 403 or 504 |
| ss3 | Heavy Chain | WT | 404 |
| | Light Chain | C214Δ | 502 or 505 |
| ss4 | Heavy Chain | WT | 404 |
| | Light Chain | C214S | 503 or 506 |
| ss5 | Heavy Chain | C220S or C220Δ | 500 or 501 |
| | Light Chain | E213D | 550 |
| ss6 | Heavy Chain | C220S or C220Δ | 500 or 501 |
| | Light Chain | E213EE | 551 |
| ss7 | Heavy Chain | C220S or C220Δ | 500 or 501 |
| | Light Chain | E213C, C214E | 552 |
| ss8 | Heavy Chain | C220S or C220Δ | 500 or 501 |
| | Light Chain | G212C, E212E C214Δ | 553 |
| ss9 | Heavy Chain | C220S or C220Δ | 500 or 501 |
| | Light Chain | D122C, C214S | 554 |
| ss10 | Heavy Chain | C220S or C220Δ | 500 or 501 |
| | Light Chain | K190C, C214S | 555 |
| ss11 | Heavy Chain | C220S or C220Δ | 500 or 501 |
| | Light Chain | T206C, C214S | 556 |
| ss12 | Heavy Chain | C220S or C220Δ | 500 or 501 |
| | Light Chain | S208C, C214S | 557 |
| ss13 | Heavy Chain | C220S or C220Δ | 500 or 501 |
| | Light Chain | N210C, C214S | 558 |
| ss14 | Heavy Chain | C220S or C220Δ | 500 or 501 |
| | Light Chain | R211C, C214S | 559 |
| ss15 | Heavy Chain | C220S or C220Δ | 500 or 501 |
| | Light Chain | G212C, C214S | 560 |
| ss16 | Heavy Chain | C220S or C220Δ | 500 or 501 |
| | Light Chain | E213C, C214S | 561 |
| ss17 | Heavy Chain | C220S or C220Δ | 500 or 501 |
| | Light Chain | R211C, G212-C214Δ | 562 |
| ss18 | Heavy Chain | C220S or C220Δ | 500 or 501 |
| | Light Chain | R211C, G212E, E213-C214Δ | 563 |
| ss19 | Heavy Chain | C220S or C220Δ | 500 or 501 |
| | Light Chain | G212C, E213-C214Δ | 564 |

As set forth herein in certain embodiments the free cysteine is introduced into or added to the amino acid sequence comprising the heavy or light chain of the antibody. Included in such embodiments are instances in which certain non-cysteine residues are mutated or substituted with cysteine to provide the desired free cysteine. In selected embodiments set forth in the Examples below the free cysteine residue will be incorporated in one of the exposed loop structures of the constant region of an antibody light chain. In such embodiments the free cysteine residue will be positioned (by incorporation or substitution) in residues 121-128, residues 182-191 or residues 201-213 (Kabat numbering). In other preferred embodiments the free cysteine residue will comprise (by incorporation or substitution) an exposed residue on the beta sheet of the CL region. In yet other embodiments the free cysteine residue will be positioned within the CH1 domain. In still other embodiments the free cysteine residue will be positioned in the CH2 domain. In yet other embodiments the free cysteine residue will be positioned in the CH3 domain. In still other preferred embodiments any one or more of the following residues may be substituted with cysteine: V205 (Kabat numbering) of the light chain; A118 (EU numbering) of the heavy chain; and S400 (EU numbering) of the heavy chain Fc region. Other compatible positions that may comprise free cysteines include (according to EU numbering): 41, 88, 116, 120, 171, 282 or 375. It is important to emphasize that regardless of the positioning of the free cysteine each such construct comprising an introduced or substituted cysteine residue, including each of the aforementioned constructs, may be selectively conjugated as set forth herein and is expressly included within the scope of the instant invention.

The strategy for generating antibody-drug conjugates with defined sites and stoichiometries of drug loading, as disclosed herein, is broadly applicable to other antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention.

3. Antibody Generation

a. Polyclonal Antibodies

The production of polyclonal antibodies in various host animals, including rabbits, mice, rats, etc. is well known in the art. In some embodiments, polyclonal antibody-containing serum is obtained by bleeding or sacrificing the animal. The serum may be used for research purposes in the form obtained from the animal or, in the alternative, the antibodies may be partially or fully purified to provide immunoglobulin fractions or homogeneous antibody preparations.

Briefly the selected animal is immunized with an immunogen (e.g., soluble DLL3 or sDLL3) which may, for example, comprise selected isoforms, domains and/or peptides, or live cells or cell preparations expressing DLL3 or immunoreactive fragments thereof. Art known adjuvants that may be used to increase the immunological response, depending on the inoculated species include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants may protect the antigen from rapid dispersal by sequestering it in a local deposit, or they may contain substances that stimulate the host to secrete factors that are chemotactic for macrophages and other components of the immune system. Preferably the immunization schedule will involve two or more administrations of the selected immunogen spread out over a predetermined period of time.

By way of example the amino acid sequence of a DLL3 protein can be analyzed to select specific regions of the DLL3 protein for generating antibodies. For instance, hydrophobicity and hydrophilicity analyses of a DLL3 amino acid sequence are used to identify hydrophilic regions in the DLL3 structure. Regions of a DLL3 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Average Flexibility profiles can be generated using the method of Bhaskaran R., Ponnuswamy P. K., 1988, Int. J. Pept. Protein Res. 32:242-255. Beta-turn profiles can be generated using the method of Deleage, G., Roux B., 1987, Protein Engineering 1:289-294. Thus, each DLL3 region, domain or motif identified by any of these programs or methods is within the scope of the present invention and may be isolated or engineered to provide immunogens giving rise to modulators comprising desired properties. Preferred methods for the generation of DLL3 antibodies are further illustrated by way of the Examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents are effective. Administration of a DLL3 immunogen is often conducted by injection over a suitable time period and with use of a suitable adjuvant, as is understood in the art. During the immunization schedule, titers of antibodies can be taken as described in the Examples below to determine adequacy of antibody formation.

b. Monoclonal Antibodies

In addition, the invention contemplates use of monoclonal antibodies. As known in the art, the term “monoclonal antibody” (or mAb) refers to an antibody obtained from a population of substantially homogeneous antibodies, i.e., the individual antibodies comprising the population are identical except for possible mutations (e.g., naturally occurring mutations) that may be present in minor amounts. In certain embodiments, such a monoclonal antibody includes an antibody comprising a polypeptide sequence that binds or associates with an antigen wherein the antigen-binding polypeptide sequence was obtained by a process that includes the selection of a single target binding polypeptide sequence from a plurality of polypeptide sequences.

More generally, and as set forth in the Examples herein, monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and art-recognized biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981) each of which is incorporated herein in its entirety by reference. It should be understood that a selected binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also an antibody of this invention. Murine monoclonal antibodies compatible with the instant invention are provided as set forth in the Examples below.

c. Chimeric and Humanized Antibodies

In another embodiment, the antibodies of the invention may comprise chimeric antibodies derived from covalently joined protein segments from at least two different species or class of antibodies. The term “chimeric” antibodies is directed to constructs in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies (U.S. Pat. No. 4,816,567; Morrison et al., 1984, PMID: 6436822).

In one embodiment, a chimeric antibody may comprise murine V_{H} and V_{L} amino acid sequences and constant regions derived from human sources, for example, humanized antibodies as described below. In some embodiments, the antibodies can be “CDR-grafted”, where the antibody comprises one or more CDRs from a particular species or belonging to a particular antibody class or subclass, while the remainder of the antibody chain(s) is/are identical with or homologous to a corresponding sequence in antibodies derived from another species or belonging to another antibody class or subclass. For use in humans, selected rodent CDRs, e.g., mouse CDRs may be grafted into a human antibody, replacing one or more of the naturally occurring CDRs of the human antibody. These constructs generally have the advantages of providing full strength antibody functions, e.g., complement dependent cytotoxicity (CDC) and antibody-dependent cell-mediated cytotoxicity (ADCC) while reducing unwanted immune responses to the antibody by the subject.

Similar to the CDR-grafted antibody is a “humanized” antibody. As used herein, “humanized” forms of non-human (e.g., murine) antibodies are chimeric antibodies that comprise amino acids sequences derived from one or more non-human immunoglobulins. In one embodiment, a humanized antibody is a human immunoglobulin (recipient or acceptor antibody) in which residues from one or more CDRs of the recipient are replaced by residues from one or more CDRs of a non-human species (donor antibody) such as mouse, rat, rabbit, or non-human primate. In certain preferred embodiments, residues in one or more FRs in the variable domain of the human immunoglobulin are replaced by corresponding non-human residues from the donor antibody to help maintain the appropriate three-dimensional configuration of the grafted CDR(s) and thereby improve affinity. This can be referred to as the introduction of “back mutations”. Furthermore, humanized antibodies may comprise residues that are not found in the recipient antibody or in the donor antibody to, for example, further refine antibody performance. Humanized anti-DLL3 antibodies compatible with the instant invention are provided in Example 3 below with resulting humanized light and heavy chain amino acid sequences shown in FIGS. 2A and 2B. Humanized anti-SEZ6 antibodies are provided as per Example 4 with resulting humanized light and heavy chain amino acid sequences shown in FIGS. 3A and 3B while a humanized anti-CD324 antibody was provided as per Example 5 with corresponding sequences shown in FIG. 4. FIGS. 5A and 5B, 6A and 6B and 7 show, respectively, site-specific exemplary humanized antibody heavy and light chain annotated amino acid sequences for the three antigens.

Various sources can be used to determine which human sequences to use in the humanized antibodies. Such sources include human germline sequences that are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638; the V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK); or consensus human FRs described, for example, in U.S. Pat. No. 6,300,064.

CDR grafting and humanized antibodies are described, for example, in U.S. Pat. Nos. 6,180,370 and 5,693,762. For further details, see, e.g., Jones et al., 1986, PMID: 3713831); and U.S. Pat. Nos. 6,982,321 and 7,087,409.

Another method is termed “humaneering” which is described, for example, in U.S.P.N. 2005/0008625. In another embodiment a non-human antibody may be modified by specific deletion of human T-cell epitopes or “deimmunization” by the methods disclosed in WO 98/52976 and WO 00/34317.

As discussed above in selected embodiments at least 60%, 65%, 70%, 75%, or 80% of the humanized or CDR grafted antibody heavy or light chain variable region amino acid residues will correspond to those of the recipient human sequences. In other embodiments at least 83%, 85%, 87% or 90% of the humanized antibody variable region residues will correspond to those of the recipient human sequences. In a further preferred embodiment, greater than 95% of each of the humanized antibody variable regions will correspond to those of the recipient human sequences.

The sequence identity or homology of the humanized antibody variable region to the human acceptor variable region may be determined as previously discussed and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

d. Human Antibodies

In another embodiment, the antibodies may comprise fully human antibodies. The term “human antibody” refers to an antibody which possesses an amino acid sequence that corresponds to that of an antibody produced by a human and/or has been made using any of the techniques for making human antibodies.

Human antibodies can be produced using various techniques known in the art. One technique is phage display in which a library of (preferably human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; and Barbas et al., Proc. Natl. Acad. Sci. USA 88:7978-7982 (1991)).

In one embodiment, recombinant human antibodies may be isolated by screening a recombinant combinatorial antibody library prepared as above. In one embodiment, the library is a scFv phage display library, generated using human V_{L} and V_{H} cDNAs prepared from mRNA isolated from B-cells.

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)). Additionally, affinity maturation can be performed by randomly mutating one or more CDRs, e.g. using PCR with primers carrying random sequence spanning the CDR of interest, in selected individual Fv clones and screening for higher-affinity clones. WO 9607754 described a method for inducing mutagenesis in a CDR of an immunoglobulin light chain to create a library of light chain genes. Another effective approach is to recombine the V_{H} or V_{L} domains selected by phage display with repertoires of naturally occurring V domain variants obtained from unimmunized donors and to screen for higher affinity in several rounds of chain reshuffling as described in Marks et al., Biotechnol., 10: 779-783 (1992). This technique allows the production of antibodies and antibody fragments with a dissociation constant K_{D} (k_{off}/kₒₙ) of about 10⁻⁹M or less.

In other embodiments, similar procedures may be employed using libraries comprising eukaryotic cells (e.g., yeast) that express binding pairs on their surface. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. USA 95:6157-6162 (1998). In other embodiments, human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J. Immunol, 147 (l):86-95 (1991); and U.S. Pat. No. 5,750,373.

4. Recombinant Production of Antibodies

The site-specific antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc. (supplemented through 2006); and U.S. Pat. No. 7,709,611).

More particularly, another aspect of the invention pertains to engineered nucleic acid molecules that encode the site-specific antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA or RNA and may or may not contain intronic sequences. More generally the term “nucleic acid”, as used herein, includes genomic DNA, cDNA, RNA and artificial variants thereof (e.g., peptide nucleic acids), whether single-stranded or double-stranded. In a preferred embodiment, the nucleic acid is a cDNA molecule.

Nucleic acids of the invention can be obtained and manipulated using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques (e.g., see Example 1). For antibodies obtained from an immunoglobulin gene library (e.g., using phage display techniques), nucleic acid encoding the antibody can be recovered from the library.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3) which may or may not be engineered as described herein. The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. As discussed in more detail below an exemplary IgG1 constant region that is compatible with the teachings herein is set forth as SEQ ID NO: 404 in the appended sequence listing with compatible engineered IgG1 constant regions set forth in SEQ ID NOS: 500 and 501. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth as SEQ ID NO: 403 in the appended sequence listing while a compatible lambda light chain constant region is set forth in SEQ ID NO: 504. Compatible engineered versions of the kappa and lambda light chain regions are shown in SEQ ID NOS: 502, 503 and 550-564 and 505, 506 respectively.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any kind of cellular system which can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK-293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under certain conditions. The GS system is discussed in whole or part in connection with U.S. Pat. Nos. 5,591,639 and 5,879,936. Another preferred expression system for the development of stable cell lines is the Freedom™ CHO-S Kit (Life Technologies).

Once an antibody of the invention has been produced by recombinant expression or any other of the disclosed techniques, it may be purified or isolated by methods known in the art, meaning that it is identified and separated and/or recovered from its natural environment and separated from contaminants that would interfere with conjugation or diagnostic or therapeutic uses for the antibody. Isolated antibodies include antibodies in situ within recombinant cells.

These isolated preparations may be purified using various art recognized techniques, such as, for example, ion exchange and size exclusion chromatography, dialysis, diafiltration, and affinity chromatography, particularly Protein A or Protein G affinity chromatography.

5. Antibody Fragments and Derivatives

a. Fragments

Regardless of which form of site-specific antibody (e.g. chimeric, humanized, etc.) is selected to practice the invention it will be appreciated that immunoreactive fragments of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody comprising at least one free cysteine that immunospecifically binds or reacts with a selected antigen or immunogenic determinant thereof or competes with the intact antibody from which the fragments were derived for specific antigen binding.

Exemplary site-specific fragments include: VL, VH, scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency).

In other embodiments, a site-specific antibody fragment is one that comprises the Fc region and that retains at least one of the biological functions normally associated with the Fc region when present in an intact antibody, such as FcRn binding, antibody half-life modulation, ADCC function and complement binding. In one embodiment, a site-specific antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

b. Multivalent Antibodies

In one embodiment, the site-specific conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105. In each case at least one of the binding sites will comprise an epitope, motif or domain associated with a DLL3 isoform.

In one embodiment, the modulators are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As alluded to above, multivalent antibodies may immunospecifically bind to different epitopes of the desired target molecule or may immunospecifically bind to both the target molecule as well as a heterologous epitope, such as a heterologous polypeptide or solid support material. While preferred embodiments of the anti-DLL3 antibodies only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

In yet other embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

c. Fc Region Modifications

In addition to the various modifications, substitutions, additions or deletions to the variable or binding region of the disclosed site-specific conjugates set forth above, including those generating a free cysteine, those skilled in the art will appreciate that selected embodiments of the present invention may also comprise substitutions or modifications of the constant region (i.e. the Fc region). More particularly, it is contemplated that the site-specific antibodies of the invention may contain inter alia one or more additional amino acid residue substitutions, mutations and/or modifications which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced “ADCC” (antibody-dependent cell mediated cytotoxicity) or “CDC” (complement-dependent cytotoxicity) activity, altered glycosylation and/or disulfide bonds and modified binding specificity. In this regard it will be appreciated that these Fc variants may advantageously be used to enhance the effective anti-neoplastic properties of the disclosed modulators.

To this end certain embodiments of the invention may comprise substitutions or modifications of the Fc region beyond those required to generate a free cysteine, for example the addition of one or more amino acid residue, substitutions, mutations and/or modifications to produce a compound with enhanced or preferred Fc effector functions. For example, changes in amino acid residues involved in the interaction between the Fc domain and an Fc receptor (e.g., FcγRI, FcγRIIA and B, FcγRIII and FcRn) may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995) each of which is incorporated herein by reference).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311. With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

d. Altered Glycosylation

Still other embodiments comprise one or more engineered glycoforms, e.g., a DLL3 site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the modulator for a target or facilitating production of the modulator. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTI11)), by expressing a molecule comprising an Fc region in various organisms or cell lines from various organisms or by modifying carbohydrate(s) after the molecule comprising Fc region has been expressed (see, for example, WO 2012/117002).

e. Additional Processing

The site-specific antibodies or conjugates may be differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Various post-translational modifications also encompassed by the invention include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Moreover, the modulators may also be modified with a detectable label, such as an enzymatic, fluorescent, radioisotopic or affinity label to allow for detection and isolation of the modulator.

6. Site-Specific Antibody Characteristics

No matter how obtained or which of the aforementioned forms the site-specific conjugate takes, various embodiments of the disclosed antibodies may exhibit certain characteristics. In selected embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted or influenced by selecting a particular antigen (e.g., a specific DLL3 isoform) or immunoreactive fragment of the target antigen for inoculation of the animal. In still other embodiments the selected antibodies may be engineered as described above to enhance or refine immunochemical characteristics such as affinity or pharmacokinetics.

a. Neutralizing Antibodies

In certain embodiments, the conjugates will comprise “neutralizing” antibodies or derivatives or fragments thereof. That is, the present invention may comprise antibody molecules that bind specific domains, motifs or epitopes and are capable of blocking, reducing or inhibiting the biological activity of, for example, DLL3. More generally the term “neutralizing antibody” refers to an antibody that binds to or interacts with a target molecule or ligand and prevents binding or association of the target molecule to a binding partner such as a receptor or substrate, thereby interrupting a biological response that otherwise would result from the interaction of the molecules.

It will be appreciated that competitive binding assays known in the art may be used to assess the binding and specificity of an antibody or immunologically functional fragment or derivative thereof. With regard to the instant invention an antibody or fragment will be held to inhibit or reduce binding of DLL3 to a binding partner or substrate when an excess of antibody reduces the quantity of binding partner bound to DLL3 by at least about 20%, 30%, 40%, 50%, 60%, 70%, 80%, 85%, 90%, 95%, 97%, 99% or more as measured, for example, by Notch receptor activity or in an in vitro competitive binding assay. In the case of antibodies to DLL3 for example, a neutralizing antibody or antagonist will preferably alter Notch receptor activity by at least about 20%, 30%, 40%, 50%, 60%, 70%, 80%, 85%, 90%, 95%, 97%, 99% or more. It will be appreciated that this modified activity may be measured directly using art-recognized techniques or may be measured by the impact the altered activity has downstream (e.g., oncogenesis, cell survival or activation or suppression of Notch responsive genes). Preferably, the ability of an antibody to neutralize DLL3 activity is assessed by inhibition of DLL3 binding to a Notch receptor or by assessing its ability to relieve DLL3 mediated repression of Notch signaling.

b. Internalizing Antibodies

There is evidence that a number of compatible determinants remain associated with a tumorigenic cell surface, thereby allowing for localization and internalization of the disclosed site-specific conjugates. In preferred embodiments such modulators will be associated with, or conjugated to, one or more drugs through engineered free cysteine site(s) that kill the cell upon internalization. In particularly preferred embodiments the site-specific conjugates will comprise an internalizing ADC.

As used herein, a modulator that “internalizes” is one that is taken up (along with any payload) by the cell upon binding to an associated antigen or receptor. As will be appreciated, the internalizing antibody may, in select embodiments, comprise antibody fragments and derivatives thereof, as well as antibody conjugates comprising a DAR of approximately 2. Internalization may occur in vitro or in vivo. For therapeutic applications, internalization will preferably occur in vivo in a subject in need thereof. The number of site-specific antibody conjugates internalized may be sufficient or adequate to kill an antigen-expressing cell, especially an antigen-expressing cancer stem cell. Depending on the potency of the payload or site-specific antibody conjugate as a whole, in some instances, the uptake of a single engineered antibody molecule into the cell is sufficient to kill the target cell to which the antibody binds. For example, certain drugs are so highly potent that the internalization of a few molecules of the toxin conjugated to the antibody is sufficient to kill the tumor cell. Whether an antibody internalizes upon binding to a mammalian cell can be determined by various art-recognized assays including Fab-Zap and Mab-Zab assays (Advanced Targeting Systems, Kit-48). Methods of detecting whether an antibody internalizes into a cell are also described in U.S. Pat. No. 7,619,068 which is incorporated herein by reference in its entirety.

c. Depleting Antibodies

In other embodiments the site-specific conjugate will comprise depleting antibodies or derivatives or fragments thereof. The term “depleting” antibody refers to an antibody that preferably binds to or associates with an antigen on or near the cell surface and induces, promotes or causes the death or elimination of the cell (e.g., by CDC, ADCC or introduction of a cytotoxic agent). In preferred embodiments, the selected depleting antibodies will be associated or conjugated to a drug.

Preferably a depleting antibody will be able to remove, incapacitate, eliminate or kill at least 20%, 30%, 40%, 50%, 60%, 70%, 80%, 85%, 90%, 95%, 97%, or 99% of DLL3 expressing cells in a defined cell population. In some embodiments the cell population may comprise enriched, sectioned, purified or isolated tumor perpetuating cells. In other embodiments the cell population may comprise whole tumor samples or heterogeneous tumor extracts that comprise cancer stem cells. Those skilled in the art will appreciate that standard biochemical techniques may be used to monitor and quantify the depletion of tumorigenic cells or tumor perpetuating cells in accordance with the teachings herein.

d. Binning and Epitope Mapping

It will further be appreciated the disclosed site-specific antibody conjugates will associate with, or bind to, discrete epitopes or immunogenic determinants presented by the selected target or fragment thereof. In certain embodiments, epitope or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. Thus, as used herein the term “epitope” includes any protein determinant capable of specific binding to an immunoglobulin or T-cell receptor or otherwise interacting with a molecule. In certain embodiments, an antibody is said to specifically bind (or immunospecifically bind or react) an antigen when it preferentially recognizes its target antigen in a complex mixture of proteins and/or macromolecules. In preferred embodiments, an antibody is said to specifically bind an antigen when the equilibrium dissociation constant (K_{D}) is less than or equal to 10⁻⁶M or less than or equal to 10⁻⁷M, more preferably when the equilibrium dissociation constant is less than or equal to 10⁻⁸M, and even more preferably when the dissociation constant is less than or equal to 10⁻⁹M

More directly the term “epitope” is used in its common biochemical sense and refers to that portion of the target antigen capable of being recognized and specifically bound by a particular antibody modulator. When the antigen is a polypeptide such as DLL3, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. In any event an antibody epitope typically includes at least 3, and more usually, at least 5 or 8-10 amino acids in a unique spatial conformation.

In this respect it will be appreciated that, in certain embodiments, an epitope may be associated with, or reside in, one or more regions, domains or motifs of, for example, the DLL3 protein. As discussed in more detail herein the extracellular region of the DLL3 protein comprises a series of generally recognized domains including six EGF-like domains and a DSL domain. For the purposes of the instant disclosure the term “domain” will be used in accordance with its generally accepted meaning and will be held to refer to an identifiable or definable conserved structural entity within a protein that exhibits a distinctive secondary structure content. In many cases, homologous domains with common functions will usually show sequence similarities and be found in a number of disparate proteins (e.g., EGF-like domains are reportedly found in at least 471 different proteins). Similarly, the art-recognized term “motif” will be used in accordance with its common meaning and shall generally refer to a short, conserved region of a protein that is typically ten to twenty contiguous amino acid residues. As discussed throughout, selected embodiments comprise site-specific antibodies that associate with or bind to an epitope within specific regions, domains or motifs of DLL3.

As discussed in more detail in PCT/US14/17810 particularly preferred epitopes of human DLL3 bound by exemplary site-specific antibody conjugates are set forth in Table 3 immediately below.

TABLE 3

| Antibody Clone | Epitope | SEQ ID NO: |
|---|---|---|
| SC16.23 | Q93, P94, G95, A96, P97 | 3 |
| SC16.34 | G203, R205, P206 | 4 |
| SC16.56 | G203, R205, P206 | 4 |

Following a similar line of reasoning epitopes of the SEZ6 antigen were determined for selected antibodies. In this respect, and as set forth in PCT/US2013/027476 which is incorporated herein as to the same, site-specific anti-SEZ6 conjugates of the invention may comprise an antibody that specifically binds to an epitope on a SEZ6 protein wherein the epitope comprises amino acid residues selected from the group consisting of (i) residues R762, L764, Q777, 1779, D781 and Q782; (ii) residues R342 and K389 and (iii) residues T352, S353 and H375.

In any event once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that competitively bind with one another, i.e. the antibodies compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

As used herein, the term “binning” refers to methods used to group or classify antibodies based on their antigen binding characteristics and competition. While the techniques are useful for defining and categorizing modulators of the instant invention, the bins do not always directly correlate with epitopes and such initial determinations of epitope binding may be further refined and confirmed by other art-recognized methodology as described herein. However it will be appreciated that empirical assignment of antibody modulators to individual bins provides information that may be indicative of the therapeutic potential of the disclosed modulators.

More specifically, one can determine whether a selected reference antibody (or fragment thereof) binds to the same epitope or cross competes for binding with a second test antibody (i.e., is in the same bin) by using methods known in the art and set forth in the Examples herein. In one embodiment, a reference antibody modulator is associated with DLL3 antigen under saturating conditions and then the ability of a secondary or test antibody modulator to bind to DLL3 is determined using standard immunochemical techniques. If the test antibody is able to substantially bind to DLL3 at the same time as the reference anti-DLL3 antibody, then the secondary or test antibody binds to a different epitope than the primary or reference antibody. However, if the test antibody is not able to substantially bind to DLL3 at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment under test prevents or inhibits specific binding of a reference antibody to a common antigen. Typically, such an assay involves the use of purified antigen (e.g., DLL3 or a domain or fragment thereof) bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess and/or allowed to bind first. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody (i.e., a DLL3 modulator) by at least 30%, 40%, 45%, 50%, 55%, 60%, 65%, 70% or 75%. In some instance, binding of the test antibody is inhibited by at least 80%, 85%, 90%, 95%, or 97% or more.

With regard to the instant invention, and as set forth in PCT/US14/17810 which is incorporated herein as to the anti-DLL3 antibody bins, it has been determined (via surface plasmon resonance or bio-layer interferometry) that the extracellular domain of DLL3 defines at least nine bins by competitive binding termed “bin A” to “bin I” herein. Given the resolution provided by modulator binning techniques, it is believed that these nine bins comprise the majority of the bins that are present in the extracellular region of the DLL3 protein.

In this respect, and as known in the art the desired binning or competitive binding data can be obtained using solid phase direct or indirect radioimmunoassay (RIA), solid phase direct or indirect enzyme immunoassay (EIA or ELISA), sandwich competition assay, a Biacore™ 2000 system (i.e., surface plasmon resonance—GE Healthcare), a ForteBio® Analyzer (i.e., bio-layer interferometry—ForteBio, Inc.) or flow cytometric methodology. The term “surface plasmon resonance,” as used herein, refers to an optical phenomenon that allows for the analysis of real-time specific interactions by detection of alterations in protein concentrations within a biosensor matrix. The term “bio-layer interferometry” refers to an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. In particularly preferred embodiments the analysis (whether surface plasmon resonance, bio-layer interferometry or flow cytometry) is performed using a Biacore or ForteBio instrument or a flow cytometer (e.g., FACSAria II) as known in the art.

In order to further characterize the epitopes that the disclosed DLL3 antibody modulators associate with or bind to, domain-level epitope mapping may be performed using a modification of the protocol described by Cochran et al. (J Immunol Methods. 287 (1-2):147-158 (2004) which is incorporated herein by reference). Briefly, individual domains of DLL3 comprising specific amino acid sequences were expressed on the surface of yeast and binding by each DLL3 antibody was determined through flow cytometry.

Other compatible epitope mapping techniques include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9: 487-496) (herein specifically incorporated by reference in its entirety). In other embodiments Modification-Assisted Profiling (MAP), also known as Antigen Structure-based Antibody Profiling (ASAP) provides a method that categorizes large numbers of monoclonal antibodies (mAbs) directed against the same antigen according to the similarities of the binding profile of each antibody to chemically or enzymatically modified antigen surfaces (U.S.P.N. 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. It will be appreciated that MAP may be used to sort the hDLL3 antibody modulators of the invention into groups of antibodies binding different epitopes

Agents useful for altering the structure of the immobilized antigen include enzymes such as proteolytic enzymes (e.g., trypsin, endoproteinase Glu-C, endoproteinase Asp-N, chymotrypsin, etc.). Agents useful for altering the structure of the immobilized antigen may also be chemical agents, such as, succinimidyl esters and their derivatives, primary amine-containing compounds, hydrazines and carbohydrazines, free amino acids, etc.

The antigen protein may be immobilized on either biosensor chip surfaces or polystyrene beads. The latter can be processed with, for example, an assay such as multiplex LUMINEX™ detection assay (Luminex Corp.). Because of the capacity of LUMINEX to handle multiplex analysis with up to 100 different types of beads, LUMINEX provides almost unlimited antigen surfaces with various modifications, resulting in improved resolution in antibody epitope profiling over a biosensor assay.

e. Binding Affinity

Besides epitope specificity the disclosed site-specific antibodies may be characterized using physical characteristics such as, for example, binding affinities. In this regard the present invention further encompasses the use of antibodies that have a high binding affinity for one or more determinant isoforms or, in the case of pan-antibodies, more than one member of the determinant family. As used herein, the term “high affinity” for an IgG antibody refers to an antibody having a K_{D} of 10⁻⁸ M or less, more preferably 10⁻⁹M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷M or less, more preferably 10⁻⁸ M or less, even more preferably 10⁻⁹M or less.

The term “K_{D}”, as used herein, is intended to refer to the dissociation constant of a particular antibody-antigen interaction. An antibody of the invention is said to immunospecifically bind its target antigen when the dissociation constant K_{D} (k_{off}/kₒₙ) is ≤10⁻⁷M. The antibody specifically binds antigen with high affinity when the K_{D} is ≤5×10⁻⁹M, and with very high affinity when the K_{D} is ≤5×10⁻¹° M. In one embodiment of the invention, the antibody has a K_{D} of ≤10⁻⁹M and an off-rate of about 1×10⁻⁴/sec. In one embodiment of the invention, the off-rate is <1×10⁻⁵/sec. In other embodiments of the invention, the antibodies will bind to DLL3 with a K_{D} of between about 10⁻⁷M and 10⁻¹° M, and in yet another embodiment it will bind with a K_{D}≤2×10⁻¹° M. Still other selected embodiments of the present invention comprise antibodies that have a disassociation constant or K_{D} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹° M, less than 5×10⁻¹° M, less than 10⁻¹¹M⁻¹, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M or less than 5×10⁻¹⁵M.

In specific embodiments, an antibody of the invention that immunospecifically binds to DLL3 has an association rate constant or kₒₙ (or kₐ) rate ((Ab)+antigen (Ag)^{k}ₒₙ←Ab-Ag) of at least 10⁵M⁻¹s⁻¹, at least 2×10⁵M⁻¹s⁻¹, at least 5×10⁵M⁻¹s⁻¹, at least 10⁶M⁻¹s¹ at least 10⁶M⁻¹s⁻¹ at least 10⁷M⁻¹s⁻¹ at least 5×10⁷M⁻¹s⁻¹, or at least 10⁸M⁻¹.

In another embodiment, an antibody of the invention that immunospecifically binds to DLL3 has a disassociation rate constant or k_{off} (or k_{d}) rate ((Ab)+antigen (Ag)^{k}_{off}←Ab-Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹ less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹ or less than 10⁻¹⁰s⁻¹.

In other selected embodiments of the present invention antibodies will have an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10 at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹ or at least 5×10¹⁵M⁻¹.

Besides the aforementioned modulator characteristics antibodies of the instant invention may further be characterized using additional physical characteristics including, for example, thermal stability (i.e, melting temperature; Tm), and isoelectric points. (See, e.g., Bjellqvist et al., 1993, Electrophoresis 14:1023; Vermeer et al., 2000, Biophys. J. 78:394-404; Vermeer et al., 2000, Biophys. J. 79: 2150-2154 each of which is incorporated herein by reference).

### IV. Site-Specific Conjugates

The site-specific conjugates of the instant invention may be used to deliver cytotoxins or other payloads to the target location (e.g., tumorigenic cells). As used herein the terms “drug” or “warhead” may be used interchangeably and will mean a biologically active or detectable molecule or drug, including anti-cancer agents as described below. A “payload” may comprise a drug or “warhead” in combination with an optional linker compound. The “warhead” on the conjugate may comprise peptides, proteins or prodrugs which are metabolized to an active agent in vivo, polymers, nucleic acid molecules, small molecules, binding agents, mimetic agents, synthetic drugs, inorganic molecules, organic molecules and radioisotopes. In an advantageous embodiment, the disclosed ADCs will direct the bound payload to the target site in a relatively unreactive, non-toxic state before releasing and activating the warhead. This targeted release of the warhead is preferably achieved through stable conjugation of the payloads (e.g., via one or more cysteines on the engineered antibody) and the relatively homogeneous composition of the ADC preparations which minimize over-conjugated toxic species. Coupled with drug linkers that are designed to largely release the warhead once it has been delivered to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index when compared with conventional drug conjugates.

In this regard the term “engineered conjugate” or “site-specific conjugate” or simply “conjugate” will be used broadly and held to mean any site-specific construct comprising a biologically active or detectable molecule or drug associated with the disclosed targeting moiety through one or more free cysteines. Thus, it will be appreciated that, while some embodiments of the invention comprise payloads incorporating therapeutic moieties (e.g., cytotoxins), other payloads incorporating diagnostic agents and biocompatible modifiers may benefit from the targeted release provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to, the antibody and exhibit various stoichiometric molar ratios depending, at least in part, on the method used to effect the conjugation.

Essentially any payload that may be linked to a cysteine residue in a conventional antibody using art-recognized techniques may be associated with free cysteines of the engineered constructs of the instant invention using the novel techniques disclosed herein.

More specifically, once the disclosed site-specific antibodies of the invention have been generated and/or fabricated and selected according to the teachings herein they may be linked with, fused to, conjugated to, or otherwise associated with one or more pharmaceutically active or diagnostic moieties or biocompatible modifiers as described below. In this regard it will be appreciated that, unless otherwise dictated by context, the site-specific conjugates of the instant invention may be represented by the formula:

Ab-[L-D]n or a pharmaceutically acceptable salt thereof wherein
a) Ab comprises an antibody comprising one or more unpaired cysteine residue(s) wherein the unpaired cysteine residue(s) are exclusive of cysteines that form native interchain disulfide bonds;
b) L comprises an optional linker;
c) D comprises a drug; and
d) n is an integer from about 1 to about 12.

Those of skill in the art will appreciate that site-specific conjugates according to the aforementioned formula may be fabricated using a number of different linkers and drugs and that fabrication or conjugation methodology will vary depending on the selection of components. As such, any drug or drug linker compound that reacts with a thiol on the reactive cysteine(s) of the site-specific antibody is compatible with the teachings herein. Similarly, any reaction conditions that allow for site-specific conjugation of the selected drug to the engineered antibody are within the scope of the present invention. Notwithstanding the foregoing, particularly preferred embodiments of the instant invention comprise selective conjugation of the drug or drug linker using stabilization agents in combination with mild reducing agents as described herein and set forth in the Examples below. Such reaction conditions tend to provide more homogeneous preparations with less non-specific conjugation and contaminants and correspondingly less toxicity.

Exemplary payloads compatible with the teachings herein are listed below:

1. Therapeutic Agents

As indicated the site-specific antibodies of the invention may be conjugated, linked or fused to or otherwise associated with a pharmaceutically active moiety which is a therapeutic moiety or a drug such as an anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents (including homologs and derivatives thereof) comprise 1-dehydrotestosterone, anthramycins, actinomycin D, bleomycin, calicheamicin, colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In one embodiment the antibodies of the instant invention may be associated with anti-CD3 binding molecules to recruit cytotoxic T-cells and have them target tumorigenic cells (BiTE technology; see e.g., Fuhrmann et. al. (2010) Annual Meeting of AACR Abstract No. 5625).

In further embodiments the engineered conjugates of the invention may comprise therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I), carbon (¹⁴C), copper (⁶²Cu, ⁶⁴Cu, ⁶⁷Cu), sulfur (³⁵S), radium (²²³Ra), tritium (³H), indium (¹¹⁵In, ¹³In, ¹¹²In, ¹¹¹In), bismuth (²¹²Bi, ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷Lu, ¹⁵⁹Gd, ¹⁴⁹Pm, ¹⁴⁰La, ¹⁷⁵Yb, ¹⁶⁶Ho, ⁸⁹Zr ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Sn, ²²⁵Ac, ⁷⁶Br and ²¹¹At. Other radionuclides are also available as diagnostic and therapeutic agents, especially those in the energy range of 60 to 4,000 keV.

In certain some embodiments, the ADCs of the invention may comprise PBDs, and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157 and PCT filings WO2011/130613, WO2011/128650, WO2011/130616, WO2014/057073 and WO2014/057074.

In other selected embodiments the site-specific ADCs of the instant invention will be conjugated to a cytotoxic benzodiazepine derivative warhead. Compatible benzodiazepine derivatives (and optional linkers) that may be conjugated to the disclosed antibodies are described, for example, in U.S. Pat. No. 8,426,402 and PCT filings WO2012/128868 and WO2014/031566. As with the aforementioned PBDs, compatible benzodiazepine derivatives are believed to bind in the minor grove of DNA and inhibit nucleic acid synthesis. Such compounds reportedly have potent antitumor properties and, as such, are particularly suitable for use in the ADCs of the instant invention.

In addition to the aforementioned agents the antibodies of the present invention may also be conjugated to biological response modifiers. For example, in some embodiments the drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-α or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other preferred embodiments, site-specific antibodies of the present invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis analysis and/or detection can be accomplished by coupling the modulator to detectable substances including, but not limited to, various enzymes comprising for example horseradish peroxidase, alkaline phosphatase, beta-galactosidase, or acetyl cholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I), carbon (¹⁴C), sulfur (³⁵S), tritium (³H), indium (¹¹⁵In, ¹¹³In, ¹¹²In, ¹¹¹In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁸⁹Zr, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabeled or conjugated to specific radioisotopes. In such embodiments appropriate detection methodology is well known in the art and readily available from numerous commercial sources.

As indicated above, in other embodiments the site-specific antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In preferred embodiments, the marker comprises a his-tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments engineered antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

4. Linker Compounds

As with the aforementioned payloads numerous linker compounds are compatible with the instant invention and may be successfully used in combination with the teachings herein to provide the disclosed site-specific conjugates. In a broad sense the linkers merely need to covalently bind with the reactive thiol provided by the free cysteine and the selected drug compound. However, in other embodiments compatible linkers may covalently bind the selected drug at any accessible site including any substituents. Accordingly, any linker that reacts with the free cysteine(s) of the engineered antibody and may be used to provide the relatively stable site-specific conjugates of the instant invention is compatible with the teachings herein.

With regard to effectively binding to the selectively reduced free cysteine a number of art-recognized compounds take advantage of the good nucleophilicity of thiols and thus are available for use as part of a compatible linker. Free cysteine conjugation reactions include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein and shown in the Examples below, thiol-maleimide bioconjugation is one of the most widely used approaches due to its fast reaction rates and mild conjugation conditions. One issue with this approach is possibility of the retro-Michael reaction and loss or transfer of the maleimido-linked payload from the antibody or other target protein to other proteins in the plasma, such as, for example, human serum albumin. However, the use of selective reduction and site-specific antibodies as set forth herein may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

With regard to compatible linkers the compounds incorporated into the disclosed ADCs are preferably stable extracellularly, prevent aggregation of ADC molecules and keep the ADC freely soluble in aqueous media and in a monomeric state. Before transport or delivery into a cell, the antibody-drug conjugate is preferably stable and remains intact, i.e. the antibody remains linked to the drug moiety. While the linkers are stable outside the target cell they are designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety. As discussed in more detail in the appended Examples stability of the ADC may be measured by standard analytical techniques such as mass spectroscopy, hydrophobic interaction chromatography (HIC), HPLC, and the separation/analysis technique LC/MS. As set forth above covalent attachment of the antibody and the drug moiety requires the linker to have two reactive functional groups, i.e. bivalency in a reactive sense. Bivalent linker reagents which are useful to attach two or more functional or biologically active moieties, such as MMAE and site-specific antibodies are known, and methods have been described to provide their resulting conjugates.

Linkers compatible with the present invention may broadly be classified as cleavable and non-cleavable linkers. Cleavable linkers, which may include acid-labile linkers, protease cleavable linkers and disulfide linkers, take advantage of internalization by the target cell and cleavage in the endosomal-lysosomal pathway. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethyleneglycol or alkyl spacers liberate toxic payloads during lysosomal degradation of the antibody-drug conjugate within the target cell. In some respects the selection of linker will depend on the particular drug used in the site-specific conjugate.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells. Exemplary peptidyl linkers that are cleavable by the thiol-dependent protease Cathepsin-B are peptides comprising Phe-Leu since cathepsin-B has been found to be highly expressed in cancerous tissue. Other examples of such linkers are described, for example, in U.S. Pat. No. 6,214,345 which incorporated herein by reference as to such linkers. In a specific preferred embodiment, the peptidyl linker cleavable by an intracellular protease is a Val-Cit linker, a Val-Ala linker or a Phe-Lys linker such as is described in U.S. Pat. No. 6,214,345. One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, oxime, semicarbazone, thiosernicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable at below pH 5.5 or 5.0, the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10): 1305-12).

In particularly preferred embodiments (set forth in U.S.P.N. 2011/0256157 which is incorporated herein as to the linkers) compatible peptidyl linkers will comprise:

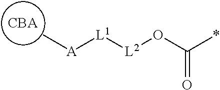

where the asterisk indicates the point of attachment to the drug, CBA is the site-specific antibody, L¹ is a linker, A is a connecting group connecting L¹ to an unpaired cysteine on the site specific antibody, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidising conditions may also find use in the present invention.

L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In one embodiment, ^{L} comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from:
-Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from:
-Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala-.

In one embodiment, L² is present and together with —C(═O)O— forms a self-immolative linker. In one embodiment, L² is a substrate for enzymatic activity, thereby allowing release of the drug.

In one embodiment, where L¹ is cleavable by the action of an enzyme and L² is present, the enzyme cleaves the bond between L¹ and L².

L¹ and L², where present, may be connected by a bond selected from:
—C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

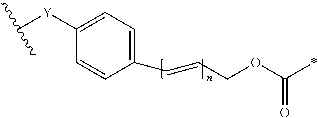

where the asterisk indicates the point of attachment to the drug or cytotoxic agent position, the wavy line indicates the point of attachment to the linker L, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In another particularly preferred embodiments the linker may include a self-immolative linker and the dipeptide together form the group —NH-Val-Ala-CO—NH-PABC-, which is illustrated below:

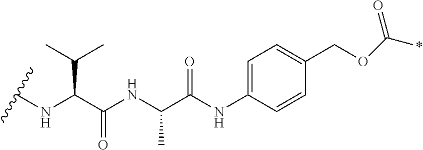 [SMILES: *N[C@H](C(=O)N[C@@H](C)C(=O)Nc1ccc(COC(*)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide the self-immolative linker will allow for clean release of the protected compound (i.e., the cytotoxin) when a remote site is activated, proceeding along the lines shown below:

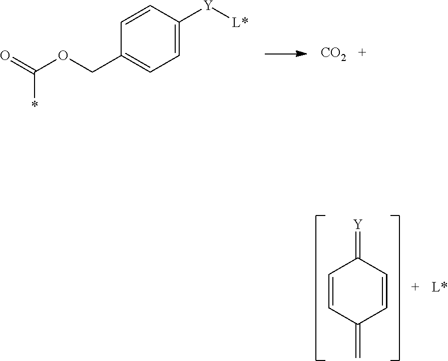

where L* is the activated form of the remaining portion of the linker comprising the now cleaved peptidyl unit. The clean release of the drug ensures they will maintain the desired toxic activity.

In one embodiment, A is a covalent bond. Thus, L¹ and the cell binding agent are directly connected. For example, where L¹ comprises a contiguous amino acid sequence, the N-terminus of the sequence may connect directly to the free cysteine.

In another embodiment, A is a spacer group. Thus, L¹ and the cell binding agent are indirectly connected.

L¹ and A may be connected by a bond selected from:
—C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below and set forth in Examples 10-13 below the drug linkers of the instant invention will be linked to reactive thiol nucleophiles on free cysteines. To this end the free cysteines site-specific antibodies may be made reactive for conjugation with linker reagents by treatment with various reducing agent such as DTT or TCEP or mild reducing agents as set forth herein.

Preferably, the linker contains an electrophilic functional group for reaction with a nucleophilic functional group on the modulator. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones, carboxyl, and, some of which are exemplified as follows:

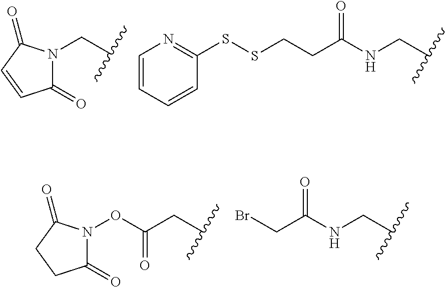

In particularly preferred embodiments the connection between the site-specific antibody and the drug-linker moiety is through a thiol residue of a free cysteine of the engineered antibody and a terminal maleimide group of present on the linker. In such embodiments, the connection between the cell binding agent and the drug-linker is:

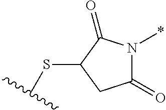 [SMILES: *SC1CC(=O)N(*)C1=O]

where the asterisk indicates the point of attachment to the remaining portion of drug-linker and the wavy line indicates the point of attachment to the remaining portion of the engineered antibody. In this embodiment, the S atom is preferably derived from the free cysteine antibody. With regard to other compatible linkers the binding moiety comprises a terminal iodoacetamide that may be reacted with activated thiols to provide the desired site-specific conjugate. The preferred conjugation procedure for this linker is slightly different from the preferred conjugation procedure for the maleimide binding group comprising selective reduction found in the other embodiments and set forth in the Examples below. In any event one skilled in the art could readily conjugate each of the disclosed drug-linker compounds with a compatible site-specific antibody in view of the instant disclosure.

5. Conjugation

As discussed above, the conjugate preparations provided by the instant invention exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites, the present invention advantageously provides for the selective reduction of certain prepared free cysteine sites and direction of the drug-linker to the same. The conjugation specificity promoted by the engineered sites and attendant selective reduction allows for a high percentage of site directed conjugation at the desired positions. Significantly some of these conjugation sites, such as those present in the terminal region of the light chain constant region, are typically difficult to conjugate effectively as they cross-react with other free cysteines. However, through molecular engineering and selective reduction of the resulting free cysteines efficient conjugation rates may be obtained which considerably reduces unwanted high-DAR contaminants and non-specific toxicity. More generally the engineered constructs and disclosed novel conjugation methods comprising selective reduction apparently provide ADC preparations having improved pharmacokinetics and/or pharmacodynamics and, potentially, an improved therapeutic index.

In this respect the site-specific constructs present free cysteine(s) which, when reduced, comprise thiol groups that are nucleophilic and capable of reacting to form covalent bonds with electrophilic groups on linker moieties such as those disclosed immediately above. Preferred antibodies of the instant invention will have reducible unpaired interchain or intrachain cysteines, i.e. cysteines providing such nucleophilic groups. In other preferred embodiments the unpaired or free cysteines will be provided by cysteines residues introduced or added to the antibody heavy or light chains. Thus, in certain preferred embodiments the reaction of free sulfhydryl groups of the reduced free cysteines and the terminal maleimido or haloacetamide groups of the disclosed drug-linkers will provide the desired conjugation. In such cases, and as set forth in Examples 10 and 11 below, the free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are compatible it will be appreciated that conjugation of the site-specific antibodies may be effected using various reactions, conditions and reagents known to those skilled in the art.

Conversely, the present inventors have discovered that the free cysteines of the engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this may be effected by certain reducing agents. In other preferred embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced with a cytotoxin as described herein. In this respect, and as demonstrated in Examples 12-13, the use of such stabilizing agents in combination with reducing agents can markedly improve the efficiency of site-specific conjugation as determined by extent of conjugation on the heavy and light antibody chains and DAR distribution of the preparation.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site. Such agents (e.g., certain amino acids) are known to form salt bridges (via hydrogen bonding and electrostatic interactions) and may modulate protein-protein interactions in such a way as to impart a stabilizing effect which may cause favorable conformation changes and/or may reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since the reaction conditions do not provide for the significant reduction of intact native disulfide bonds the conjugation reaction is naturally driven to the relatively few reactive thiols on the free cysteines (e.g., preferably 2 free thiols). As alluded to this considerably reduces the levels of non-specific conjugation and corresponding impurities in conjugate preparations fabricated as set forth herein.

In selected embodiments stabilizing agents compatible with the present invention will generally comprise compounds with at least one amine moiety having a basic pKa. In certain embodiments the amine moiety will comprise a primary amine while in other preferred embodiments the amine moiety will comprise a secondary amine. In still other preferred embodiments the amine moiety will comprise a tertiary amine. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In particularly preferred embodiments, compatible stabilizing agents may comprise arginine, lysine, proline and cysteine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other preferred embodiments will comprise stabilizing agents where the amine moiety will have a pKa of greater than about 9.5 while certain other embodiments will comprise stabilizing agents exhibiting at least one amine moiety having a pKa of greater than about 10.0. In still other preferred embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the engineered antibody native disulfide bonds. Under such conditions, provided by the combination of selected stabilizing and reducing agents, the activated drug linker is largely limited to binding to the desired free site-specific cysteine site. Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In preferred embodiments mild reducing agents may comprise compounds having one or more free thiols while in particularly preferred embodiments mild reducing agents will comprise compounds having a single free thiol. Non-limiting examples of reducing agents compatible with the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site (in this invention the free cysteine on the c-terminus of the light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that the engineered antibodies capable of conjugation may contain free cysteine residues that comprise sulfhydryl groups that are blocked or capped as the antibody is produced or stored. Such caps include proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed in the Examples such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction)

6. DAR Distribution and Purification

One of the advantages of the present invention is the ability to generate relatively homogeneous conjugate preparations comprising a narrowly tailored DAR distribution. In this regard the disclosed constructs and/or selective conjugation provides for homogeneity of the ADC species within a sample in terms of the stoichiometric ratio between the drug and the engineered antibody. As briefly discussed above the term “drug to antibody ratio” or “DAR” refers to the molar ratio of drug to site-specific antibody. In some embodiments a conjugate preparation may be substantially homogeneous with respect to its DAR distribution, meaning that within the preparation is a predominant species of site-specific ADC with a particular DAR (e.g., a DAR of 2 or 4) that is also uniform with respect to the site of loading (i.e., on the free cysteines). In certain embodiments of the invention it is possible to achieve the desired homogeneity through the use of site-specific antibodies or selective combination. In other preferred embodiments the desired homogeneity may be achieved through the use of site-specific constructs in combination with selective reduction. In yet other particularly preferred embodiments the preparations may be further purified using analytical or preparative chromatography techniques. In each of these embodiments the homogeneity of the ADC sample can be analyzed using various techniques known in the art including but not limited to SDS-PAGE, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As demonstrated in the Examples below liquid chromatography methods such as reverse phase (RP) and hydrophobic interaction chromatography (HIC) may separate compounds in the mixture by drug loading value. In some cases, mixed-mode chromatography (MMC) may also be used to isolate species with a specific drug load. More generally, once insoluble contaminants are removed the modulator preparation may be further purified using standard techniques such as, for example, hydroxylapatite chromatography, gel electrophoresis, dialysis, and affinity chromatography, with affinity chromatography of particular interest. In this regard protein A can be used to purify antibodies that are based on human IgG1, IgG2 or IgG4 heavy chains while protein G is recommended for all mouse isotypes and for human IgG3. Other techniques for protein purification such as fractionation on an ion-exchange column, ethanol precipitation, chromatography on silica, chromatography on heparin, sepharose chromatography on an anion or cation exchange resin (such as a polyaspartic acid column), chromatofocusing, SDS-PAGE and ammonium sulfate precipitation are also available depending on the antibody or conjugate to be recovered.

In this regard the disclosed site-specific conjugates and preparations thereof may comprise drug and antibody moieties in various stoichiometric molar ratios depending on the configuration of the engineered construct and, at least in part, on the method used to effect conjugation. Depending on how many and which interchain and intrachain disulfide bonds are disrupted theoretical drug loading may be relatively high though practical limitations such as free cysteine cross reactivity would limit the generation of homogeneous preparations comprising such DAR due to aggregates and other contaminants. That is, higher drug loading, e.g. >6, may cause aggregation, insolubility, toxicity, or loss of cellular permeability of certain antibody-drug conjugates. In view of such concerns practical drug loading provided by the instant invention may range from 1 to 12 drugs per engineered conjugate, i.e. where 1, 2, 3, 4, 5, 6, 7, 8, 9, 10, 11 or 12 drugs are covalently attached to each site specific antibody (e.g., for IgG1, other antibodies may have different loading capacity depending the number of disulfide bonds). Selected embodiments will comprise engineered conjugates where 1, 2, 3, 4, 5, 6, 7 or 8 drugs are covalently attached to each site specific antibody. More preferably the DAR of compositions of the instant invention will be approximately 2, 4 or 6 and in particularly preferred embodiments the DAR will comprise approximately 2.

Despite the relatively high level of homogeneity provided by the instant invention the disclosed compositions actually comprise a mixture engineered conjugates with a range of drugs compounds, from 1 to 8 (in the case of a IgG1). As such, the disclosed ADC compositions include mixtures of conjugates where most of the constituent antibodies are covalently linked to one or more drug moieties and (despite the conjugate specificity of selective reduction) where the drug moieties may be attached to the antibody by various thiol groups. That is, following conjugation ADC compositions of the invention will comprise a mixture of conjugates with different drug loads (e.g., from 1 to 8 drugs per IgG1 antibody) at various concentrations (along with certain reaction contaminants primarily caused by free cysteine cross reactivity). Using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in certain preferred embodiments the present invention will comprise compositions having an average DAR of 1, 2, 3, 4, 5, 6, 7 or 8 each +/−0.5. In other preferred embodiments the present invention will comprise an average DAR of 2, 4, 6 or 8+/−0.5. Finally, in selected preferred embodiments the present invention will comprise an average DAR of 2+/−0.5. It will be appreciated that the range or deviation may be less than 0.4 in certain preferred embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other preferred embodiments the ADC composition will comprise an average DAR of 2, 4, 6 or 8 each +/−0.4 with relatively low levels (<30%) of non-predominant ADC species. In particularly preferred embodiments the ADC composition will comprise an average DAR of 2+/−0.4 with relatively low levels (<30%) of non-predominant ADC species. In yet other embodiments the predominant ADC species (e.g., DAR of 2) will be present at a concentration of greater than 70%, a concentration of greater than 75%, a concentration of greater that 80%, a concentration of greater than 85%, a concentration of greater than 90%, a concentration of greater than 93%, a concentration of greater than 95% or even a concentration of greater than 97% when measured against other DAR species.

In still other embodiments the engineered conjugate compositions of the instant invention will comprise a DAR within a range of 1 to 12. Such embodiments may comprise compositions having an average DAR within the range of 1 to 3, an average DAR within the range of 2 to 4, an average DAR within the range of 3 to 5, an average DAR within the range of 4 to 6, an average DAR within the range of 5 to 7, an average DAR within the range of 6 to 8, an average DAR within the range of 7 to 9, an average DAR within the range of 8 to 10, an average DAR within the range of 9 to 11 or an average DAR within the range of 10 to 12. Preferably the compositions will have an average DAR within the range of 1 to 3 or an average DAR within the range of 3 to 5.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. residues.

### V. Pharmaceutical Preparations and Therapeutic Uses

1. Formulations and Routes of Administration

Depending on the form of the selected site-specific conjugate, the mode of intended delivery, the disease being treated or monitored and numerous other variables, compositions of the invention may be formulated as desired using art-recognized techniques. In some embodiments, the therapeutic compositions of the invention may be administered neat or with a minimum of additional components while others may optionally be formulated to contain suitable pharmaceutically acceptable carriers comprising excipients and auxiliaries that are well known in the art (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents and the like, are also available. Certain non-limiting exemplary carriers include saline, buffered saline, dextrose, water, glycerol, ethanol, and combinations thereof.

More particularly it will be appreciated that, in some embodiments, the therapeutic compositions of the invention may be administered neat or with a minimum of additional components. Conversely the site-specific ADCs of the present invention may optionally be formulated to contain suitable pharmaceutically acceptable carriers comprising excipients and auxiliaries that are well known in the art and are relatively inert substances that facilitate administration of the conjugate or which aid processing of the active compounds into preparations that are pharmaceutically optimized for delivery to the site of action. For example, an excipient can give form or consistency or act as a diluent to improve the pharmacokinetics or stability of the ADC. Suitable excipients or additives include, but are not limited to, stabilizing agents, wetting and emulsifying agents, salts for varying osmolarity, encapsulating agents, buffers, and skin penetration enhancers. In certain preferred embodiments the pharmaceutical compositions may be provided in a lyophilized form and reconstituted in, for example, buffered saline prior to administration. Such reconstituted compositions are preferably administered intravenously.

Disclosed ADCs for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000). Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, hexylsubstituted poly(lactide), sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection.

Compatible formulations for parenteral administration (e.g., intravenous injection) will comprise ADC concentrations of from about 10 μg/ml to about 100 mg/ml. In certain selected embodiments ADC concentrations will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments ADC concentrations will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

In general the compounds and compositions of the invention, comprising site-specific ADCs may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen. In particularly preferred embodiments the compounds of the instant invention will be delivered intravenously.

2. Dosages

Similarly, the particular dosage regimen, i.e., dose, timing and repetition, will depend on the particular individual and that individual's medical history, as well as empirical considerations such as pharmacokinetics (e.g., half-life, clearance rate, etc.). Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In other embodiments the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the site-specific ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the site-specific ADCs will be administered (preferably intravenously) at approximately 10, 20, 30, 40, 50, 60, 70, 80, 90 or 100 μg/kg body weight per dose. Other embodiments will comprise the administration of ADCs at about 200, 300, 400, 500, 600, 700, 800, 900, 1000, 1100, 1200, 1300, 1400, 1500, 1600, 1700, 1800, 1900 or 2000 μg/kg body weight per dose. In other preferred embodiments the disclosed conjugates will be administered at 2.5, 3, 3.5, 4, 4.5, 5, 5.5, 6, 6.5, 7, 7.58, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for various site-specific ADCs based on preclinical animal studies, clinical observations and standard medical and biochemical techniques and measurements. In particularly preferred embodiments such conjugate dosages will be administered intravenously over a period of time. Moreover, such dosages may be administered multiple times over a defined course of treatment.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

In any event, the disclosed conjugates are preferably administered as needed to subjects in a manner that provides a beneficial therapeutic index. Determination of the frequency of administration may be made by persons skilled in the art, such as an attending physician based on considerations of the condition being treated, age of the subject being treated, severity of the condition being treated, general state of health of the subject being treated and the like. Generally, an effective dose of the DLL3 conjugate is administered to a subject one or more times. More particularly, an effective dose of the ADC is administered to the subject once a month, more than once a month, or less than once a month. In certain embodiments, the effective dose of the DLL3 ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed modulators.

In certain preferred embodiments the course of treatment involving conjugated modulators will comprise multiple doses of the selected drug product over a period of weeks or months. More specifically, conjugated modulators of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

3. Combination Therapies

In accordance with the instant invention combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the ADCs of the instant invention may function as sensitizing or chemosensitizing agents by removing the CSCs that would otherwise prop up and perpetuate the tumor mass and thereby allow for more effective use of current standard of care debulking or anti-cancer agents. That is, the disclosed ADCs may, in certain embodiments provide an enhanced effect (e.g., additive or synergistic in nature) that potentiates the mode of action of another administered therapeutic agent. In the context of the instant invention “combination therapy” shall be interpreted broadly and merely refers to the administration of a site-specific ADC and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., ADC and anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

In practicing combination therapy, the conjugate and anti-cancer agent may be administered to the subject simultaneously, either in a single composition, or as two or more distinct compositions using the same or different administration routes. Alternatively, the ADC may precede, or follow, the anti-cancer agent treatment by, e.g., intervals ranging from minutes to weeks. The time period between each delivery is such that the anti-cancer agent and conjugate are able to exert a combined effect on the tumor. In at least one embodiment, both the anti-cancer agent and the ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the ADC and the anti-cancer agent.

The combination therapy may be administered once, twice or at least for a period of time until the condition is treated, palliated or cured. In some embodiments, the combination therapy is administered multiple times, for example, from three times daily to once every six months. The administering may be on a schedule such as three times daily, twice daily, once daily, once every two days, once every three days, once weekly, once every two weeks, once every month, once every two months, once every three months, once every six months or may be administered continuously via a minipump. The combination therapy may be administered via any route, as noted previously. The combination therapy may be administered at a site distant from the site of the tumor.

In one embodiment a site-specific ADC is administered in combination with one or more anti-cancer agents for a short treatment cycle to a subject in need thereof. The invention also contemplates discontinuous administration or daily doses divided into several partial administrations. The conjugate and anti-cancer agent may be administered interchangeably, on alternate days or weeks; or a sequence of antibody treatments may be given, followed by one or more treatments of anti-cancer agent therapy. In any event, as will be understood by those of ordinary skill in the art, the appropriate doses of chemotherapeutic agents and the disclosed conjugates will be generally around those already employed in clinical therapies wherein the chemotherapeutics are administered alone or in combination with other chemotherapeutics.

In another preferred embodiment the site-specific conjugates of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed conjugates one or more times even though there is little or no indication of disease using standard diagnostic procedures. In some embodiments, the ADCs will be administered on a regular schedule over a period of time, such as weekly, every two weeks, monthly, every six weeks, every two months, every three months every six months or annually. Given the teachings herein, one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the ADCs of the present invention may be used to prophylactically or as an adjuvant therapy to prevent or reduce the possibility of tumor metastasis following a debulking procedure. As used in the instant disclosure a “debulking procedure” is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed ADCs may be administered as suggested by clinical, diagnostic or theragnostic procedures to reduce tumor metastasis. The conjugates may be administered one or more times at pharmaceutically effective dosages as determined using standard techniques. Preferably the dosing regimen will be accompanied by appropriate diagnostic or monitoring techniques that allow it to be modified.

Yet other embodiments of the invention comprise administering the disclosed conjugates to subjects that are asymptomatic but at risk of developing a proliferative disorder. That is, the conjugates of the instant invention may be used in a truly preventative sense and given to patients that have been examined or tested and have one or more noted risk factors (e.g., genomic indications, family history, in vivo or in vitro test results, etc.) but have not developed neoplasia. In such cases those skilled in the art would be able to determine an effective dosing regimen through empirical observation or through accepted clinical practices.

4. Anti-Cancer Agents

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant invention. In other embodiments the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the instant invention a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present invention (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium 25 Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

In other embodiments the site-specific conjugates of the instant invention may be used in combination with any one of a number of antibodies (or immunotherapeutic agents) presently in clinical trials or commercially available. To this end the disclosed conjugates may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, ramucirumab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8 and combinations thereof.

Still other particularly preferred embodiments will comprise the use of antibodies in testing or approved for cancer therapy including, but not limited to, rituximab, trastuzumab, gemtuzumab ozogamcin, alemtuzumab, ibritumomab tiuxetan, tositumomab, bevacizumab, cetuximab, panitumumab, ramucirumab, ofatumumab, ipilimumab and brentuximab vedotin. Those skilled in the art will be able to readily identify additional anti-cancer agents that are compatible with the teachings herein.

5. Radiotherapy

The present invention also provides for the combination of site-specific conjugates with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VI. Indications

It will be appreciated that the ADCs of the instant invention may be used to treat, prevent, manage or inhibit the occurrence or recurrence of any proliferative disorder. Accordingly, whether administered alone or in combination with an anti-cancer agent or radiotherapy, the ADCs of the invention are particularly useful for generally treating neoplastic conditions in patients or subjects which may include benign or malignant tumors (e.g., adrenal, liver, kidney, bladder, breast, gastric, ovarian, colorectal, prostate, pancreatic, lung, thyroid, hepatic, cervical, endometrial, esophageal and uterine carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic, immunologic disorders and disorders caused by pathogens. Particularly, key targets for treatment are neoplastic conditions comprising solid tumors, although hematologic malignancies are within the scope of the invention.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

More specifically, neoplastic conditions subject to treatment in accordance with the instant invention may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

In certain preferred embodiments the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In other preferred embodiments, and as shown in the Examples below, the disclosed ADCs are especially effective at treating small cell lung cancer (SCLC) and non-small cell lung cancer (NSCLC) (e.g., squamous cell non-small cell lung cancer or squamous cell small cell lung cancer). In one embodiment, the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel).

In particularly preferred embodiments the disclosed ADCs may be used to treat small cell lung cancer. With regard to such embodiments the conjugated modulators may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent small cell lung cancer patients. Still other embodiments comprise the administration of the disclosed ADCs to sensitive patients (i.e., those whose relapse is longer than 2-3 months after primary therapy. In each case it will be appreciated that compatible ADCs may be used in combination with other anti-cancer agents depending the selected dosing regimen and the clinical diagnosis.

As discussed above the disclosed ADCs may further be used to prevent, treat or diagnose tumors with neuroendocrine features or phenotypes including neuroendocrine tumors. True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

While the disclosed ADCs may be advantageously used to treat neuroendocrine tumors they may also be used to treat, prevent or diagnose pseudo neuroendocrine tumors (pNETs) that genotypically or phenotypically mimic, resemble or exhibit common traits with canonical neuroendocrine tumors. Pseudo neuroendocrine tumors or tumors with neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors, including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. For the purposes of the instant invention commonly expressed histological markers or genetic markers that may be used to define neuroendocrine and pseudo neuroendocrine tumors include, but are not limited to, chromogranin A, CD56, synaptophysin, PGP9.5, ASCL1 and neuron-specific enolase (NSE).

Accordingly the ADCs of the instant invention may beneficially be used to treat both pseudo neuroendocrine tumors and canonical neuroendocrine tumors. In this regard the ADCs may be used as described herein to treat neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). Moreover, the ADCs of the instant invention may be used to treat tumors expressing one or more markers selected from the group consisting of NSE, CD56, synaptophysin, chromogranin A, ASCL1 and PGP9.5 (UCHL1). That is, the present invention may be used to treat a subject suffering from a tumor that is NSE⁺ or CD56⁺ or PGP9.5⁺ or ASCL1⁺ or SYP⁺ or CHGA⁺ or some combination thereof.

With regard to hematologic malignancies it will be further be appreciated that the compounds and methods of the present invention may be particularly effective in treating a variety of B-cell lymphomas, including low grade/NHL follicular cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, Waldenstrom's Macroglobulinemia, lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic, follicular, diffuse large cell, diffuse small cleaved cell, large cell immunoblastic lymphoblastoma, small, non-cleaved, Burkitt's and non-Burkitt's, follicular, predominantly large cell; follicular, predominantly small cleaved cell; and follicular, mixed small cleaved and large cell lymphomas. See, Gaidono et al., “Lymphomas”, IN CANCER: PRINCIPLES & PRACTICE OF ONCOLOGY, Vol. 2: 2131-2145 (DeVita et al., eds., 5.sup.th ed. 1997). It should be clear to those of skill in the art that these lymphomas will often have different names due to changing systems of classification, and that patients having lymphomas classified under different names may also benefit from the combined therapeutic regimens of the present invention.

The present invention also provides for a preventative or prophylactic treatment of subjects who present with benign or precancerous tumors. Beyond being a DLL3 associated disorder it is not believed that any particular type of tumor or proliferative disorder should be excluded from treatment using the present invention. However, the type of tumor cells may be relevant to the use of the invention in combination with secondary therapeutic agents, particularly chemotherapeutic agents and targeted anti-cancer agents.

Preferably the “subject” or “patient” to be treated will be human although, as used herein, the terms are expressly held to comprise any species including all mammals. Accordingly the subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

### VII. Diagnostics and Screening

1. Diagnostics

The invention provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with an antibody as described herein and detecting presence or absence, or level of association, of the antibody to bound or free target molecules in the sample. In some embodiments the antibody will comprise a detectable label or reporter molecule as described herein.

In some embodiments, the association of the antibody with particular cells in the sample can denote that the sample may contain tumorigenic cells, thereby indicating that the individual having cancer may be effectively treated with an antibody as described herein.

Samples can be analyzed by numerous assays, for example, radioimmunoassays, enzyme immunoassays (e.g. ELISA), competitive-binding assays, fluorescent immunoassays, immunoblot assays, Western Blot analysis and flow cytometry assays. Compatible in vivo theragnostic or diagnostic assays can comprise art recognized imaging or monitoring techniques, for example, magnetic resonance imaging, computerized tomography (e.g. CAT scan), positron tomography (e.g., PET scan), radiography, ultrasound, etc., as would be known by those skilled in the art.

In a particularly preferred embodiment the antibodies of the instant invention may be used to detect and quantify levels of a particular determinant (e.g., SEZ6, DLL3 or CD324) in a patient sample (e.g., plasma or blood) which may, in turn, be used to detect, diagnose or monitor proliferative disorders that are associated with the relevant determinant. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In certain embodiments of the invention, the tumorigenic cells in a subject or a sample from a subject may be assessed or characterized using the disclosed antibodies prior to therapy or regimen to establish a baseline. In other examples, the tumorigenic cells can be assessed from a sample that is derived from a subject that was treated.

2. Screening

In certain embodiments, the antibodies can be used to screen samples in order to identify compounds or agents (e.g., drugs for the treatment of proliferative diseases) that alter a function or activity of tumor cells by interacting with a determinant. In one embodiment, a system or method includes tumor cells expressing a certain determinant (e.g. SEZ6, DLL3 or CD324) and a compound or agent (e.g., drug), wherein the cells and compound or agent are in contact with each other. In such embodiments the subject cells may have been identified, monitored and/or enriched using the disclosed antibodies.

In yet another embodiment, a method includes contacting, directly or indirectly, tumor cells with a test agent or compound and determining if the test agent or compound modulates an activity or function of the determinant-associated tumor cells for example, changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Screening methods include high throughput screening, which can include arrays of cells (e.g., microarrays) positioned or placed, optionally at pre-determined locations, for example, on a culture dish, tube, flask, roller bottle or plate. High-throughput robotic or manual handling methods can probe chemical interactions and determine levels of expression of many genes in a short period of time. Techniques have been developed that utilize molecular signals, for example via fluorophores or microarrays and automated analyses that process information at a very rapid rate. Libraries that can be screened include, for example, small molecule libraries, phage display libraries, fully human antibody yeast display libraries (Adimab, LLC), siRNA libraries, and adenoviral transfection vectors.

### VIII. Articles of Manufacture

Pharmaceutical packs and kits comprising one or more containers, comprising one or more doses of a site-specific ADC are also provided. In certain embodiments, a unit dosage is provided wherein the unit dosage contains a predetermined amount of a composition comprising, for example, an anti-DLL3 conjugate, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present invention also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the site-specific conjugates of the invention such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed ADCs, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody conjugate and any optional components to an animal or patient, e.g., one or more needles, I.V. bags or syringes, or even an eye dropper, pipette, or other such like apparatus, from which the formulation may be injected or introduced into the animal or applied to a diseased area of the body. The kits of the present invention will also typically include a means for containing the vials, or such like, and other component in close confinement for commercial sale, such as, e.g., injection or blow-molded plastic containers into which the desired vials and other apparatus are placed and retained. Any label or package insert indicates that the engineered conjugate composition is used for treating cancer, for example small cell lung cancer.

In other preferred embodiments the conjugates of the instant invention may be used in conjunction with, or comprise, diagnostic or therapeutic devices useful in the prevention or treatment of proliferative disorders. For example, in on preferred embodiment the compounds and compositions of the instant invention may be combined with certain diagnostic devices or instruments that may be used to detect, monitor, quantify or profile cells or marker compounds involved in the etiology or manifestation of proliferative disorders. For selected embodiments the marker compounds may comprise NSE, CD56, synaptophysin, chromogranin A, and PGP9.5.

In particularly preferred embodiments the devices may be used to detect, monitor and/or quantify circulating tumor cells either in vivo or in vitro (see, for example, WO 2012/0128801 which is incorporated herein by reference). In still other preferred embodiments, and as discussed above, circulating tumor cells may comprise cancer stem cells.

### IX. Miscellaneous

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In addition, ranges provided in the specification and appended claims include both end points and all points between the end points. Therefore, a range of 2.0 to 3.0 includes 2.0, 3.0, and all points between 2.0 and 3.0.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art. Moreover, any section headings used herein are for organizational purposes only and are not to be construed as limiting the subject matter described.

As used herein, tumor cell types are abbreviated as follows: adenocarcinoma (Adeno), adrenal (AD), breast (BR), estrogen receptor positive breast (BR-ER+), estrogen receptor negative breast (BR-ER−), progesterone receptor positive breast (BR-PR+), progesterone receptor negative breast (BR-PR−), ERb2/Neu positive breast (BR-ERB2/Neu+), Her2 positive breast (BR-Her2+), claudin-low breast (BR-CLDN-lo), triple-negative breast cancer (BR-TNBC), colorectal (CR), endometrial (EM), gastric (GA), head and neck (HN), kidney (KDY), large cell neuroendocrine (LCNEC), liver (LIV), lymph node (LN), lung (LU), lung-carcinoid (LU-CAR), lung-spindle cell (LU-SPC), melanoma (MEL), non-small cell lung (NSCLC), ovarian (OV), ovarian serous (OV-S), ovarian papillary serous (OV-PS), ovarian malignant mixed mesodermal tumor (OV-MMMT), ovarian mucinous (OV-MUC), ovarian clear cell (OV-CC), neuroendocrine tumor (NET), pancreatic (PA), prostate (PR), squamous cell (SCC), small cell lung (SCLC) and tumors derived from skin (SK).

### X. References

Unless The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### XI. Sequence Listing Summary

Appended to the instant application is a sequence listing comprising a number of nucleic acid and amino acid sequences. The following Table 4 provides a summary of the included sequences.

TABLE 4

| SEQ | |
|---|---|
| ID NO. | Description |
| 1 | SEZ6 isoform 1 mRNA sequence |
| 2 | SEZ6 isoform 2 mRNA sequence |
| 3 | SEZ6 isoform 1 protein sequence |
| 4 | SEZ6 isoform 2 protein sequence |
| 5 | cDNA sequence of human SEZ6 ORF |
| 6 | Human SEZ6 protein |
| 7 | cDNA sequence of a commercial SEZ6 clone (BC146292) |
| 8 | Human SEZ6-Fc ORF |
| 9 | Human SEZ6-Fc protein |
| 10 | cDNA sequence of mouse SEZ6 ORF |
| 11 | Mouse SEZ6 protein |
| 12 | cDNA sequence of rat SEZ6 ORF |
| 13 | Rat SEZ6 protein |
| 14 | cDNA sequence of cynomolgus SEZ6 ORF |
| 15 | Cynomolgus SEZ6 protein |
| 16 | cDNA sequence of human SEZ6L ECD |
| 17 | Human SEZ6L ECD protein |
| 18 | cDNA sequence of human SEZ6L2 ECD |
| 19 | Human SEZ6L2 ECD protein |
| 20 | SC17.1 VL protein |
| 21 | SC17.1 VH protein |
| 22-169 | Additional murine VL and VH proteins as in SEQ ID NOs 20-21 |
| 170 | hSC17.16 VL protein |
| 171 | hSC17.16 VH protein |
| 172-199 | Additional humanized VL and VH proteins as in SEQ ID NOs |
| | 170-171 |
| 200 | Asn-Pro-Thr-Tyr (motif on the SEZ6 C-terminal cytoplasmic |
| | domain) |
| 201 | 9-Histidine Tag |
| 202-219 | Reserved |
| 220 | SC17.1 VL nucleic acid |
| 221 | SC17.1 VH nucleic acid |
| 222-369 | Additional murine VL and VH nucleic acids as in SEQ ID NOs |
| | 220-221 |
| 370 | hSC17.16 VL nucleic acid |
| 371 | hSC17.16 VH nucleic acid |
| 372-399 | Additional humanized VL and VH nucleic acids as in SEQ ID |
| | NOs 270-271 |
| 400 | hSC17.200 full length light chain amino acid sequence |
| 401 | hSC17.200 full length heavy chain amino acid sequence |
| 402 | hSC17.200vL1 full length light chain amino acid sequence |
| 403 | Kappa constant region protein |
| 404 | IgG1 constant region protein |
| 405 | hSC17.16 CDRL1 |
| 406 | hSC17.16 CDRL2 |
| 407 | hSC17.16 CDRL3 |
| 408 | hSC17.16 CDRH1 |
| 409 | hSC17.16 CDRH2 |
| 410 | hSC17.16 CDRH3 |
| 411 | hSC17.17 CDRL1 |
| 412 | hSC17.17 CDRL2 |
| 413 | hSC17.17 CDRL3 |
| 414 | hSC17.17 CDRH1 |
| 415 | hSC17.17 CDRH2 |
| 416 | hSC17.17 CDRH3 |
| 417 | hSC17.24 CDRL1 |
| 418 | hSC17.24 CDRL2 |
| 419 | hSC17.24 CDRL3 |
| 420 | hSC17.24 CDRH1 |
| 421 | hSC17.24 CDRH2 |
| 422 | hSC17.24 CDRH3 |
| 423 | hSC17.28 CDRL1 |
| 424 | hSC17.28 CDRL2 |
| 425 | hSC17.28 CDRL3 |
| 426 | hSC17.28 CDRH1 |
| 427 | hSC17.28 CDRH2 |
| 428 | hSC17.28 CDRH3 |
| 429 | hSC17.34 CDRL1 |
| 430 | hSC17.34 CDRL2 |
| 431 | hSC17.34 CDRL3 |
| 432 | hSC17.34 CDRH1 |
| 433 | hSC17.34 CDRH2 |
| 434 | hSC17.34 CDRH3 |
| 435 | hSC17.46 CDRL1 |
| 436 | hSC17.46 CDRL2 |
| 437 | hSC17.46 CDRL3 |
| 438 | hSC17.46 CDRH1 |
| 439 | hSC17.46 CDRH2 |
| 440 | hSC17.46 CDRH1 |
| 441 | hSC17.151 CDRL1 |
| 442 | hSC17.151 CDRL2 |
| 443 | hSC17.151 CDRL3 |
| 444 | hSC17.151 CDRH1 |
| 445 | hSC17.151 CDRH2 |
| 446 | hSC17.151 CDRH3 |
| 447 | hSC17.155 and hSC17.155vH1-6 CDRL1 |
| 448 | hSC17.155 and hSC17.155vH1-6 CDRL2 |
| 449 | hSC17.155 and hSC17.155vH1-6 CDRL3 |
| 450 | hSC17.155 and hSC17.155vH1, vH2 and vH4-6 CDRH1 |
| 451 | hSC17.155 and hSC17.155vH1-3 CDRH2 |
| 452 | hSC17.155 and hSC17.155vH1-6 CDRH3 |
| 453 | hSC17.156 CDRL1 |
| 454 | hSC17.156 CDRL2 |
| 455 | hSC17.156 CDRL3 |
| 456 | hSC17.156 CDRH1 |
| 457 | hSC17.156 CDRH2 |
| 458 | hSC17.156 CDRH3 |
| 459 | hSC17.161 and hSC17.161vL1 CDRL1 |
| 460 | hSC17.161 and hSC17.161vL1 CDRL2 |
| 461 | hSC17.161 and hSC17.161vL1 CDRL3 |
| 462 | hSC17.161 and hSC17.161vL1 CDRH1 |
| 463 | hSC17.161 and hSC17.161vL1 CDRH2 |
| 464 | hSC17.161 and hSC17.161vL1 CDRH3 |
| 465 | hSC17.200 CDRL1 |
| 466 | hSC17.200 and hSC17.200vL1 CDRL2 |
| 467 | hSC17.200 and hSC17.200vL1 CDRL3 |
| 468 | hSC17.200 and hSC17.200vL1 CDRH1 |
| 469 | hSC17.200 and hSC17.200vL1 CDRH2 |
| 470 | hSC17.200 and hSC17.200vL1 CDRH3 |
| 471 | hSC17.155vH1 FR1 |
| 472 | hSC17.155vH2 FR1 |
| 473 | hSC17.155vH3 CDRH1 |
| 474 | hSC17.155vH4 CDRH2 |
| 475 | hSC17.155vH5 CDRH2 |
| 476 | hSC17.155vH6 CDRH2 |
| 477 | hSC17.161vH1 FR1 |
| 478 | hSC17.161vH1 FR2 |
| 479 | hSC17.161vH1 FR3 |
| 480 | hSC17.200vL1 CDRL1 |
| 481-499 | Reserved |
| 500 | C220S IgG1 heavy constant region protein |
| 501 | C220Δ IgG1 heavy constant region protein |
| 502 | C214Δ Kappa light chain constant region protein |
| 503 | C214S Kappa light chain constant region protein |
| 504 | Lambda light chain constant region protein |
| 505 | C214Δ Lambda light chain constant region protein |
| 506 | C214S Lambda light chain constant region protein |
| 507 | SC16.56 ss1 and ss2 full length light chain protein |
| 508 | SC16.56 ss3 and ss4 full length heavy chain protein |
| 509 | SC16.56 ss1 full length heavy chain protein |
| 510 | SC16.56 ss2 full length heavy chain protein |
| 511 | SC16.56 ss3 full length light chain protein |
| 512 | SC16.56 ss4 full length light chain protein |
| 513 | SC17.200 ss1 and ss2 full length light chain protein |
| 514 | SC17.200 ss3 and ss4 full length heavy chain protein |
| 515 | SC17.200 ss1 full length heavy chain protein |
| 516 | SC17.200 ss2 full length heavy chain protein |
| 517 | SC17.200 ss3 full length light chain protein |
| 518 | SC17.200 ss4 full length light chain protein |
| 519 | hSC16.13 light chain variable region protein |
| 520 | hSC16.15 light chain variable region protein |
| 521 | hSC16.25 light chain variable region protein |
| 522 | hSC16.34 light chain variable region protein |
| 523 | hSC16.56 light chain variable region protein |
| 524 | hSC16.13 heavy chain variable region protein |
| 525 | hSC16.15 heavy chain variable region protein |
| 526 | hSC16.25 heavy chain variable region protein |
| 527 | hSC16.34 heavy chain variable region protein |
| 528 | hSC16.56 heavy chain variable region protein |
| 529 | SC10.17 light chain variable region protein |
| 530 | SC10.17 heavy chain variable region protein |
| 531 | hSC10.17 light chain variable region protein |
| 532 | hSC10.17 heavy chain variable region protein |
| 533 | SC10.17 light chain variable region nucleic acid |
| 534 | SC10.17 heavy chain variable region nucleic acid |
| 535 | hSC10.17 light chain variable region nucleic acid |
| 536 | hSC10.17 heavy chain variable region nucleic acid |
| 537 | SC17.17 ss1 and ss2 full length light chain protein |
| 538 | SC17.17 ss3 and ss4 full length heavy chain protein |
| 539 | SC17.17 ss1 full length heavy chain protein |
| 540 | SC17.17 ss2 full length heavy chain protein |
| 541 | SC17.17ss3 full length light chain protein |
| 542 | SC17.17ss4 full length light chain protein |
| 543 | SC10.17ss3 full length heavy chain protein |
| 544 | SC10.17ss3 full length light chain protein |
| 545-549 | Reserved |
| 550 | ss5 light chain constant region protein |
| 551 | ss6 light chain constant region protein |
| 552 | ss7 light chain constant region protein |
| 553 | ss8 light chain constant region protein |
| 554 | ss9 light chain constant region protein |
| 555 | ss10 light chain constant region protein |
| 556 | ss11 light chain constant region protein |
| 557 | ss12 light chain constant region protein |
| 558 | ss13 light chain constant region protein |
| 559 | ss14 light chain constant region protein |
| 560 | ss15 light chain constant region protein |
| 561 | ss16 light chain constant region protein |
| 562 | ss17 light chain constant region protein |
| 563 | ss18 light chain constant region protein |
| 564 | ss19 light chain constant region protein |

### EXAMPLES

The present invention, thus generally described, will be understood more readily by reference to the following Examples, which are provided by way of illustration and are not intended to be limiting of the instant invention. The Examples are not intended to represent that the experiments below are all or the only experiments performed.

### Example 1

### Generation of Anti-DLL3 Antibodies

Anti-DLL3 murine antibodies were produced as follows. In a first immunization campaign, three mice (one from each of the following strains: Balb/c, CD-1, FVB) were inoculated with human DLL3-fc protein (hDLL3-Fc) emulsified with an equal volume of TiterMax® or alum adjuvant. The hDLL3-Fc fusion construct was purchased from Adipogen International (Catalog No. AG-40A-0113). An initial immunization was performed with an emulsion of 10 μg hDLL3-Fc per mouse in TiterMax. Mice were then boosted biweekly with 5 μg hDLL3-Fc per mouse in alum adjuvant. The final injection prior to fusion was with 5 μg hDLL3-Fc per mouse in PBS.

In a second immunization campaign six mice (two each of the following strains: Balb/c, CD-1, FVB), were inoculated with human DLL3-His protein (hDLL3-His), emulsified with an equal volume of TiterMax® or alum adjuvant. Recombinant hDLL3-His protein was purified from the supernatants of CHO-S cells engineered to overexpress hDLL3-His. The initial immunization was with an emulsion of 10 μg hDLL3-His per mouse in TiterMax. Mice were then boosted biweekly with 5 μg hDLL3-His per mouse in alum adjuvant. The final injection was with 2×10⁵ HEK-293T cells engineered to overexpress hDLL3.

Solid-phase ELISA assays were used to screen mouse sera for mouse IgG antibodies specific for human DLL3. A positive signal above background was indicative of antibodies specific for DLL3. Briefly, 96 well plates (VWR International, Cat. #610744) were coated with recombinant DLL3-His at 0.5 μg/ml in ELISA coating buffer overnight. After washing with PBS containing 0.02% (v/v) Tween 20, the wells were blocked with 3% (w/v) BSA in PBS, 200 μL/well for 1 hour at room temperature (RT). Mouse serum was titrated (1:100, 1:200, 1:400, and 1:800) and added to the DLL3 coated plates at 50 μL/well and incubated at RT for 1 hour. The plates are washed and then incubated with 50 μL/well HRP-labeled goat anti-mouse IgG diluted 1:10,000 in 3% BSA-PBS or 2% FCS in PBS for 1 hour at RT. Again the plates were washed and 40 μL/well of a TMB substrate solution (Thermo Scientific 34028) was added for 15 minutes at RT. After developing, an equal volume of 2N H₂SO₄ was added to stop substrate development and the plates were analyzed by spectrophotometer at OD 450.

Sera-positive immunized mice were sacrificed and draining lymph nodes (popliteal, inguinal, and medial iliac) were dissected and used as a source for antibody producing cells. Cell suspensions of B cells (approximately 229×10⁶ cells from the hDLL3-Fc immunized mice, and 510×10⁶ cells from the hDLL3-His immunized mice) were fused with non-secreting P3x63Ag8.653 myeloma cells at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM Condimed (Roche Applied Sciences), 1 mM nonessential amino acids, 1 mM HEPES, 100 IU penicillin-streptomycin, and 50 LM 2-mercaptoethanol, and were cultured in four T225 flasks in 100 mL selection medium per flask. The flasks were placed in a humidified 37° C. incubator containing 5% CO₂ and 95% air for six to seven days.

On day six or seven after the fusions the hybridoma library cells were collected from the flasks and plated at one cell per well (using the FACSAria I cell sorter) in 200 μL of supplemented hybridoma selection medium (as described above) into 64 Falcon 96-well plates, and 48 96-well plates for the hDLL3-His immunization campaign. The rest of the library was stored in liquid nitrogen.

The hybridomas were cultured for 10 days and the supernatants were screened for antibodies specific to hDLL3 using flow cytometry performed as follows. 1×10⁵ per well of HEK-293T cells engineered to overexpress human DLL3, mouse DLL3 (pre-stained with dye), or cynomolgus DLL3 (pre-stained with Dylight800) were incubated for 30 minutes with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS. After a 15 minute incubation cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

The hDLL3-His immunization campaign yielded approximately 50 murine anti-hDLL3 antibodies and the hDLL3-Fc immunization campaign yielded approximately 90 murine anti-hDLL3 antibodies.

### Example 2

### Sequencing of Anti-DLL3 Antibodies

Based on the foregoing, a number of exemplary distinct monoclonal antibodies that bind immobilized human DLL3 or h293-hDLL3 cells with apparently high affinity were selected for sequencing and further analysis. Sequence analysis of the light chain variable regions and heavy chain variable regions from selected monoclonal antibodies generated in Example 1 confirmed that many had novel complementarity determining regions and often displayed novel VDJ arrangements.

Initially selected hybridoma cells expressing the desired antibodies were lysed in Trizol® reagent (Trizol® Plus RNA Purification System, Life Technologies) to prepare the RNA encoding the antibodies. Between 10⁴ and 10⁵ cells were re-suspended in 1 mL Trizol and shaken vigorously after addition of 200 μL chloroform. Samples were then centrifuged at 4° C. for 10 minutes and the aqueous phase was transferred to a fresh microfuge tube and an equal volume of 70% ethanol was added. The sample was loaded on an RNeasy Mini spin column, placed in a 2 mL collection tube and processed according to the manufacturer's instructions. Total RNA was extracted by elution, directly to the spin column membrane with 100 μL RNase-free water. The quality of the RNA preparations was determined by fractionating 3 μL in a 1% agarose gel before being stored at −80° C. until used.

The variable region of the Ig heavy chain of each hybridoma was amplified using a 5′ primer mix comprising 32 mouse specific leader sequence primers designed to target the complete mouse V_{H} repertoire in combination with a 3′ mouse Cγ primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. For antibodies containing a lambda light chain, amplification was performed using three 5′ V_{L} leader sequences in combination with one reverse primer specific to the mouse lambda constant region. The V_{H} and V_{L} transcripts were amplified from 100 ng total RNA using the Qiagen One Step RT-PCR kit as follows. A total of eight RT-PCR reactions were run for each hybridoma, four for the Vκ light chain and four for the Vγ heavy chain. PCR reaction mixtures included 3 μL of RNA, 0.5 μL of 100 LM of either heavy chain or kappa light chain primers (custom synthesized by Integrated Data Technologies), 5 μL of 5×RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 50° C. for 30 minutes, 95° C. for 15 minutes followed by 30 cycles of (95° C. for 30 seconds, 48° C. for 30 seconds, 72° C. for 1 minute). There was then a final incubation at 72° C. for 10 minutes.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands (MCLAB).

Selected nucleotide sequences were analyzed using the IMGT sequence analysis tool (http://www.imgt.org/IMGTmedical/sequence_analysis.html) to identify germline V, D and J gene members with the highest sequence homology. These derived sequences were compared to known germline DNA sequences of the Ig V- and J-regions by alignment of V_{H} and V_{L} genes to the mouse germline database using a proprietary antibody sequence database.

The derived sequences of the murine heavy and light chain variable regions are provided in the appended sequence listing and, in an annotated form, PCT/US14/17810 which is incorporated herein by reference with respect to such sequences.

### Example 3

### Generation of Humanized Anti-DLL3 Antibodies

Certain murine antibodies generated as per Example 1 (termed SC16.13, SC16.15, SC16.25, SC16.34 and SC16.56) were used to derive humanized antibodies comprising murine CDRs grafted into a human acceptor antibody. In preferred embodiments the humanized heavy and light chain variable regions described in the instant Example may be incorporated in the disclosed site-specific conjugates as described below.

In this respect the murine antibodies were humanized with the assistance of a proprietary computer-aided CDR-grafting method (Abysis Database, UCL Business) and standard molecular engineering techniques as follows. Total RNA was extracted from the hybridomas and amplified as set forth in Example 2. Data regarding V, D and J gene segments of the V_{H} and V_{L} chains of the murine antibodies was obtained from the derived nucleic acid sequences. Human framework regions were selected and/or designed based on the highest homology between the framework sequences and CDR canonical structures of human germline antibody sequences, and the framework sequences and CDRs of the selected murine antibodies. For the purpose of the analysis the assignment of amino acids to each of the CDR domains was done in accordance with Kabat et al. numbering. Once the human receptor variable region frameworks are selected and combined with murine CDRs, the integrated heavy and light chain variable region sequences are generated synthetically (Integrated DNA Technologies) comprising appropriate restriction sites.

The humanized variable regions are then expressed as components of engineered full length heavy and light chains to provide the site-specific antibodies as described herein. More specifically, humanized anti-DLL3 engineered antibodies were generated using art-recognized techniques as follows. Primer sets specific to the leader sequence of the V_{H} and V_{L} chain of the antibody were designed using the following restriction sites: AgeI and XhoI for the V_{H} fragments, and XmaI and DraIII for the V_{L} fragments. PCR products were purified with a Qiaquick PCR purification kit (Qiagen), followed by digestion with restriction enzymes AgeI and XhoI for the V_{H} fragments and XmaI and DraIII for the V_{L} fragments. The V_{H} and V_{L} digested PCR products were purified and ligated, respectively, into a human IgG1 heavy chain constant region expression vector or a kappa C_{L} human light chain constant region expression vector. As discussed in detail below the heavy and/or light chain constant regions may be engineered to present site-specific conjugation sites on the assembled antibody.

The ligation reactions were performed as follows in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the V_{H} fragment was cloned into the AgeI-XhoI restriction sites of the pEE6.4HuIgG1 expression vector (Lonza) and the V_{L} fragment was cloned into the XmaI-DraIII restriction sites of the pEE12.4Hu-Kappa expression vector (Lonza) where either the HuIgG1 and/or Hu-Kappa expression vector may comprise either a native or an engineered constant region.

The humanized antibodies were expressed by co-transfection of HEK-293T cells with pEE6.4HuIgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HuIgG1 and pEE12.4Hu-Kappa vector DNA were added to 50 μL HEK-293T transfection reagent in 1.5 mL Opti-MEM. The mix was incubated for 30 minutes at room temperature and plated. Supernatants were harvested three to six days after transfection. Culture supernatants containing recombinant humanized antibodies were cleared from cell debris by centrifugation at 800×g for 10 minutes and stored at 4° C. Recombinant humanized antibodies were purified by MabSelect SuRe Protein A affinity chromatography (GE Life Sciences). For larger scale antibody expression, CHO-S cells were transiently transfected in 1 L volumes, seeded at 2.2e6 cells per mL Polyethylenimine (PEI) was used as a transfection reagent. After 7-10 days of antibody expression, culture supernatants containing recombinant antibodies were cleared from cell debris by centrifugation and purified by MabSelect SuRe Protein A affinity chromatography.

The genetic composition for the selected human acceptor variable regions are shown in Table 5 immediately below for each of the humanized DLL3 antibodies. The sequences depicted in Table 5 correspond to the annotated heavy and light chain sequences set forth in FIGS. 2A and 2B for the subject clones. Note that the complementarity determining regions and framework regions set forth in FIGS. 2A and 2B are defined as per Kabat et al. (supra) using a proprietary version of the Abysis database (Abysis Database, UCL Business).

More specifically, the entries in Table 5 below correspond to the contiguous variable region sequences set forth SEQ ID NOS: 519 and 524 (hSC16.13), SEQ ID NOS: 520 and 525 (hSC16.15), SEQ ID NOS: 521 and 526 (hSC16.25), SEQ ID NOS: 522 and 527 (hSC16.34) and SEQ ID NOS: 523 and 528 (hSC16.56). Besides the genetic composition Table 5 shows that, in these selected embodiments, no framework changes or back mutations were necessary to maintain the favorable binding properties of the selected antibodies. Of course, in other CDR grafted constructs it will be appreciated that such framework changes or back mutations may be desirable and as such, are expressly contemplated as being within the scope of the instant invention.

TABLE 5

| | human | human | FW | human | human | FW |
|---|---|---|---|---|---|---|
| mAb | VH | JH | changes | VK | JK | changes |
| hSC16.13 | IGHV2- | JH6 | None | IGKV1- | JK1 | None |
| | 5*01 | | | 39*01 | | |
| hSC16.15 | IGHV1- | JH4 | None | IGKV1- | JK4 | None |
| | 46*01 | | | 13*02 | | |
| hSC16.25 | IGHV2- | JH6 | None | IGKV6- | JK2 | None |
| | 5*01 | | | 21*01 | | |
| hSC16.34 | IGHV1- | JH4 | None | IGKV1- | JK1 | None |
| | 3*02 | | | 27*01 | | |
| hSC16.56 | IGHV1- | JH4 | None | IGKV3- | JK2 | None |
| | 18*01 | | | 15*01 | | |

Though no residues were altered in the framework regions, in one of humanized clones (hSC16.13) mutations were introduced into heavy chain CDR2 to address stability concerns. The binding affinity of the antibody with the modified CDR was evaluated to ensure that it was equivalent to either the corresponding murine antibody.

Following humanization of all selected antibodies by CDR grafting, the resulting light and heavy chain variable region amino acid sequences were analyzed to determine their homology with regard to the murine donor and human acceptor light and heavy chain variable regions. The results, shown in Table 6 immediately below, reveal that the humanized constructs consistently exhibited a higher homology with respect to the human acceptor sequences than with the murine donor sequences. More particularly, the murine heavy and light chain variable regions show a similar overall percentage homology to a closest match of human germline genes (85%-93%) compared with the homology of the humanized antibodies and the donor hybridoma protein sequences (74%-83%).

TABLE 6

| | Homology to Human | Homology to Murine Parent |
|---|---|---|
| mAb | (CDR acceptor) | (CDR donor) |
| hSC16.13 HC | 93% | 81% |
| hSC16.13 LC | 87% | 77% |
| hSC16.15 HC | 85% | 83% |
| hSC16.15 LC | 85% | 83% |
| hSC16.25 HC | 91% | 83% |
| hSC16.25 LC | 85% | 79% |
| hSC16.34 HC | 87% | 79% |
| hSC16.34 LC | 85% | 81% |
| hSC16.56 HC | 87% | 74% |
| hSC16.56 LC | 87% | 76% |

Upon testing each of the derived humanized constructs exhibited favorable binding characteristics roughly comparable to those shown by the murine parent antibodies.

### Example 4

### Generation and Humanization of Anti-SEZ6 Antibodies

A SEZ6 antigen was generated by fusing the ECD portion of the human SEZ6 protein to a human IgG2 Fc domain using standard molecular techniques. A more detailed description of the production of the SEZ6 antigen is provided in PCT/US2013/0027391, which is incorporated herein by reference as to the same. Following inoculation of six female mice antibody producing hybridomas were generated substantially as set forth in Example 1. The hybridomas were screened as previously discussed and genetic material obtained from those of interest. Sequences of the heavy and light chain variable regions of the anti-SEZ6 antibodies were determined substantially as set forth in Example 2.

A number of anti-SEZ6 murine antibodies were humanized using similar techniques to those set out in the previous Example. Human frameworks for heavy and light chains were selected based on sequence and structure similarity with respect to functional human germline genes. In this regard structural similarity was evaluated by comparing the mouse canonical CDR structure to human candidates with the same canonical structures as described in Chothia et al. (supra).

More particularly eleven murine antibodies SC17.16, SC17.17, SC17.24, SC17.28, SC17.34, SC17.46, SC17.151, SC17.155, SC17.156, SC17.161 and SC17.200 were humanized with the assistance of a computer-aided CDR-grafting analysis (Abysis Database, UCL Business Plc.) and standard molecular engineering techniques to provide hSC17.16, hSC17.17, hSC17.24, hSC17.28, hSC17.34, hSC17.46, hSC17.151, hSC17.155, hSC17.156, hSC17.161 and hSC17.200 modulators. The human framework regions of the variable regions were selected based on their highest sequence homology to the subject mouse framework sequence and its canonical structure. For the purposes of the humanization analysis, the assignment of amino acids to each of the CDR domains is in accordance with Kabat et al. numbering (supra).

From the nucleotide sequence information, data regarding V, D and J gene segments of the heavy and light chains of subject murine antibodies were obtained. Based on the sequence data new primer sets specific to the leader sequence of the Ig V_{H} and V_{K} light chain of the antibodies were designed for cloning of the recombinant monoclonal antibody. Subsequently the V-(D)-J sequences were aligned with mouse Ig germ line sequences. The resulting genetic arrangements for each of the eleven humanized constructs are shown in Table 7 immediately below.

TABLE 7

| | human | human | human | FW | human | human | FW |
|---|---|---|---|---|---|---|---|
| mAb | VH | DH | JH | changes | VK | JK | changes |
| hSC17.16 | IGHV1-2 | IGHD3-16 | JH5 | None | IGKV-O2 | JK1 | none |
| hSC17.17 | IGHV1-2 | IGHD4-11 | JH4 | none | IGKV-L6 | JK2 | none |
| hSC17.24 | VH1-f | IGHD5-12 | JH4 | 48I, 73K | VKB3 | JK1 | none |
| hSC17.28 | IGHV1-2 | IGHD3-16 | JH4 | none | IGKV-A10 | JK4 | none |
| hSC17.34 | IGHV1-3 | IGHD3-10 | JH4 | 71V | IGKV-L1 | JK1 | 71Y |
| hSC17.46 | IGHV1-2 | IGHD4-23 | JH4 | 48I, 69L | IGKV-L11 | JK1 | 87F |
| hSC17.151 | IGHV1-46 | IGHD1-14 | JH4 | none | VKL6 | JK2 | none |
| hSC17.155 | IGHV1-46 | IGHD2-2 | JH4 | none | VKB3 | JK1 | none |
| hSC17.156 | IGHV2-26 | IGHD4-17 | JH4 | none | VKO1 | JK4 | none |
| hSC17.161 | IGHV1-2 | IGHD1-14 | JH4 | none | VKB3 | JK2 | none |
| hSC17.200 | IGHV5-51 | IGHD4-17 | JH4 | none | IGKV-L6 | JK4 | none |

The humanized antibodies listed in Table 7 correspond to the annotated light and heavy chain variable region sequences set forth in FIGS. 3A and 3B (SEQ ID NOS: 170-199). The corresponding nucleic acid sequences of the light and heavy chain variable regions are set forth in the appended sequence listing. Table 7 further demonstrates that very few framework changes were necessary to maintain the favorable properties of the binding modulators. In this respect framework changes or back mutations were only made in three of the heavy chain variable regions and only two framework modifications were undertaken in the light chain variable regions.

Note that, for some humanized light and heavy chain variable regions (e.g. hSC17.200, hSC17.155 and hSC17.161), conservative amino acid mutations were introduced in the CDRs to address stability concerns while maintaining antigen binding. In each case, the binding affinity of the antibodies with modified CDR's was found to be equivalent to either the corresponding chimeric or murine antibody. The sequences of nine exemplary humanized variant chains (light and heavy) are listed at the end of FIGS. 3A and 3B (SEQ ID NOS: 192-199) where they retain the designation of the humanized parent chain with notation to indicate they have been altered (e.g. hSC17.200vL1, hSC17.155vH1-6 and hSC17.161vH1).

Following humanization of all selected antibodies by CDR grafting, the resulting light and heavy chain variable region amino acid sequences were analyzed to determine their homology with regard to the murine donor and human acceptor light and heavy chain variable regions. The results, shown in Table 8 below, reveal that the humanized constructs consistently exhibited a higher homology with respect to the human acceptor sequences than with the murine donor sequences. More specifically, the humanized heavy and light chain variable regions generally show a higher percentage homology to a closest match of human germline genes (84%-95%) as compared to the homology of the humanized variable region sequences and the donor hybridoma protein sequences (74%-89%).

TABLE 8

| | Homology to Human | Homology to Murine Parent |
|---|---|---|
| mAb | (CDR acceptor) | (CDR donor) |
| hSC17.16 HC | 91% | 80% |
| hSC17.16 LC | 86% | 85% |
| hSC17.17 HC | 93% | 80% |
| hSC17.17 LC | 87% | 77% |
| hSC17.24 HC | 86% | 79% |
| hSC17.24 LC | 93% | 89% |
| hSC17.28 HC | 89% | 77% |
| hSC17.28 LC | 92% | 78% |
| hSC17.34 HC | 85% | 83% |
| hSC17.34 LC | 84% | 86% |
| hSC17.46 HC | 85% | 83% |
| hSC17.46 LC | 84% | 80% |
| hSC17.151 HC | 90% | 79% |
| hSC17.151 LC | 87% | 80% |
| hSC17.155 HC | 90% | 80% |
| hSC17.155 LC | 95% | 87% |
| hSC17.156 HC | 89% | 79% |
| hSC17.156 LC | 86% | 93% |
| hSC17.161 HC | 89% | 86% |
| hSC17.161 LC | 93% | 87% |
| hSC17.200 HC | 90% | 74% |
| hSC17.200 LC | 88% | 82% |

Upon testing each of the humanized constructs exhibited favorable binding characteristics roughly comparable to those shown by the murine parent antibodies (Data not shown).

### Example 5

### Generation of Humanized Anti-CD324 Antibodies

Anti-CD324 humanized antibodies were generated substantially as set forth in Examples 1-3 above. A more detailed description of the production of the CD324 antigen and corresponding antibodies is provided in PCT/US2013/25356, which is incorporated herein by reference as to the same. Following inoculation of six female mice antibody producing hybridomas were generated substantially as set forth in Example 1. The hybridomas were screened as previously discussed and genetic material obtained from those of interest. Sequences of the heavy and light chain variable regions of the anti-SEZ6 antibodies were determined substantially as set forth in Example 2.

FIG. 4 shows the contiguous amino acid sequences of the light (SEQ ID NO: 529) and heavy (SEQ ID NO: 530) chain variable regions of an exemplary anti-CD324 murine antibody, SC10.17. Nucleic acid sequences corresponding to the murine heavy and light chains are provided in the sequence listing appended hereto (SEQ ID NOS: 531 and 532). Sequences of SC10.17 and other compatible light and heavy chain variable regions from anti-CD324 antibodies are shown in PCT/US2013/25356 which is incorporated herein as to these sequences.

The SC10.17 anti-CD324 murine antibody was humanized, substantially as set forth in Example 3 above using standard molecular engineering techniques. Using Kabat numbering, FIG. 4 denotes the CDRs and framework regions, as determined using the Abysis Database, of the heavy and light chains of the murine parent antibody and the derived humanized construct. A review of FIG. 4 shows the murine heavy and light CDRs were transferred to the human acceptor molecule with only minor alterations in the CDRs. More particularly FIG. 4 shows amino acid sequences of the light (SEQ ID NO: 531) and heavy (SEQ ID NO: 532) chains of an exemplary humanized anti-CD324 antibody, termed hSC10.17. As with the parent murine antibody corresponding nucleic acid sequences are set forth in the appended sequence listing (SEQ ID NOS: 535 and 536). The light and heavy chain variable regions of hSC10.17 exhibited higher homology with the light and heavy chain variable regions of the human acceptor sequence compared to the murine donor sequence (data not shown).

### Example 6

### Fabrication of Site-Specific Anti-DLL3 Antibodies

Four engineered human IgG1/kappa anti-DLL3 site-specific antibodies were constructed. Two of the four engineered antibodies comprised a native light chain constant regions and had mutations in the heavy chain, wherein cysteine 220 (C220) in the upper hinge region of the heavy chain, which forms an interchain disulfide bond with cysteine 214 in the light chain, was either substituted with serine (C220S) or removed (C220Δ). The remaining two engineered antibodies comprised a native heavy chain constant regions and a mutated light chain, wherein cysteine 214 of the light chain was either substituted with serine (C214S) or removed (C214Δ). When assembled the heavy and light chains form antibodies comprising two free cysteines that are suitable for conjugation to a therapeutic agent. Amino acid sequences for the heavy and light antibody chains for each of the exemplary hSC16.56 constructs are shown in FIGS. 5A and 5B while Table 9 immediately below summarizes the alterations. With regard to FIGS. 5A and 5B the reactive (or free) cysteine is underlined as is the mutated residue (in ss1 and ss4) at position 220 for the heavy chain and position 214 for the light chain. Unless otherwise noted, all numbering of constant region residues is in accordance with the EU numbering scheme as set forth in Kabat et al.

TABLE 9

| | Antibody | | Const. Reg. | SC16.56 |
|---|---|---|---|---|
| Designation | Component | Alteration | SEQ ID NO: | SEQ ID NO: |
| ss1 | Heavy Chain | C220S | 500 | 509 |
| | Light Chain | WT | 403 | 507 |
| ss2 | Heavy Chain | C220Δ | 501 | 510 |
| | Light Chain | WT | 403 | 507 |
| ss3 | Heavy Chain | WT | 404 | 508 |
| | Light Chain | C214Δ | 502 | 511 |
| ss4 | Heavy Chain | WT | 404 | 508 |
| | Light Chain | C214S | 503 | 512 |

The engineered antibodies were generated as follows.

An expression vector encoding the humanized anti-DLL3 antibody hSC16.56 light chain (SEQ ID NO: 507) or heavy chain (SEQ ID NO: 508) derived as set forth in Example 3 were used as a template for PCR amplification and site directed mutagenesis. Site directed mutagenesis was performed using the Quick-change® system (Agilent Technologies) according to the manufacturer's instructions.

For the two heavy chain mutants, the vector encoding the mutant C220S or C220Δ heavy chain of hSC16.56 was co-transfected with the native IgG1 kappa light chain of hSC16.56 in CHO-S cells and expressed using a mammalian transient expression system. The engineered anti-DLL3 site-specific antibodies containing the C220S or C220Δ mutants were termed hSC16.56ss1 (SEQ ID NOS: 509 and 507) or hSC16.56ss2 (SEQ ID NOS: 510 and 507) respectively.

For the two light chain mutants, the vector encoding the mutant C214S or C214Δ light chain of hSC16.56 was co-transfected with the native IgG1 heavy chain of hSC16.56 in CHO-S cells and expressed using a mammalian transient expression system. The engineered antibodies were purified using protein A chromatography (MabSelect SuRe) and stored in appropriate buffer. The engineered anti-DLL3 site-specific antibodies containing the C214S or C214Δ mutants were termed hSC16.56ss3 (SEQ ID NOS: 508 and 511) or hSC16.56ss4 (SEQ ID NOS: 508 and 512) respectively.

The engineered anti-DLL3 antibodies were characterized by SDS-PAGE to confirm that the correct mutants had been generated. SDS-PAGE was conducted on a pre-cast 10% Tris-Glycine mini gel from life technologies in the presence and absence of a reducing agent such as DTT (dithiothreitol). Following electrophoresis, the gels were stained with a colloidal coomassie solution (data not shown).

Band patterns of the two heavy chain (HC) mutants, hSC16.56ss1 (C220S) and hSC16.56ss2 (C220Δ) and the two light chain (LC) mutants, hSC16.56ss3 (C214S) and hSC16.56ss4 (C214Δ) were observed. Under reducing conditions, for each antibody, two bands corresponding to the free LCs and free HCs, were observed. This pattern is typical of IgG molecules in reducing conditions. Under non-reducing conditions, the four engineered antibodies (hSC16.56ss1-hSC16.56ss4) exhibited band patterns that were different from native IgG molecules, indicative of the absence of a disulfide bond between the HC and LC. All four mutants exhibited a band around 98 kD corresponding to the HC-HC dimer. The mutants with a deletion or mutation on the LC (hSC16.56ss3 and hSC16.56ss4) exhibited a single band around 24 kD corresponding to a free LC. The engineered antibodies containing a deletion or mutation on the heavy chain (hSC16.56ss1 and hSC16.56ss2) had a faint band corresponding to the free LC and a predominant band around 48 kD that corresponded to a LC-LC dimer. The formation of some amount of LC-LC species is expected with the ss1 and ss2 constructs due to the free cysteines on the c-terminus of each light chain.

### Example 7

### Fabrication of Site-Specific Anti-SEZ6 Antibodies

Four engineered human IgG1/kappa anti-SEZ6 site-specific antibodies were constructed substantially as set forth in Example 6 using the humanized antibody hSC17.200 as a starting point. Two of the four engineered antibodies comprised a native light chain constant regions and had mutations in the heavy chain, wherein cysteine 220 (C220) in the upper hinge region of the heavy chain, which forms an interchain disulfide bond with cysteine 214 in the light chain, was either substituted with serine (C220S) or removed (C220Δ). The remaining two engineered antibodies comprised a native heavy chain constant regions and a mutated light chain, wherein cysteine 214 of the light chain was either substituted with serine (C214S) or removed (C214Δ). When assembled the heavy and light chains form antibodies comprising two free cysteines that are suitable for conjugation to a therapeutic agent. Amino acid sequences for the heavy and light antibody chains for each of the exemplary hSC17.200 constructs are shown in FIGS. 6A and 6B while Table 10 immediately below summarizes the alterations. With regard to FIGS. 6A and 6B the reactive cysteine is underlined as is the mutated residue (in ss1 and ss4) at position 220 for the heavy chain and position 214 for the light chain. Unless otherwise noted, all numbering of constant region residues is in accordance with the EU numbering scheme as set forth in Kabat et al.

TABLE 10

| | Antibody | | Const. Reg. | SC17.200 |
|---|---|---|---|---|
| Designation | Component | Alteration | SEQ ID NO: | SEQ ID NO: |
| ss1 | Heavy Chain | C220S | 500 | 515 |
| | Light Chain | WT | 403 | 513 |
| ss2 | Heavy Chain | C220Δ | 501 | 516 |
| | Light Chain | WT | 403 | 513 |
| ss3 | Heavy Chain | WT | 404 | 514 |
| | Light Chain | C214Δ | 502 | 517 |
| ss4 | Heavy Chain | WT | 404 | 514 |
| | Light Chain | C214S | 503 | 518 |

Expression vectors comprising the heavy and light chains of site-specific engineered hSC17.200 antibodies were introduced into CHO or 293 cells which where then used to produce the site-specific antibodies as describe herein.

In addition to hSC17.200 site-specific antibodies hSC17.17 antibodies may be produced and expressed in substantially the same manner. Exemplary hSC17.17 site-specific antibodies would be as summarized in Table 11 set forth immediately below with the full length heavy and light chain amino acid sequences included in the appended sequence listing as indicated.

TABLE 11

| | Antibody | | Const. Reg. | SC17.17 |
|---|---|---|---|---|
| Designation | Component | Alteration | SEQ ID NO: | SEQ ID NO: |
| ss1 | Heavy Chain | C220S | 500 | 539 |
| | Light Chain | WT | 403 | 537 |
| ss2 | Heavy Chain | C220Δ | 501 | 540 |
| | Light Chain | WT | 403 | 537 |
| ss3 | Heavy Chain | WT | 404 | 538 |
| | Light Chain | C214Δ | 502 | 541 |
| ss4 | Heavy Chain | WT | 404 | 538 |
| | Light Chain | C214S | 503 | 542 |

### Example 8

### Fabrication of Site-Specific Anti-CD324 Antibodies

Four engineered human IgG1/kappa anti-CD324 site-specific antibodies were constructed. Two of the four engineered antibodies comprised a native light chain and had mutations in the heavy chain, wherein cysteine 220 (C220) in the upper hinge region of the heavy chain, which forms an interchain disulfide bond with cysteine 214 in the light chain, was either substituted with serine (C220S) or removed (C220Δ). The remaining two engineered antibodies comprised a native heavy chain and a mutated light chain, wherein cysteine 214 of the light chain was either substituted with serine (C214S, see FIG. 7) or removed (C214Δ). The engineered antibodies were generated as follows.

Expression vectors encoding humanized anti-CD324 hSC17.10 antibody light chain or heavy chain comprising appropriate variable regions (SEQ ID NOS: 531 and 532) were used as templates for PCR amplification and site directed mutagenesis. Site directed mutagenesis was performed using the Quick-change® system (Agilent Technologies) according to the manufacturer's instructions.

For the two heavy chain mutants, the vector encoding the mutant C220S or C220Δ heavy chain of hSC10.17 was co-transfected with the native IgG1 kappa light chain of hSC10.17 in CHO-S cells and expressed using a mammalian transient expression system. The engineered anti-CD324 site-specific antibodies containing the C220S or C220Δ mutants were termed SC10.17ss1 or SC10.17ss2 respectively.

For the two light chain mutants, the vector encoding the mutant C214S or C214Δ light chain of hSC10.17 was co-transfected with the native IgG1 heavy chain of hSC10.17 in CHO-S cells and expressed using a mammalian transient expression system. The engineered antibodies were purified using protein A chromatography (MabSelectSure protein A resin) and stored in appropriate buffer. The engineered anti-CD324 site-specific antibodies containing the C214S or C214Δ mutants were termed SC10.17ss3 or SC10.17ss4 respectively.

The amino acid sequence of the entire native heavy chain of hSC10.17ss3 is shown in FIG. 7 as SEQ ID NO: 543 while the amino acid sequence of the entire engineered light chain is shown in the same figure as SEQ ID NO: 544. The C214S (Kabat numbering) position in the kappa light chain is denoted by an * as is the free cysteine at position 220 of the heavy chain (again EU index of Kabat numbering).

### Example 9

### Site-Specific Constructs Retain Binding Characteristics

Site-specific anti-DLL3 antibodies fabricated as set forth in the previous Examples were screened by an ELISA assay to determine whether they bound to DLL3 purified protein. The parental native antibody was used as a control and run alongside the site-specific anti-DLL3 antibody. Binding of the antibodies to DLL3 was detected with a monoclonal antibody (mAb) reporter antibody conjugated to horseradish peroxidase (HRP), (Southern Biotech, Cat. No. SB9052-05), which binds to an epitope present on human IgG1 molecules. HRP reacts with its substrate tetramethyl benzidine (TMB). The amount of hydrolyzed TMB is directly proportional to the amount of test antibody bound to DLL3.

ELISA plates were coated with 1 μg/ml purified DLL3 in PBS and incubated overnight at 4° C. Excess protein was removed by washing and the wells were blocked with 2% (w/v) BSA in PBS with 0.05% tween 20 (PBST), 200 μL/well for 1 hour at room temperature. After washing, 100 μL/well serially diluted antibody or ADC were added in PBST for 1 hour at room temperature. The plates were washed again and 0.5 ug/ml of 100 μL/well of the appropriate reporter antibody was added in PBST for 1 hour at room temperature. After another washing, plates were developed by the addition of 100 μL/well of the TMB substrate solution (Thermo Scientific) for 15 minutes at room temperature. An equal volume of 2 M H₂SO₄ was added to stop substrate development. The samples were then analyzed by spectrophotometer at OD 450.

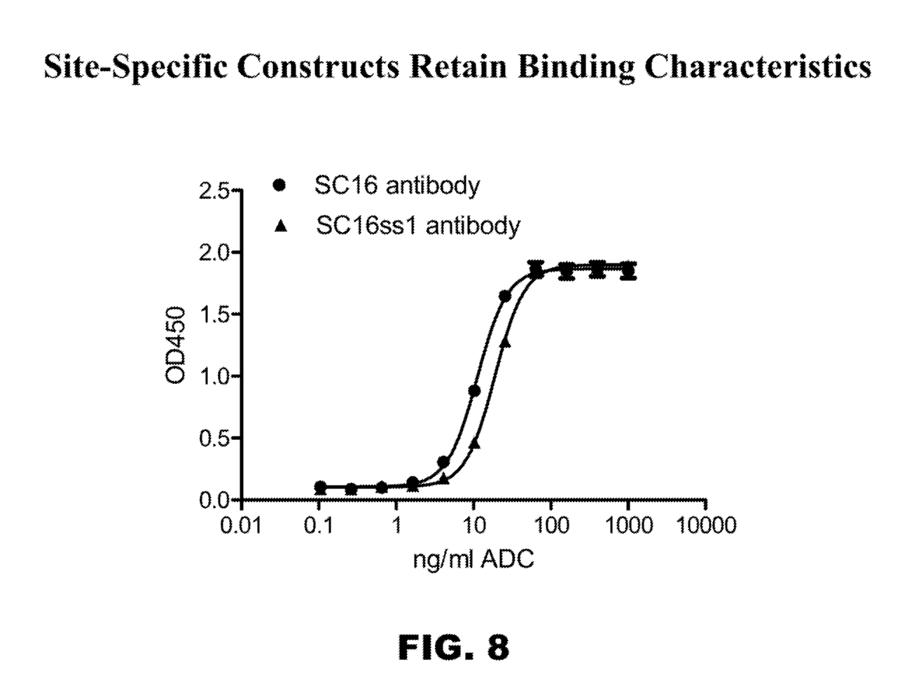

The results of the ELISAs are shown in FIG. 8 as a binding curve. A review of the data demonstrates that engineering of the heavy chain CH1 domain to provide a free cysteine on the light chain constant region did not adversely impact the binding of the antibodies to the target antigen. Similar assays (data not shown) conducted with various site-specific constructs shows that engineering of the light chain constant region or the CH1 region to provide free cysteines has little impact on the binding characteristics of the resulting antibody or ADC.

### Example 10

### Conjugation of Site-Specific Anti-SEZ6 ADC

Site-specific antibody conjugation was undertaken in which engineered anti-SEZ6 antibodies such as those described in Example 7 were conjugated to thiol reactive monomethyl auristatin E via a val cit linker (vcMMAE, see e.g., U.S. Pat. No. 7,659,241). The site-specific conjugation gives rise to a population of ADCs having reduced heterogeneity and complexity of species. As discussed above a homologous population of ADCs comprising a homogeneous composition can have a favorable impact on stability, pharmacokinetics, aggregation and ultimately safety profile.

More specifically an engineered human IgG1/kappa anti-SEZ6 antibody was constructed, wherein the cysteine in the upper hinge region of the heavy chain (C220), which forms an interchain disulfide bond with the light chain, was substituted with serine (C220S) resulting in an antibody (hSC17.200ss1) having two unpaired cysteines to which cytotoxins could be conjugated. The amino acid sequence of the entire engineered heavy chain is shown in FIG. 6A as SEQ ID NO: 515 while the amino acid sequence of the entire light chain is shown in the same figure as SEQ ID NO: 513. The C220S (as per the EU index of Kabat) position in the heavy chain is denoted in bold and underlined as is the free cysteine at position 214 of the kappa light chain (again numbering as per Kabat).

hSC17.200S was conjugated with vcMMAE in three distinct stages; a reduction step, a re-oxidation step and a conjugation step. A schematic diagram of the process can be seen in FIG. 9.

hSC17.200S was fully reduced with a 40 molar equivalent addition of 10 mM DTT in water. The reduction reaction was allowed to proceed overnight (>12 h) at room temperature. The reduced antibody was then buffer exchanged into a Tris pH 7.5 buffer using a 30 kd membrane (Millipore Amicon Ultra) and the equivalent of 10 diavolumes of buffer exchange. The reduced hSC17.200S was then re-oxidized with either a 4.5 molar equivalent addition of 10 mM dehydroascorbic acid (DHAA) in Dimethylacetamide (DMA). The re-oxidation reaction was allowed to proceed at room temperature for 60 minutes. The re-oxidized antibody was then conjugated by the addition of 1.2 moles of vcMMAE per mole of free thiol from a 10 mM stock of vcMMAE in DMA. Additional DMA was added prior to conjugation such that the final concentration of DMA in the reaction mixture was approximately 6% v/v. Conjugation was allowed to proceed for a minimum of 30 minutes before the reaction was quenched with the addition of 1.2 molar excess of N-acetyl cysteine (NAC), from a 10 mM stock solution prepared in water. After a minimum quench time of 20 minutes, the pH was adjusted to 5.5±0.3 with the addition of 4% v/v of 0.5 M acetic acid. Conjugated hSC17.200SvcMMAE was diafiltered into 20 mM histidine chloride pH 6.0 by constant-volume diafiltration using a 10 kDa membrane and a total of 10 diavolumes of buffer exchange prior to sterile filtration and final formulation. The resulting ADC exhibited binding to the SEZ6 antigen comparable to that of the conjugated native SC17.200 antibody and a relatively high percentage of DAR=2 compounds.

### Example 11

### Conjugation of Site-Specific Antibodies

Site-specific antibodies (hSC16.56ss1 and hSC17.200ss1) fabricated as set forth in Examples 6 and 7 above were completely reduced using DTT or partially reduced using TCEP (tris(2-carboxyehy)phosphine) prior to conjugation with linker-drug comprising a vcMMAE in order to demonstrate site-specific conjugation.

Again a schematic diagram of the process can be seen in FIG. 9. The target conjugation site for this construct is the unpaired cysteine (C214) on each light chain constant region. Conjugation efficiency (on-target and off-target conjugation) can be monitored using a reverse-phase HPLC (RP-HPLC) assay that can track on-target conjugation on the light chain vs. off-target conjugation on the heavy chain. A hydrophobic interaction chromatography (HIC) assay may be used to monitor the distribution of drug to antibody ratio species (DAR). In this example, the desired product is an ADC that is maximally conjugated on the light chain (on-target) as determined by reverse-phase chromatography and that minimizes over-conjugated (DAR>2) species while maximizing DAR=2 species.

Different preparations of hSC16.56ss1 or hSC17.200ss1 were either completely reduced with a 40 molar equivalent addition of 10 mM DTT or partially reduced with a 2.6 molar equivalent addition of 10 mM TCEP.

Samples reduced with 10 mM DTT were reduced overnight (>12 h) at room temperature prior to buffer exchange into a Tris pH 7.5 buffer using a 30 kDa membrane (Millipore Amicon Ultra) and the equivalent of 10 diavolumes of buffer exchange. The resulting fully reduced preparations were then re-oxidized with 4.0 molar equivalent addition of 10 mM dehydroascorbic acid (DHAA) in dimethylacetamide (DMA). When the free thiol concentrations (number of free thiols per antibody, as measured by Ellman's method) of the samples were between 1.9 and 2.3, the free cysteines of the antibodies were conjugated to MMAE cytotoxins via a maleimido linker for a minimum of 30 minutes at room temperature. The reaction was then quenched with the addition of 1.2 molar excess of N-acetyl-cysteine (NAC) using a 10 mM stock solution prepared in water. After a minimum quench time of 20 minutes, the pH was adjusted to 6.0 with the addition of 0.5 M acetic acid. The various conjugated preparations of antibody-MMAE were then buffer exchanged into 20 mM histidine chloride pH 6.0 by diafiltration using a 30 kDa membrane.

The samples partially reduced with 10 mM TCEP were reduced for a minimum of 90 minutes at room temperature. When the free thiol concentrations of the samples were between 1.9 and 2.3, the partially reduced antibodies were conjugated to MMAE, a gain via a maleimido linker, for a minimum of 30 minutes at room temperature. The reaction was then quenched with the addition of 1.2 molar excess NAC from a 10 mM stock solution prepared in water. After a minimum quench time of 20 minutes, the pH was adjusted to 6.0 with the addition of 0.5 M acetic acid. The preparations of conjugated antibody-MMAE were then buffer exchanged into 20 mM histidine chloride pH 6.0 by diafiltration using a 30 kDa membrane.

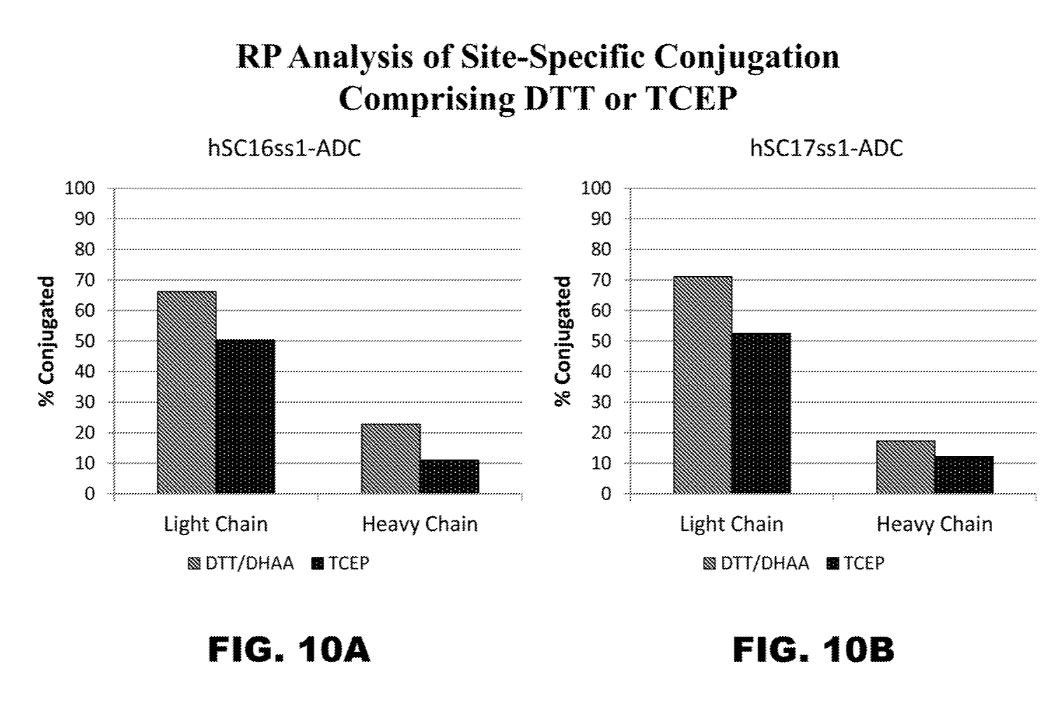

The final antibody-drug preparations (both DTT reduced and TCEP reduced) were analyzed using RP-HPLC to quantify heavy vs. light chain conjugation sites in order to determine the percentage of on-target light-chain conjugation for hSC16.56ss1-MMAE (FIG. 10A) or hSC17.200ss1-MMAE (FIG. 10B). The analysis employed an Aeris WIDEPORE 3.6 m C4 column (Phenomenex) with 0.1% v/v TFA in water as mobile phase A, and 0.1% v/v TFA in 90% v/v acetonitrile as mobile phase B. Samples were fully reduced with DTT prior to analysis, then injected onto the column, where a gradient of 30-50% mobile phase B was applied over 10 minutes. UV signal at 214 nm was collected and then used to calculate the extent of heavy and light chain conjugation.

More particularly the distribution of payloads between heavy and light chains in hSC16.56ss1-MMAE and hSC17.200ss1-MMAE conjugated using DTT and TCEP are shown in FIGS. 10A and 10B. Percent conjugation on the heavy and light chains were performed by integrating the area under the RP-HPLC curve of the previously established peaks (light chain, light chain+1 drug, heavy chain, heavy chain+1 drug, heavy chain+2 drugs, etc.) and calculating the % conjugated for each chain separately. As discussed throughout the instant specification selected embodiments of the invention comprise conjugation procedures that favor placement of the payload on the light chain.

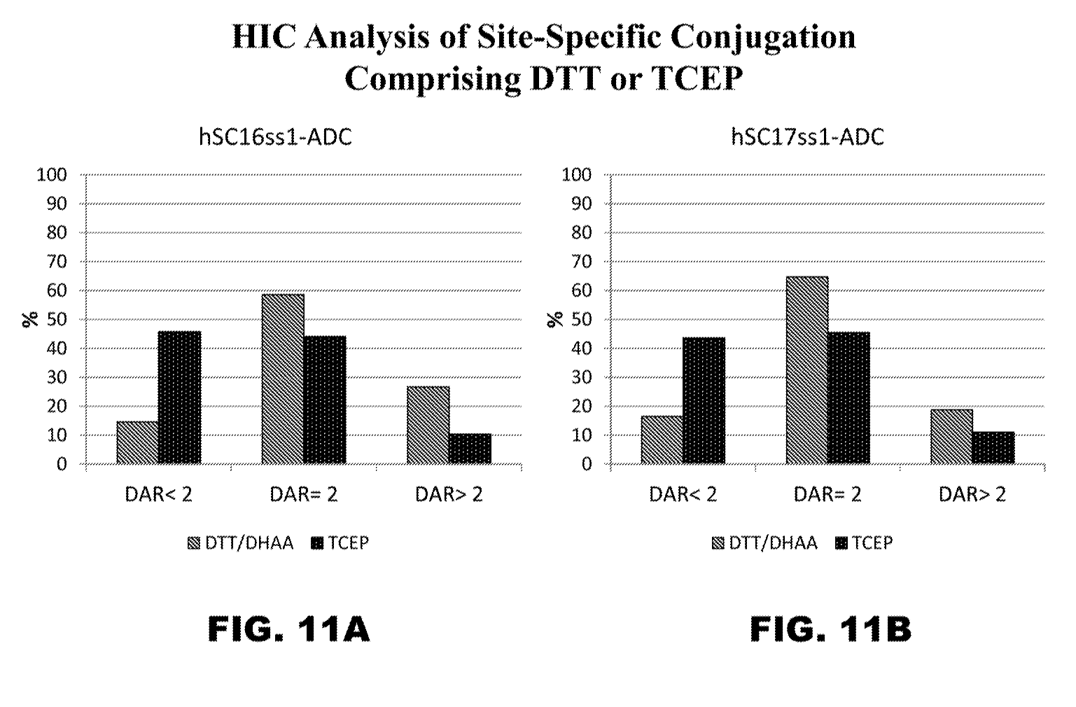

The same preparations were also analyzed using HIC to determine the amount of DAR=2 species relative to the unwanted DAR>2 species for hSC16.56ss1-MMAE (FIG. 11Δ) and hSC17.200ss1-MMAE (FIG. 11B). In this regard HIC was conducted using a PolyPROPYL A 3 m column (PolyLC) with 1.5M ammonium sulfate and 25 mM potassium phosphate in water as mobile phase A, and 0.25% w/v CHAPS and 25 mM potassium phosphate in water as mobile phase B. Samples were injected directly onto the column, where a gradient of 0-100% mobile phase B was applied over 15 minutes. UV signal at 280 nm was collected, and the chromatogram analyzed for unconjugated antibody and higher DAR species. DAR calculations were performed by integrating the area under the HIC curve of the previously established peaks (DAR=0, DAR=1, DAR=2, DAR=4, etc) and calculating the % of each peak. The resulting DAR distribution in hSC16.56ss1-MMAE and hSC17.200ss1-MMAE conjugated using DTT and TCEP are shown in FIGS. 11A and 11B respectively.

The DAR distributions as determined by HIC of the hSC16 site-specific conjugate preparations indicate that the DTT/DHAA full reduction and reoxidation method results in ˜60% DAR=2 species, whereas the typical partial TCEP reduction method results in ˜50% DAR=2. The full reduction and reoxidation method also results in higher unwanted DAR>2 species (20-25%) while the partial TCEP reduction method results in 10-15% DAR>2 (FIGS. 11A and 11B). Note that while the TCEP partial reduction had lower levels of DAR>2 species, the DAR=2 percentage is only 50%. Driving up the % DAR=2 species in the TCEP system would result in a corresponding increase in the unwanted DAR>2 species. The increase in high DAR species for the DTT/DHAA full reduction samples can be attributed to higher off-target conjugation on the heavy chain as shown by RP-HPLC (FIGS. 10A and 10B), which is due to non-specific reduction of the hinge region cysteine residues as the driving force for reduction is increased. Thus, while the disclosed site-specific constructs provide improved DAR and less unwanted higher DAR impurities relative to native antibodies, conventional reduction methods generate at least some non-specific conjugates comprising cytotoxic agents on cysteine residues that are different from the intended engineered sites.

### Example 12

### Conjugation of Engineered Antibodies Using a Selective Reduction Process

In order to further improve the specificity of the conjugation and homogeneity of the final product site-specific antibodies fabricated as set forth in Examples 6 and 7 were selectively reduced using a novel process comprising a stabilizing agent (e.g. L-arginine) and a mild reducing agent (e.g. glutathione) prior to conjugation with linker-drug comprising MMAE. As discussed above, selective conjugation preferentially conjugates the cytotoxin on the free cysteine with a little non-specific conjugation.

Per Examples 6 and 7, the target conjugation site for the hSC16.56ss1 construct is the unpaired cysteine on each light chain. In order to direct conjugation to these engineered sites, preparations of hSC16.56ss1 and hSC17.200ss1 were partially reduced in a buffer containing 1M L-arginine/5 mM glutathione, reduced (GSH)/5 mM EDTA, pH 8.0 for a minimum of one hour at room temperature. Additionally, as controls, each antibody preparation was separately incubated in 1M L-arginine/5 mM EDTA, pH 8.0 and 20 mM Tris/3.2 mM EDTA/5 mM GSH, pH 8.2 buffers for one hour or longer. All preparations were then buffer exchanged into a 20 mM Tris/3.2 mM EDTA, pH 8.2 buffer using a 30 kDa membrane (Millipore Amicon Ultra). The resulting partially reduced preparations (for samples incubated in arginine and glutathione together) had free thiol concentrations between 1.9 and 2.3, and all preparations were then conjugated to MMAE via a maleimido linker for a minimum of 30 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of antibody-MMAE were then diafiltered into 20 mM histidine chloride, pH 6.0 by diafiltration using a 30 kDa membrane.

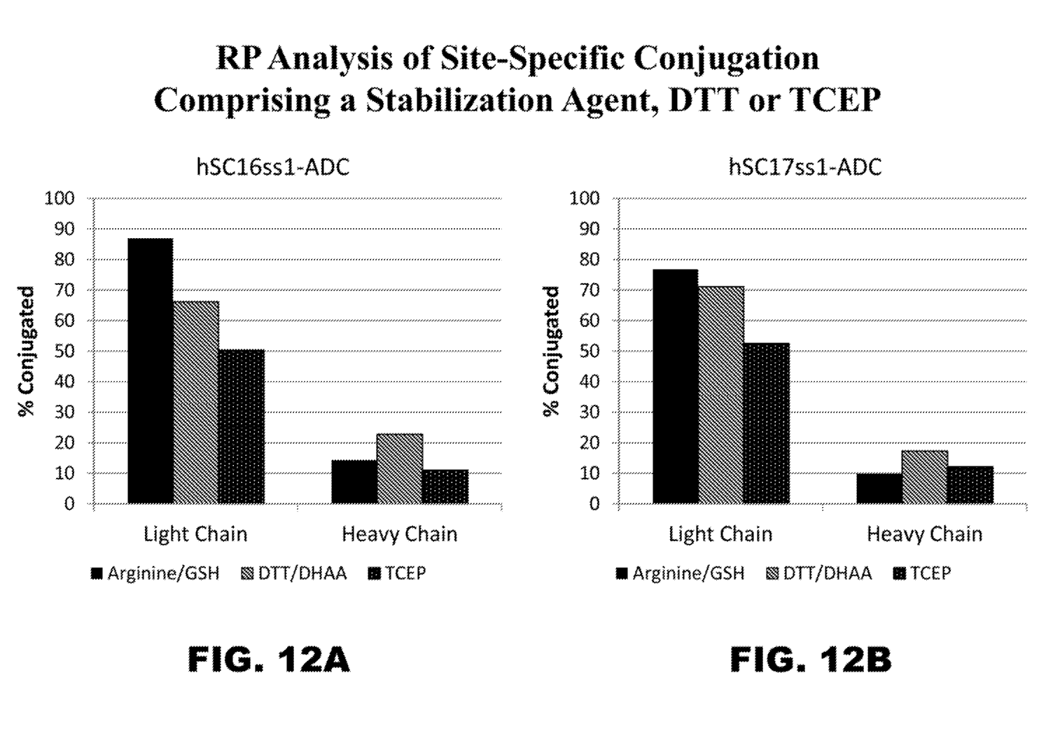

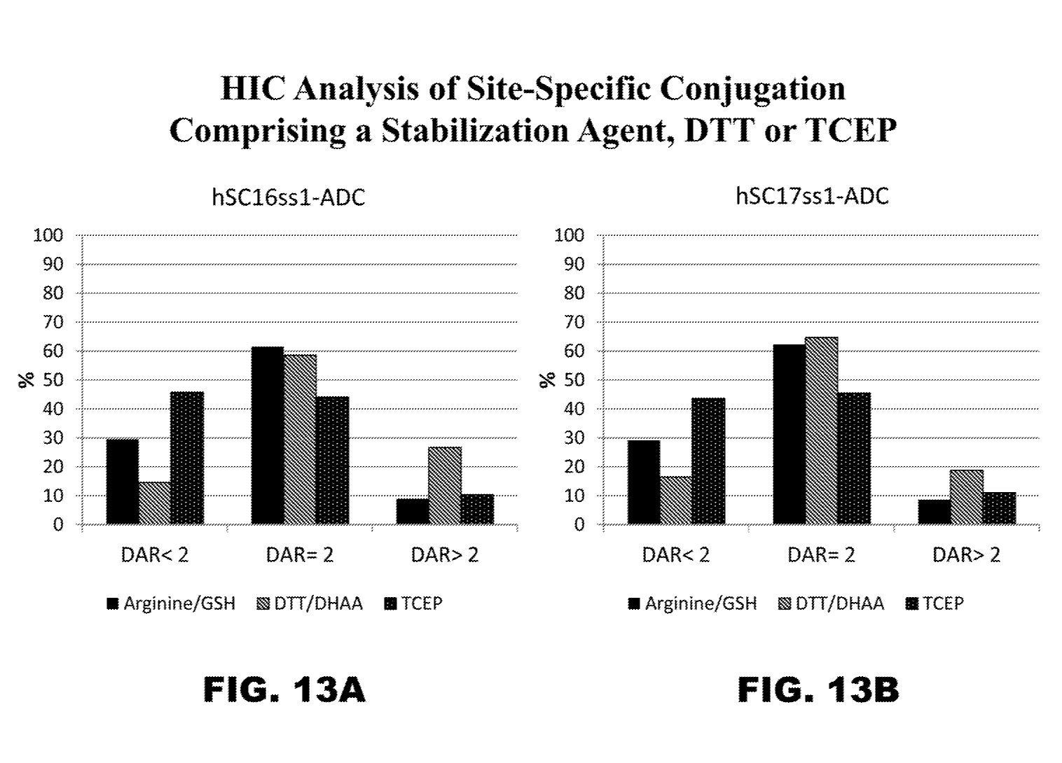

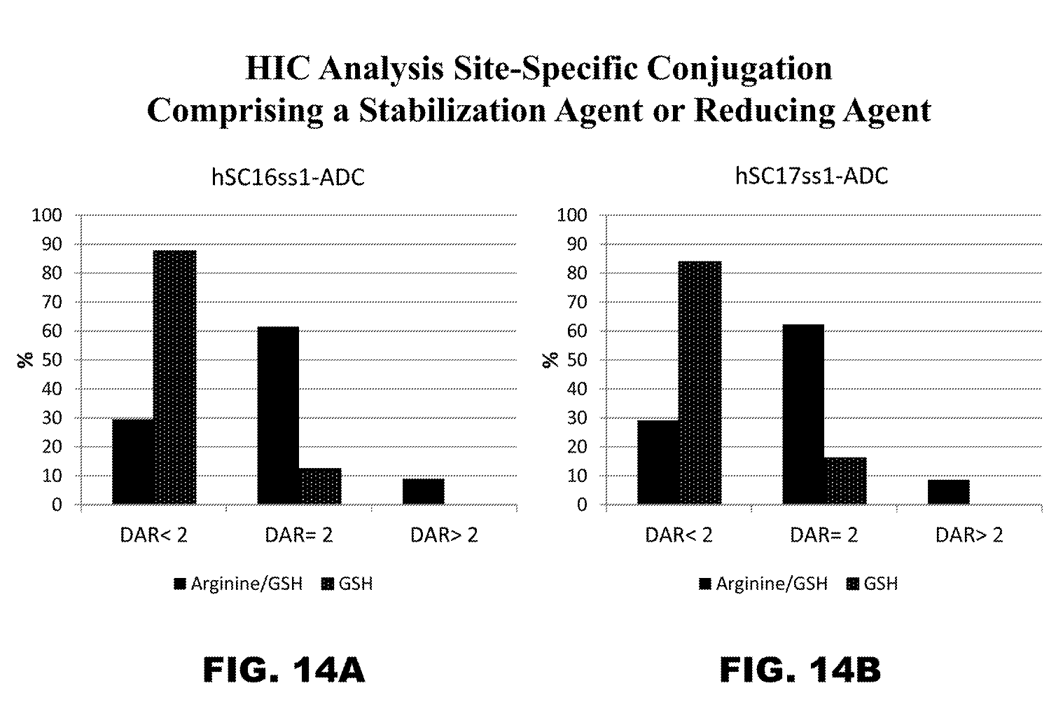

The final antibody-drug preparations were analyzed using RP-HPLC as previously discussed to quantify heavy vs. light chain conjugation sites in order to determine the percentage of on-target light-chain conjugation (FIGS. 12A and 12B). The samples were also analyzed using hydrophobic interaction chromatography to determine the amount of DAR=2 species relative to the unwanted DAR>2 species (FIGS. 13A and 13B). For comparative purposes results obtained in the previous Example are included in FIGS. 12 and 13 for DTT/DHAA and TCEP reduced samples. HIC analysis of the EDTA/GSH controls are presented in FIGS. 14A and 14B where they are shown next to the selectively reduced samples.

FIGS. 12 and 13 summarize the HIC DAR distributions and the % conjugated light chain of the antibodies reduced using the selective reduction process compared to standard complete or partial reduction processes (as described in Examples 10 and 11). The benefit of the selective conjugation method in combination with the engineered constructs is readily apparent, resulting in superior selectivity of the desired light chain conjugation site (FIGS. 12A and 12B) and providing an average DAR=2 level of 60-75% while maintaining unwanted DAR>2 species below 10% (FIGS. 13A and 13B). The results shown in FIGS. 12 and 13 demonstrate that selective reduction drives the reaction to provide higher levels of DAR=2 and less of the undesired DAR>2 species than the standard partial or complete reduction procedures. Control procedures shown in FIGS. 14A and 14B demonstrate that the mild reducing agent (e.g. GSH) cannot effect the desired conjugation in the absence of a stabilizing agent (e.g. L-arginine).

These data demonstrate that selective reduction provides advantages over conventional partial and complete reduction conjugation methods. This is particularly true when the novel selective reduction procedures are used in conjunction with antibodies engineered to provide unpaired (or free) cysteine residues. Mild reduction in combination with a stabilizing agent (i.e., selective reduction) produced stable free thiols that were readily conjugated to various linker-drugs, whereas DHAA reoxidation is time sensitive and TCEP reduction was not as successful, particularly for the engineered constructs described herein.

### Example 13

### Selective Reduction with Different Systems

To further demonstrate the advantages of selective reduction using various combinations of stabilizing agents and reducing agents, hSC16.56ss1 were selectively reduced using different stabilizing agents (e.g. L-lysine) in combination with different mild reducing agents (e.g. N-acetyl-cysteine or NAC) prior to conjugation.

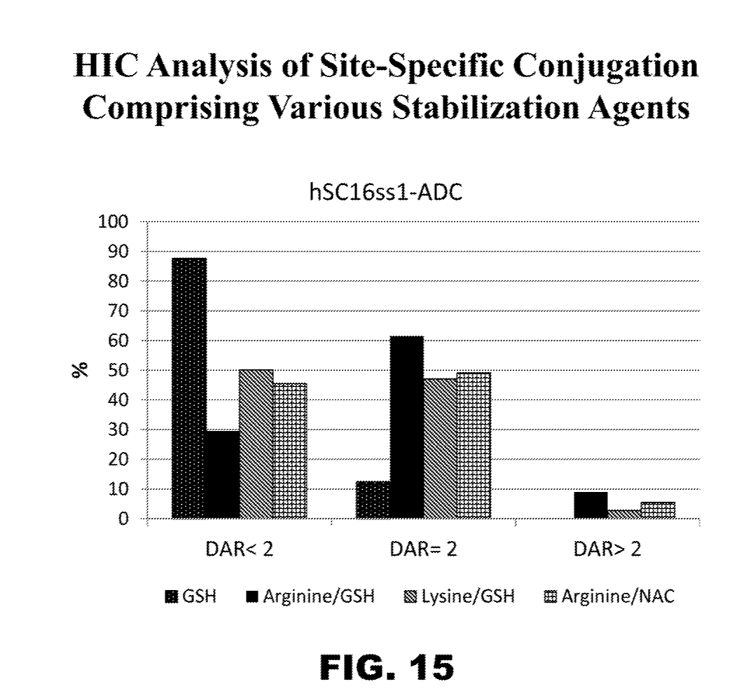

Three preparations each of hSC16.56ss1 were selectively reduced using three different buffer systems: (1) 1M L-arginine/6 mM GSH/5 mM EDTA, pH 8.0, (2) 1M L-arginine/10 mM NAC/5 mM EDTA, pH 8.0, and (3) 1M L-Lysine/5 mM GSH/5 mM EDTA, pH 8.0. Additionally, as controls, the antibody preparations were separately incubated in 20 mM Tris/5 mM EDTA/10 mM NAC, pH 8.0 and 20 mM Tris/3.2 mM EDTA/5 mM GSH, pH 8.2 buffers. All preparations were incubated for a minimum of one hour at room temperature, and then buffer exchanged into a 20 mM Tris/3.2 mM EDTA, pH 8.2 buffer by diafiltration using a 30 kDa membrane (Millipore Amicon Ultra). The resulting selectively reduced preparations, which were found to have free thiol concentrations between 1.7 and 2.4, were then conjugated to MMAE via a maleimido linker. After allowing the conjugation reaction to proceed for a minimum of 30 minutes at room temperature, the reaction was quenched with the addition of 1.2 molar excess of NAC using a 10 mM stock solution. Following a minimum quench time of 20 minutes, the pH was adjusted to 6.0 with the addition of 0.5 M acetic acid. The various conjugated preparations of antibody-MMAE were then buffer exchanged into 20 mM histidine chloride pH 6.0 by diafiltration using a 30 kDa membrane. Final antibody-drug preparations were then analyzed using hydrophobic interaction chromatography to determine DAR distribution (FIG. 15).

DAR distributions as determined by HIC show similar results for the three different selective reduction systems employed (Arg/GSH, Lys/GSH and Arg/NAC). More particularly, DAR=2 levels are 60-65% for the different preparations, and high-DAR species (DAR>2) are maintained below 20% for all selective reduction systems and linker-drug combinations, indicating high selectivity for the engineered cysteine residues in the constant region of the light chain. Again, as previously shown in Example 12, mild reducing agents alone (e.g. GSH or NAC) did not provide sufficient conjugation selectivity while the addition of the stabilizing agent results in significant improvement.

### Example 14

### Site-Specific Conjugates Retain Binding Characteristics

Site-specific anti-DLL3 ADCs prepared as set forth in the previous Examples are screened to determine whether they bind to DLL3 purified protein. A representative screening assay is an ELISA assay, performed essentially as described below. The ELISAs are used to select engineered antibodies that retain binding characteristics.

The parental non-engineered antibody is used, in conjugated and non-conjugated forms, as a control and run alongside the site-specific anti-DLL3 antibody and anti-DLL3 antibody drug conjugate. Binding of the antibodies to DLL3 is detected with a monoclonal antibody (mAb) reporter antibody conjugated to horseradish peroxidase (HRP), (Southern Biotech, Cat. No. SB9052-05), which binds to an epitope present on human IgG1 molecules. Binding of the ADCs (site-specific or conventional) to DLL3 is detected using an antibody conjugated to horseradish peroxidase (HRP) which binds to the drug or drug linker on the ADC. HRP reacts with its substrate tetramethyl benzidine (TMB). The amount of hydrolyzed TMB is directly proportional to the amount of test article bound to DLL3.

ELISA plates are coated with 1 g/ml purified DLL3 in PBS and incubated overnight at 4° C. Excess protein is removed by washing and the wells are blocked with 2% (w/v) BSA in PBS with 0.05% tween 20 (PBST), 200 μL/well for 1 hour at room temperature. After washing, 100 μL/well serially diluted antibody or ADC are added in PBST for 1 hour at room temperature. The plates are washed again and 0.5 ug/ml of 100 μL/well of the appropriate reporter antibody is added in PBST for 1 hour at room temperature. After another washing, plates are developed by the addition of 100 μL/well of the TMB substrate solution (Thermo Scientific) for 15 minutes at room temperature. An equal volume of 2 M H₂SO₄ is added to stop substrate development. The samples are then analyzed by spectrophotometer at OD 450.

### Example 15

### In Vitro Cytotoxicity of Site-Specific Conjugates

Assays are performed to demonstrate the ability of site-specific conjugates to effectively kill cells expressing the human DLL3 antigen in vitro. For example, an assay can be used to measure the ability of an anti-DLL3 site-specific conjugate to kill HEK293T cells engineered to express human DLL3. In this assay killing requires binding of the ADC (site-specific or control) to its DLL3 target on the cell surface followed by internalization of ADC. Upon internalization the linker (e.g., a Val-Ala protease cleavable linker as described above) is cleaved and releases the cytotoxin inside the cells leading to cell death. Cell death is measured using CellTiter-Glo reagent that measures ATP content as a surrogate for cell viability.

A representative assay is performed essentially as follows. Cells are plated into 96 well tissue culture treated plates, with 500 cells per well in DMEM supplemented with 10% fetal bovine serum and penicillin/streptomycin (DMEM complete media), one day before the addition of antibody drug conjugates. 24 hours post plating cells are treated with serially diluted SCAb-cytotoxin control or SCAbss1-cytotoxin in DMEM complete media. The cells are cultured for 96 hours post treatment, after which, viable cell numbers are enumerated using Cell Titer Glo® (Promega) as per manufacturer's instructions.

### Example 16

### Stability of Site-Specific Conjugates in Serum

In order to demonstrate improved stability provided by the site-specific conjugates of the instant invention, selected conjugates are exposed to human serum in vitro for extended periods. Degradation of the ADCs is measured over time. For example, a representative assay is performed essentially as follows.

SCAb ADC and SCAbss1 ADC, each comprising a same cytotoxin, are added to human serum obtained commercially (Bioreclamation) and incubated at 37° C., 5% C02 for extended periods. Samples are collected at 0, 24, 48, 96 and 168 hours post addition and stability is measured using a sandwich ELISA to measure both total antibody content and ADC levels.

With regard to the measurement of total antibody content the ELISA is configured to detect both conjugated and unconjugated SCAb or SCAbss1 antibodies. This assay employs a pair of anti-idiotypic antibodies which specifically capture and detect SCAb and SCAbss1 with or without conjugated cytotoxins. Mechanically the assay is run using the MSD Technology Platform (Meso Scale Diagnostics, LLC) which uses electrochemiluminescence for increased sensitivity and linearity.

To this end MSD high bind plates are coated overnight at 4° C. with 2 ug/mL capture anti-idiotypic (ID-16) antibody. The next day, plates are washed with PBST (PBS+0.05% Tween20) and blocked with 150 uL 3% BSA in PBST. 25 uL serum samples, along with ADC standard curve are added to the plate and allowed to incubate for 2 hours at room temperature. After incubation, plates are washed with PBST and 25 uL sulfo-tagged detection anti-idiotypic (ID-36) antibody at 0.5 ug/mL is added to each well and incubated for 1 hour at room temperature. Plates are then washed and 150 uL 1×MSD read buffer is added per well and read out with the MSD reader. Data is graphed as a percentage of total ADC initially added into the human serum.

In addition to monitoring the total antibody concentration, ELISA assays are run on the collected samples to determine levels of antibody drug conjugate remaining. That is, the assay measures the levels of intact SCAb-cytotoxin and SCAbss1-cytotoxin using the ELISA methodology generally as described immediately above. However, unlike the previous ELISA assay this ELISA quantifies the SCAb or SCAbss1 antibody conjugated to one or more cytotoxin molecules, but cannot determine the number of cytotoxin molecules actually present on the detected ADC. Unlike the total antibody assay, this assay uses a combination of an anti-idiotypic mAb and an anti-cytotoxin specific mAb and does not detect the unconjugated SCAb antibody.

This ELISA assay uses the MSD Technology Platform to generate the data, and a representative assay is performed essentially as follows. MSD standard bind plates are coated overnight at 4° C. with 4 ug/mL anti-cytotoxin specific mAb. The next day, plates are washed with PBST (PBS+0.05% Tween20) and blocked with 150 uL 3% BSA in PBST. 25 uL serum samples, along with ADC standard curve and QC samples are added to the plate and allowed to incubate for 2 hours at room temperature. After incubation, plates are washed with PBST and 25 uL sulfo-tagged detection anti-idiotypic antibody (ID-36) at 0.5 ug/mL is added to each well and incubated for 1 hour at room temperature. Plates are then washed and 150 uL 1×MSD read buffer is added per well and read out with the MSD reader. The data is analyzed to select ADCs showing minimal degradation of the ADC so as to avoid non-specific toxicity resulting from the free cytotoxin and corresponding reduction in the therapeutic index.

### Example 17

### Albumin Transfer of Site-Specific Conjugates in Serum

With conventional ADCs it has been noted that albumin in serum can leach the conjugated cytotoxin thereby increasing non-specific cytotoxicity. In order to determine the amount of site-specific ADC degradation mediated by albumin transfer, an ELISA assay was developed to measure the amount of albumin-cytotoxin (hAlb-cytotoxin) in serum exposed to SCAb-cytotoxin and SCAbss1-cytotoxin. This ELISA uses an anti-cytotoxin specific mAb to capture hAlb-cytotoxin and an anti-human albumin mAb is used as detection antibody. As free ADC will compete with the hAlb-cytotoxin, serum samples are depleted of the ADC prior to testing. Quantitation is extrapolated from a hAlb-cytotoxin standard curve. Along with the previous Example this assay uses the MSD Technology Platform to generate the data. A representative assay is performed essentially as follows.

Initially the serum samples are inoculated with SCAb-cytotoxin or SCAbss1-cytotoxin to a final concentration of 10 μg along with the relevant controls. As with the previous Example, samples are taken at 0, 24, 48, 96 and 168 hours post addition. MSD standard bind plates are coated overnight at 4° C. with 4 ug/mL anti-cytotoxin specific mAb. The next day, plates are washed with PBST (PBS+0.05% Tween20) and blocked with 25 uL MSD Diluent 2+0.05% Tween-20 for 30 minutes at room temperature. Serum samples are diluted 1:10 in MSD Diluent 2+0.1% Tween-20 (10 uL serum+90 uL diluent) and incubated with 20 uL GE's MabSelect SuRe Protein A resin for 1 hour on vortex shaker. After depletion of intact SCAb-cytotoxin or SCAbss1-cytotoxin by anti-idiotypic antibodies, samples are separated from resin using 96-well 3M filter plate. 25 uL of depleted serum samples are then added to the blocked plate along with an hAlb-6.5 standard curve and incubated for 1 hour at room temperature. After incubation, the plates are washed with PBST and 25 uL of 1 ug/mL sulfo-tagged anti-human albumin mAb (Abcam ab10241) diluted in MSD Diluent 3+0.05% Tween-20 are added. The plates are then incubated for 1 hour, washed with PBST and read out with 150 uL 1×MSD read buffer. The data is analyzed to select ADCs showing minimal albumin transfer rates.

### Example 18

### Site-Specific Constructs Demonstrate In Vivo Efficacy

In vivo experiments are conducted to confirm the cell killing ability of the site-specific constructs described herein. To this end site-specific DLL3 ADCs prepared as set forth in the previous Examples are tested for in vivo therapeutic effects in immunocompromised NODSCID mice bearing subcutaneous patient-derived xenograft (PDX) small cell lung cancer (SCLC) tumors essentially as follows. Anti-DLL3-cytotoxin conjugates (SCAb-ADC), HIC purified anti-DLL3-cytotoxin conjugates (SCAb-ADCD2), and HIC purified site-specific anti-DLL3-cytotoxin conjugates (SCAbss1-ADCD2) are each tested in three different SCLC models.

SCLC-PDX lines, LU129, LU64, and LU117 are each injected as a dissociated cell inoculum under the skin near the mammary fat pad region, and measured weekly with calipers (ellipsoid volume=a×b²/2, where a is the long diameter, and b is the short diameter of an ellipse). After tumors grew to an average size of 200 mm³ (range, 100-300 mm³), the mice are randomized into treatment groups (n=5 mice per group) of equal tumor volume averages. Mice are treated with a single dose (100 μL) with either vehicle (5% glucose in sterile water), control human IgG1 ADC (IgG-ADC; 1 mg/kg), or SCAb-ADC preparations (0.75-1.5 mg/kg) via an intraperitoneal injection, with therapeutic effects assessed by weekly tumor volume (with calipers as above) and weight measurements. Endpoint criteria for individual mice or treatment groups includes health assessment (any sign of sickness), weight loss (more than 20% weight loss from study start), and tumor burden (tumor volumes>1000 mm³). Efficacy is monitored by weekly tumor volume measurements (mm³) until groups reach an average of approximately 800-1000 mm³. Tumor volumes are calculated as an average with standard error mean for all mice in treatment group and are plotted versus time (days) since initial treatment. Results of the treatments are depicted as mean tumor volumes with standard error mean (SEM) in 5 mice per treatment group.

DLL3-binding ADCs conjugated using either conventional (SCAb-cytotoxin or SCAb-ADCD2) or site-specific strategies (SCAbss1-ADCD2) with HIC purification (in two preparations) of molecular species containing 2 drug molecules per antibody are evaluated in mice bearing SCLC PDX-LU129, PDX-LU64, or PDX-LU117. The results are analyzed to assess the effect of HIC purification and/or site-specific conjugation of DLL3-binding ADCs on therapeutic effect.

### Example 19

### Site-Specific Conjugates Demonstrate Reduced Toxicity

In order to further expand the therapeutic index of the disclosed conjugate preparations, studies are run to document their toxicity profile. In particular, these studies are performed to select anti-DLL3 site-specific conjugates that are better tolerated (e.g., no mortality for the same number of doses, reduced incidence of skin toxicity, reduced bone marrow toxicity, reduced severity of lymphoid tissue findings, etc.). Significantly, a reduction in toxicity substantially increases the therapeutic index in that it provides for markedly higher dosing and corresponding higher localized concentrations of the cytotoxin at the tumor site. A representative assay is performed essentially as follows.

The toxicity of DAR2 purified site-specific ADC (SCAbss1-ADCD2) is compared to that of conventional conjugates (SCAb-ADC) or DAR2 purified versions of the same (SCAb-ADCD2). Each of the preparations comprise a same cytotoxin. The studies are conducted using cynomolgus monkeys as a test system. Survival, clinical signs, body weights, food consumption, clinical pathology (hematology, coagulation, clinical chemistry, and urinalysis), toxicokinetics, gross necropsy findings, organ weights, and histopathologic examinations are documented and compared.

### Example 20

### Engineered Site-Specific Conjugates

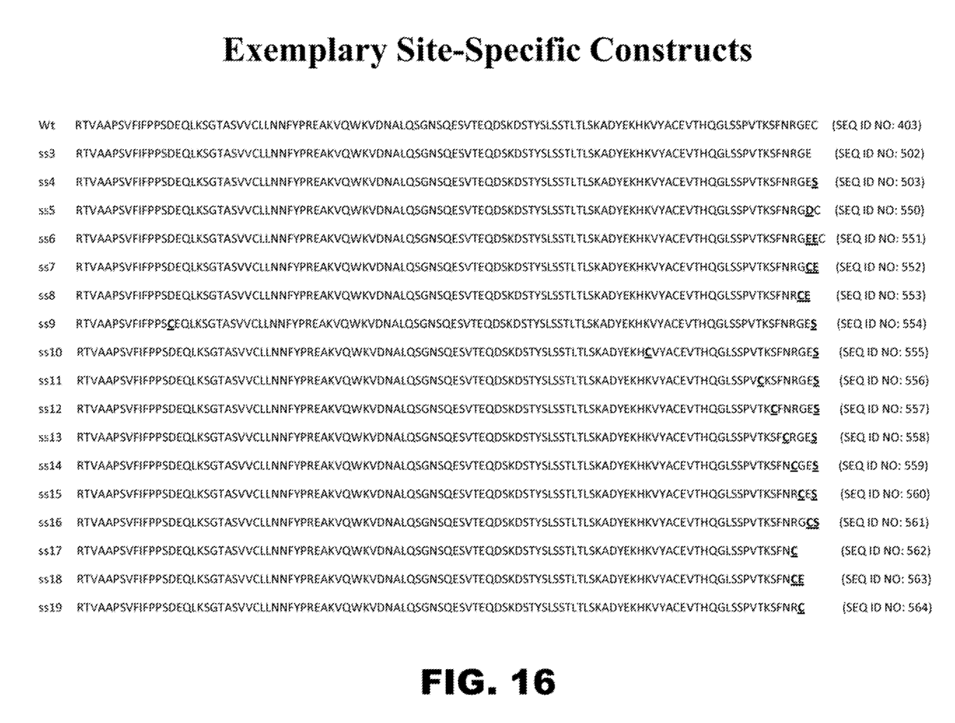

In addition to the site-specific antibodies fabricated and characterized in the Examples above, several exemplary engineered constructs comprising introduced cysteines or modified cysteine proximal residues were fabricated. More specifically, the kappa CL region was modified several different ways according to the instant invention to provide engineered antibodies and conjugates. In this regard the each CL domain was associated with a control VL domain recognizing DLL3 and the light chain was paired with a modified anti-DLL3 heavy chain comprising the C220S substitution. The sequences of the modified CL domains are shown in FIG. 16 (SEQ ID NOS: 502, 503 and 550-564) along with the wild type kappa CL (SEQ ID NO: 403) wherein the altered/introduced residues are underlined. Although a kappa CL was used for demonstration purposes it will be appreciated that compatible lambda CLs could readily be selected and engineered as described herein to provide engineered antibodies of the instant invention. In any event all numbering in this Example is in accordance with the EU numbering scheme.

The modifications to the CL domain generally fall under one of three categories depending on the type of incorporated mutation. The first category incorporates additional or modified negative charge in order to mimic or enhance the positive effects of selective reduction. In this regard ss5 contains an E213D mutation while ss6 contains an insertion of an additional glutamate at between E213 and C214 of the parent light chain.

The second category incorporates alternative sites on the flexible loops of the kappa constant region that are in proximity to the parental C214 residue and are solvent exposed. All of these constructs have a C214S mutation or a deletion of C214, in order to effectively move the cysteine site of conjugation from C214 to other locations on the light chain. This includes the region between positions 182 and 191, for which ss10 (K190C C214S) is a representative construct. This can also include the region between positions 121 and 128, for which ss9 (D122C C214S) is a representative construct. This can also include the region between positions 210 and 213 which are represented by ss13 (N210C C214S), ss14 (R211C C214S), ss15 (G212C C214S), ss16 (E213C C214S) and ss19 (G212C, delete E213-C214).

The third category demonstrates that exposed residues on the beta sheets of the Ig fold of the kappa constant region in proximity to the parental C214 residue provide potential free cysteine sites as discussed herein. In such embodiments the C214 is preferentially eliminated or substituted with serine to remove a potentially competing cysteine. ss11 (T206C C214S) and ss12 (S208C C214S) are representative constructs of this third category.

Combinations of strategies from the three categories can also make further improvements to the engineered construct and enhance site-specific conjugation. For example, ss7 (E213C C214E) and ss8 (G212C C214delete) combine strategies from category 1 with a C terminal negatively charged residue and category 2 with a novel position for the cysteine for site specific conjugation. Several constructs combine truncation of residues from the C terminus with strategies from one or more of the three categories. ss17 (R211C G212Δ, E213Δ, C214Δ) employs category 2 with a cysteine in a different location from the C214 site, and truncation of residues on the C terminal side of the cysteine to create a C-terminal cysteine for conjugation. ss18 (R211C, G212E, E213-C214Δ) employs a combination of category 2 with a cysteine in position 211, category 1 with a C terminal glutamic acid residue, and truncation of the remaining C terminus.

A summary of the aforementioned light chain constant region mutations is shown in Table 12 immediately below.

TABLE 12

| | | Related |
|---|---|---|
| Site-Specific construct | Mutation | SEQ ID NO |
| kappa constant region | no mutation | SEQ ID NO: 403 |
| ss3 | C214Δ | SEQ ID NO: 502 |
| ss4 | C214S | SEQ ID NO: 503 |
| ss5 | E213D | SEQ ID NO: 550 |
| ss6 | E213EE | SEQ ID NO: 551 |
| ss7 | E213C, C214E | SEQ ID NO: 552 |
| ss8 | G212C, E213E C214Δ | SEQ ID NO: 553 |
| ss9 | D122C, C214S | SEQ ID NO: 554 |
| ss10 | K190C, C214S | SEQ ID NO: 555 |
| ss11 | T206C, C214S | SEQ ID NO: 556 |
| ss12 | S208C, C214S | SEQ ID NO: 557 |
| ss13 | N210C, C214S | SEQ ID NO: 558 |
| ss14 | R211C, C214S | SEQ ID NO: 559 |
| ss15 | G212C, C214S | SEQ ID NO: 560 |
| ss16 | E213C, C214S | SEQ ID NO: 561 |
| ss17 | R211C, G212-C214Δ | SEQ ID NO: 562 |
| ss18 | R211C, G212E, E213-C214Δ | SEQ ID NO: 563 |
| ss19 | G212C, E213-C214Δ | SEQ ID NO: 564 |

All of the light chain constructs comprising a selected VL were made using either the Quikchange mutagenesis kit (Stratagene), or by custom gene synthesis (IDT). As previously described these light chain constructs were cloned into expression vectors and co-transfected with the selected heavy chain vector into CHO-S cells. In this regard 100 μg total vector DNA was added to 300 μg PEI transfection reagent in Opti-MEM and the mix was incubated for 10 min. at room temperature before being added to cells. Supernatants comprising the assembled site specific constructs were harvested six to eight days after transfection.

As described above light chains comprising the altered kappa CLs comprising only a deleted or substituted native cysteine (SEQ ID NOS: 502 and 503) are co-expressed with heavy chains comprising a wild-type constant region with the C220 residue (e.g., SEQ ID NO: 404) to provide antibodies comprising two unpaired cysteines on the antibody heavy chains. Conversely, light chains comprising the remaining CL constructs described in FIG. 16 (SEQ ID NOS: 550-564) are co-expressed with a heavy chain missing the C220 residue (e.g., SEQ ID NOS: 500 and 501) to provide two free cysteines in the CL domain of the light chains. See generally Table 2 above. In any event culture supernatants containing recombinant antibodies were cleared of cell debris by centrifugation at 800×g for 10 min. Recombinant antibodies were then purified with Protein A beads and stored at 4° C. until use.

### Example 21

### Engineered Site-Specific Conjugates

To further demonstrate the usefulness of the novel process for the selective reduction of engineered free cysteine residues, various site-specific antibodies (SC16ss5, SC16ss6, SC16ss8, SC16ss11, SC16ss14, and SC16ss17) constructed as set forth in Example 20 were selectively reduced prior to conjugation with linker-drug comprising a terminal maleimido group.

Preparations of SC16ss5, SC16ss6, SC16ss8, SC16ss11, SC16ss14, and SC16ss17 were partially reduced in a buffer containing 1M L-arginine/5 mM glutathione, reduced (GSH)/5 mM EDTA, pH 8.0 for a minimum of one hour at room temperature. All preparations were then buffer exchanged into a 20 mM Tris/3.2 mM EDTA, pH 8.2 buffer using a 30 kDa membrane (Millipore Amicon Ultra). The resulting partially reduced preparations were then conjugated to LD6.5 (PBD) for a minimum of 30 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of ADC were then diafiltered into 20 mM Histidine Chloride (His Cl) pH 6.0 by diafiltration using a 30 kDa membrane.

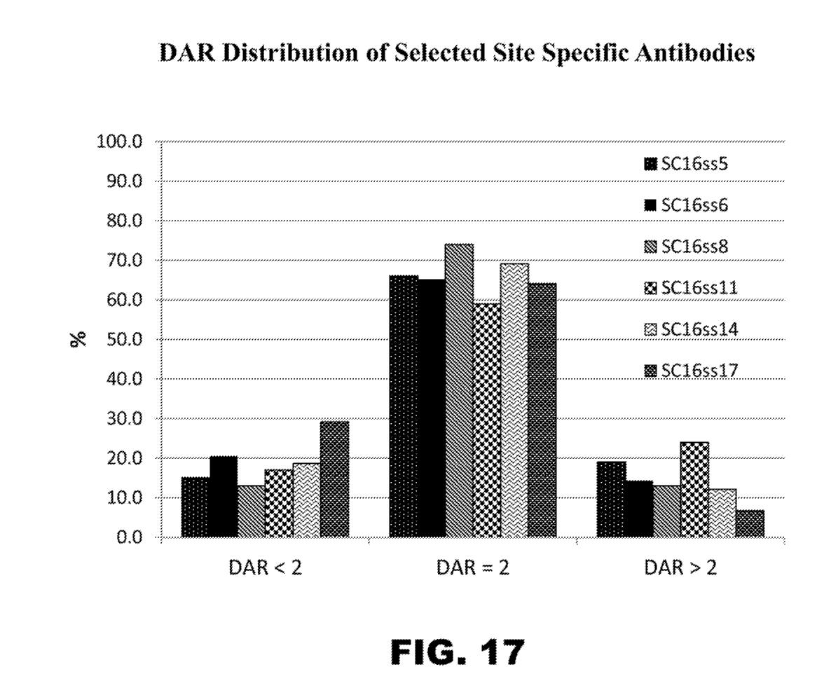

The final antibody-drug preparations were analysed using hydrophobic interaction chromatography (HIC) to determine the amount of DAR=2 species relative to the unwanted DAR>2 species (FIG. 17). The results clearly show the applicability of the selective reduction process to a variety of site-specific antibodies with unpaired cysteine residues. The extent of DAR=2 conjugation for each of the selected engineered antibodies is greater or equal to 60% demonstrating the advantages of using such constructs to provide ADCs.

### Example 22

### Selective Conjunction of Engineered Site-Specific Antibodies

In order to further demonstrate the specificity of the conjugation and homogeneity of the final products of the instant invention site-specific antibodies fabricated as set forth in Example 20 were selectively reduced using a novel process comprising a stabilizing agent (e.g. L-arginine) and a mild reducing agent (e.g. glutathione) prior to conjugation with linker-drug comprising a terminal maleimido group.

As described in Example 20, the target conjugation site of each SC16ss5-19 construct is the unpaired cysteine on each light chain. In order to direct conjugation to these engineered sites, preparations of hSC16ss 5-19 were partially reduced in a buffer containing 1M L-arginine/5 mM glutathione, reduced (GSH)/5 mM EDTA, pH 8.0 for a minimum of one hour at room temperature. All preparations were then buffer exchanged into a 20 mM Tris/3.2 mM EDTA, pH 8.2 buffer using a 30 kDa membrane (Millipore Amicon Ultra). The resulting partially reduced preparations had free thiol concentrations between 1.9 and 2.3, and all preparations were then conjugated to the drug linker via a maleimido group for a minimum of 30 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations were then diafiltered into 20 mM histidine chloride, pH 6.0 by diafiltration using a 30 kDa membrane.

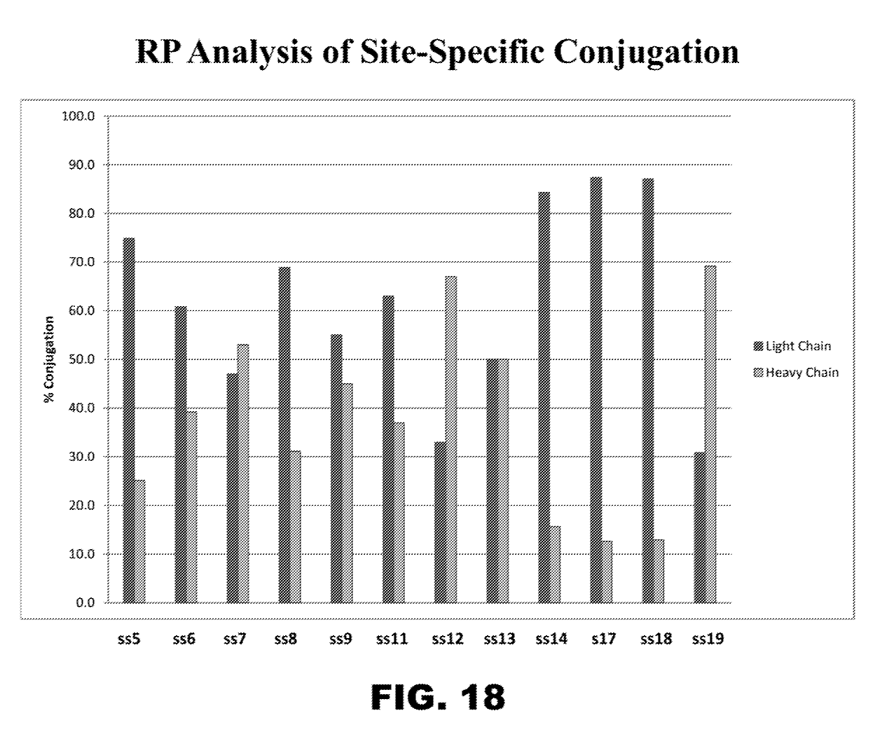

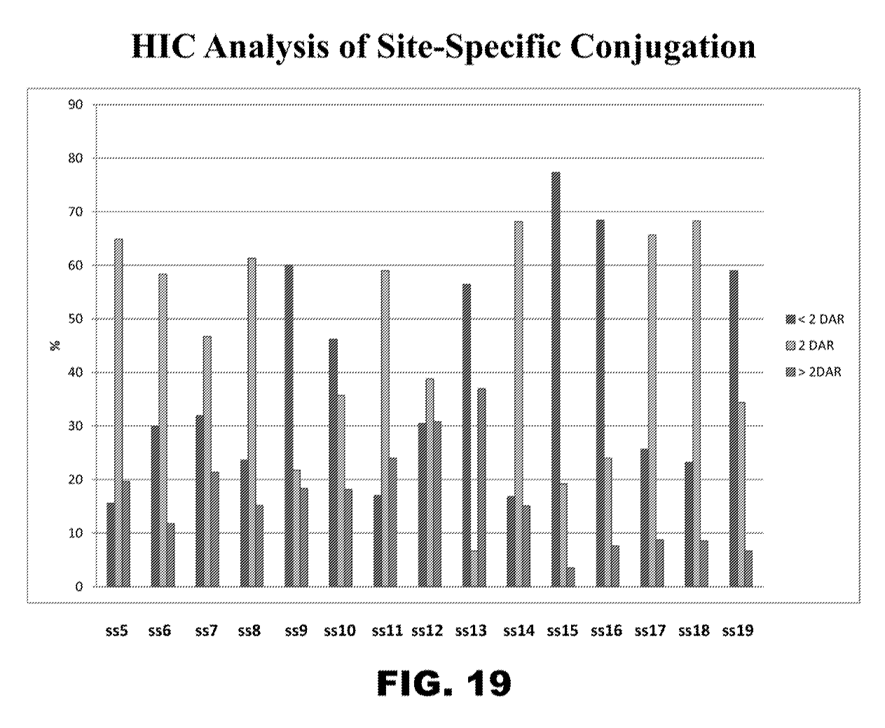

The final antibody-drug preparations were analyzed using RP-HPLC to quantify heavy vs. light chain conjugation sites in order to determine the percentage of on-target light-chain conjugation (FIG. 18). The samples were also analyzed using hydrophobic interaction chromatography to determine the amount of DAR=2 species relative to the unwanted DAR>2 species (FIG. 19).

As shown in FIG. 19, ss5, ss8, ss14, ss17 and ss18 constructs provided an average DAR=2 level of greater than 60% while maintaining unwanted DAR>2 species below 20%. These constructs also provided the highest desired light chain conjugation (>70%) as shown in FIG. 18. Constructs ss15 and ss16 provided the lowest level of DAR 2 species and did not resolve on RP-HPLC whereas constructs ss12 and ss19 provided the lowest light chain conjugation (<40%) and exhibited instability during buffer exchange, thereby reducing the overall yield and DAR. Such results further demonstrate the effective and homogeneous conjugation achieved using selected engineered constructs.

### Example 23

### Selective Conjunction of Engineered Site-Specific Antibodies to MMAE

In order to further improve the specificity of the conjugation and homogeneity of the final product, a few selected site-specific antibodies fabricated as set forth in Examples 20 were selectively reduced using a stabilizing agent (e.g. L-arginine) and a mild reducing agent (e.g. glutathione) prior to conjugation with linker-drug comprising MMAE.

As described in Example 20, the target conjugation site of each hSC16ss 5-19 construct is the unpaired cysteine on each light chain. In order to direct conjugation to these engineered sites, preparations of ss5, ss6, ss8, ss14, ss17, ss18 and ss19 were partially reduced in a buffer containing 1M L-arginine/5 mM glutathione, reduced (GSH)/5 mM EDTA, pH 8.0 for a minimum of one hour at room temperature. All preparations were then buffer exchanged into a 20 mM Tris/3.2 mM EDTA, pH 8.2 buffer using a 30 kDa membrane (Millipore Amicon Ultra). The resulting partially reduced preparations had free thiol concentrations between 1.9 and 2.3, and all preparations were then conjugated to MMAE via a maleimido linker for a minimum of 30 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of antibody-PBD were then diafiltered into 20 mM histidine chloride, pH 6.0 by diafiltration using a 30 kDa membrane.

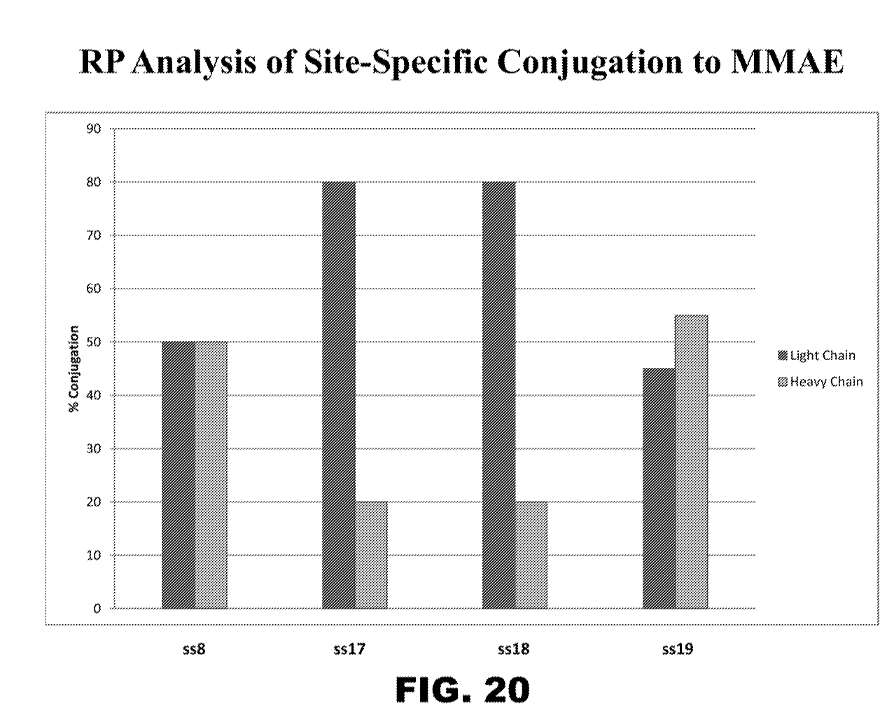

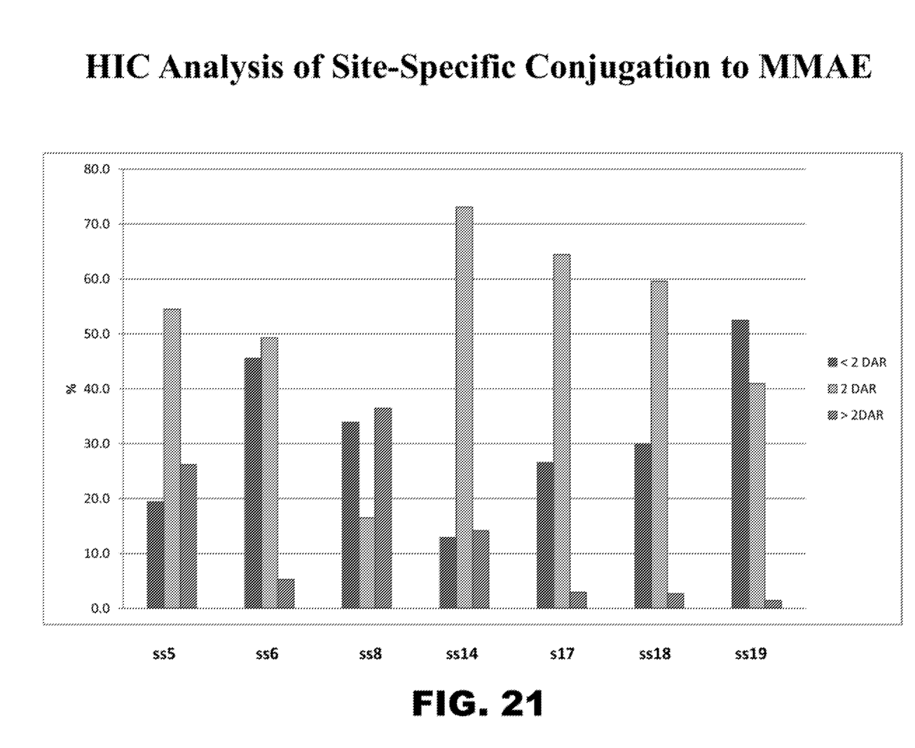

The final antibody-drug preparations were analyzed using RP-HPLC quantify heavy vs. light chain conjugation sites in order to determine the percentage of on-target light-chain conjugation (FIG. 20). The samples were also analyzed using hydrophobic interaction chromatography to determine the amount of DAR=2 species relative to the unwanted DAR>2 species (FIG. 21).

As shown in FIG. 21, ss14, ss17 and ss18 constructs provided an average DAR=2 level of greater than/equal to 60% while maintaining unwanted DAR>2 species below 20%. As shown in FIG. 20, ss8, ss17, ss18 and ss19 all showed acceptable resolution on RP-HPLC. Of these 4 constructs, ss17 and ss18 provided the highest level of desired light chain conjugation (80%), correlating with the data obtained with these constructs in the previous Example. Again this demonstrates that engineered constructs may be selectively reduced to provide highly homogeneous compositions having an effective therapeutic index.

### Example 24

### Selective Conjunction of Engineered Site-Specific Antibodies to Calicheamicin

In order to demonstrate the versatility of the instant invention site-specific antibodies fabricated as set forth in Examples 20 were selectively reduced using a stabilizing agent (e.g. L-arginine) and a mild reducing agent (e.g. glutathione) prior to conjugation with linker-drug comprising calicheamicin.

As described in Example 20, the target conjugation site of each SC16ss5-19 construct is the unpaired cysteine on each light chain. In order to direct conjugation to these engineered sites, preparations of ss5, ss6, ss8, ss14, ss17 and ss18 were partially reduced in a buffer containing 1M L-arginine/6 mM glutathione, reduced (GSH)/5 mM EDTA, pH 8.0 for a minimum of 90 minutes at room temperature. All preparations were then buffer exchanged into a 20 mM Tris/3.2 mM EDTA, pH 7 buffer using a 30 kDa membrane (Millipore Amicon Ultra). The resulting partially reduced preparations had free thiol concentrations between 1.1 and 2.0, and all preparations were then conjugated to calicheamicin via a maleimido linker overnight for a minimum of 12 hours at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of antibody-calicheamicin were then diafiltered into 20 mM histidine chloride, pH 6.0 by diafiltration using a 30 kDa membrane.

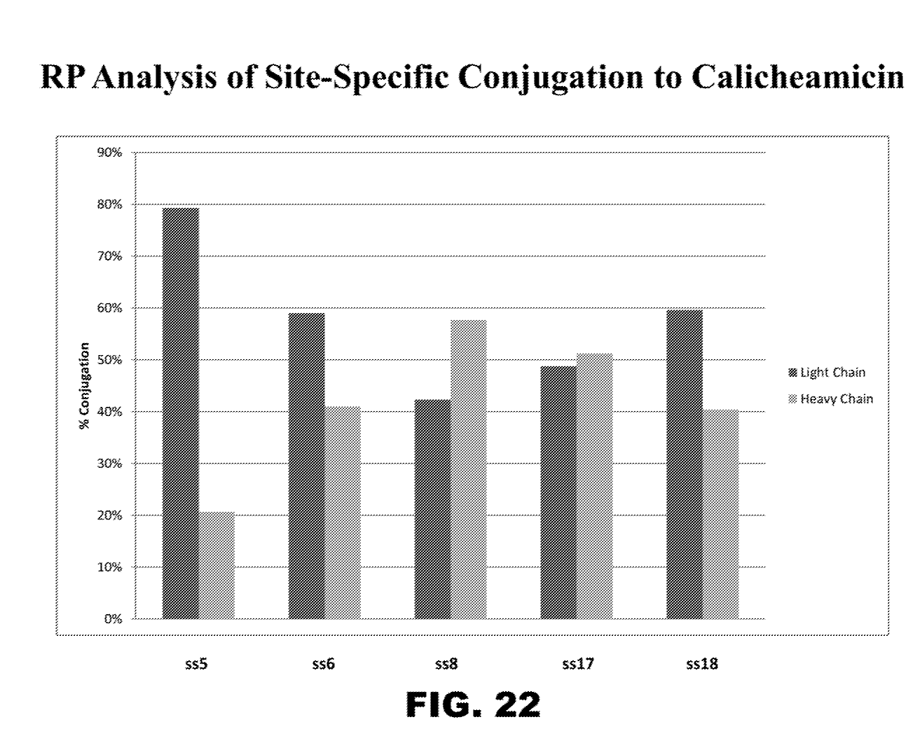

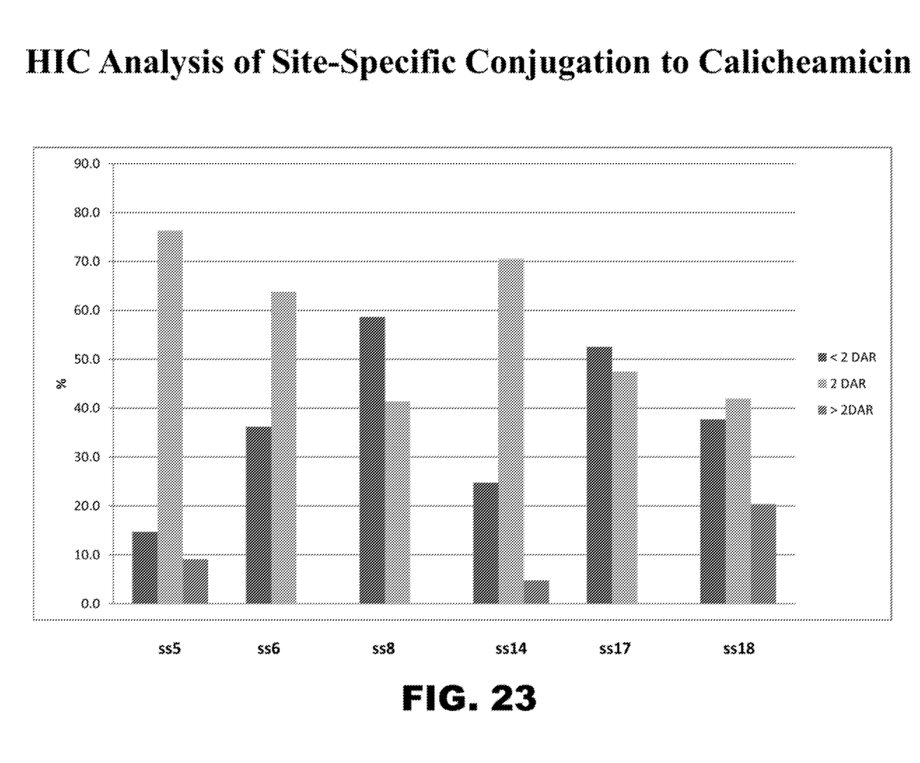

The final antibody-drug preparations were analyzed using RP-HPLC to quantify heavy vs. light chain conjugation sites in order to determine the percentage of on-target light-chain conjugation (FIG. 22). The samples were also analyzed using hydrophobic interaction chromatography to determine the amount of DAR=2 species relative to the unwanted DAR>2 species (FIG. 23).

As shown in FIG. 23, ss5 construct provided an average DAR=2 level of greater than 75% while maintaining unwanted DAR>2 species below 10%. As shown in FIG. 22, ss5, ss6, ss8, ss17 and ss18 all showed acceptable resolution on RP-HPLC. Of these 5 constructs, ss5 provided the highest level of desired light chain conjugation (79%), correlating with the data obtained with this construct in the previous Example. Again this demonstrates that engineered constructs may be selectively reduced to provide highly homogeneous compositions having an effective therapeutic index.

### Example 25

### Selective Conjunction of Engineered Site-Specific Antibodies to Dolastatin

To further illustrate the wide range of cytotoxic agents that are compatible with the instant invention site-specific antibodies fabricated as set forth in Examples 20 were selectively reduced using a stabilizing agent (e.g. L-arginine) and a mild reducing agent (e.g. glutathione) prior to conjugation with linker-drug comprising dolastatin.

As described in Example 20, the target conjugation site of each SC16ss5-19 construct is the unpaired cysteine on each light chain. In order to direct conjugation to these engineered sites, preparations of ss5, ss6, ss8, ss14, ss17 and ss18 were partially reduced in a buffer containing 1M L-arginine/8 mM glutathione, reduced (GSH)/5 mM EDTA, pH 8.0 for a minimum of 90 minutes at room temperature. All preparations were then buffer exchanged into a 20 mM Tris/3.2 mM EDTA, pH 7 buffer using a 30 kDa membrane (Millipore Amicon Ultra). The resulting partially reduced preparations had free thiol concentrations between 1.6 and 2.3, and all preparations were then conjugated to dolastatin via a maleimido linker for a minimum of 60 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of antibody-dolastatin were then diafiltered into 20 mM histidine chloride, pH 6.0 by diafiltration using a 30 kDa membrane.

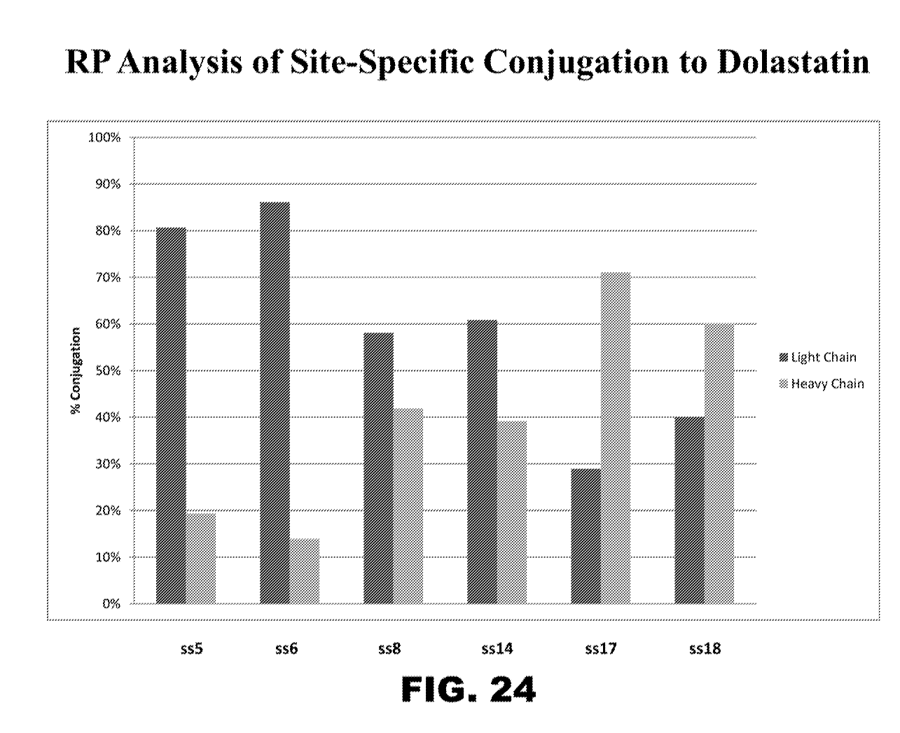

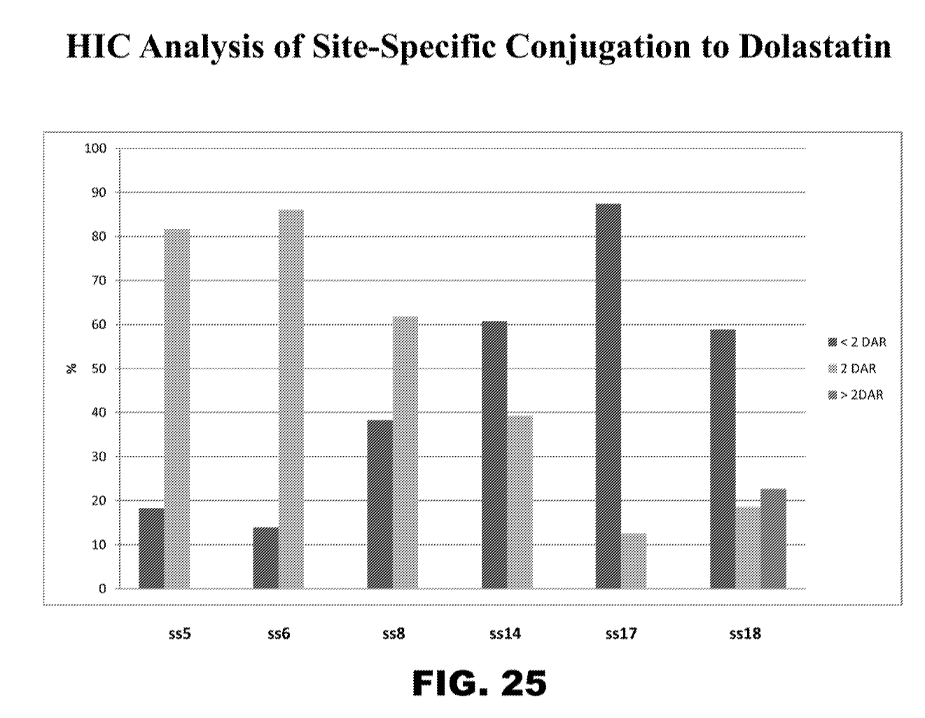

The final antibody-drug preparations were analyzed using RP-HPLC to quantify heavy vs. light chain conjugation sites in order to determine the percentage of on-target light-chain conjugation (FIG. 24). The samples were also analyzed using hydrophobic interaction chromatography to determine the amount of DAR=2 species relative to the unwanted DAR>2 species (FIG. 25).

As shown in FIG. 25, ss6 construct provided an average DAR=2 level of greater than 85% while maintaining unwanted DAR>2 species below 5%. As shown in FIG. 24, ss5, ss6, ss8, ss14, ss17 and ss18 all showed acceptable resolution on RP-HPLC. Of these 6 constructs, ss5 and ss6 provided the highest level of desired light chain conjugation (>80%), correlating with the data obtained with these constructs in the previous Example. Again this demonstrates that engineered constructs may be selectively reduced to provide highly homogeneous compositions having an effective therapeutic index.

### Example 26

### Site-Specific Conjugates Effectively Kill DLL3⁺ Cells In Vitro

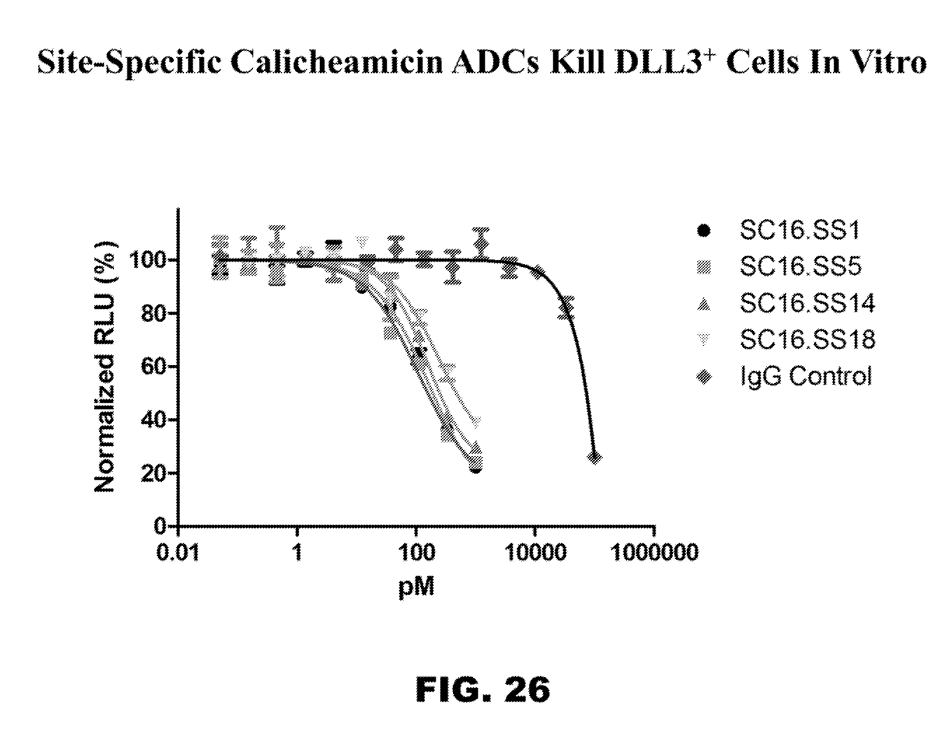

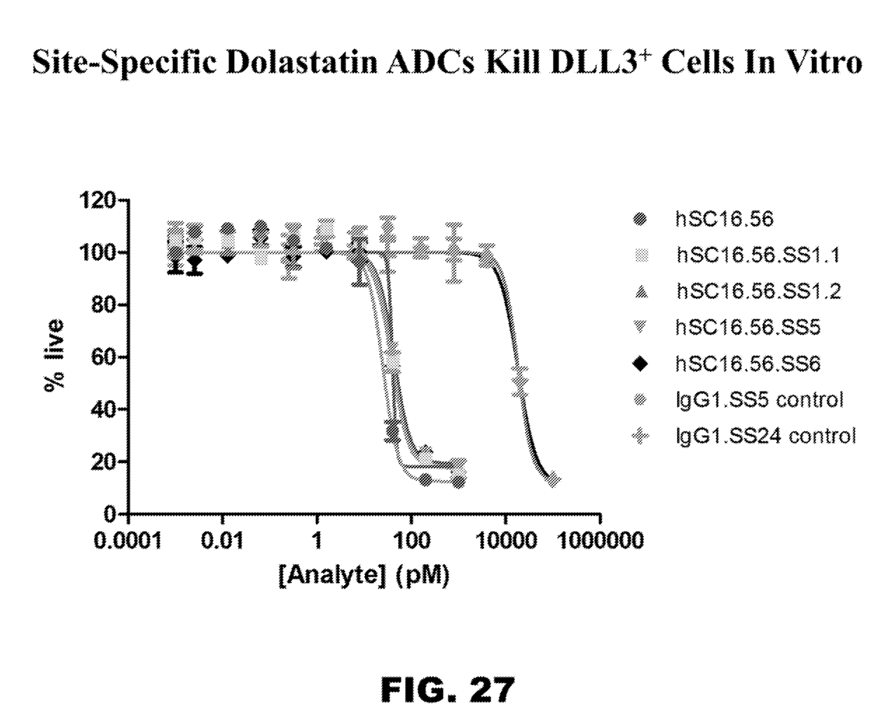

In view of the successful conjugations above assays are performed to demonstrate the ability of site-specific conjugates to effectively kill cells expressing the human DLL3 antigen in vitro. More specifically selected site-specific ADCs comprising calicheamicin or dolastatin were tested to access their ability to kill DLL3+ cells in vitro. To this end DLL3⁺ 293 cells were exposed to calicheamicin site-specific conjugates comprising SC16.ss1, SC16ss5, SC16ss14 and SC16ss18 and dolastatin site-specific conjugates comprising SC16.ss1, SC16ss5 and SC16ss6. The results for the calicheamicin conjugates are shown in FIG. 26 while the results for the dolastatin conjugates are shown in FIG. 27.

To conduct the assays single cell suspensions of HEK-293T cells overexpressing hDLL3 were plated at 500 cells per well into BD Tissue Culture plates (BD Biosciences). One day later, various concentrations of the purified hSC16.56 site-specific ADCs were added to the cultures. The cells were incubated for 96 hours. After the incubation viable cells were enumerated using CellTiter-Glo® (Promega) as per the manufacturer's instructions. Raw luminescence counts using cultures containing non-treated cells were set as 100% reference values and all other counts were calculated as a percentage of the reference value.

A review of FIG. 26 shows that the four DLL3⁺ site-specific calicheamicin conjugates (SC16.ss1, SC16ss5, SC16ss14 and SC16ss18) effectively killed the DLL3⁺ cells when compared to the IgG1 control calicheamicin conjugate. Similarly FIG. 27 graphically illustrates that the dolastatin DLL3⁺ site specific conjugates (SC16.ss1, SC16ss5 and SC16ss6 with SC16ss1 comprising two different dolastatin toxin lots) kill DLL3⁺ cells at substantially lower concentrations than the IgG1 site specific dolastatin controls.

Taken together the results presented in FIGS. 26 and 27 conclusively demonstrate the ability of a variety of site-specific conjugates with different conjugation sites and different toxins to specifically mediate internalization and delivery of cytotoxic warheads to cells expressing a selected determinant. These results indicate that the disclosed site-specific conjugates may effectively be used as targeted therapies in a clinical setting.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. An engineered antibody comprising one or more unpaired cysteine residue(s) wherein the unpaired cysteine residue(s) are exclusive of cysteines that form native interchain disulfide bonds.

2. An engineered antibody according to claim 1 wherein the engineered antibody comprises an IgG1 antibody.

3. An engineered antibody according to claim 1 or 2 wherein the engineered antibody comprises a CL domain and the CL domain comprises a kappa CL domain.

4. An engineered antibody according to claim 1 or 2 wherein the engineered antibody comprises a CL domain and the CL domain comprises a lambda CL domain.

5. An engineered antibody according to any one of claims 1 to 4 wherein the engineered antibody comprises a monoclonal antibody.

6. An engineered antibody according to any one of claims 1 to 5 wherein the engineered antibody comprises a humanized antibody or a CDR grafted antibody.

7. An engineered antibody according to any one of claims 1 to 6 wherein the engineered antibody comprises two unpaired cysteine residues.

8. An engineered antibody according to claim 7 comprising two light chains, wherein each light chain comprises one of the two unpaired cysteine residues.

9. An engineered antibody according to claim 8 wherein each of the two unpaired cysteine residues is present in the CL domains of the light chains.

10. An engineered antibody of claim 8 wherein the each of the two unpaired cysteine residues is present in the C-terminal region of the CL domains of the light chains.

11. An engineered antibody of claim 8 wherein each of the two unpaired cysteine residues is positioned in the exposed loop structures of the CL domain.

12. An engineered antibody of claim 8 wherein each of two unpaired cysteine residues is at a position selected from any one of residues 121-128, residues 182-191 or residues 201-213 of the CL domain wherein the residues are numbered according to Kabat.

13. An engineered antibody of claim 8 wherein the antibody light chain comprises a CL having an amino acid sequence selected from the group consisting of SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563 and SEQ ID NO: 564.

14. An engineered antibody of claim 7 comprising two heavy chains, wherein each heavy chain comprises one of the two unpaired cysteine residues.

15. An engineered antibody of claim 14 wherein each of the two unpaired cysteine residues is located within an antibody domain selected from the group consisting of a CH1 domain, a CH2 domain and a CH3 domain.

16. An engineered antibody according to any of claims 1 to 15 further comprising a drug conjugated to the one or more unpaired cysteine residues(s).

17. An engineered antibody according to any of claims 1 to 16 wherein the engineered antibody reacts with a determinant selected from the group consisting of DLL3, SEZ6 and CD324.

18. An antibody drug conjugate of the formula:
Ab-[L-D]n or a pharmaceutically acceptable salt thereof wherein
a) Ab comprises an engineered antibody comprising one or more unpaired cysteine residue(s) wherein the one or more unpaired cysteine residue(s) are exclusive of cysteines that form native interchain disulfide bonds.
b) L comprises an optional linker;
c) D comprises a drug; and
n is an integer from about 1 to about 12.

19. An antibody drug conjugate of claim 18 wherein the engineered antibody comprises a monoclonal antibody.

20. An antibody drug conjugate of claim 18 or 19 wherein the engineered antibody comprises a humanized antibody or a CDR grafted antibody.

21. An antibody drug conjugate of any one of claims 18 to 20 wherein the engineered antibody comprises two unpaired cysteine residues.

22. An antibody drug conjugate according to claim 21, wherein the engineered antibody comprises two light chains, and wherein each light chain comprises one of the two unpaired cysteine residues.

23. An antibody drug conjugate according to claim 22 wherein each of said unpaired cysteines is present in the CL domains of the light chains.

24. An antibody drug conjugate according to any one of claims 18 to 23 wherein the drug comprises a cytotoxin selected from the group consisting of dolastatins, auristatins, calicheamicins, maytansinoids and pyrrolobenzodiazepines

25. A pharmaceutical composition comprising the engineered antibody of any one of claims 1 to 17 or the antibody drug conjugate of any one of claims 18 to 24; and a pharmaceutically acceptable carrier.

26. A method of treating cancer in a subject comprising administering to said subject a pharmaceutical composition of claim 25.

27. A method of preparing an antibody drug conjugate comprising the steps of:
a) providing an engineered antibody comprising one or more unpaired cysteine residue(s) wherein the one or more unpaired cysteine residue(s) are exclusive of cysteines that form native interchain disulfide bonds;
b) selectively reducing the engineered antibody; and
c) conjugating the selectively reduced engineered antibody to a drug.

28. The method of claim 27 wherein the step of selectively reducing the engineered antibody comprises the step of contacting the engineered antibody with a stabilizing agent.

29. The method of claim 27 wherein the one or more unpaired cysteine residue(s) are located in the CL domain.

30. The method of claim 27 wherein the one or more unpaired cysteine residue(s) comprises an introduced cysteine residue(s) or a substituted cysteine residue(s).

31. An engineered antibody comprising one or more unpaired cysteine residue(s) wherein the one or more unpaired cysteine residue(s) are not native interchain disulfide bond cysteine(s).
